(19) United States
(12) Patent Application Publication
(10) Pub. No.: US 2026/0185092 A1
(43) Pub. Date: Jul. 2, 2026

(54) COMPOSITIONS AND METHODS FOR INHIBITING MAPT EXPRESSION

(71) Applicant: Dicerna Pharmaceuticals, Inc., Lexington, MA (US)

(72) Inventors: Bob Dale Brown, Littleton, MA (US); Henryk T. Dudek, Belmont, MA (US); Seongmoon Cheong, Harvard, MA (US); Shiyu Wang, Belmont, MA (US); Travis Grim, Maynard, MA (US); Matthew Guese Costales, Lexington, MA (US); Maire Jung, Lincoln, MA (US)

(21) Appl. No.: 19/383,237

(22) Filed: Nov. 7, 2025

Related U.S. Application Data
(63) Continuation of application No. 18/316,529, filed on May 12, 2023.
(60) Provisional application No. 63/364,609, filed on May 12, 2022.

(51) Int. Cl. C12N 15/113

(52) U.S. Cl. CPC C12N 15/113; C12N 2310/14; C12N 2310/315; C12N 2310/321; C12N 2310/322; C12N 2310/351; C12N 2310/3515

(57) ABSTRACT

Oligonucleotides are provided herein that inhibit MAPT gene expression, including oligonucleotides conjugated to a targeting ligand (e.g., lipid moiety). Also provided are compositions including the same and uses thereof, particularly uses relating to treating diseases, disorders, and/or conditions associated with MAPT gene expression.

### CROSS-REFERENCE TO RELATED APPLICATIONS

The present application is a continuation application of U.S. application Ser. No. 18/316,529, filed May 12, 2023, which claims priority under 35 U.S.C. § 119 (e) from U.S. Provisional Application No. 63/364,609, filed May 12, 2022, each of which is incorporated herein by reference in its entirety.

### SEQUENCE LISTING

[001.1] The instant application contains a Sequence Listing which has been submitted electronically in XML format and is hereby incorporated by reference in its entirety. Said XML copy, created on Nov. 5, 2025, is named 400930-034USC1_222236_SL.xml and is 2,876,440 bytes in size.

### TECHNICAL FIELD

The disclosure relates generally to biology and medicine, and more particularly it relates to oligonucleotides and compositions including the same for inhibiting or reducing (i.e., modulating) microtubule-associated protein tau (MAPT) gene expression, as well as their use for treating diseases and disorders associated with MAPT gene expression.

### BACKGROUND

Microtubules perform several essential roles within cells throughout the body. Within the central nervous system (CNS), microtubules provide structural support and assist in transporting substances throughout cells. Changes in microtubule mass, structure, and pattern are known factors leading to the development of many neurodegenerative diseases. Tau is an essential protein for forming microtubules, whose abnormal expression leads to neurodegenerative diseases. Tau proteins combine with tubulin to form microtubules. Alternative splicing of MAPT generates different Tau proteins used in microtubule assembly. Mutations (e.g., insertions and mismatches) in MAPT that alter Tau function and expression are known causes of several diseases and disorders impacting the CNS (e.g., Alzheimer's disease (AD), Parkinson's disease (PD), and tauopathies). Strategies for targeting MAPT gene expression to prevent such diseases and disorders are needed.

The mammalian CNS is a complex system of tissues, including cells, fluids, and chemicals that interact in concert to enable a wide variety of functions, including movement, navigation, cognition, speech, vision, and emotion. Unfortunately, a variety of diseases and disorders of the CNS are known (e.g., neurological disorders) and affect or disrupt some or all of these functions. Typically, treatments for diseases and disorders of the CNS have been limited to small molecule drugs, antibodies, and/or to adaptive or behavioral therapies. There exists an ongoing need to develop treatments for diseases and disorders of the CNS associated with inappropriate MAPT gene expression.

### BRIEF SUMMARY

To address this need, the disclosure describes compositions for and methods of treating a disease, disorder, or condition associated with MAPT gene expression. The disclosure is based, at least in part, on discovering and developing double-stranded (ds) oligonucleotides such as RNAi oligonucleotides that effectively target and reduce MAPT gene expression in tissues of the CNS. Specifically, target sequences within MAPT mRNA were identified, and oligonucleotides that bind to these target sequences and inhibit MAPT mRNA expression were generated. As demonstrated herein, the oligonucleotides inhibit human and non-human primate (NHP) MAPT gene expression in CNS tissue. Further, MAPT mRNA expression was reduced in CNS tissue associated with AD or progressive supranuclear palsy (PSP) with both N-acetylgalactosamine (GalNAc)-conjugated and lipid-conjugated MAPT mRNA-targeting oligonucleotides. Without being bound by theory, the oligonucleotides described herein are useful for treating a disease, disorder, or condition associated with MAPT gene expression.

Accordingly, and in some aspects, the disclosure provides a RNAi oligonucleotide for reducing MAPT gene expression, the oligonucleotide comprising a sense strand and an antisense strand, wherein the sense strand and the antisense strand form a duplex region, wherein the antisense strand comprises a region of complementarity to a MAPT mRNA target sequence of any one of SEQ ID NOs: 912-1295, and wherein the region of complementarity is at least about 15 contiguous nucleotides in length.

In any of the foregoing or related aspects, the sense strand is about 15 to about 50 nucleotides in length. In some aspects, the sense strand is 18 to 36 nucleotides in length. In some aspects, the antisense strand is about 15 to about 30 nucleotides in length. In some aspects, the antisense strand is 22 nucleotides in length, wherein the antisense strand and the sense strand form a duplex region of at least about 19 nucleotides in length, optionally at least 20 nucleotides in length. In some aspects, the region of complementarity is at least about 19 contiguous nucleotides in length. In some aspects, the region of complementarity is at least about 20 contiguous nucleotides in length.

In other aspects, the disclosure provides a ds RNAi oligonucleotide for reducing MAPT gene expression, the oligonucleotide comprising:
(i) an antisense strand of about 19-30 nucleotides in length, wherein the antisense strand comprises a nucleotide sequence comprising a region of complementarity to a MAPT mRNA target sequence, wherein the region of complementarity is selected from SEQ ID NOs: 1296-1679, and
(ii) a sense strand of about 19-50 nucleotides in length comprising a region of complementarity to the antisense strand, wherein the antisense and sense strands are separate strands that form an asymmetric duplex region having an overhang of 1-4 nucleotides at the 3′ terminus of the antisense strand.

In some aspects, the 3′ end of the sense strand comprises a stem-loop set forth as S1-L-S2, wherein S1 is complementary to S2, and wherein L forms a loop between S1 and S2 of 3-5 nucleotides in length. In some aspects, L is a triloop (triL) or a tetraloop (tetraL). In some aspects, L is a tetraL. In some aspects, the tetraL comprises the sequence 5′-GAAA-3′. In some aspects, S1 and S2 are about 1 to about 10 nucleotides in length and have the same length. In some aspects, S1 and S2 are 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides in length. In some aspects, S1 and S2 are 6 nucleotides in length. In some aspects, the stem-loop comprises the sequence 5′-GCAGCCGAAAGGCUGC-3′ (SEQ ID NO: 1680).

In other aspects, the oligonucleotide comprises a blunt end. In some aspects, the blunt end comprises the 3′ end of the sense strand. In some aspects, the sense strand is about 20-22 nucleotides. In some aspects, the sense strand is 20 nucleotides.

In any of the foregoing or related aspects, the antisense strand comprises a 3′ overhang sequence of one or more nucleotides in length. In some aspects, the overhang comprises purine nucleotides. In some aspects, the 3′ overhang sequence is 2 nucleotides in length. In some aspects, the 3′ overhang is selected from AA, GG, AG, and GA. In some aspects, the overhang is GG or AA. In some aspects, the overhang is GG.

In any of the foregoing or related aspects, the oligonucleotide comprises at least one modified nucleotide. In some aspects, the modified nucleotide comprises a 2′-modification. In some aspects, the 2′-modification is a modification selected from 2′-aminoethyl (EA), 2′-fluoro (2′-F), 2′-O-methyl(2′-OMe), 2′-O-methoxyethyl(2′-OME), and 2′-deoxy-2′-fluoro-β-d-arabinonucleic acid (2′-FANA). In some aspects, the modification is a 2′-modification selected from 2′-F and 2′-OMe. In some aspects, about 18% to about 23%, or 18%, 19%, 20%, 21%, 22%, or 23% of the nucleotides of the sense strand comprise a 2′-F modification. In other aspects, about 38% to about 43%, or 38%, 39%, 40%, 41%, 42%, or 43% of the nucleotides of the sense strand comprise a 2′-F modification. In some aspects, about 25% to about 35%, or 25%, 26%, 27%, 28%, 29%, 30%, 31%, 32%, 33%, 34%, or 35% of the nucleotides of the antisense strand comprise a 2′-F modification. In some aspects, about 25% to about 35%, or 25%, 26%, 27%, 28%, 29%, 30%, 31%, 32%, 33%, 34%, or 35% of the nucleotides of the oligonucleotide comprise a 2′-F modification. In some aspects, about 35-45%, 35%, 36%, 37%, 38%, 39%, 40%, 41%, 42%, 43%, 44% or 45% of the nucleotides of the oligonucleotide comprise a 2′-F modification. In some aspects, the sense strand comprises 20 nucleotides with positions 1-20 from 5′ to 3′, wherein each of positions 3, 5, 8, 10, 12, 13, 15, and 17 comprise a 2′-F modification. In other aspects, the sense strand comprises 36 nucleotides with positions 1-36 from 5′ to 3′, wherein each of positions 3, 5, 8, 10, 12, 13, 15, and 17 comprise a 2′-F modification. In some aspects, the antisense strand comprises 22 nucleotides with positions 1-22 from 5′ to 3′, and wherein each of positions 2, 3, 4, 5, 7, 10 14, 16, and 19 comprise a 2′-F modification. In some aspects, the remaining nucleotides comprise a 2′-OMe modification.

In any of the foregoing or related aspects, the oligonucleotide comprises at least one modified internucleotide linkage. In some aspects, the at least one modified internucleotide linkage is a phosphorothioate linkage. In some aspects, the antisense strand comprises a phosphorothioate linkage (i) between positions 1 and 2, and between positions 2 and 3; or (ii) between positions 1 and 2, between positions 2 and 3, and between positions 3 and 4, wherein positions are numbered 1-4 from 5′ to 3′. In some aspects, the antisense strand is 22 nucleotides in length, and wherein the antisense strand comprises a phosphorothioate linkage between positions 20 and 21 and between positions 21 and 22, wherein positions are numbered 1-22 from 5′ to 3′. In some aspects, the sense strand comprises a phosphorothioate linkage between positions 1 and 2, wherein positions are numbered 1-2 from 5′ to 3′. In some aspects, the sense strand is 20 nucleotides in length, and wherein the sense strand comprises a phosphorothioate linkage between positions between positions 1 and 2, between positions 18 and 19 and between positions 19 and 20, wherein positions are numbered 1-20 from 5′ to 3′.

In any of the foregoing or related aspects, the 4′-carbon of the sugar of the 5′-nucleotide of the antisense strand comprises a phosphate analog. In some aspects, the phosphate analog is oxymethyl phosphonate, vinyl phosphonate or malonyl phosphonate, optionally wherein the phosphate analog is a 4′-phosphate analog comprising 4′-oxymethylphosphonate.

In any of the foregoing or related aspects, at least one nucleotide of the oligonucleotide is conjugated to one or more targeting ligands. In some aspects, each targeting ligand comprises a carbohydrate, amino sugar, lipid, cholesterol, or polypeptide. In some aspects, the stem-loop comprises one or more targeting ligands conjugated to one or more nucleotides of the stem-loop. In some aspects, the one or more targeting ligands is conjugated to one or more nucleotides of the loop. In some aspects, the loop comprises 4 nucleotides numbered 1-4 from 5′ to 3′, wherein nucleotides at positions 2, 3, and 4 each comprise one or more targeting ligands, wherein the targeting ligands are the same or different. In some aspects, each targeting ligand comprises a GalNAc moiety. In some aspects, the GalNac moiety is a monovalent GalNAc moiety, a bivalent GalNAc moiety, a trivalent GalNAc moiety, or a tetravalent GalNAc moiety. In some aspects, up to 4 nucleotides of L of the stem-loop are each conjugated to a monovalent GalNAc moiety.

In other aspects, the one or more targeting ligands is a lipid moiety. In some aspects, the lipid moiety is conjugated to the 5′ terminal nucleotide of the sense strand. In some aspects, the lipid moiety is a hydrocarbon chain. In some aspects, the hydrocarbon chain is a C₈-C₃₀ hydrocarbon chain. In some aspects, the hydrocarbon chain is a C₁₆ hydrocarbon chain. In some aspects, the C₁₆ hydrocarbon chain is represented by:

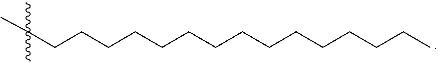 [SMILES: *CCCCCCCCCCCCCCC]

In some aspects, the lipid moiety is conjugated to the 2′ carbon of the ribose ring of the 5′ terminal nucleotide.

In any of the foregoing or related aspects, the region of complementarity is fully complementary to the MAPT mRNA target sequence at nucleotide positions 2-8 of the antisense strand, wherein nucleotide positions are numbered 5′ to 3′. In some aspects, the region of complementarity is fully complementary to the MAPT mRNA target sequence at nucleotide positions 2-11 of the antisense strand, wherein nucleotide positions are numbered 5′ to 3′.

In any of the foregoing or related aspects, the sense strand comprises a nucleotide sequence of any one of SEQ ID NOs: 769-803 and 1681. In some aspects, the antisense strand comprises a nucleotide sequence of any one of SEQ ID NOs: 804-838.

In some aspects, the sense strand and antisense strands comprise nucleotide sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
s) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
w) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively;
ii) SEQ ID NOs: 803 and 838, respectively; and
jj) SEQ ID NOs: 1681 and 815, respectively.

In some aspects, the sense strand and antisense strands comprise nucleotide sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively;
k) SEQ ID NOs: 1681 and 815, respectively.

In some aspects, the sense strand and antisense strands comprise nucleotide sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively;
f) SEQ ID NOs: 803 and 838, respectively; and
g) SEQ ID NOs: 1681 and 815, respectively.

In some aspects, the sense strand comprises the nucleotide sequence as set forth in SEQ ID NO: 771, and the antisense strand comprises the nucleotide sequence as set forth in SEQ ID NO: 806. In some aspects, the sense strand comprises the nucleotide sequence as set forth in SEQ ID NO: 780, and the antisense strand comprises the nucleotide sequence as set forth in SEQ ID NO: 815. In some aspects, the sense strand comprises the nucleotide sequence as set forth in SEQ ID NO: 781, and the antisense strand comprises the nucleotide sequence as set forth in SEQ ID NO: 816. In some aspects, the sense strand comprises the nucleotide sequence as set forth in SEQ ID NO: 798, and the antisense strand comprises the nucleotide sequence as set forth in SEQ ID NO: 833. In some aspects, the sense strand comprises the nucleotide sequence as set forth in SEQ ID NO: 799, and the antisense strand comprises the nucleotide sequence as set forth in SEQ ID NO: 834. In some aspects, the sense strand comprises the nucleotide sequence as set forth in SEQ ID NO: 803, and the antisense strand comprises the nucleotide sequence as set forth in SEQ ID NO: 838. In some aspects, the sense strand comprises the nucleotide sequence as set forth in SEQ ID NO: 1681, and the antisense strand comprises a nucleotide sequence as set forth in SEQ ID NO: 815.

In any of the foregoing or related aspects, the antisense strand is 22 nucleotides in length. In some aspects, the antisense strand comprises a nucleotide sequence selected from SEQ ID NOs: 806, 815, 816, 833, 834, and 838. In some aspects, the sense strand is 36 nucleotides in length. In some aspects, the sense strand comprises a nucleotide sequence selected from SEQ ID NOs: 1130, 1095, 1096, 1119, 1120, and 1124. In some aspects, the sense strand comprises a nucleotide sequence selected from SEQ ID NOs: 771, 780, 781, 798, 799, and 803.

In any of the foregoing or related aspects, the sense strand comprises a nucleotide sequence of any one of SEQ ID NOs: 839-873 and 1682. In some aspects, the antisense strand comprises a nucleotide sequence of any one of SEQ ID NOs: 874-908.

In some aspects, the sense strand and antisense strands comprise nucleotide sequences selected from:
a) SEQ ID NOs: 839 and 874, respectively;
b) SEQ ID NOs: 840 and 875, respectively;
c) SEQ ID NOs: 841 and 876, respectively;
d) SEQ ID NOs: 842 and 877, respectively;
e) SEQ ID NOs: 843 and 878, respectively;
f) SEQ ID NOs: 844 and 879, respectively;
g) SEQ ID NOs: 845 and 880, respectively;
h) SEQ ID NOs: 846 and 881, respectively;
i) SEQ ID NOs: 847 and 882, respectively;
j) SEQ ID NOs: 848 and 883, respectively;
k) SEQ ID NOs: 849 and 884, respectively;
l) SEQ ID NOs: 850 and 885, respectively;
m) SEQ ID NOs: 851 and 886, respectively;
n) SEQ ID NOs: 852 and 887, respectively;
o) SEQ ID NOs: 853 and 888, respectively;
p) SEQ ID NOs: 854 and 889, respectively;
q) SEQ ID NOs: 855 and 890, respectively;
r) SEQ ID NOs: 856 and 891, respectively;
s) SEQ ID NOs: 857 and 892, respectively;
t) SEQ ID NOs: 858 and 893, respectively;
u) SEQ ID NOs: 859 and 894, respectively;
v) SEQ ID NOs: 860 and 895, respectively;
w) SEQ ID NOs: 861 and 896, respectively;
x) SEQ ID NOs: 862 and 897, respectively;
y) SEQ ID NOs: 863 and 898, respectively;
z) SEQ ID NOs: 864 and 899, respectively;
aa) SEQ ID NOs: 865 and 900, respectively;
bb) SEQ ID NOs: 866 and 901, respectively;
cc) SEQ ID NOs: 867 and 902, respectively;
dd) SEQ ID NOs: 868 and 903, respectively;
ee) SEQ ID NOs: 869 and 904, respectively;
ff) SEQ ID NOs: 870 and 905, respectively;
gg) SEQ ID NOs: 871 and 906, respectively;
hh) SEQ ID NOs: 872 and 907, respectively;
ii) SEQ ID NOs: 873 and 908, respectively; and
jj) SEQ ID NOs: 1682 and 885, respectively.

In other aspects, the sense strand and antisense strands comprise nucleotide sequences selected from the group consisting of:
a) SEQ ID NOs: 860 and 895, respectively;
b) SEQ ID NOs: 865 and 900, respectively;
c) SEQ ID NOs: 868 and 903, respectively;
d) SEQ ID NOs: 869 and 904, respectively;
e) SEQ ID NOs: 873 and 908, respectively;
f) SEQ ID NOs: 841 and 876, respectively;
g) SEQ ID NOs: 846 and 881, respectively;
h) SEQ ID NOs: 850 and 885, respectively;
i) SEQ ID NOs: 851 and 886, respectively;
j) SEQ ID NOs: 852 and 887, respectively; and
k) SEQ ID NOs: 1682 and 885, respectively.

In certain aspects, the sense strand and antisense strands comprise nucleotide sequences selected from the group consisting of:
a) SEQ ID NOs: 841 and 876, respectively;
b) SEQ ID NOs: 850 and 885, respectively;
c) SEQ ID NOs: 851 and 886, respectively;
d) SEQ ID NOs: 868 and 903, respectively;
e) SEQ ID NOs: 869 and 904, respectively;
f) SEQ ID NOs: 873 and 908, respectively; and
g) SEQ ID NOs: 1682 and 885, respectively.

In some aspects, the sense strand comprises the nucleotide sequence as set forth in SEQ ID NO: 841, and the antisense strand comprises the nucleotide sequence as set forth in SEQ ID NO: 876. In some aspects, the sense strand comprises the nucleotide sequence as set forth in SEQ ID NO: 850, and the antisense strand comprises the nucleotide sequence as set forth in SEQ ID NO: 885. In some aspects, the sense strand comprises the nucleotide sequence as set forth in SEQ ID NO: 851, and the antisense strand comprises the nucleotide sequence as set forth in SEQ ID NO: 886. In some aspects, the sense strand comprises the nucleotide sequence as set forth in SEQ ID NO: 868, and the antisense strand comprises the nucleotide sequence as set forth in SEQ ID NO: 903. In some aspects, the sense strand comprises the nucleotide sequence as set forth in SEQ ID NO: 869, and the antisense strand comprises the nucleotide sequence as set forth in SEQ ID NO: 904. In some aspects, the sense strand comprises the nucleotide sequence as set forth in SEQ ID NO: 873, and the antisense strand comprises the nucleotide sequence as set forth in SEQ ID NO: 908. In some aspects, the sense strand comprises the nucleotide sequence as set forth in SEQ ID NO: 1682, and the antisense strand comprises the nucleotide sequence as set forth in SEQ ID NO: 885.

In some aspects, the sense strand comprises the sequence and all of the modifications of 5′-[mAs][mG][fA][mG][fU][mG][mU][fG][mG][fA][mA][fA][fA][mA][fA][mA][fA][mA][mG][mA][mG][mC][mA][mG][mC][mC][mG][ademA-GalNAc][ademA-GalNAc][ademAGalNAc][mG][mG][mC][mU][mG][mC]-3′ (SEQ ID NO: 841), and wherein the antisense strand comprises the sequence and all of the modifications of 5′-[MePhosphonate-40-mUs][fCs][fU][fU][fU][mU][fU][mU][mU][fU][mU][mC][mC][fA][mC][fA][mC][mU][fC][m Us][mGs][mG]-3′ (SEQ ID NO: 876), wherein mC, mA, mG, mU=2′-OMe ribonucleosides; fA, fC, fG, fU=2′-F ribonucleosides; s=phosphorothioate, and wherein ademA-GalNAc=

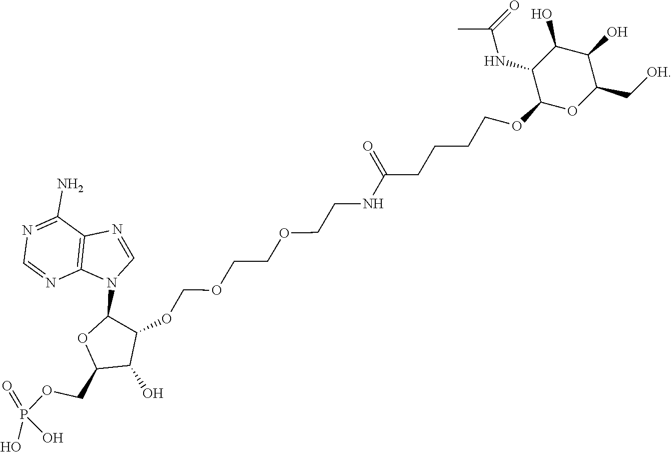 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the sense strand comprises the sequence and all of the modifications of 5′-[mCs][mA][fG][mG][fU][mG][mG][fA][mA][fG][mU][fA][fA][mA][fA][mU][fC][mU][mG][mA][mG][mC][mA][mG][mC][mC][mG][ademA-GalNAc][ademA-GalNAc][ademA-GalNAc][mG][mG][mC][mU][mG][mC]-3′ (SEQ ID NO: 850), and wherein the antisense strand comprises the sequence and all of the modifications of 5′-[MePhosphonate-40-mUs][fCs][fA][fG][fA][mU][fU][mU][mU][fA][mC][mU][mU][fC][mC][fA][mC][mC][fU][m Gs][mGs][mG]-3′ (SEQ ID NO: 885), wherein mC, mA, mG, mU=2′-OMe ribonucleosides; fA, fC, fG, fU=2′-F ribonucleosides; s-phosphorothioate, and wherein ademA-GalNAc=

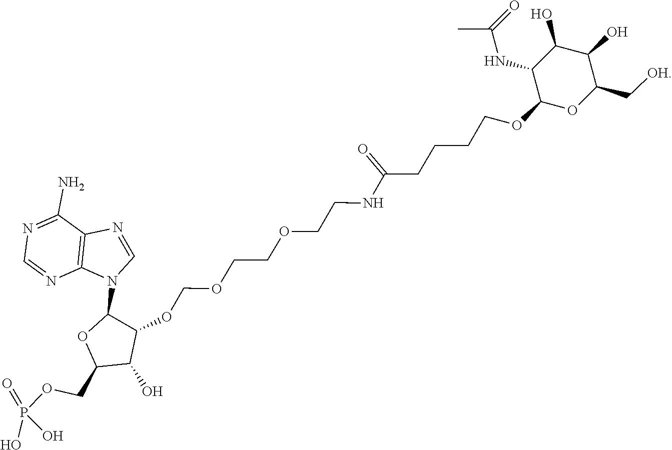 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the sense strand comprises the sequence and all of the modifications of 5′-[mAs][mG][fG][mU][fG][mG][mA][fA][mG][fU][mA][fA][fA][mA][fU][mC][fU][mG][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 851), and wherein the antisense strand comprises the sequence and all of the modifications of 5′-[MePhosphonate-40-mUs][fUs][fC][fA][fG][mA][fU][mU][mU][fU][mA][mC][mU][fU][mC][fC][mA][mC][fC][m Us][mGs][mG]-3′ (SEQ ID NO: 886), wherein mC, mA, mG, mU=2′-OMe ribonucleosides; fA, fC, fG, fU=2′-F ribonucleosides; s-phosphorothioate, and wherein ademA-GalNAc=

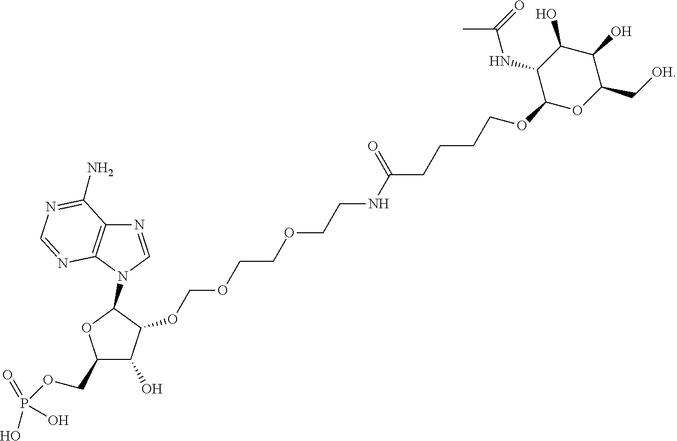 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the sense strand comprises the sequence and all of the modifications of 5′-[mAs][mG][fG][mA][fA][mA][mU][fA][mA][fA][mA][fA][fG][mA][fU][mU][fG][mA][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 868), and wherein the antisense strand comprises the sequence and all of the modifications of 5′-[MePhosphonate-40-mUs][fUs][fU][fC][fA][mA][fU][mC][mU][fU][mU][mU][mU][fA][mU][fU][mU][mC][fC][m Us][mGs][mG]-3′ (SEQ ID NO: 903), wherein mC, mA, mG, mU=2′-OMe ribonucleosides; fA, fC, fG, fU=2′-F ribonucleosides; s=phosphorothioate, and wherein ademA-GalNAc=

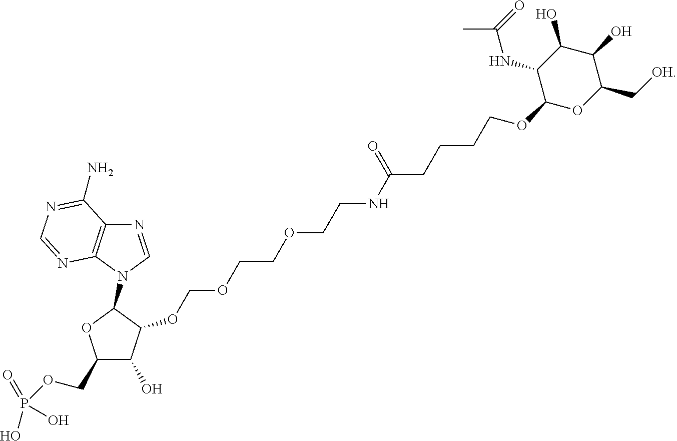 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the sense strand comprises the sequence and all of the modifications of 5′-[mGs][mG][fA][mA][fA][mU][mA][fA][mA][fA][mA][fG][fA][mU][fU][mG][fA][mA][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 869), and wherein the antisense strand comprises the sequence and all of the modifications of 5′-[MePhosphonate-40-mUs][fUs][fU][fU][fC][mA][fA][mU][mC][fU][mU][mU][mU][fU][mA][fU][mU][mU][fC][m Cs][mGs][mG]-3′ (SEQ ID NO: 904), wherein mC, mA, mG, mU=2′-OMe ribonucleosides; fA, fC, fG, fU=2′-F ribonucleosides; s-phosphorothioate, and wherein ademA-GalNAc=

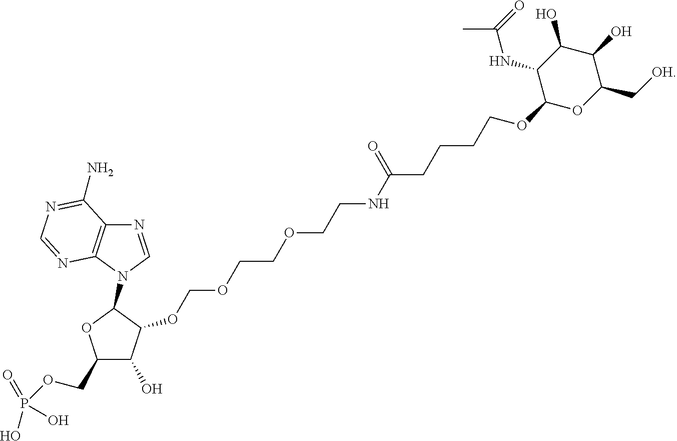 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the sense strand comprises the sequence and all of the modifications of 5′-[mAs][mU][fA][mA][fA][mA][mA][fG][mA][fU][mU][fG][fA][mA][fA][mC][fC][mC][mA][mA][mG][mC][mA][mG][mC][mC][mG][ademA-GalNAc][ademA-GalNAc][ademA-GalNAc][mG][mG][mC][mU][mG][mC]-3′ (SEQ ID NO: 873), and wherein the antisense strand comprises the sequence and all of the modifications of 5′-[MePhosphonate-40-mUs][fUs][fG][fG][fG][mU][fU][mU][mC][fA][mA][mU][mC][fU][mU][fU][mU][mU][fA][m Us][mGs][mG]-3′ (SEQ ID NO: 908), wherein mC, mA, mG, mU=2′-OMe ribonucleosides; fA, fC, fG, fU=2′-F ribonucleosides; s-phosphorothioate, and wherein ademA-GalNAc=

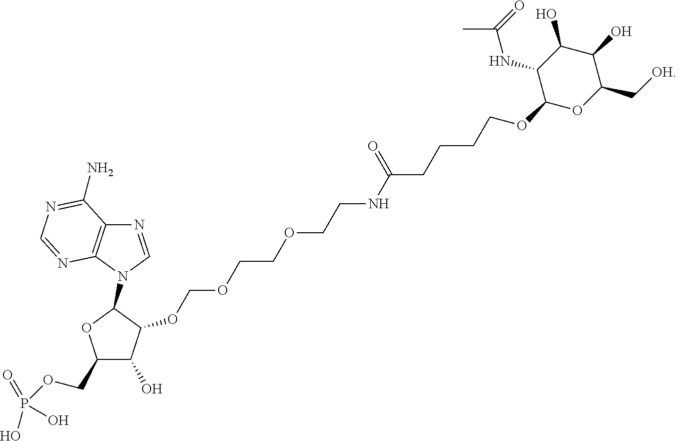 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some aspects, the sense strand comprises the sequence and all of the modifications of 5′-[ademC5-C₁₆][mA][fG][mG][fU][mG][mG][fA][mA][fG][mU][fA][fA][mA][fA][mU][fC][mUs][mGs][mA]-3′ (SEQ ID NO: 1682), and wherein the antisense strand comprises the sequence and all of the modifications of 5′-[MePhosphonate-40-mUs][fCs][fA][fG][fA][mU][fU][mU][mU][fA][mC][mU][mU][fC][mC][fA][mC][mC][fU][m Gs][mGs][mG]-3′ (SEQ ID NO: 885), wherein mC, mA, mG, mU=2′-OMe ribonucleosides; fA, fC, fG, fU=2′-F ribonucleosides; s=phosphorothioate, and [ademC5-C₁₆]-cytosine conjugated to C₁₆ hydrocarbon chain:

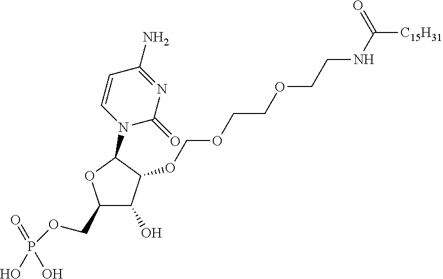

In some aspects, the disclosure provides a pharmaceutical composition comprising a RNAi oligonucleotide described herein, and a pharmaceutically acceptable carrier, delivery agent or excipient.

In other aspects, the disclosure provides a method for treating a subject having a disease, disorder or condition associated with MAPT gene expression, the method comprising administering to the subject a therapeutically effective amount of a RNAi oligonucleotide described herein, or pharmaceutical composition thereof, thereby treating the subject.

In further aspects, the disclosure provides a method of delivering an oligonucleotide to a subject, the method comprising administering a pharmaceutical composition described herein to the subject.

In yet further aspects, the disclosure provides, a method for reducing MAPT gene expression in a cell, a population of cells or a subject, the method comprising the step of:
i. contacting the cell or the population of cells with a RNAi oligonucleotide or pharmaceutical composition described herein; or
ii. administering to the subject a RNAi oligonucleotide or pharmaceutical composition described herein.

In some aspects, reducing MAPT gene expression comprises reducing an amount or level of MAPT mRNA, an amount or level of Tau protein, or both. In some aspects, a RNAi oligonucleotide or pharmaceutical composition described herein the subject has a disease, disorder, or condition associated with MAPT gene expression. In some aspects, the disease, disorder, or condition associated with MAPT gene expression is AD, frontotemporal dementia (FTD), PSP, PD, Tau protein-associated diseases, primary age-related tauopathy, chronic traumatic encephalopathy, corticobasal degeneration, Lytico-bodig disease, ganglioglioma, meningioangiomatosis, postencephalitic parkinsonism, or subacute sclerosing panencephalitis.

In any of the foregoing or related aspects, MAPT gene expression is reduced in tissue of one or more regions of the CNS, wherein the tissue is associated with AD. In some aspects, tissue associated with AD is selected from: prefrontal cortex, motor cortex, temporal cortex, parietal cortex, and hippocampus. In some aspects, MAPT gene expression is reduced in tissue of one or more regions of the CNS, wherein the tissue is associated with PSP. In some aspects, tissue associated with PSPy is selected from: caudate nucleus, globus pallidus, thalamus, midbrain tegmentum, substantia nigra, pons, cerebellar white matter, cerebellar dentate nucleus, medulla, cervical spinal cord, thoracic spinal cord, and lumbar spinal cord. In some aspects, MAPT gene expression is reduced in one or more regions of the CNS selected from: cervical spinal cord, thoracic spinal cord, lumbar spinal cord, frontal cortex, temporal cortex, cerebellum, midbrain, occipital cortex, parietal cortex, hippocampus, caudate nucleus, thalamus, brainstem, motor cortex, globus pallidus, midbrain tegmentum, substantia nigra, pons, cerebellar white matter, and cerebellar dentate nucleus.

In any of the foregoing or related aspects, the RNAi oligonucleotide, or pharmaceutical composition is administered in combination with a second composition or therapeutic agent.

In other aspects, the disclosure provides use of a RNAi oligonucleotide or pharmaceutical composition described herein in the manufacture of a medicament for the treatment of a disease, disorder, or condition associated with MAPT gene expression.

In further aspects, the disclosure provides a RNAi oligonucleotide or pharmaceutical composition described herein for use, or adaptable for use, in the treatment of a disease, disorder, or condition associated with MAPT gene expression.

In some aspects, the disclosure provides a kit comprising the a RNAi oligonucleotide described herein, an optional pharmaceutically acceptable carrier, and a package insert comprising instructions for administration to a subject having a disease, disorder, or condition associated with MAPT gene expression.

In any of the foregoing or related aspects, the disease, disorder, or condition associated with MAPT gene expression is AD, FTD, PD, PSP, Tau protein-associated diseases, primary age-related tauopathy, chronic traumatic encephalopathy, corticobasal degeneration, Lytico-bodig disease, ganglioglioma, meningioangiomatosis, postencephalitic parkinsonism, or subacute sclerosing panencephalitis.

### BRIEF DESCRIPTION OF THE FIGURES

The advantages, effects, features, and objects other than those set forth above will become more readily apparent when consideration is given to the detailed description below. Such detailed description refers to the following drawings, where:

FIGS. 1A and 1B provide graphs depicting the percent (%) of human MAPT mRNA remaining in the liver of mice exogenously expressing human MAPT (hydrodynamic injection model) after treatment with GalNAc-conjugated MAPT oligonucleotides specific for human (Hs) MAPT or human and NHP (Hs-Mf; “double-common”) MAPT. CD-1 mice were dosed subcutaneously with 3 mg/kg of the indicated GalNAc-conjugated MAPT-targeting oligonucleotide formulated in PBS. Four days post-dose, mice were hydrodynamically injected (HDI) with a DNA plasmid encoding human MAPT. The level of human MAPT mRNA was determined from livers collected 18 hours later.

FIGS. 2A and 2B provide graphs depicting the dose response of GalNAc-conjugated MAPT-targeting oligonucleotides selected based on inhibitory efficacy shown in FIGS. 1A-1B in addition to GalNAc-conjugated MAPT-targeting oligonucleotides specific for human (Hs), NHP (Mf), and murine (Mm) MAPT. The percent (%) of MAPT mRNA remaining in liver tissue was measured in CD-1 HDI mice as described in FIGS. 1A-1B. Following injection with 0.3 mg/kg, 1.0 mg/kg, or 3.0 mg/kg of the indicated GalNAc-conjugated MAPT-targeting oligonucleotide, percent (%) mRNA remaining was determined in two cohorts, FIG. 2A (Set I) and FIG. 2B (Set II). Hs=construct is human MAPT specific; Hs-Mf=construct is human and monkey MAPT specific.

FIGS. 3A-3M provide graphs depicting the percent (%) of human MAPT mRNA remaining in the CNS of NHP after treatment with GalNAc-conjugated MAPT-targeting oligonucleotides. NHPs were dosed by intra cisterna magna (i.e.m) injection with 50 mg of the indicated GalNAc-conjugated MAPT-targeting oligonucleotide formulated in artificial cerebrospinal fluid (aCSF) on study days 0 and 7. The level of MAPT mRNA was determined relative to the percent (%) of MAPT mRNA remaining in aCSF-treated animals. CNS tissues measured included cervical spinal cord (FIG. 3A), thoracic spinal cord (FIG. 3B), lumbar spinal cord (FIG. 3C), frontal cortex (FIG. 3D), temporal cortex (FIG. 3E), cerebellum (FIG. 3F), midbrain (FIG. 3G), occipital cortex (FIG. 3H), parietal cortex (FIG. 3I), hippocampus (FIG. 3J), caudate nucleus (FIG. 3K), thalamus (FIG. 3L), and brainstem (FIG. 3M).

FIGS. 4A-4B provide schematics of a lipid-conjugated RNAi oligonucleotide (FIG. 4A) and a GalNAc-conjugated RNAi oligonucleotide (FIG. 4B). FIG. 4A discloses SEQ ID NO: 1693 and FIG. 4B discloses SEQ ID NO: 1694.

FIGS. 5A-5B provide graphs depicting the percent (%) of NHP (Mf) MAPT mRNA remaining (FIG. 5A) and concentration of oligonucleotide (FIG. 5B) in CNS tissue associated with AD. NHPs were intrathecally administered MAPT-2357 conjugated to a C₁₆ lipid or GalNAc, as shown in the modification patterns of FIGS. 4A-4B. Tissue was collected and analyzed 28 days after administration of the indicated oligonucleotide.

FIGS. 6A-6B provide graphs depicting the percent (%) of NHP (Mf) MAPT mRNA remaining (FIG. 6A) and concentration of oligonucleotide (FIG. 6B) in CNS tissue associated with PSP. NHPs were intrathecally administered MAPT-2357 conjugated to a C₁₆ lipid or GalNAc, as shown in the modification patterns of FIGS. 4A-4B. Tissue was collected and analyzed 28 days after administration of the indicated oligonucleotide.

### DETAILED DESCRIPTION

According to some aspects, the disclosure provides oligonucleotides such as RNAi oligonucleotides that reduce MAPT gene expression in the CNS. In some embodiments, the oligonucleotides provided herein are designed to treat diseases associated with MAPT gene expression in the CNS. In some respects, the disclosure provides methods of treating a disease associated with MAPT by reducing MAPT gene expression in cells (e.g., cells of the CNS).

### Oligonucleotide Inhibitors of MAPT Gene Expression

The disclosure provides, inter alia, oligonucleotides that inhibit MAPT gene expression (e.g., RNAi oligonucleotides). In some embodiments, the oligonucleotide that inhibits MAPT gene expression is targeted to a MAPT mRNA.

### MAPT Target Sequences

In some embodiments, an oligonucleotide herein (e.g., a RNAi oligonucleotide) is targeted to a target sequence comprising a MAPT mRNA. In some embodiments, the oligonucleotide is targeted to a target sequence within a MAPT mRNA sequence.

In some embodiments, the oligonucleotide corresponds to a target sequence within a MAPT mRNA sequence. In some embodiments, the oligonucleotide, or a portion, fragment, or strand thereof (e.g., an antisense strand or a guide strand of a RNAi oligonucleotide) binds or anneals to a target sequence comprising MAPT mRNA, thereby inhibiting MAPT gene expression.

In some embodiments, the oligonucleotide is targeted to a MAPT target sequence for the purpose of inhibiting MAPT gene expression in vivo. In some embodiments, the amount or extent of inhibition of MAPT gene expression by the oligonucleotide targeted to a MAPT target sequence correlates with the potency of the oligonucleotide. In some embodiments, the amount or extent of MAPT gene expression inhibition by the oligonucleotide targeted to a MAPT target sequence correlates with the amount or extent of therapeutic benefit in a subject or patient having a disease, disorder, or condition associated with MAPT gene expression treated with the oligonucleotide.

In some embodiments, a sense strand of the oligonucleotide comprises a MAPT target sequence. In some embodiments, a portion or region of the sense strand of the oligonucleotide (e.g., a RNAi oligonucleotide) comprises a MAPT target sequence. In some embodiments, the MAPT target sequence comprises, or consists of, a nucleotide sequence of any one of SEQ ID NOs: 912-1295. In some embodiments, the MAPT target sequence comprises, or consists of, a nucleotide sequence of any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, and 924. In some embodiments, the MAPT target sequence comprises, or consists of, a nucleotide sequence of any one of SEQ ID NOs: 1061, 1108, 1119, 1120, 1124, 1130, 1065, 1095, 1096, and 1102. In some embodiments, the MAPT target sequence comprises, or consists of, a nucleotide sequence of any one of SEQ ID NOs: 1130, 1095, 1096, 1119, 1120, and 1124. In some embodiments, the MAPT target sequence comprises the nucleotide sequence set forth in SEQ ID NO: 1130. In some embodiments, the MAPT target sequence comprises the nucleotide sequence set forth in SEQ ID NO: 1095. In some embodiments, the MAPT target sequence comprises the nucleotide sequence set forth in SEQ ID NO: 1096. In some embodiments, the MAPT target sequence comprises the nucleotide sequence set forth in SEQ ID NO: 1119. In some embodiments, the MAPT target sequence comprises the nucleotide sequence set forth in SEQ ID NO: 1120. In some embodiments, the MAPT target sequence comprises the nucleotide sequence set forth in SEQ ID NO: 1124.

MAPT mRNA Targeting Sequences

In some embodiments, an oligonucleotide herein (e.g., a RNAi oligonucleotide) has a region of complementarity to MAPT mRNA (e.g., within a target sequence of MAPT mRNA) for purposes of targeting the mRNA in cells and inhibiting its expression. In some embodiments, the oligonucleotide comprises a MAPT mRNA target sequence (e.g., an antisense strand or a guide strand of a ds oligonucleotide such as a RNAi oligonucleotide) having a region of complementarity that binds or anneals to a MAPT target sequence by complementary (Watson-Crick) base pairing. The targeting sequence or region of complementarity is generally of suitable length and base content to enable binding or annealing of the oligonucleotide (or a strand thereof) to a MAPT mRNA for purposes of inhibiting its expression. In some embodiments, the targeting sequence or region of complementarity is at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29 or at least about 30 nucleotides in length. In some embodiments, the targeting sequence or region of complementarity is at least 12, at least 13, at least 14, at least 15, at least 16, at least 17, at least 18, at least 19, or at least 20 nucleotides. In some embodiments, the targeting sequence or region of complementarity is about 12 to about 30 (e.g., 12 to 30, 12 to 22, 15 to 25, 17 to 21, 18 to 27, 19 to 27, or 15 to 30) nucleotides in length. In some embodiments, the targeting sequence or region of complementarity is about 12, 13, 14, 15, 16, 17, 18, 19, 20, 21, 22, 23, 24, 25, 26, 27, 28, 29, or 30 nucleotides in length. In some embodiments, the targeting sequence or region of complementarity is 18 nucleotides in length. In some embodiments, the targeting sequence or region of complementarity is 19 nucleotides in length. In some embodiments, the targeting sequence or region of complementarity is 20 nucleotides in length. In some embodiments, the targeting sequence or region of complementarity is 21 nucleotides in length. In some embodiments, the targeting sequence or region of complementarity is 22 nucleotides in length. In some embodiments, the targeting sequence or region of complementarity is 23 nucleotides in length. In some embodiments, the targeting sequence or region of complementarity is 24 nucleotides in length. In some embodiments, the oligonucleotide comprises a target sequence or region of complementarity complementary to a sequence of any one of SEQ ID NOs: 912-1295, and the targeting sequence or region of complementarity is 18 nucleotides in length. In some embodiments, the oligonucleotide comprises a target sequence or region of complementarity complementary to a sequence of any one of SEQ ID NOs: 912-1295, and the targeting sequence or region of complementarity is 19 nucleotides in length. In some embodiments, the oligonucleotide comprises a target sequence or region of complementarity complementary to a sequence of any one of SEQ ID NOs: 1-384, and the targeting sequence or region of complementarity is 20 nucleotides in length. In some embodiments, the oligonucleotide comprises a target sequence or region of complementarity complementary to a sequence of any one of SEQ ID NOs: 1-384, and the targeting sequence or region of complementarity is 21 nucleotides in length. In some embodiments, the oligonucleotide comprises a target sequence or region of complementarity complementary to a sequence of any one of SEQ ID NOs: 1-384, and the targeting sequence or region of complementarity is 22 nucleotides in length. In some embodiments, the oligonucleotide comprises a target sequence or region of complementarity complementary to a sequence of any one of SEQ ID NOs: 1-384, and the targeting sequence or region of complementarity is 23 nucleotides in length. In some embodiments, the oligonucleotide comprises a target sequence or region of complementarity complementary to a sequence of any one of SEQ ID NOs: 1-384, and the targeting sequence or region of complementarity is 24 nucleotides in length.

In some embodiments, the oligonucleotide comprises a targeting sequence or a region of complementarity (e.g., an antisense strand or a guide strand of a ds oligonucleotide) that is fully complementary to a MAPT mRNA target sequence. In some embodiments, the targeting sequence or region of complementarity is partially complementary to a MAPT mRNA target sequence. In some embodiments, the oligonucleotide comprises a targeting sequence or region of complementarity that is fully complementary to a sequence of any one of SEQ ID NOs: 912-1295. In some embodiments, the oligonucleotide comprises a targeting sequence or region of complementarity that is partially complementary to a sequence of any one of SEQ ID NOs: 912-1295. In some embodiments, the oligonucleotide comprises a targeting sequence or region of complementarity that is fully complementary to a sequence of any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, or 924. In some embodiments, the oligonucleotide comprises a targeting sequence or region of complementarity that is partially complementary to a sequence of any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, or 924. In some embodiments, the oligonucleotide comprises a targeting sequence or region of complementarity that is fully complementary to a sequence of any one of SEQ ID NOs: 1061, 1108, 1119, 1120, 1124, 1130, 1065, 1095, 1096, or 1102. In some embodiments, the oligonucleotide comprises a targeting sequence or region of complementarity that is partially complementary to a sequence of any one of SEQ ID NOs: 1061, 1108, 1119, 1120, 1124, 1130, 1065, 1095, 1096, or 1102. In some embodiments, the oligonucleotide comprises a targeting sequence or region of complementarity that is fully complementary to a sequence of any one of SEQ ID NOs: 1130, 1095, 1096, 1119, 1120, and 1124. In some embodiments, the oligonucleotide comprises a targeting sequence or region of complementarity that is partially complementary to a sequence of any one of SEQ ID NOs: 1130, 1095, 1096, 1119, 1120, and 1124. In some embodiments, the oligonucleotide comprises a targeting sequence or region of complementarity that is fully complementary to the sequence set forth in SEQ ID NO: 1130. In some embodiments, the oligonucleotide comprises a targeting sequence or region of complementarity that is fully complementary to the sequence set forth in SEQ ID NO: 1095. In some embodiments, the oligonucleotide comprises a targeting sequence or region of complementarity that is fully complementary to the sequence set forth in SEQ ID NO: 1096. In some embodiments, the oligonucleotide comprises a targeting sequence or region of complementarity that is fully complementary to the sequence set forth in SEQ ID NO: 1119. In some embodiments, the oligonucleotide comprises a targeting sequence or region of complementarity that is fully complementary to the sequence set forth in SEQ ID NO: 1120. In some embodiments, the oligonucleotide comprises a targeting sequence or region of complementarity that is fully complementary to the sequence set forth in SEQ ID NO: 1124. In some embodiments, the oligonucleotide comprises a targeting sequence or region of complementarity that is partially complementary to a sequence of any one of SEQ ID NOs: 1130. In some embodiments, the oligonucleotide comprises a targeting sequence or region of complementarity that is partially complementary to the sequence of SEQ ID NO: 1095. In some embodiments, the oligonucleotide comprises a targeting sequence or region of complementarity that is partially complementary to the sequence of SEQ ID NO: 1096. In some embodiments, the oligonucleotide comprises a targeting sequence or region of complementarity that is partially complementary to the sequence of SEQ ID NO: 1119. In some embodiments, the oligonucleotide comprises a targeting sequence or region of complementarity that is partially complementary to the sequence of SEQ ID NO: 1120. In some embodiments, the oligonucleotide comprises a targeting sequence or region of complementarity that is partially complementary to the sequence of SEQ ID NO: 1124.

In some embodiments, the oligonucleotide comprises a targeting sequence or region of complementarity that is complementary to a contiguous sequence of nucleotides comprising a MAPT mRNA, wherein the contiguous sequence of nucleotides is about 12 to about 30 nucleotides in length (e.g., 12 to 30, 12 to 28, 12 to 26, 12 to 24, 12 to 20, 12 to 18, 12 to 16, 14 to 22, 16 to 20, 18 to 20, or 18 to 19 nucleotides in length). In some embodiments, the oligonucleotide comprises a targeting sequence or region of complementarity that is complementary to a contiguous sequence of nucleotides comprising a MAPT mRNA, wherein the contiguous sequence of nucleotides is 10, 11, 12, 13, 14, 15, 16, 17, 18, 19, or 20 nucleotides in length. In some embodiments, the oligonucleotide comprises a targeting sequence or region of complementarity that is complementary to a contiguous sequence of nucleotides comprising a MAPT mRNA, wherein the contiguous sequence of nucleotides is 19 nucleotides in length. In some embodiments, the oligonucleotide comprises a targeting sequence or region of complementarity that is complementary to a contiguous sequence of nucleotides comprising a MAPT mRNA, wherein the contiguous sequence of nucleotides is 20 nucleotides in length.

In some embodiments, the oligonucleotide comprises a targeting sequence or a region of complementarity that is complementary to a contiguous sequence of nucleotides of any one of SEQ ID NOs: 912-1295, optionally where the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, and 924, optionally wherein the contiguous sequence of nucleotides is 19 nucleotides in length. In some embodiments, the oligonucleotide comprises a targeting sequence or a region of complementarity that is complementary to a contiguous sequence of nucleotides of any one of SEQ ID NOs: 1061, 1108, 1119, 1120, 1124, 1130, 1065, 1095, 1096, and 1102, optionally where the contiguous sequence of nucleotides is 19 nucleotides in length. In some embodiments, the oligonucleotide comprises a targeting sequence or a region of complementarity that is complementary to a contiguous sequence of nucleotides of SEQ ID NO: 1130, 1095, 1096, 1119, 1120, and 1124, optionally where the contiguous sequence of nucleotides is 19 nucleotides in length.

In some embodiments, the targeting sequence or region of complementarity of the oligonucleotide is complementary to contiguous nucleotides of a sequence as set forth in any one of SEQ ID NOs: 912-1295 and spans the entire length of the antisense strand. In some embodiments, the targeting sequence or region of complementarity of the oligonucleotide is complementary to contiguous nucleotides of a sequence as set forth in any one of SEQ ID NOs: 912-1295 and spans a portion of the entire length of an antisense strand. In some embodiments, the oligonucleotide comprises a region of complementarity (e.g., on an antisense strand of a ds oligonucleotide) that is at least partially (e.g., fully) complementary to a contiguous stretch of nucleotides spanning nucleotides 1-20 of a sequence as set forth in any one of SEQ ID NOs: 912-1295. In some embodiments, the targeting sequence or region of complementarity of the oligonucleotide is complementary to contiguous nucleotides of a sequence as set forth in any one of SEQ ID NOs: 1-384 and spans the entire length of an antisense strand. In some embodiments, the region of complementarity of the oligonucleotide is complementary to contiguous nucleotides of a sequence as set forth in any one of SEQ ID NOs: 1-384 and spans a portion of the entire length of an antisense strand. In some embodiments, the oligonucleotide comprises the region of complementarity (e.g., on an antisense strand of a ds oligonucleotide) that is at least partially (e.g., fully) complementary to a contiguous stretch of nucleotides spanning nucleotides 1-19 of a sequence as set forth in any one of SEQ ID NOs: 1-384.

In some embodiments, the oligonucleotide comprises a targeting sequence or region of complementarity having one or more base pair (bp) mismatches with the corresponding MAPT mRNA target sequence. In some embodiments, the targeting sequence or region of complementarity may have up to about 1, up to about 2, up to about 3, up to about 4, up to about 5, etc. mismatches with the corresponding MAPT mRNA target sequence provided that the ability of the targeting sequence or region of complementarity to bind or anneal to MAPT mRNA under appropriate hybridization conditions and/or the ability of the oligonucleotide to inhibit MAPT gene expression is maintained. Alternatively, in some embodiments, the targeting sequence or region of complementarity comprises no more than 1, no more than 2, no more than 3, no more than 4, or no more than 5 mismatches with the corresponding MAPT mRNA target sequence provided that the ability of the targeting sequence or region of complementarity to bind or anneal to MAPT mRNA under appropriate hybridization conditions and/or the ability of the oligonucleotide to inhibit MAPT gene expression is maintained. In some embodiments, the oligonucleotide comprises a targeting sequence or region of complementarity having 1 mismatch with the corresponding target sequence. In some embodiments, the oligonucleotide comprises a targeting sequence or region of complementarity having 2 mismatches with the corresponding target sequence. In some embodiments, the oligonucleotide comprises a targeting sequence or region of complementarity having 3 mismatches with the corresponding target sequence. In some embodiments, the oligonucleotide comprises a targeting sequence or region of complementarity having 4 mismatches with the corresponding target sequence. In some embodiments, the oligonucleotide comprises a targeting sequence or region of complementarity having 5 mismatches with the corresponding target sequence. In some embodiments, the oligonucleotide comprises a targeting sequence or region of complementarity more than one mismatch (e.g., 2, 3, 4, 5, or more mismatches) with the corresponding target sequence, wherein at least 2 (e.g., all) of the mismatches are positioned consecutively (e.g., 2, 3, 4, 5, or more mismatches in a row), or wherein the mismatches are interspersed in any position throughout the targeting sequence or region of complementarity. In some embodiments, the oligonucleotide comprises a targeting sequence or region of complementarity more than one mismatch (e.g., 2, 3, 4, 5, or more mismatches) with the corresponding target sequence, wherein at least 2 (e.g., all) of the mismatches are positioned consecutively (e.g., 2, 3, 4, 5, or more mismatches in a row), or wherein at least one or more non-mismatched base pair is located between the mismatches, or a combination thereof.

In some embodiments, the oligonucleotide comprises a targeting sequence or a region of complementary that is complementary to a contiguous sequence of nucleotides of any one of SEQ ID NOs: 912-1295, wherein the targeting sequence or region of complementarity may have up to about 1, up to about 2, up to about 3, up to about 4, up to about 5, etc. mismatches with the corresponding MAPT mRNA target sequence. In some embodiments, the oligonucleotide comprises a targeting sequence or a region of complementary that is complementary to a contiguous sequence of nucleotides of any one of SEQ ID NOs: 912-1295, wherein the targeting sequence or region of complementarity may have no more than 1, no more than 2, no more than 3, no more than 4, or no more than 5 mismatches with the corresponding MAPT mRNA target sequence. In some embodiments, the oligonucleotide comprises a targeting sequence or a region of complementary that is complementary to a contiguous sequence of nucleotides of any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, and 924, wherein the targeting sequence or region of complementarity may have up to about 1, up to about 2, up to about 3, up to about 4, up to about 5, etc. mismatches with the corresponding MAPT mRNA target sequence. In some embodiments, the oligonucleotide comprises a targeting sequence or a region of complementary that is complementary to a contiguous sequence of nucleotides of any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, and 924, wherein the targeting sequence or region of complementarity may have no more than 1, no more than 2, no more than 3, no more than 4, or no more than 5 mismatches with the corresponding MAPT mRNA target sequence. In some embodiments, the oligonucleotide comprises a targeting sequence or a region of complementary that is complementary to a contiguous sequence of nucleotides of any one of SEQ ID NOs: 1061, 1108, 1119, 1120, 1124, 1130, 1065, 1095, 1096, and 1102, wherein the targeting sequence or region of complementarity may have up to about 1, up to about 2, up to about 3, up to about 4, up to about 5, etc. mismatches with the corresponding MAPT mRNA target sequence. In some embodiments, the oligonucleotide comprises a targeting sequence or a region of complementary that is complementary to a contiguous sequence of nucleotides of any one of SEQ ID NOs: 1061, 1108, 1119, 1120, 1124, 1130, 1065, 1095, 1096, and 1102, wherein the targeting sequence or region of complementarity may have no more than 1, no more than 2, no more than 3, no more than 4, or no more than 5 mismatches with the corresponding MAPT mRNA target sequence. In some embodiments, the oligonucleotide comprises a targeting sequence or a region of complementary that is complementary to a contiguous sequence of nucleotides of any one of SEQ ID NOs: 1130, 1095, 1096, 1119, 1120, and 1124, wherein the targeting sequence or region of complementarity may have up to about 1, up to about 2, up to about 3, up to about 4, up to about 5, etc. mismatches with the corresponding MAPT mRNA target sequence. In some embodiments, the oligonucleotide comprises a targeting sequence or a region of complementary that is complementary to a contiguous sequence of nucleotides of any one of SEQ ID NOs: 1130, 1095, 1096, 1119, 1120, and 1124, wherein the targeting sequence or region of complementarity may have no more than 1, no more than 2, no more than 3, no more than 4, or no more than 5 mismatches with the corresponding MAPT mRNA target sequence. In some embodiments, the oligonucleotide comprises a targeting sequence or a region of complementary that is complementary to a contiguous sequence of nucleotides of SEQ ID NO: 1095, wherein the targeting sequence or region of complementarity may have up to about 1, up to about 2, up to about 3, up to about 4, up to about 5, etc. mismatches with the corresponding MAPT mRNA target sequence. In some embodiments, the oligonucleotide comprises a targeting sequence or a region of complementary that is complementary to a contiguous sequence of nucleotides of SEQ ID NO: 1095, wherein the targeting sequence or region of complementarity may have no more than 1, no more than 2, no more than 3, no more than 4, or no more than 5 mismatches with the corresponding MAPT mRNA target sequence.

### Types of Oligonucleotides

A variety of oligonucleotide types and/or structures are useful for targeting MAPT mRNA in the methods herein including, but not limited to, RNAi oligonucleotides. Any of the oligonucleotide types described herein or elsewhere are contemplated for use as a framework to incorporate a MAPT mRNA targeting sequence herein for the purposes of inhibiting MAPT gene expression.

In some embodiments, the oligonucleotides herein inhibit MAPT gene expression by engaging with RNA interference (RNAi) pathways upstream or downstream of Dicer involvement (e.g., a RNAi oligonucleotide). For example, RNAi oligonucleotides have been developed with each strand having sizes of about 19-25 nucleotides with at least one 3′ overhang of about 1 to about 5 nucleotides (see, e.g., U.S. Pat. No. 8,372,968). Longer oligonucleotides also have been developed that are processed by Dicer to generate active RNAi products (see, e.g., U.S. Pat. No. 8,883,996). Further work produced extended ds oligonucleotides where at least one end of at least one strand is extended beyond a duplex targeting region, including structures where one of the strands includes a thermodynamically-stabilizing tetraL structure (see, e.g., U.S. Pat. Nos. 8,513,207 and 8,927,705, as well as Intl. Patent Application Publication No. WO 2010/033225). Such structures may include single-stranded (ss) extensions (on one or both sides of the molecule) as well as ds extensions.

In some embodiments, the oligonucleotide engages with the RNAi pathway downstream of the involvement of Dicer (e.g., Dicer cleavage). In some embodiments, the oligonucleotide has an overhang (e.g., of 1, 2, or 3 nucleotides in length) in the 3′ end of the sense strand. In some embodiments, the oligonucleotide comprises a 21-nucleotide antisense strand that is antisense to a target mRNA (e.g., MAPT mRNA) and a complementary sense strand, in which both strands anneal to form a 19-bp duplex and 2 nucleotide overhangs at either or both 3′ ends. Longer oligonucleotide designs also are contemplated including oligonucleotides having an antisense strand of 23 nucleotides and a sense strand of 21 nucleotides, where there is a blunt end on the right side of the oligonucleotide (3′ end of sense strand/5′ end of antisense strand) and a two nucleotide 3′ guide strand overhang on the left side of the oligonucleotide (5′ end of the sense strand/3′ end of the antisense strand). In such molecules, there is a 21 bp duplex region. See, e.g., U.S. Pat. Nos. 9,012,138; 9,012,621; and 9,193,753.

In some embodiments, the oligonucleotide comprises sense and antisense strands that are both in the range of about 17 to about 36 (e.g., 17 to 26, 20 to 25, or 21-23) nucleotides in length. In some embodiments, the oligonucleotide comprises an antisense strand of 19-30 nucleotides in length and a sense strand of 19-50 nucleotides in length, where the antisense and sense strands are separate strands that form an asymmetric duplex region having an overhang of 1-4 nucleotides at the 3′ terminus of the antisense strand. In some embodiments, the oligonucleotide comprises sense and antisense strands that are both in the range of about 19-22 nucleotides in length. In some embodiments, the sense and antisense strands are of equal length. In some embodiments, the oligonucleotide comprises sense and antisense strands, such that there is a 3′ overhang on either the sense strand or the antisense strand, or both the sense and antisense strand. In some embodiments, for oligonucleotides that have sense and antisense strands that are both in the range of about 21-23 nucleotides in length, a 3′ overhang on the sense, antisense, or both is/are 1 or 2 nucleotides in length. In some embodiments, the oligonucleotide has a guide strand of 22 nucleotides and a passenger strand of 20 nucleotides, where there is a blunt end on the right side of the molecule (3′ end of passenger strand/5′ end of guide strand) and a 2 nucleotide 3′ guide strand overhang on the left side of the molecule (5′ end of the passenger strand/3′ end of the guide strand). In such molecules, there is a 20 bp duplex region.

Other oligonucleotide designs for use with the compositions and methods herein include: 16-mer siRNAs (see, e.g., NUCLEIC ACIDS IN CHEMISTRY AND BIOLOGY, Blackburn (ed.), Royal Society of Chemistry, 2006), shRNAs (e.g., having 19 bp or shorter stems; see, e.g., Moore et al. (2010) METHODS MOL. BIOL. 629:141-58), blunt siRNAs (e.g., of 19 bps in length; see, e.g., Kraynack & Baker (2006) RNA 12:163-76), asymmetrical siRNAs (aiRNA; see, e.g., Sun et al. (2008) NAT. BIOTECHNOL. 26:1379-82), asymmetric shorter-duplex siRNA (see, e.g., Chang et al. (2009) MOL. THER. 17:725-732), fork siRNAs (see, e.g., Hohjoh (2004) FEBS LETT. 557:193-98), single-stranded siRNAs (Elsner (2012) NAT. BIOTECHNOL. 30:1063), dumbbell-shaped circular siRNAs (see, e.g., Abe et al. (2007) J. AM. CHEM. SOC. 129:15108-09), and small internally segmented interfering RNA (siRNA; see, e.g., Bramsen et al. (2007) NUCLEIC ACIDS RES. 35:5886-97). Further non-limiting examples of an oligonucleotide structure that may be used in some embodiments to reduce or inhibit the expression of MAPT are microRNA (miRNA), short hairpin RNA (shRNA), and short siRNA (see, e.g., Hamilton et al. (2002) EMBO J. 21:4671-79; see also, US Patent Application Publication No. 2009/0099115).

Still, in some embodiments, the oligonucleotide for reducing or inhibiting MAPT gene expression herein is ss. Such structures may include, but are not limited to, ss RNAi molecules. Recent efforts have demonstrated the activity of ss RNAi molecules (see, e.g., Matsui et al. (2016) Mol. Ther. 24:946-955). However, in some embodiments, the oligonucleotide is an antisense oligonucleotide (ASO). An ASO is a ss oligonucleotide that has a nucleobase sequence which, when written or depicted in the 5′ to 3′ direction, comprises the reverse complement of a targeted segment of a particular nucleic acid and is suitably modified (e.g., as a gapmer) so as to induce RNaseH-mediated cleavage of its target RNA in cells or (e.g., as a mixmer) so as to inhibit translation of the target mRNA in cells. ASOs for use herein may be modified in any suitable manner known in the art including, for example, as shown in U.S. Pat. No. 9,567,587 (including, e.g., length, sugar moieties of the nucleobase (pyrimidine, purine), and alterations of the heterocyclic portion of the nucleobase). Further, ASOs have been used for decades to reduce expression of specific target genes (see, e.g., Bennett et al. (2017) Annu. Rev. Pharmacol. 57:81-105).

In some embodiments, the ASO shares a region of complementarity with MAPT mRNA. In some embodiments, the ASO targets various areas of the human MAPT identified as NM 001123066.3. In some embodiments, the ASO is about 15-50 nucleotides in length. In some embodiments, the ASO is about 15-25 nucleotides in length. In some embodiments, the ASO is 22 nucleotides in length. In some embodiments, the ASO is complementary to any one of SEQ ID NOs: 912-1295. In some embodiments, the ASO is at least 15 contiguous nucleotides in length. In some embodiments, the ASO is at least 19 contiguous nucleotides in length. In some embodiments, the ASO is at least 20 contiguous nucleotides in length. In some embodiments, the ASO differs by 1, 2, or 3 nucleotides from the target sequence.

### Double-Stranded RNAi Oligonucleotides

In some aspects, the disclosure provides ds RNAi oligonucleotides for targeting MAPT mRNA and inhibiting MAPT gene expression (e.g., via the RNAi pathway) comprising a sense strand (also referred to herein as a passenger strand) and an antisense strand (also referred to herein as a guide strand). In some embodiments, the sense strand and antisense strand are separate strands and are not covalently linked. In some embodiments, the sense strand and the antisense strand are covalently linked. In some embodiments, the sense strand and the antisense strand form a duplex region, wherein the sense strand and the antisense strand, or a portion thereof, binds with one another in a complementary fashion (e.g., by Watson-Crick base pairing).

In some embodiments, the sense strand has a first region (R1) and a second region (R2), wherein R2 comprises a first subregion (S1), a L (e.g., tetraL or triL), and a second subregion (S2), wherein L is located between S1 and S2, and wherein S1 and S2 form a second duplex (D2). D2 may have various lengths. In some embodiments, D2 is about 1 to about 6 bp in length. In some embodiments, D2 is 2-6, 3-6, 4-6, 5-6, 1-5, 2-5, 3-5, or 4-5 bp in length. In some embodiments, D2 is 1, 2, 3, 4, 5, or 6 bp in length. In some embodiments, D2 is 6 bp in length.

In some embodiments, R1 of the sense strand and the antisense strand form a first duplex (D1). In some embodiments, D1 is at least about 15 (e.g., at least 15, at least 16, at least 17, at least 18, at least 19, at least 20, or at least 21) nucleotides in length. In some embodiments, D1 is in the range of about 12 to about 30 nucleotides in length (e.g., 12 to 30, 12 to 27, 15 to 22, 18 to 22, 18 to 25, 18 to 27, 18 to 30, or 21 to 30 nucleotides in length). In some embodiments, D1 is at least 12 nucleotides in length (e.g., at least 12, at least 15, at least 20, at least 25, or at least 30 nucleotides in length). In some embodiments, D1 is 12, 13, 14, 15, 16, 17, 18, 19, 20, 21, 22, 23, 24, 25, 26, 27, 28, 29, or 30 nucleotides in length. In some embodiments, D1 is 19 nucleotides in length. In some embodiments, D1 is 20 nucleotides in length. In some embodiments, D1 comprising the sense strand and the antisense strand does not span the entire length of the sense strand and/or the antisense strand. In some embodiments, D1 comprising the sense strand and the antisense strand spans the entire length of either the sense strand or the antisense strand or both. In certain embodiments, D1 comprising the sense strand and the antisense strand spans the entire length of both the sense strand and the antisense strand.

In some embodiments, a sense strand described here is 36 nucleotides in length and positions are numbered 1-36 from 5′ to 3′. In some embodiments, an antisense strand described herein is 22 nucleotides in length and positions are numbered 1-22 from 5′ to 3′. In some embodiments, position numbers described herein adhere to this numbering format.

In some embodiments, the RNAi oligonucleotide comprises a sense strand having a sequence of any one of SEQ ID NOs: 1-384 and an antisense strand comprising a complementary sequence of any one of SEQ ID NOs: 385-768. In some embodiments, the RNAi oligonucleotide comprises a sense strand having a sequence of SEQ ID NOs: 912-1295 and an antisense strand comprising a complementary sequence of any one of SEQ ID NOs: 1296-1679.

In some embodiments, the RNAi oligonucleotide comprises a sense strand having a sequence of any one of SEQ ID NOs: 769-803 and an antisense strand comprising a complementary sequence of any one of SEQ ID NOs: 804-838. In some embodiments, the RNAi oligonucleotide comprises a sense strand having a sequence of any one of SEQ ID NOs: 769-803 and 1681 and an antisense strand comprising a complementary sequence of any one of SEQ ID NOs: 804-838.

In some embodiments, the RNAi oligonucleotide comprises a sense strand and an antisense strand comprising nucleotide sequences selected from:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
S) SEQ ID NOs: 787 and 822, respectively;
t) SEQ ID NOs: 788 and 823, respectively;
u) SEQ ID NOs: 789 and 824, respectively;
V) SEQ ID NOs: 790 and 825, respectively;
w) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively;
ii) SEQ ID NOs: 803 and 838, respectively; and
jj) SEQ ID NOs: 1681 and 815, respectively.

In some embodiments, the RNAi oligonucleotide comprises a sense strand and an antisense strand comprising nucleotide sequences selected from:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively; and
k) SEQ ID NOs: 1681 and 815, respectively.

In some embodiments, the RNAi oligonucleotide comprises a sense strand and an antisense strand comprising nucleotide sequences selected from:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively;
f) SEQ ID NOs: 803 and 838, respectively; and
g) SEQ ID NOs: 1681 and 815, respectively.

In some embodiments, the sense strand comprises the sequence of SEQ ID NO: 771, and the antisense strand comprises the sequence of SEQ ID NO: 806. In some embodiments, the sense strand comprises the sequence of SEQ ID NO: 780, and the antisense strand comprises the sequence of SEQ ID NO: 815. In some embodiments, the sense strand comprises the sequence of SEQ ID NO: 781, and the antisense strand comprises the sequence of SEQ ID NO: 816. In some embodiments, the sense strand comprises the sequence of SEQ ID NO: 798, and the antisense strand comprises the sequence of SEQ ID NO: 833. In some embodiments, the sense strand comprises the sequence of SEQ ID NO: 799, and the antisense strand comprises the sequence of SEQ ID NO: 834. In some embodiments, the sense strand comprises the sequence of SEQ ID NO: 803, and the antisense strand comprises the sequence of SEQ ID NO: 838. In some embodiments, the sense strand comprises the sequence of SEQ ID NO: 1681, and the antisense strand comprises the sequence of SEQ ID NO: 815.

It should be appreciated that, in some embodiments, sequences presented in the Sequence Listing may be referred to in describing the structure of the oligonucleotide (e.g., a RNAi oligonucleotide) or other nucleic acid. In such embodiments, the actual oligonucleotide or other nucleic acid may have one or more alternative nucleotides (e.g., a RNA counterpart of a DNA nucleotide or a DNA counterpart of an RNA nucleotide) and/or one or more modified nucleotides and/or one or more modified internucleotide linkages and/or one or more other modification when compared with the specified sequence while retaining essentially same or similar complementary properties as the specified sequence.

In some embodiments, a RNAi oligonucleotide herein comprises a 25-nucleotide sense strand and a 27-nucleotide antisense strand that when acted upon by a Dicer enzyme results in an antisense strand that is incorporated into the mature RNA-induced silencing complex (RISC). In some embodiments, the 25-nucleotide sense strand comprises a sequence selected from SEQ ID NOs: 1-384. In some embodiments, the 27-nucleotide antisense strand comprises a sequence selected from SEQ ID NOs: 385-768. In some embodiments, the sense strand of the RNAi oligonucleotide is longer than 27 nucleotides (e.g., 28, 29, 30, 31, 32, 33, 34, 35, 36, 37, 38, 39, 40, 41, 42, 43, 44, 45, 46, 47, 48, 49, or 50 nucleotides). In some embodiments, the sense strand of the RNAi oligonucleotide is longer than 25 nucleotides (e.g., 26, 27, 28, 29 or 30 nucleotides). In some embodiments, the sense strand of the RNAi oligonucleotide comprises a nucleotide sequence selected from SEQ ID NOs: 912-1295, wherein the nucleotide sequence is longer than 27 nucleotides (e.g., 28, 29, 30, 31, 32, 33, 34, 35, 36, 37, 38, 39, 40, 41, 42, 43, 44, 45, 46, 47, 48, 49, or 50 nucleotides). In some embodiments, the sense strand of the RNAi oligonucleotide comprises a nucleotide sequence selected from SEQ ID NOs: 912-1295, wherein the nucleotide sequence is longer than 25 nucleotides (e.g., 26, 27, 28, 29, or 30 nucleotides).

In some embodiments, the RNAi oligonucleotide has one 5′ end that is thermodynamically less stable when compared to the other 5′ end. In some embodiments, an asymmetric RNAi oligonucleotide is provided that comprises a blunt end at the 3′ end of a sense strand and a 3′ overhang at the 3′ end of an antisense strand. In some embodiments, the 3′ overhang on the antisense strand is about 1-8 nucleotides in length (e.g., 1, 2, 3, 4, 5, 6, 7, or 8 nucleotides in length). Typically, the RNAi oligonucleotide has a two-nucleotide overhang on the 3′ end of the antisense (guide) strand; however, other overhangs are possible. In some embodiments, the overhang is a 3′ overhang comprising a length of between about 1 to about 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′ overhang comprising a length of between about 1 to about 6 nucleotides, optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5, or 6 nucleotides. In some embodiments, the RNAi oligonucleotide comprises a targeting sequence or a region of complementary that is complementary to a contiguous sequence of nucleotides of any one of SEQ ID NOs: 912-1295, and a 5′ overhang comprising a length of between about 1 and about 6 nucleotides. In some embodiments, the RNAi oligonucleotide comprises a sense strand comprising a nucleotide sequence selected from SEQ ID NOs: 912-1295, wherein the RNAi oligonucleotide comprises a 5′ overhang comprising a length of between about 1 and about 6 nucleotides. In some embodiments, the RNAi oligonucleotide comprises an antisense strand comprising a nucleotide sequence selected from SEQ ID NOs: 1296-1679, wherein the RNAi oligonucleotide comprises a 5′ overhang comprising a length of between about 1 and about 6 nucleotides. In some embodiments, the RNAi oligonucleotide comprises a sense strand comprising a nucleotide sequence selected from SEQ ID NOs: 912-1295 and antisense strand comprising a nucleotide sequence selected from SEQ ID NOs: 1296-1679, wherein the RNAi oligonucleotide comprises a 5′ overhang comprising a length of between about 1 and about 6 nucleotides.

In some embodiments, two terminal nucleotides on the 3′ end of an antisense strand are modified. In some embodiments, the two terminal nucleotides on the 3′ end of the antisense strand are complementary with the target mRNA (e.g., MAPT mRNA). In some embodiments, the two terminal nucleotides on the 3′ end of the antisense strand are not complementary with the target mRNA. In some embodiments, the two terminal nucleotides on the 3′ end of the antisense strand of a RNAi oligonucleotide herein are unpaired. In some embodiments, the two terminal nucleotides on the 3′ end of the antisense strand of the RNAi oligonucleotide herein comprise an unpaired GG. In some embodiments, the two terminal nucleotides on the 3′ end of the antisense strand of the RNAi oligonucleotide herein are not complementary to the target mRNA. In some embodiments, two terminal nucleotides on each 3′ end of the RNAi oligonucleotide are GG. Typically, one or both of the two terminal GG nucleotides on each 3′ end of the RNAi oligonucleotide is not complementary with the target mRNA. In some embodiments, two terminal nucleotides on each 3′ end of the oligonucleotide are GG. In some embodiments, one or both of the two terminal GG nucleotides on each 3′ end of the RNAi oligonucleotide is not complementary with the target mRNA. In some embodiments, the RNAi oligonucleotide comprises a targeting sequence or a region of complementary that is complementary to a contiguous sequence of nucleotides of any one of SEQ ID NOs: 912-1295, wherein the two terminal nucleotides on the 3′ end of the antisense strand of the oligonucleotide herein comprises an unpaired GG. In some embodiments, the RNAi oligonucleotide comprises an antisense strand comprising a nucleotide sequence selected from SEQ ID NOs: 1296-1679, wherein the two terminal nucleotides on the 3′ end of the antisense strand of the RNAi oligonucleotide comprises an unpaired GG. In some embodiments, the RNAi oligonucleotide comprises a sense strand comprising a nucleotide sequence selected from SEQ ID NOs: 912-1295 and antisense strand comprising a nucleotide sequence selected from SEQ ID NOs: 1296-1679, wherein the two terminal nucleotides on the 3′ end of the antisense strand of the RNAi oligonucleotide comprises an unpaired GG.

In some embodiments, there is one or more (e.g., 1, 2, 3, 4, or 5) mismatch(s) between a sense strand and an antisense strand comprising the RNAi oligonucleotide. If there is more than one mismatch between the sense and antisense strands, they may be positioned consecutively (e.g., 2, 3, or more in a row), or interspersed throughout the region of complementarity. In some embodiments, the 3′ end of the sense strand contains one or more mismatches. In one embodiment, two mismatches are incorporated at the 3′ end of the sense strand. In some embodiments, base mismatches, or destabilization of segments at the 3′ end of the sense strand of the RNAi oligonucleotide improves or increases the potency of the oligonucleotide.

In some embodiments, the RNAi oligonucleotide comprises a sense strand and an antisense strand comprising nucleotide sequences selected from:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
s) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
w) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively;
ii) SEQ ID NOs: 803 and 838, respectively; and
jj) SEQ ID NOs: 1681 and 815, respectively, wherein there is one or more (e.g., 1, 2, 3, 4, or 5) mismatch(s) between the sense and antisense strands.

In some embodiments, the RNAi oligonucleotide comprises a sense strand and an antisense strand comprising nucleotide sequences selected from:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively; and
k) SEQ ID NOs: 1681 and 815, respectively, wherein there is one or more (e.g., 1, 2, 3, 4, or 5) mismatch(s) between the sense and antisense strands.

In some embodiments, the RNAi oligonucleotide comprises a sense strand and an antisense strand comprising sequence selected from:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively;
f) SEQ ID NOs: 803 and 838, respectively; and
g) SEQ ID NOs: 1681 and 815, respectively, wherein there is one or more (e.g., 1, 2, 3, 4, or 5) mismatch(s) between the sense and antisense strands.

### Antisense Strands

In some embodiments, an antisense strand of an oligonucleotide herein (e.g., a RNAi oligonucleotide) is referred to as a “guide strand.” The antisense strand engages with RISC and binds to an Argonaute protein such as Ago2, or engages with or binds to one or more similar factors, and directs silencing of a target gene. In some embodiments, a sense strand complementary to a guide strand is referred to as a “passenger strand.”

In some embodiments, an oligonucleotide comprises an antisense strand of up to about 50 nucleotides in length (e.g., up to 50, up to 40, up to 35, up to 30, up to 27, up to 25, up to 21, up to 19, up to 17, up to 15, or up to 12 nucleotides in length). In some embodiments, the oligonucleotide comprises an antisense strand of at least about 12 nucleotides in length (e.g., at least 12, at least 15, at least 19, at least 21, at least 22, at least 25, at least 27, at least 30, at least 35, or at least 38 nucleotides in length). In some embodiments, the oligonucleotide comprises an antisense strand in a range of about 12 to about 40 (e.g., 12 to 40, 12 to 36, 12 to 32, 12 to 28, 15 to 40, 15 to 36, 15 to 32, 15 to 30, 15 to 28, 17 to 22, 17 to 25, 19 to 27, 19 to 30, 20 to 40, 22 to 40, 25 to 40, or 32 to 40) nucleotides in length. In some embodiments, the oligonucleotide comprises an antisense of about 15 to about 30 nucleotides in length. In some embodiments, an antisense strand of any one of the oligonucleotides disclosed herein is 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the oligonucleotide comprises an antisense strand of 22 nucleotides in length.

In some embodiments, an oligonucleotide for targeting MAPT comprises an antisense strand comprising or consisting of a sequence as set forth in any one of SEQ ID NOs: 1296-1679. In some embodiments, the oligonucleotide comprises an antisense strand comprising at least about 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 1296-1679. In some embodiments, the oligonucleotide for targeting MAPT comprises an antisense strand comprising or consisting of a sequence as set forth in any one of SEQ ID NOs: 385-768. In some embodiments, the oligonucleotide comprises an antisense strand comprising at least about 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 385-768. In some embodiments, the oligonucleotide for targeting MAPT comprises an antisense strand comprising or consisting of a sequence as set forth in any one of SEQ ID NOs: 804-838. In some embodiments, the oligonucleotide comprises an antisense strand comprising at least about 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 804-838. In some embodiments, the oligonucleotide for targeting MAPT comprises an antisense strand comprising or consisting of a sequence as set forth in any one of SEQ ID NOs: 1509, 1511, 1514, 1403, 1415, 1428, 1448, 1449, 1451, 1467, 1299, 1479, 1480, 1486, 1494, 1307, 1309, 1409, 1423, 1433, 1445, 1454, 1456, 1459, 1465, 1492, 1495, 1498, 1503, 1504, 1505, 1506, 1507, 1508, and 1308. In some embodiments, the oligonucleotide comprises an antisense strand comprising at least about 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 1509, 1511, 1514, 1403, 1415, 1428, 1448, 1449, 1451, 1467, 1299, 1479, 1480, 1486, 1494, 1307, 1309, 1409, 1423, 1433, 1445, 1454, 1456, 1459, 1465, 1492, 1495, 1498, 1503, 1504, 1505, 1506, 1507, 1508, and 1308. In some embodiments, the oligonucleotide for targeting MAPT comprises an antisense strand comprising or consisting of a sequence as set forth in any one of SEQ ID NOs: 806, 811, 815, 816, 817, 825, 830, 833, 834, and 838. In some embodiments, the oligonucleotide comprises an antisense strand comprising at least about 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 806, 811, 815, 816, 817, 825, 830, 833, 834, and 838. In some embodiments, the oligonucleotide for targeting MAPT comprises an antisense strand comprising or consisting of a sequence as set forth in any one of SEQ ID NOs: 806, 815, 816, 833, 834, and 838. In some embodiments, the oligonucleotide comprises an antisense strand comprising at least about 12 (e.g., at least 12, at least 13, at least 14, at least 15, at least 16, at least 17, at least 18, at least 19, at least 20, at least 21, at least 22, or at least 23) contiguous nucleotides of a sequence as set forth in any one of SEQ ID NOs: 806, 815, 816, 833, 834, and 838.

### Sense Strands

In some embodiments, an oligonucleotide (e.g., a RNAi oligonucleotide) for targeting MAPT mRNA comprises a sense strand comprising or consisting of a sequence as set forth in in any one of SEQ ID NOs: 912-1295. In some embodiments, the oligonucleotide has a sense strand that comprise at least about 12 (e.g., at least 13, at least 14, at least 15, at least 16, at least 17, at least 18, at least 19, at least 20, at least 21, at least 22, or at least 23) contiguous nucleotides of a sequence as set forth in in any one of SEQ ID NOs: 912-1295. In some embodiments, the oligonucleotide comprises a sense strand sequence a set forth in any one of SEQ ID NOs: 1-384. In some embodiments, the oligonucleotide has a sense strand that comprise at least about 12 (e.g., at least 13, at least 14, at least 15, at least 16, at least 17, at least 18, at least 19, at least 20, at least 21, at least 22, or at least 23) contiguous nucleotides of a sequence as set forth in in any one of SEQ ID NOs: 1-384. In some embodiments, the oligonucleotide comprises a sense strand sequence as set forth in any one of SEQ ID NOs: 769-803. In some embodiments, the oligonucleotide comprises a sense strand sequence as set forth in any one of SEQ ID NOs: 769-803 and 1681. In some embodiments, the oligonucleotide comprises the sense strand sequence as set forth in SEQ ID NO: 1681. In some embodiments, the oligonucleotide has a sense strand comprised of least about 12 (e.g., at least 13, at least 14, at least 15, at least 16, at least 17, at least 18, at least 19, at least 20, at least 21, at least 22, or at least 23) contiguous nucleotides of a sequence as set forth in any one of SEQ ID NOs: 769-803. In some embodiments, the oligonucleotide comprises a sense strand sequence as set forth in any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, and 924. In some embodiments, the oligonucleotide has a sense strand comprised of least about 12 (e.g., at least 13, at least 14, at least 15, at least 16, at least 17, at least 18, at least 19, at least 20, at least 21, at least 22, or at least 23) contiguous nucleotides of a sequence as set forth in any one of SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, 1124, and 924. In some embodiments, the oligonucleotide comprises a sense strand sequence as set forth in any one of SEQ ID NOs: 771, 776, 780, 781, 782, 790, 795, 798, 799, and 803. In some embodiments, the oligonucleotide has a sense strand that comprise at least about 12 (e.g., at least 13, at least 14, at least 15, at least 16, at least 17, at least 18, at least 19, at least 20, at least 21, at least 22, or at least 23) contiguous nucleotides of a sequence as set forth in any one of SEQ ID NOs: 771, 776, 780, 781, 782, 790, 795, 798, 799, and 803. In some embodiments, the oligonucleotide comprises a sense strand sequence as set forth in any one of SEQ ID NOs: 771, 780, 781, 798, 799, and 803. In some embodiments, the oligonucleotide has a sense strand that comprise at least about 12 (e.g., at least 13, at least 14, at least 15, at least 16, at least 17, at least 18, at least 19, at least 20, at least 21, at least 22, or at least 23) contiguous nucleotides of a sequence as set forth in any one of SEQ ID NOs: 771, 780, 781, 798, 799, and 803.

In some embodiments, the oligonucleotide comprises a sense strand sequence as set forth in any one of SEQ ID NOs: 771, 776, 780, 781, 782, 790, 795, 798, 799, 803, and 1681. In some embodiments, the oligonucleotide has a sense strand that comprises at least about 12 (e.g., at least 13, at least 14, at least 15, at least 16, at least 17, at least 18, at least 19, at least 20, at least 21, at least 22, or at least 23) contiguous nucleotides of a sequence as set forth in any one of SEQ ID NOs: 771, 776, 780, 781, 782, 790, 795, 798, 799, 803, and 1681. In some embodiments, the oligonucleotide comprises a sense strand sequence as set forth in any one of SEQ ID NOs: 771, 780, 781, 798, 799, 803, and 1681. In some embodiments, the oligonucleotide has a sense strand that comprise at least about 12 (e.g., at least 13, at least 14, at least 15, at least 16, at least 17, at least 18, at least 19, at least 20, at least 21, at least 22, or at least 23) contiguous nucleotides of a sequence as set forth in any one of SEQ ID NOs: 771, 780, 781, 798, 799, 803, and 1681.

In some embodiments, the oligonucleotide comprises a sense strand of up to about 50 nucleotides in length (e.g., up to 50, up to 40, up to 36, up to 30, up to 27, up to 25, up to 21, up to 19, up to 17, or up to 12 nucleotides in length). In some embodiments, the oligonucleotide may have a sense strand of at least about 12 nucleotides in length (e.g., at least 12, at least 15, at least 19, at least 21, at least 25, at least 27, at least 30, at least 36, or at least 38 nucleotides in length). In some embodiments, the oligonucleotide may have a sense strand in a range of about 12 to about 50 (e.g., 12 to 50, 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, the oligonucleotide comprises a sense strand about 15 to about 50 nucleotides in length. In some embodiments, the oligonucleotide comprises a sense strand 18 to 36 nucleotides in length. In some embodiments, the oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the oligonucleotide comprises a sense strand of 36 nucleotides in length.

In some embodiments, the oligonucleotide comprises a sense strand comprising a stem-loop structure at the 3′ end of the sense strand. In some embodiments, the stem-loop is formed by intrastrand base pairing. In some embodiments, the sense strand comprises a stem-loop structure at its 5′ end. In some embodiments, the stem of the stem-loop comprises a duplex of 2, 3, 4, 5, 6, 7, 8, 9, 10, 11, 12, 13, or 14 nucleotides in length. In some embodiments, the stem of the stem-loop comprises a duplex of 2 nucleotides in length. In some embodiments, the stem of the stem-loop comprises a duplex of 3 nucleotides in length. In some embodiments, the stem of the stem-loop comprises a duplex of 4 nucleotides in length. In some embodiments, the stem of the stem-loop comprises a duplex of 5 nucleotides in length. In some embodiments, the stem of the stem-loop comprises a duplex of 6 nucleotides in length. In some embodiments, the stem of the stem-loop comprises a duplex of 7 nucleotides in length. In some embodiments, the stem of the stem-loop comprises a duplex of 8 nucleotides in length. In some embodiments, the stem of the stem-loop comprises a duplex of 9 nucleotides in length. In some embodiments, the stem of the stem-loop comprises a duplex of 10 nucleotides in length. In some embodiments, the stem of the stem-loop comprises a duplex of 11 nucleotides in length. In some embodiments, the stem of the stem-loop comprises a duplex of 12 nucleotides in length. In some embodiments, the stem of the stem-loop comprises a duplex of 13 nucleotides in length. In some embodiments, the stem of the stem-loop comprises a duplex of 14 nucleotides in length.

In some embodiments, a stem-loop provides oligonucleotide protection against degradation (e.g., enzymatic degradation), facilitates or improves targeting and/or delivery to a target cell, tissue, or organ (e.g., the liver or brain), or both. For example, in some embodiments, the loop of a stem-loop provides nucleotides comprising one or more modifications that facilitate, improve, or increase targeting to a target mRNA (e.g., a MAPT mRNA), inhibition of target gene expression (e.g., MAPT gene expression), and/or delivery to a target cell, tissue, or organ (e.g., the CNS), or a combination thereof. In some embodiments, the stem-loop itself or modification(s) to the stem-loop do not substantially affect the inherent gene expression inhibition activity of the oligonucleotide, but facilitates, improves, or increases stability (e.g., provides protection against degradation) and/or delivery of the oligonucleotide to a target cell, tissue, or organ (e.g., the CNS). In certain embodiments, the oligonucleotide comprises a sense strand comprising (e.g., at its 3′ end) a stem-loop set forth as: S1-L-S2, in which S1 is complementary to S2, and in which loop (L) forms a ss loop between S1 and S2 of up to about 10 nucleotides in length (e.g., 3, 4, 5, 6, 7, 8, 9, or 10 nucleotides in length). In some embodiments, the L is 3 nucleotides in length. In some embodiments, the L is 4 nucleotides in length. In some embodiments, the L is 5 nucleotides in length. In some embodiments, the L is 6 nucleotides in length. In some embodiments, the L is 7 nucleotides in length. In some embodiments, the L is 8 nucleotides in length. In some embodiments, the L is 9 nucleotides in length. In some embodiments, the L is 10 nucleotides in length.

In some embodiments, the oligonucleotide comprises a targeting sequence or a region of complementary that is complementary to a contiguous sequence of nucleotides of any one of SEQ ID NOs: 912-1295, and the oligonucleotide comprises a sense strand comprising (e.g., at its 3′ end) a stem-loop set forth as: S1-L-S2, in which S1 is complementary to S2, and in which L forms a ss loop between S1 and S2 of up to about 10 nucleotides in length (e.g., 3, 4, 5, 6, 7, 8, 9, or 10 nucleotides in length). In some embodiments, the oligonucleotide comprises a targeting sequence or a region of complementary that is complementary to a contiguous sequence of nucleotides of any one of SEQ ID NOs: 912-1295, and the oligonucleotide comprises a sense strand comprising (e.g., at its 3′ end) a stem-loop set forth as: S1-L-S2, in which S1 is complementary to S2, and in which L forms a ss loop between S1 and S2 of 4 nucleotides in length (i.e., a tetraL).

In some embodiments, the tetraL comprises the sequence 5′-GAAA-3′. In some embodiments, the stem-loop comprises the sequence 5′-GCAGCCGAAAGGCUGC-3′ (SEQ ID NO: 1680).

In some embodiments, the L of a stem-loop having the structure S1-L-S2 as described above is a triL. In some embodiments, the oligonucleotide comprises a targeting sequence or a region of complementary that is complementary to a contiguous sequence of nucleotides of any one of SEQ ID NOs: 912-1295 and a triL. In some embodiments, the triL comprises ribonucleotides, deoxyribonucleotides, modified nucleotides, delivery ligands, and combinations thereof.

In some embodiments, the L of a stem-loop having the structure S1-L-S2 as described above is a tetraL as described in U.S. Pat. No. 10,131,912, incorporated herein by reference (e.g., within a nicked tetraL structure). In some embodiments, the oligonucleotide comprises a targeting sequence or a region of complementary that is complementary to a contiguous sequence of nucleotides of any one of SEQ ID NOs: 912-1295 and a tetraL. In some embodiments, the tetraL comprises ribonucleotides, deoxyribonucleotides, modified nucleotides, delivery ligands, and combinations thereof.

### Duplex Length

In some embodiments, a duplex is formed between a sense and antisense strand and is at least about 12 (e.g., at least 12, at least 13, at least 14, at least 15, at least 16, at least 17, at least 18, at least 19, at least 20, or at least 21) nucleotides in length. In some embodiments, the duplex formed between the sense and antisense strands is in the range of about 12 to about 30 nucleotides in length (e.g., 12 to 30, 12 to 27, 12 to 22, 15 to 25, 18 to 30, 18 to 22, 18 to 25, 18 to 27, 18 to 30, 19 to 30, or 21 to 30 nucleotides in length). In some embodiments, the duplex formed between the sense and antisense strands is 12, 13, 14, 15, 16, 17, 18, 19, 29, 21, 22, 23, 24, 25, 26, 27, 28, 29, or 30 nucleotides in length. In some embodiments, the duplex formed between the sense and antisense strands is 12 nucleotides in length. In some embodiments, the duplex formed between the sense and antisense strands is 13 nucleotides in length. In some embodiments, the duplex formed between the sense and antisense strands is 14 nucleotides in length. In some embodiments, the duplex formed between the sense and antisense strands is 15 nucleotides in length. In some embodiments, the duplex formed between the sense and antisense strands is 16 nucleotides in length. In some embodiments, the duplex formed between the sense and antisense strands is 17 nucleotides in length. In some embodiments, the duplex formed between the sense and antisense strands is 18 nucleotides in length. In some embodiments, the duplex formed between the sense and antisense strands is 19 nucleotides in length. In some embodiments, the duplex formed between the sense and antisense strands is 20 nucleotides in length. In some embodiments, the duplex formed between the sense and antisense strands is 21 nucleotides in length. In some embodiments, the duplex formed between the sense and antisense strands is 22 nucleotides in length. In some embodiments, the duplex formed between the sense and antisense strands is 23 nucleotides in length. In some embodiments, the duplex formed between the sense and antisense strands is 24 nucleotides in length. In some embodiments, the duplex formed between the sense and antisense strands is 25 nucleotides in length. In some embodiments, the duplex formed between the sense and antisense strands is 26 nucleotides in length. In some embodiments, the duplex formed between the sense and antisense strands is 27 nucleotides in length. In some embodiments, the duplex formed between the sense and antisense strands is 28 nucleotides in length. In some embodiments, the duplex formed between the sense and antisense strands is 29 nucleotides in length. In some embodiments, the duplex formed between the sense and antisense strands is 30 nucleotides in length. In some embodiments, the duplex formed between the sense and antisense strands does not span the entire length of the sense strand and/or antisense strand. In some embodiments, the duplex between the sense and antisense strand spans the entire length of either the sense or antisense strands. In some embodiments, the duplex between the sense and antisense strands spans the entire length of both the sense strand and the antisense strand.

In some embodiments, the duplex between the sense and antisense strands spans the entire length of both the sense strand and the antisense strand. In some embodiments, the sense and antisense strands of the oligonucleotide comprise nucleotides sequences selected from the group consisting of:
a) SEQ ID NO: 769 and 804, respectively;
b) SEQ ID NO: 770 and 805, respectively;
c) SEQ ID NO: 771 and 806, respectively;
d) SEQ ID NO: 772 and 807, respectively;
e) SEQ ID NO: 773 and 808, respectively;
f) SEQ ID NO: 774 and 809, respectively;
g) SEQ ID NO: 775 and 810, respectively;
h) SEQ ID NO: 776 and 811, respectively;
i) SEQ ID NO: 777 and 812, respectively;
j) SEQ ID NO: 778 and 813, respectively;
k) SEQ ID NO: 779 and 814, respectively;
l) SEQ ID NO: 780 and 815, respectively;
m) SEQ ID NO: 781 and 816, respectively;
n) SEQ ID NO: 782 and 817, respectively;
o) SEQ ID NO: 783 and 818, respectively;
p) SEQ ID NO: 784 and 819, respectively;
q) SEQ ID NO: 785 and 820, respectively;
r) SEQ ID NO: 786 and 821, respectively;
S) SEQ ID NO: 787 and 822, respectively;
t) SEQ ID NO: 788 and 823, respectively;
u) SEQ ID NO: 789 and 824, respectively;
v) SEQ ID NO: 790 and 825, respectively;
w) SEQ ID NO: 791 and 826, respectively;
x) SEQ ID NO: 792 and 827, respectively;
y) SEQ ID NO: 793 and 828, respectively;
z) SEQ ID NO: 794 and 829, respectively;
aa) SEQ ID NO: 795 and 830, respectively;
bb) SEQ ID NO: 796 and 831, respectively;
cc) SEQ ID NO: 797 and 832, respectively;
dd) SEQ ID NO: 798 and 833, respectively;
ee) SEQ ID NO: 799 and 834, respectively;
ff) SEQ ID NO: 800 and 835, respectively;
gg) SEQ ID NO: 801 and 836, respectively;
hh) SEQ ID NO: 802 and 837, respectively;
ii) SEQ ID NO: 803 and 838, respectively; and
jj) SEQ ID NOs: 1681 and 815, respectively, wherein the duplex formed between the sense and antisense strand is in the range of about 12 to about 30 nucleotides in length (e.g., 12 to 30, 12 to 27, 12 to 22, 15 to 25, 18 to 30, 18 to 22, 18 to 25, 18 to 27, 18 to 30, 19 to 30, or 21 to 30 nucleotides in length).

In some embodiments, the duplex between the sense and antisense strands spans the entire length of both the sense strand and the antisense strand. In some embodiments, the sense and antisense strands of the oligonucleotide comprise nucleotides sequences selected from the group consisting of:
a) SEQ ID NO: 771 and 806, respectively;
b) SEQ ID NO: 776 and 811, respectively;
c) SEQ ID NO: 780 and 815, respectively;
d) SEQ ID NO: 781 and 816, respectively;
e) SEQ ID NO: 782 and 817, respectively;
f) SEQ ID NO: 790 and 825, respectively;
g) SEQ ID NO: 795 and 830, respectively;
h) SEQ ID NO: 798 and 833, respectively;
i) SEQ ID NO: 799 and 834, respectively;
j) SEQ ID NO: 803 and 838, respectively; and
k) SEQ ID NOs: 1681 and 815, respectively, wherein the duplex formed between the sense and antisense strand is in the range of about 12 to about 30 nucleotides in length (e.g., 12 to 30, 12 to 27, 12 to 22, 15 to 25, 18 to 30, 18 to 22, 18 to 25, 18 to 27, 18 to 30, 19 to 30, or 21 to 30 nucleotides in length).

In some embodiments, the duplex between the sense and antisense strands spans the entire length of both the sense strand and the antisense strand. In some embodiments, the sense and antisense strands of the oligonucleotide comprise nucleotides sequences selected from the group consisting of:
a) SEQ ID NO: 771 and 806, respectively;
b) SEQ ID NO: 780 and 815, respectively;
c) SEQ ID NO: 781 and 816, respectively;
d) SEQ ID NO: 798 and 833, respectively;
e) SEQ ID NO: 799 and 834, respectively;
f) SEQ ID NO: 803 and 838, respectively; and
g) SEQ ID NOs: 1681 and 815, respectively, wherein the duplex formed between the sense and antisense strand is in the range of about 12 to about 30 nucleotides in length (e.g., 12 to 30, 12 to 27, 12 to 22, 15 to 25, 18 to 30, 18 to 22, 18 to 25, 18 to 27, 18 to 30, 19 to 30, or 21 to 30 nucleotides in length).

### Oligonucleotide Termini

In some embodiments, an oligonucleotide herein (e.g., a RNAi oligonucleotide) comprises a sense strand and an antisense strand, wherein termini of either or both strands comprise a blunt end. In some embodiments, the oligonucleotide comprises sense and antisense strands that are separate strands that form an asymmetric duplex region having an overhang at the 3′ terminus of the antisense strand. In some embodiments, the oligonucleotide comprises a sense strand and an antisense strand, wherein termini of either or both strands comprise an overhang comprising one or more nucleotides. In some embodiments, the one or more nucleotides comprising the overhang are unpaired nucleotides. In some embodiments, the oligonucleotide comprises a sense strand and an antisense strand, wherein a 3′ terminus of the sense strand and a 5′ terminus of the antisense strand comprise a blunt end. In some embodiments, the oligonucleotide comprises a sense strand and an antisense strand, wherein a 5′ terminus of the sense strand and a 3′ terminus of the antisense strand comprise a blunt end.

In some embodiments, the oligonucleotide comprises a sense strand and an antisense strand, wherein a 3′ terminus of either or both strands comprises a 3′ overhang comprising one or more nucleotides. In some embodiments, the oligonucleotide comprises a sense strand and an antisense strand, wherein the sense strand comprises a 3′ overhang comprising one or more nucleotides. In some embodiments, the oligonucleotide comprises a sense strand and an antisense strand, wherein the antisense strand comprises a 3′ overhang comprising one or more nucleotides.

In some embodiments, the oligonucleotide comprises a sense strand and an antisense strand, wherein both the sense strand and the antisense strand comprise a 3′ overhang comprising one or more nucleotides.

In some embodiments, the 3′ overhang is about 1 to about 20 nucleotides in length (e.g., about 1, 2, 3, 4, 5, 6, 7, 8, 9, 10, 11, 12, 13, 14, 15, 16, 17, 18, 19, or 20 nucleotides in length). In some embodiments, the 3′ overhang is 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2 nucleotides in length). In some embodiments, the 3′ overhang is 1 nucleotide in length. In some embodiments, the 3′ overhang is 2 nucleotides in length. In some embodiments, the 3′ overhang is 3 nucleotides in length. In some embodiments, the 3′ overhang is 4 nucleotides in length. In some embodiments, the 3′ overhang is 5 nucleotides in length. In some embodiments, the 3′ overhang is 6 nucleotides in length. In some embodiments, the 3′ overhang is 7 nucleotides in length. In some embodiments, the 3′-overhang is 8 nucleotides in length. In some embodiments, the 3′ overhang is 9 nucleotides in length. In some embodiments, the 3′ overhang is 10 nucleotides in length. In some embodiments, the 3′ overhang is 11 nucleotides in length. In some embodiments, the 3′ overhang is 12 nucleotides in length. In some embodiments, the 3′ overhang is 13 nucleotides in length. In some embodiments, the 3′ overhang is 14 nucleotides in length. In some embodiments, the 3′ overhang is 15 nucleotides in length. In some embodiments, the 3′ overhang is 16 nucleotides in length. In some embodiments, the 3′ overhang is 17 nucleotides in length. In some embodiments, the 3′ overhang is 18 nucleotides in length. In some embodiments, the 3′ overhang is 19 nucleotides in length. In some embodiments, the 3′ overhang is 20 nucleotides in length.

In some embodiments, the oligonucleotide comprises a sense strand and an antisense strand, wherein the antisense strand comprises a 3′ overhang, wherein the sense and antisense strands of the oligonucleotide comprise nucleotides sequences selected from the group consisting of:
a) SEQ ID NO: 769 and 804, respectively;
b) SEQ ID NO: 770 and 805, respectively;
c) SEQ ID NO: 771 and 806, respectively;
d) SEQ ID NO: 772 and 807, respectively;
e) SEQ ID NO: 773 and 808, respectively;
f) SEQ ID NO: 774 and 809, respectively;
g) SEQ ID NO: 775 and 810, respectively;
h) SEQ ID NO: 776 and 811, respectively;
i) SEQ ID NO: 777 and 812, respectively;
j) SEQ ID NO: 778 and 813, respectively;
k) SEQ ID NO: 779 and 814, respectively;
l) SEQ ID NO: 780 and 815, respectively;
m) SEQ ID NO: 781 and 816, respectively;
n) SEQ ID NO: 782 and 817, respectively;
o) SEQ ID NO: 783 and 818, respectively;
p) SEQ ID NO: 784 and 819, respectively;
q) SEQ ID NO: 785 and 820, respectively;
r) SEQ ID NO: 786 and 821, respectively;
S) SEQ ID NO: 787 and 822, respectively;
t) SEQ ID NO: 788 and 823, respectively;
u) SEQ ID NO: 789 and 824, respectively;
v) SEQ ID NO: 790 and 825, respectively;
W) SEQ ID NO: 791 and 826, respectively;
x) SEQ ID NO: 792 and 827, respectively;
y) SEQ ID NO: 793 and 828, respectively;
z) SEQ ID NO: 794 and 829, respectively;
aa) SEQ ID NO: 795 and 830, respectively;
bb) SEQ ID NO: 796 and 831, respectively;
cc) SEQ ID NO: 797 and 832, respectively;
dd) SEQ ID NO: 798 and 833, respectively;
ee) SEQ ID NO: 799 and 834, respectively;
ff) SEQ ID NO: 800 and 835, respectively;
gg) SEQ ID NO: 801 and 836, respectively;
hh) SEQ ID NO: 802 and 837, respectively;
ii) SEQ ID NO: 803 and 838, respectively; and
jj) SEQ ID NOs: 1681 and 815, respectively, wherein the antisense strand comprises the 3′ overhang about 1 to about 20 nucleotides in length (e.g., about 1, 2, 3, 4, 5, 6, 7, 8, 9, 10, 11, 12, 13, 14, 15, 16, 17, 18, 19, or 20 nucleotides in length), optionally wherein the 3′ overhang is 2 nucleotides in length.

In some embodiments, the oligonucleotide comprises a sense strand and an antisense strand, wherein the antisense strand comprises the 3′ overhang, wherein the sense and antisense strands of the oligonucleotide comprise nucleotides sequences selected from the group consisting of:
a) SEQ ID NO: 771 and 806, respectively;
b) SEQ ID NO: 776 and 811, respectively;
c) SEQ ID NO: 780 and 815, respectively;
d) SEQ ID NO: 781 and 816, respectively;
e) SEQ ID NO: 782 and 817, respectively;
f) SEQ ID NO: 790 and 825, respectively;
g) SEQ ID NO: 795 and 830, respectively;
h) SEQ ID NO: 798 and 833, respectively;
i) SEQ ID NO: 799 and 834, respectively;
j) SEQ ID NO: 803 and 838, respectively; and
k) SEQ ID NOs: 1681 and 815, respectively, wherein the antisense strand comprises the 3′ overhang about 1 to about 20 nucleotides in length (e.g., about 1, 2, 3, 4, 5, 6, 7, 8, 9, 10, 11, 12, 13, 14, 15, 16, 17, 18, 19, or 20 nucleotides in length), optionally wherein the 3′ overhang is 2 nucleotides in length.

In some embodiments, the oligonucleotide comprises a sense strand and an antisense strand, wherein the antisense strand comprises the 3′ overhang, wherein the sense and antisense strands of the oligonucleotide comprise nucleotides sequences selected from the group consisting of:
a) SEQ ID NO: 771 and 806, respectively;
b) SEQ ID NO: 780 and 815, respectively;
c) SEQ ID NO: 781 and 816, respectively;
d) SEQ ID NO: 798 and 833, respectively;
e) SEQ ID NO: 799 and 834, respectively;
f) SEQ ID NO: 803 and 838, respectively; and
g) SEQ ID NOs: 1681 and 815, respectively, wherein the antisense strand comprises a 3′ overhang about 1 to about 20 nucleotides in length (e.g., about 1, 2, 3, 4, 5, 6, 7, 8, 9, 10, 11, 12, 13, 14, 15, 16, 17, 18, 19, or 20 nucleotides in length), optionally wherein the 3′ overhang is 2 nucleotides in length.

In some embodiments, the oligonucleotide comprises a sense strand and an antisense strand, wherein the sense strand comprises a 5′ overhang comprising one or more nucleotides.

In some embodiments, the oligonucleotide comprises a sense strand and an antisense strand, wherein the sense and antisense strands of the oligonucleotide comprise nucleotides sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
s) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
W) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively;
ii) SEQ ID NOs: 803 and 838, respectively; and
jj) SEQ ID NOs: 1681 and 815, respectively, wherein the antisense strand comprises a 3′ overhang about 1 to about 20 nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′ overhang is 2 nucleotides in length.

In some embodiments, the oligonucleotide comprises a sense strand and an antisense strand, wherein the sense and antisense strands of the oligonucleotide comprise nucleotides sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively; and
k) SEQ ID NOs: 1681 and 815, respectively, wherein the antisense strand comprises the 3′ overhang about 1 to about 20 nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′ overhang is 2 nucleotides in length.

In some embodiments, the oligonucleotide comprises a sense strand and an antisense strand, wherein the sense and antisense strands of the oligonucleotide comprise nucleotides sequences selected from the group consisting of:
a) SEQ ID NO: 771 and 806, respectively;
b) SEQ ID NO: 780 and 815, respectively;
c) SEQ ID NO: 781 and 816, respectively;
d) SEQ ID NO: 798 and 833, respectively;
e) SEQ ID NO: 799 and 834, respectively;
f) SEQ ID NO: 803 and 838, respectively; and
g) SEQ ID NOs: 1681 and 815, respectively, wherein the antisense strand comprises the 3′ overhang about 1 to about 20 nucleotides in length (e.g., about 1, 2, 3, 4, 5, 6, 7, 8, 9, 10, 11, 12, 13, 14, 15, 16, 17, 18, 19, or about 20 nucleotides in length), optionally wherein the 3′ overhang is 2 nucleotides in length.

In some embodiments, one or more (e.g., 2, 3, 4, 5, or more) nucleotides comprising the 3′ terminus or 5′ terminus of the sense and/or antisense strand are modified. For example, in some embodiments, one or two terminal nucleotides of the 3′ terminus of the antisense strand are modified. In some embodiments, the last nucleotide at the 3′ terminus of an antisense strand is modified, for example, comprises 2′ modification (e.g., a 2′-OMe). In some embodiments, the last one or two terminal nucleotides at the 3′ terminus of an antisense strand are complementary with the target. In some embodiments, the last one or two nucleotides at the 3′ terminus of the antisense strand are not complementary with the target.

In some embodiments, the oligonucleotide comprises a sense strand and an antisense strand, wherein the 3′ terminus of the sense strand comprises a step-loop and the 3′ terminus of the antisense strand comprises the 3′ overhang. In some embodiments, the oligonucleotide comprises a sense strand and an antisense strand that form a nicked tetraL structure, wherein the 3′ terminus of the sense strand comprises the stem-loop, wherein the loop is a tetraL, and wherein the 3′ terminus of the antisense strand comprises the 3′ overhang described herein. In some embodiments, the 3′ overhang is 2 nucleotides in length. In some embodiments, the 2 nucleotides comprising the 3′ overhang both comprise guanine (G) nucleobases. Typically, one or both of the nucleotides comprising the 3′ overhang of the antisense strand are not complementary with the target mRNA.

### Oligonucleotide Modifications

In some embodiments, the oligonucleotide (e.g., a RNAi oligonucleotide) comprises a modification. Oligonucleotides may be modified in various ways to improve or control specificity, stability, delivery, bioavailability, resistance from nuclease degradation, immunogenicity, base-pairing properties, RNA distribution and cellular uptake, and other features relevant to therapeutic research use.

In some embodiments, the modification is a modified sugar. In some embodiments, the modification is a 5′ terminal phosphate group. In some embodiments, the modification is a modified internucleoside linkage. In some embodiments, the modification is a modified base. In some embodiments, the modification is a reversible modification. In some embodiments, the oligonucleotide may comprise any one of the modifications described herein or any combination thereof. For example, in some embodiments, the oligonucleotide comprises at least one modified sugar, a 5′ terminal phosphate group, at least one modified internucleoside linkage, at least one modified base, and at least one reversible modification.

In some embodiments, the oligonucleotide comprises at least one modified sugar, a 5′ terminal phosphate group, at least one modified internucleotide linkage, and at least one modified base. In some embodiments, the sense and antisense strands of the oligonucleotide comprise nucleotides sequences selected from the group consisting of:
a) SEQ ID NO: 769 and 804, respectively;
b) SEQ ID NO: 770 and 805, respectively;
c) SEQ ID NO: 771 and 806, respectively;
d) SEQ ID NO: 772 and 807, respectively;
e) SEQ ID NO: 773 and 808, respectively;
f) SEQ ID NO: 774 and 809, respectively;
g) SEQ ID NO: 775 and 810, respectively;
h) SEQ ID NO: 776 and 811, respectively;
i) SEQ ID NO: 777 and 812, respectively;
j) SEQ ID NO: 778 and 813, respectively;
k) SEQ ID NO: 779 and 814, respectively;
l) SEQ ID NO: 780 and 815, respectively;
m) SEQ ID NO: 781 and 816, respectively;
n) SEQ ID NO: 782 and 817, respectively;
o) SEQ ID NO: 783 and 818, respectively;
p) SEQ ID NO: 784 and 819, respectively;
q) SEQ ID NO: 785 and 820, respectively;
r) SEQ ID NO: 786 and 821, respectively;
s) SEQ ID NO: 787 and 822, respectively;
t) SEQ ID NO: 788 and 823, respectively;
u) SEQ ID NO: 789 and 824, respectively;
V) SEQ ID NO: 790 and 825, respectively;
W) SEQ ID NO: 791 and 826, respectively;
x) SEQ ID NO: 792 and 827, respectively;
y) SEQ ID NO: 793 and 828, respectively;
z) SEQ ID NO: 794 and 829, respectively;
aa) SEQ ID NO: 795 and 830, respectively;
bb) SEQ ID NO: 796 and 831, respectively;
cc) SEQ ID NO: 797 and 832, respectively;
dd) SEQ ID NO: 798 and 833, respectively;
ee) SEQ ID NO: 799 and 834, respectively;
ff) SEQ ID NO: 800 and 835, respectively;
gg) SEQ ID NO: 801 and 836, respectively;
hh) SEQ ID NO: 802 and 837, respectively;
ii) SEQ ID NO: 803 and 838, respectively; and
jj) SEQ ID NOs: 1681 and 815, respectively, wherein the oligonucleotide comprises at least one modified sugar, a 5′ terminal phosphate group, at least one modified internucleotide linkage, and at least one modified base.

In some embodiments, the sense and antisense strands of an oligonucleotide comprise nucleotides sequences selected from the group consisting of:
a) SEQ ID NO: 771 and 806, respectively;
b) SEQ ID NO: 776 and 811, respectively;
c) SEQ ID NO: 780 and 815, respectively;
d) SEQ ID NO: 781 and 816, respectively;
e) SEQ ID NO: 782 and 817, respectively;
f) SEQ ID NO: 790 and 825, respectively;
g) SEQ ID NO: 795 and 830, respectively;
h) SEQ ID NO: 798 and 833, respectively;
i) SEQ ID NO: 799 and 834, respectively;
j) SEQ ID NO: 803 and 838, respectively; and
k) SEQ ID NOs: 1681 and 815, respectively, wherein the oligonucleotide comprises at least one modified sugar, a 5′ terminal phosphate group, at least one modified internucleotide linkage, and at least one modified base.

In some embodiments, the sense and antisense strands of an oligonucleotide comprise nucleotides sequences selected from the group consisting of:
a) SEQ ID NO: 771 and 806, respectively;
b) SEQ ID NO: 780 and 815, respectively;
c) SEQ ID NO: 781 and 816, respectively;
d) SEQ ID NO: 798 and 833, respectively;
e) SEQ ID NO: 799 and 834, respectively;
f) SEQ ID NO: 803 and 838, respectively; and
g) SEQ ID NOs: 1681 and 815, respectively, wherein the oligonucleotide comprises at least one modified sugar, a 5′ terminal phosphate group, at least one modified internucleotide linkage, and at least one modified base.

The number of modifications on the oligonucleotide and the position of those nucleotide modifications may influence the properties of an oligonucleotide. For example, oligonucleotides may be delivered in vivo by conjugating them to encompassing them in a lipid nanoparticle (LNP) or similar carrier. However, when an oligonucleotide is not protected by an LNP or similar carrier, it may be advantageous for at least some of the nucleotides to be modified. Accordingly, in some embodiments, all or substantially all of the nucleotides of the oligonucleotides are modified. In some embodiments, more than half of the nucleotides are modified. In some embodiments, less than half of the nucleotides are modified. In some embodiments, the sugar moiety of all nucleotides comprising the oligonucleotide is modified at the 2′ position. The modifications may be reversible or irreversible. In some embodiments, an oligonucleotide as disclosed herein has a number and type of modified nucleotides sufficient to cause the desired characteristics (e.g., protection from enzymatic degradation, capacity to target a desired cell after in vivo administration, and/or thermodynamic stability).

### Sugar Modifications

In some embodiments, the oligonucleotide comprises a modified sugar. In some embodiments, the modified sugar (also referred herein to a sugar analog) includes a modified deoxyribose or ribose moiety in which, for example, one or more modifications occur at the 2′, 3′, 4′, and/or 5′ carbon position of the sugar. In some embodiments, the modified sugar may also include non-natural alternative carbon structures such as those present in locked nucleic acids (“LNA”; see, e.g., Koshkin et al. (1998) TETRAHEDON 54:3607-30), unlocked nucleic acids (“UNA”; see, e.g., Snead et al. (2013) MOL. THER-NUCL. ACIDS 2: e103), and bridged nucleic acids (“BNA”; see, e.g., Imanishi & Obika (2002) CHEM COMMUN. (CAMB) 21:1653-59).

In some embodiments, a nucleotide modification in a sugar comprises a 2′-modification. In some embodiments, the 2′-modification may be 2′-O-propargyl, 2′-O-propylamin, 2′-amino, 2′-ethyl, 2′-F, EA, 2′-OMe, 2′-MOE, 2′-O-[2-(methylamino)-2-oxoethyl] (2′-O-NMA) or 2′-FANA. In some embodiments, the modification is 2′-F, 2′-OMe or 2′-MOE. In some embodiments, the modification in a sugar comprises a modification of the sugar ring, which may comprise modification of one or more carbons of the sugar ring. For example, the modification of a sugar of a nucleotide may comprise a 2′-oxygen of a sugar is linked to a 1′-carbon or 4′-carbon of the sugar, or a 2′-oxygen is linked to the 1′-carbon or 4′-carbon via an ethylene or methylene bridge. In some embodiments, the modified nucleotide has an acyclic sugar that lacks a 2′-carbon to 3′-carbon bond. In some embodiments, the modified nucleotide has a thiol group, for example, in the 4′ position of the sugar.

In some embodiments, the oligonucleotide described herein comprises at least about 1 modified nucleotide (e.g., at least 1, at least 5, at least 10, at least 15, at least 20, at least 25, at least 30, at least 35, at least 40, at least 45, at least 50, at least 55, at least 60, or more). In some embodiments, the sense strand of the RNAi oligonucleotide comprises at least about 1 modified nucleotide (e.g., at least 1, at least 5, at least 10, at least 15, at least 20, at least 25, at least 30, at least 35, or more). In some embodiments, the antisense strand of the oligonucleotide comprises at least about 1 modified nucleotide (e.g., at least 1, at least 5, at least 10, at least 15, at least 20, or more).

In some embodiments, all the nucleotides of the sense strand of the oligonucleotide are modified. In some embodiments, all the nucleotides of the antisense strand of the oligonucleotide are modified. In some embodiments, all the nucleotides of the oligonucleotide (i.e., both the sense strand and the antisense strand) are modified. In some embodiments, the modified nucleotide comprises a 2′-modification (e.g., a 2′-F or 2′-OMe, 2′-MOE, and 2′-FANA). In some embodiments, the modified nucleotide comprises a 2′-modification (e.g., a 2′-F or 2′-OMe)

In some embodiments, the disclosure provides oligonucleotides having different modification patterns. In some embodiments, the modified oligonucleotides comprise a sense strand sequence having a modification pattern as set forth in the Examples and Sequence Listing and an antisense strand having a modification pattern as set forth in the Examples and Sequence Listing.

In some embodiments, the oligonucleotide comprises an antisense strand having nucleotides that are modified with 2′-F. In some embodiments, the oligonucleotide comprises an antisense strand comprises nucleotides that are modified with 2′-F and 2′-OMe. In some embodiments, the oligonucleotide comprises a sense strand having nucleotides that are modified with 2′-F. In some embodiments, the oligonucleotide comprises a sense strand comprising nucleotides that are modified with 2′-F and 2′-OMe.

In some embodiments, the oligonucleotide comprises a sense strand with about 10-15%, 10%, 11%, 12%, 13%, 14%, or 15% of the nucleotides of the sense strand comprising a 2′-F modification. In some embodiments, the oligonucleotide comprises a sense strand with about 18-23% (e.g., 18%, 19%, 20%, 21%, 22%, or 23%) of the nucleotides of the sense strand comprising a 2′-F modification. In some embodiments, the oligonucleotide comprises a sense strand with about 38-43% (e.g., 38%, 39%, 40%, 41%, 42%, or 43%) of the nucleotides of the sense strand comprising a 2′-F modification. In some embodiments, about 11% of the nucleotides of the sense strand comprise a 2′-F modification. In some embodiments, about 22% of the nucleotides of the sense strand comprise a 2′-F modification. In some embodiments, about 40% of the nucleotides of the sense strand comprise a 2′-F modification. In some embodiments, the oligonucleotide comprises an antisense strand with about 25% to about 35% (e.g., 25%, 26%, 27%, 28%, 29%, 30%, 31%, 32%, 33%, 34%, or 35%) of the nucleotides of the antisense strand comprising a 2′-F modification. In some embodiments, about 32% of the nucleotides of the antisense strand comprise a 2′-F modification. In some embodiments, the oligonucleotide has about 15% to about 25% (e.g., 15%, 16%, 17%, 18%, 19%, 20%, 21%, 22%, 23%, 24%, or 25%) of its nucleotides comprising a 2′-F modification. In some embodiments, the oligonucleotide has about 35-45% (e.g., 35%, 36%, 37%, 38%, 39%, 40%, 41%, 42%, 43%, 44% or 45%) of its nucleotides comprising a 2′-F modification. In some embodiments, about 19% of the nucleotides in the oligonucleotide comprise a 2′-F modification. In some embodiments, about 29% of the nucleotides in the oligonucleotide comprise a 2′-F modification. In some embodiments, about 40% of the nucleotides in the oligonucleotide comprise a 2′-F modification.

In some embodiments, one or more of positions 8, 9, 10, or 11 of a 36-nucleotide sense strand are modified with a 2′—F group. In some embodiments, one or more of positions 8, 9, 10, or 11 of a sense strand comprising a stem-loop are modified with a 2′—F group. In some embodiments, the sugar moiety at each of nucleotides at positions 1-7 and 12-20 of a 36-nucleotide sense strand is modified with a 2′-OMe. In some embodiments, the sugar moiety at each of nucleotides at positions 1-7 and 12-20 of a sense strand comprising a stem-loop is modified with a 2′-OMe. In some embodiments, the sugar moiety at each of nucleotides at positions 1-7 and 12-36 in the sense strand is modified with a 2′-OMe.

In some embodiments, one or more of positions 3, 5, 8, 10, 12, 13, 15, and 17 of the sense strand are modified with a 2′-F.

In some embodiments, the antisense strand has 3 nucleotides that are modified at the 2′-position of the sugar moiety with a 2′-F. In some embodiments, the sugar moiety at positions 2, 5 and 14 and optionally up to 3 of the nucleotides at positions 1, 3, 7, and 10 of the antisense strand are modified with a 2′-F. In some embodiments, the sugar moiety at positions 2, 5, and 14 and optionally up to 3 of the nucleotides at positions 3, 4, 7, and 10 of the antisense strand are modified with a 2′-F. In other embodiments, the sugar moiety at each of the positions at positions 2, 5, and 14 of the antisense strand is modified with the 2′-F. In other embodiments, the sugar moiety at each of the positions at positions 1, 2, 5, and 14 of the antisense strand is modified with the 2′-F. In other embodiments, the sugar moiety at each of the positions at positions 2, 4, 5, and 14 of the antisense strand is modified with the 2′-F. In still other embodiments, the sugar moiety at each of the positions at positions 1, 2, 3, 5, 7, and 14 of the antisense strand is modified with the 2′-F. In other embodiments, the sugar moiety at each of the positions at positions 2, 3, 4, 5, 7, and 14 of the antisense strand is modified with the 2′-F. In yet another embodiment, the sugar moiety at each of the positions at positions 1, 2, 3, 5, 10, and 14 of the antisense strand is modified with the 2′-F. In other embodiments, the sugar moiety at each of the positions at positions 2, 3, 4, 5, 10, and 14 of the antisense strand is modified with the 2′-F. In another embodiment, the sugar moiety at each of the positions at positions 2, 3, 5, 7, 10, and 14 of the antisense strand is modified with the 2′-F. In other embodiments, the sugar moiety at each of the positions at positions 2, 3, 4, 5, 7, 10, and 14 of an antisense strand duplexed with a 36-nucleotide sense strand is modified with the 2′-F. In other embodiments, the sugar moiety at each of the positions at positions 2, 3, 4, 5, 7, 10, and 14 of an antisense strand duplexed with a sense strand comprising a stem-loop is modified with the 2′-F.

In some embodiments, the oligonucleotide comprises an antisense strand having the sugar moiety at positions 2 and 14 modified with 2′-F. In some embodiments, the oligonucleotide comprises an antisense strand having the sugar moiety at positions 2, 5, and 14 modified with 2′-F. In some embodiments, the oligonucleotide comprises an antisense strand having the sugar moiety at positions 1, 2, 5, and 14 modified with 2′-F. In some embodiments, the oligonucleotide comprises an antisense strand having the sugar moiety at positions 2, 4, 5, and 14 modified with 2′-F. In some embodiments, the oligonucleotide comprises an antisense strand having the sugar moiety at positions 1, 2, 3, 5, 7, and 14 modified with 2′-F. In some embodiments, the oligonucleotide comprises an antisense strand having the sugar moiety at positions 2, 3, 4, 5, 7, and 14 modified with 2′-F. In some embodiments, the oligonucleotide comprises an antisense strand having the sugar moiety at positions 1, 2, 3, 5, 10, and 14 modified with 2′-F. In some embodiments, the oligonucleotide comprises an antisense strand having the sugar moiety at positions 2, 3, 4, 5, 10, and 14 modified with 2′-F. In some embodiments, the oligonucleotide comprises a 36-nucleotide sense strand and an antisense strand, wherein the antisense strand comprises a sugar moiety at positions 2, 3, 4, 5, 7, 10, and 14 modified with 2′-F. In some embodiments, the oligonucleotide comprises a sense strand comprising a stem-loop and an antisense strand, wherein the antisense strand comprises a sugar moiety at positions 2, 3, 4, 5, 7, 10, and 14 modified with 2′-F. In some embodiments, the oligonucleotide comprises an antisense strand having the sugar moiety at positions 2, 3, 4, 5, 7, 10, 14, 16, and 19 modified with 2′-F.

In some embodiments, the oligonucleotide comprises an antisense strand having the sugar moiety of each of the nucleotides at positions 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 1, 2, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 2, 4, 5, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 1, 2, 3, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 2, 3, 4, 5, 7, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 1, 2, 3, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 2, 3, 4, 5, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 2, 3, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises a 36-nucleotide sense strand and an antisense strand, wherein the antisense strand comprising a sugar moiety at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA. In some embodiments, the oligonucleotide comprises a sense strand comprising a stem-loop and an antisense strand, wherein the antisense strand comprising a sugar moiety at positions 2, 3, 4, 5, 7, 10, and 14 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 2, 3, 4, 5, 7, 10, 14, 16, and 19 of the antisense strand modified with 2′-F and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-F.

In some embodiments, the oligonucleotide comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with 2′-OMe.

In some embodiments, the oligonucleotide comprises an antisense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, or position 22 modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises a 36-nucleotide sense strand having the sugar moiety at positions 8-11 modified with 2′-F. In some embodiments, the oligonucleotide comprises a sense strand comprising a stem-loop and the sugar moiety at positions 8-11 modified with 2′-F. In some embodiments, the oligonucleotide comprises a 36-nucleotide sense strand having the sugar moiety at positions 1-7 and 12-17 or 12-20 modified with 2′-OMe. In some embodiments, the oligonucleotide comprises a sense strand comprising a stem loop and the sugar moiety at positions 1-7 and 12-17 or 12-20 modified with 2′-OMe. In some embodiments, the oligonucleotide comprises a 36-nucleotide sense strand having the sugar moiety at positions 1-7 and 12-17, 12-20 or 12-22 modified with 2′-OMe. In some embodiments, the oligonucleotide comprises a sense strand comprising a stem-loop and the sugar moiety at positions 1-7 and 12-17, 12-20, or 12-22 modified with 2′-OMe. In some embodiments, the oligonucleotide comprises a 36-nucleotide sense strand having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA. In some embodiments, the oligonucleotide comprises a sense strand comprising and stem-loop and having the sugar moiety of each of the nucleotides at positions 1-7 and 12-17 or 12-20 of the sense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA. In some embodiments, the oligonucleotide comprises a 36-nucleotide sense strand having the sugar moiety of each of the nucleotides at positions 1-7 and 12-17, 12-20, or 12-22 of the sense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA. In some embodiments, the oligonucleotide comprises a sense strand comprising a stem-loop and the sugar moiety of each of the nucleotides at positions 1-7 and 12-17, 12-20, or 12-22 of the sense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises a sense strand having the sugar moiety at positions 3, 5, 8, 10, 12, 13, 15, and 17 modified with 2′-F. In some embodiments, the oligonucleotide comprises a sense strand having the sugar moiety at positions 1, 2, 4, 6, 7, 9, 11, 14, 16, and 18-20 modified with 2′-OMe. In some embodiments, the oligonucleotide comprises a sense strand having the sugar moiety of each of the nucleotides at positions 1, 2, 4, 6, 7, 9, 11, 14, 16, and 18-20 of the sense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA. In some embodiments, the oligonucleotide comprises a sense strand having the sugar moiety of each of the nucleotides at positions 1-7 and 12-17, 12-20, or 12-22 of the sense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-F.

In some embodiments, the oligonucleotide comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with 2′-OMe.

In some embodiments, the oligonucleotide comprises a sense strand having the sugar moiety at position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, or position 36 modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the oligonucleotide comprises an antisense strand having the sugar moiety of each of the nucleotides at positions 2, 3, 4, 5, 7, 10, and 14 of the antisense strand modified with 2′-F, and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-deoxy-2′-fluoro-β-d-arabinonucleic acid (2′-FANA); and a 36-nucleotide sense strand having the sugar moiety at each of the nucleotides at positions 8-11 of the sense strand modified with 2′-F, and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-deoxy-2′-fluoro-β-d-arabinonucleic acid (2′-FANA). In some embodiments, the oligonucleotide comprises an antisense strand having the sugar moiety of each of the nucleotides at positions 2, 3, 4, 5, 7, 10, and 14 of the antisense strand modified with 2′-F, and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA; and a sense strand comprising a stem-loop and the sugar moiety at each of the nucleotides at positions 8-11 of the sense strand modified with 2′-F, and the sugar moiety of each of the remaining nucleotides of the antisense strand modified with a modification selected from the group consisting of 2′-O-propargyl, 2′-O-propylamin, 2′-amino, 2′-ethyl, 2′-EA, 2′-OMe, 2′-MOE, 2′-O-NMA, and 2′-FANA.

In some embodiments, the oligonucleotide comprises an antisense strand having the sugar moiety of each of the nucleotides at positions 2, 3, 4, 5, 7, 10, 14, 16, and 19 of the antisense strand modified with 2′-F, and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA; and a sense strand having the sugar moiety at each of the nucleotides at positions 3, 5, 8, 10, 12, 13, 15, and 17 of the sense strand modified with 2′-F, and the sugar moiety of each of the remaining nucleotides of the antisense strand modified with a modification selected from the group consisting of 2′-O-propargyl, 2′-O-propylamin, 2′-amino, 2′-ethyl, EA, 2′-OMe, 2′-MOE, 2′-O-NMA, and 2′-FANA.

In some embodiments, the sense and antisense strands of an oligonucleotide comprise nucleotides sequences selected from the group consisting of:
a) SEQ ID NO: 769 and 804, respectively;
b) SEQ ID NO: 770 and 805, respectively;
c) SEQ ID NO: 771 and 806, respectively;
d) SEQ ID NO: 772 and 807, respectively;
e) SEQ ID NO: 773 and 808, respectively;
f) SEQ ID NO: 774 and 809, respectively;
g) SEQ ID NO: 775 and 810, respectively;
h) SEQ ID NO: 776 and 811, respectively;
i) SEQ ID NO: 777 and 812, respectively;
j) SEQ ID NO: 778 and 813, respectively;
k) SEQ ID NO: 779 and 814, respectively;
l) SEQ ID NO: 780 and 815, respectively;
m) SEQ ID NO: 781 and 816, respectively;
n) SEQ ID NO: 782 and 817, respectively;
o) SEQ ID NO: 783 and 818, respectively;
p) SEQ ID NO: 784 and 819, respectively;
q) SEQ ID NO: 785 and 820, respectively;
r) SEQ ID NO: 786 and 821, respectively;
S) SEQ ID NO: 787 and 822, respectively;
t) SEQ ID NO: 788 and 823, respectively;
u) SEQ ID NO: 789 and 824, respectively;
V) SEQ ID NO: 790 and 825, respectively;
w) SEQ ID NO: 791 and 826, respectively;
x) SEQ ID NO: 792 and 827, respectively;
y) SEQ ID NO: 793 and 828, respectively;
z) SEQ ID NO: 794 and 829, respectively;
aa) SEQ ID NO: 795 and 830, respectively;
bb) SEQ ID NO: 796 and 831, respectively;
cc) SEQ ID NO: 797 and 832, respectively;
dd) SEQ ID NO: 798 and 833, respectively;
ee) SEQ ID NO: 799 and 834, respectively;
ff) SEQ ID NO: 800 and 835, respectively;
gg) SEQ ID NO: 801 and 836, respectively;
hh) SEQ ID NO: 802 and 837, respectively;
ii) SEQ ID NO: 803 and 838, respectively; and
jj) SEQ ID NOs: 1681 and 815, respectively, wherein one or more of positions 3, 5, 8, 10, 12, 13, 15, or 17 of the sense strand are modified with a 2′—F group.

In some embodiments, the sense and antisense strands of an oligonucleotide comprise nucleotides sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively; and
k) SEQ ID NOs: 1681 and 815, respectively, wherein one or more of positions 3, 5, 8, 10, 12, 13, 15, or 17 of the sense strand is modified with a 2′—F group.

In some embodiments, the sense and antisense strands of an oligonucleotide comprise nucleotides sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively;
f) SEQ ID NOs: 803 and 838, respectively; and
g) SEQ ID NOs: 1681 and 815, respectively, wherein one or more of positions 3, 5, 8, 10, 12, 13, 15, or 17 of the sense strand is modified with a 2′—F group.

### 5′ Terminal Phosphate

In some embodiments, the oligonucleotide comprises a sense strand and an antisense strand, wherein the antisense strand comprises a 5′ terminal phosphate. In some embodiments, 5′ terminal phosphate groups of the oligonucleotide enhance the interaction with Ago2. However, oligonucleotides comprising a 5′-phosphate group may be susceptible to degradation via phosphatases or other enzymes, which can limit their bioavailability in vivo. In some embodiments, the oligonucleotide includes analogs of 5′ phosphates that are resistant to such degradation. In some embodiments, the phosphate analog is oxymethyl phosphonate, vinyl phosphonate or malonyl phosphonate, or a combination thereof. In certain embodiments, the 5′ end of the oligonucleotide strand is attached to chemical moiety that mimics the electrostatic and steric properties of a natural 5′-phosphate group (“phosphate mimic”).

In some embodiments, the oligonucleotide has a phosphate analog at a 4′-carbon position of the sugar (referred to as a “4′-phosphate analog”). See, e.g., Intl. Patent Application Publication No. WO 2018/045317. In some embodiments, the oligonucleotide comprises a 4′-phosphate analog at a 5′ terminal nucleotide. In some embodiments, the phosphate analog is an oxymethyl phosphonate, in which the oxygen atom of the oxymethyl group is bound to the sugar moiety (e.g., at its 4′-carbon) or analog thereof. In other embodiments, the 4′-phosphate analog is a thiomethylphosphonate or an aminomethylphosphonate, in which the sulfur atom of the thiomethyl group or the nitrogen atom of the amino methyl group is bound to the 4′-carbon of the sugar moiety or analog thereof. In certain embodiments, the 4′-phosphate analog is an oxymethyl phosphonate. In some embodiments, the oxymethyl phosphonate is represented by the formula —O—CH₂—PO(OH)₂, —O—CH₂—PO(OR)₂, or —O—CH₂—POOH(R), in which R is independently selected from H, CH₃, an alkyl group, CH₂CH₂CN, CH₂OCOC(CH₃)₃, CH₂OCH₂CH₂Si(CH₃)₃ or a protecting group. In certain embodiments, the alkyl group is CH₂CH₃. More typically, R is independently selected from H, CH₃ or CH₂CH₃. In some embodiment, R is CH₃. In some embodiments, the 4′-phosphate analog is 4′-oxymethylphosphonate. In some embodiments, the modified nucleotide having the 4′-phosphonate analog is a uridine. In some embodiments, the modified nucleotide is 4′-O-monomethylphosphonate-2′-O-methyl uridine.

In some embodiments, the sense and antisense strands of the oligonucleotide comprise nucleotides sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
S) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
w) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively;
ii) SEQ ID NOs: 803 and 838, respectively; and
jj) SEQ ID NOs: 1681 and 815, respectively, wherein the oligonucleotide comprises a 5′ terminal phosphate, optionally a 5′ terminal phosphate analog.

In some embodiments, the sense and antisense strands of the oligonucleotide comprise nucleotides sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively; and
k) SEQ ID NOs: 1681 and 815, respectively, wherein the oligonucleotide comprises a 5′ terminal phosphate, optionally a 5′ terminal phosphate analog.

In some embodiments, the sense and antisense strands of an oligonucleotide comprise nucleotides sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively;
f) SEQ ID NOs: 803 and 838, respectively; and
g) SEQ ID NOs: 1681 and 815, respectively, wherein the oligonucleotide comprises a 5′ terminal phosphate, optionally a 5′ terminal phosphate analog.

In some embodiments, the oligonucleotide comprises an antisense strand comprising a 4′-phosphate analog at the 5′ terminal nucleotide, wherein 5′ terminal nucleotide comprises the following structure:

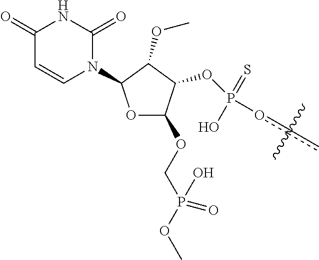

4′-O-monomethylphosphonate-2′-O-methyl uridine phosphorothioate [MePhosphonate-40-mUs].

### Modified Internucleotide Linkage

In some embodiments, an oligonucleotide herein (e.g., a RNAi oligonucleotide) comprises a modified internucleotide linkage. In some embodiments, phosphate modifications or substitutions result in an oligonucleotide that comprises at least about 1 (e.g., at least 1, at least 2, at least 3, or at least 5) modified internucleotide linkage. In some embodiments, the oligonucleotide comprises about 1 to about 10 (e.g., 1 to 10, 2 to 8, 4 to 6, 3 to 10, 5 to 10, 1 to 5, 1 to 3, or 1 to 2) modified internucleotide linkages. In some embodiments, the oligonucleotide comprises 1, 2, 3, 4, 5, 6, 7, 8, 9, or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of the oligonucleotide is a phosphorothioate linkage.

In some embodiments, the oligonucleotide has a phosphorothioate linkage between one or more of positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide described herein has a phosphorothioate linkage between each of positions 1 and 2 of the sense strand, positions 1 and 2 of the antisense strand, positions 2 and 3 of the antisense strand, positions 20 and 21 of the antisense strand, and positions 21 and 22 of the antisense strand. In some embodiments, the oligonucleotide described herein has a phosphorothioate linkage between each of (i) positions 1 and 2 of the sense strand; and (ii) positions 1 and 2, positions 2 and 3, positions 3 and 4, positions 20 and 21, and positions 21 and 22 of the antisense strand.

In some embodiments, the sense and antisense strands of an oligonucleotide comprise nucleotides sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
S) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
w) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively;
ii) SEQ ID NOs: 803 and 838, respectively; and
jj) SEQ ID NOs: 1681 and 815, respectively, wherein the oligonucleotide comprises a modified internucleotide linkage.

In some embodiments, the sense and antisense strands of an oligonucleotide comprise nucleotides sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively; and
k) SEQ ID NOs: 1681 and 815, respectively, wherein the oligonucleotide comprises a modified internucleotide linkage.

In some embodiments, the sense and antisense strands of an oligonucleotide comprise nucleotides sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively;
f) SEQ ID NOs: 803 and 838, respectively; and
g) SEQ ID NOs: 1681 and 815, respectively, wherein the oligonucleotide comprises a modified internucleotide linkage.

### Base Modifications

In some embodiments, an oligonucleotide herein (e.g., a RNAi oligonucleotide) has one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom. See, e.g., US Patent Application Publication No. 2008/0274462. In some embodiments, a modified nucleotide comprises a universal base. In some embodiments, a modified nucleotide does not contain a nucleobase (abasic).

In some embodiments, the sense and antisense strands of an oligonucleotide comprise nucleotides sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
S) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
w) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively;
ii) SEQ ID NOs: 803 and 838, respectively; and
jj) SEQ ID NOs: 1681 and 815, respectively, wherein the oligonucleotide comprises one or more modified nucleobases.

In some embodiments, the sense and antisense strands of an oligonucleotide comprise nucleotides sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively;
j) SEQ ID NOs: 803 and 838, respectively; and
k) SEQ ID NOs: 1681 and 815, respectively, wherein the oligonucleotide comprises one or more modified nucleobases.

In some embodiments, the sense and antisense strands of an oligonucleotide comprise nucleotides sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively;
f) SEQ ID NOs: 803 and 838, respectively;
g) SEQ ID NOs: 1681 and 815, respectively, wherein the oligonucleotide comprises one or more modified nucleobases.

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference ss nucleic acid (e.g., an oligonucleotide) that is fully complementary to a target nucleic acid, a ss nucleic acid containing a universal base forms a duplex with the target nucleic acid that has a lower Tₘ than a duplex formed with the complementary nucleic acid. In some embodiments, when compared to a reference ss nucleic acid in which the universal base has been replaced with a base to generate a single mismatch, the ss nucleic acid containing the universal base forms a duplex with the target nucleic acid that has a higher Tₘ than a duplex formed with the nucleic acid comprising the mismatched base.

Non-limiting examples of universal-binding nucleotides include, but are not limited to, inosine, 1-β-D-ribofuranosyl-5-nitroindole and/or 1-β-D-ribofuranosyl-3-nitropyrrole (see, US Patent Application Publication No. 2007/0254362; Van Aerschot et al. (1995) NUCLEIC ACIDS RES. 23:4363-4370; Loakes et al. (1995) NUCLEIC ACIDS RES. 23:2361-66; and Loakes & Brown (1994) NUCLEIC ACIDS RES. 22:4039-43).

### Targeting Ligands

In some embodiments, it is desirable to target the oligonucleotide (e.g., a RNAi oligonucleotide) to one or more cells or one or more organs. Such a strategy can help to avoid undesirable effects in other organs or avoid undue loss of the oligonucleotide to cells, tissue, or organs that would not benefit from the oligonucleotide. Accordingly, in some embodiments, the oligonucleotide is modified to facilitate targeting and/or delivery to a particular tissue, cell, or organ (e.g., to facilitate delivery of the oligonucleotide to the CNS). In some embodiments, the oligonucleotide comprises at least one nucleotide (e.g., 1, 2, 3, 4, 5, 6, or more nucleotides) conjugated to one or more targeting ligand(s). In some embodiments, the sense and antisense strands of the oligonucleotide comprise nucleotides sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
S) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
w) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively; and
ii) SEQ ID NOs: 803 and 838, respectively, wherein the oligonucleotide comprises a targeting ligand conjugated to at least one nucleotide.

In some embodiments, the oligonucleotide comprises at least one nucleotide (e.g., 1, 2, 3, 4, 5, 6, or more nucleotides) conjugated to one or more targeting ligand(s). In some embodiments, the sense and antisense strands of the oligonucleotide comprise nucleotides sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively; and
j) SEQ ID NOs: 803 and 838, respectively, wherein the oligonucleotide comprises a targeting ligand conjugated to at least one nucleotide.

In some embodiments, the oligonucleotide comprises at least one nucleotide (e.g., 1, 2, 3, 4, 5, 6, or more nucleotides) conjugated to one or more targeting ligand(s). In some embodiments, the sense and antisense strands of the oligonucleotide comprise nucleotides sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively; and
f) SEQ ID NOs: 803 and 838, respectively, wherein the oligonucleotide comprises a targeting ligand conjugated to at least one nucleotide.

In some embodiments, the targeting ligand comprises a carbohydrate, amino sugar, cholesterol, peptide, polypeptide, or protein or part of a protein (e.g., an antibody or antibody fragment). In some embodiments, the targeting ligand is an aptamer. For example, the targeting ligand may be an RGD peptide that is used to target tumor vasculature or glioma cells, CREKA peptide (SEQ ID NO: 1689) to target tumor vasculature or stoma, transferring, lactoferrin, or an aptamer to target transferrin receptors expressed on CNS vasculature, or an anti-EGFR antibody to target EGFR on glioma cells. In certain embodiments, the targeting ligand is one or more GalNAc moieties. In some embodiments, the targeting ligand is one or more lipid moieties.

In some embodiments, 1 or more (e.g., 1, 2, 3, 4, 5, or 6) nucleotides of the oligonucleotide are each conjugated to a separate targeting ligand. In some embodiments, 2 to 4 nucleotides of the oligonucleotide are each conjugated to a separate targeting ligand. In some embodiments, targeting ligands are conjugated to 2 to 4 nucleotides at either ends of the sense or antisense strand (e.g., targeting ligands are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, the oligonucleotide may comprise a stem-loop at either the 5′ or 3′ end of the sense strand and 1, 2, 3, or 4 nucleotides of the loop of the stem may be individually conjugated to a targeting ligand. In some embodiments, the oligonucleotide comprises a stem-loop at the 3′ end of the sense strand, wherein the loop of the stem-loop comprises a triL or a tetraL, and wherein the 3 or 4 nucleotides comprising the triL or tetraL, respectfully, are individually conjugated to a targeting ligand. In some embodiments, the oligonucleotide comprises a blunt end at the 3′ end of the oligonucleotide and one or more targeting ligands conjugated to at least one nucleotide. In some embodiments, the oligonucleotide comprises a blunt end at the 3′ end of the oligonucleotide and one or more targeting ligands conjugated to the 5′ terminal nucleotide of the sense strand.

### GalNAc Conjugation

GalNAc is a high affinity ligand for the asialoglycoprotein receptor (ASGPR), which is primarily expressed on the sinusoidal surface of hepatocyte cells and has a major role in binding, internalizing and subsequent clearing circulating glycoproteins that contain terminal galactose or GalNAc residues (asialoglycoproteins). Conjugation (either indirect or direct) of GalNAc moieties to the oligonucleotides herein can be used to target them to ASGPR expressed on cells. In some embodiments, the oligonucleotide is conjugated to at least one or more GalNAc moieties, wherein the GalNAc moieties target the oligonucleotide to ASGPR expressed on human liver cells (e.g., human hepatocytes). In some embodiments, the GalNAc moiety target the oligonucleotide to the liver.

In some embodiments, the oligonucleotide is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, the oligonucleotide is conjugated to one or more bivalent GalNAc, trivalent GalNAc, or tetravalent GalNAc moieties. In some embodiments, the bivalent, trivalent, or tetravalent GalNAc moiety is conjugated to the oligonucleotide via a branched linker. In some embodiments, the monovalent GalNAc moiety is conjugated to a first nucleotide and the bivalent, trivalent, or tetravalent GalNAc moiety is conjugated to a second nucleotide via a branched linker.

In some embodiments, 1 or more (e.g., 1, 2, 3, 4, 5, or 6) nucleotides of the oligonucleotide are each conjugated to a GalNAc moiety. In some embodiments, 2 to 4 nucleotides of a tetraL are each conjugated to a separate GalNAc. In some embodiments, 1 to 3 nucleotides of a triL are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, 4 GalNAc moieties can be conjugated to nucleotides in the tetraL of the sense strand where each GalNAc moiety is conjugated to 1 nucleotide.

In some embodiments, the oligonucleotide comprises a tetraL, wherein the tetraL is any combination of adenine (A) and guanine (G) nucleotides. In some embodiments, the tetraL comprises a monovalent GalNAc moiety attached to any one or more guanine (G) nucleotides of the tetraloop via any linker described herein, as depicted below (X=heteroatom):

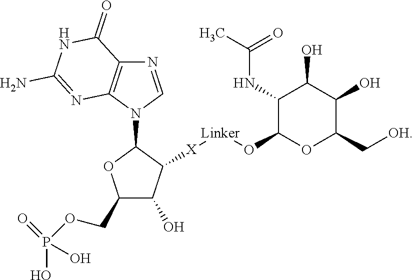

In some embodiments, the tetraL has a monovalent GalNAc attached to any one or more adenine nucleotides of the tetraloop via any linker described herein, as depicted below (X=heteroatom):

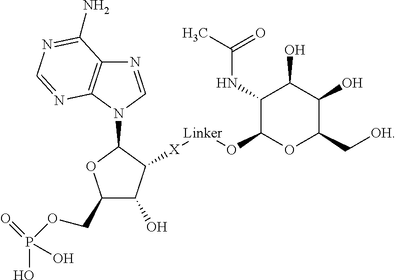

In some embodiments, an oligonucleotide herein (e.g., an RNAi oligonucleotide) comprises a monovalent GalNAc attached to a guanine nucleotide referred to as [ademG-GalNAc] or 2′-aminodiethoxymethanol-Guanine-GalNAc, as depicted below:

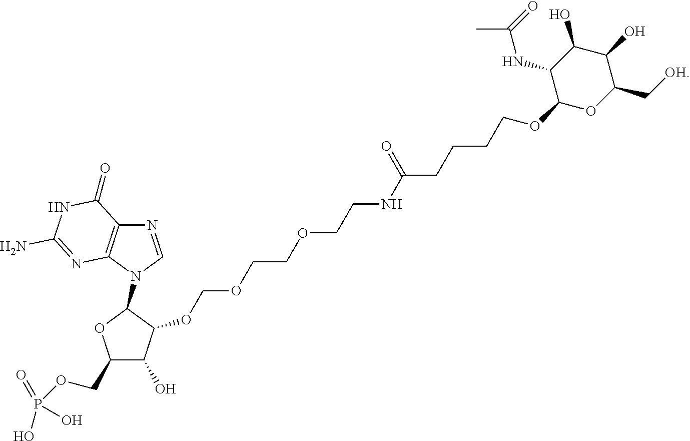 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below:

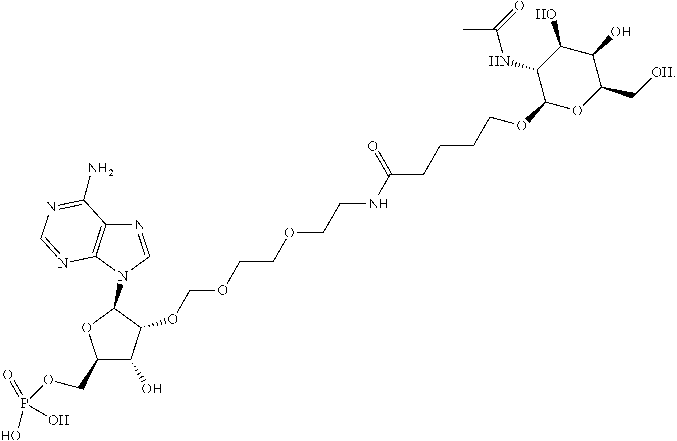

An example of such conjugation is shown below for a loop comprising from 5′ to 3′ the nucleotide sequence GAAA (L=linker, X=heteroatom) stem attachment points are shown. Such a loop may be present, for example, at positions 27-30 of the sense strand of any one of the sense strands listed in Tables 4 and 5. In the chemical formula,

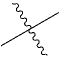

is used to describe an attachment point to the oligonucleotide strand:

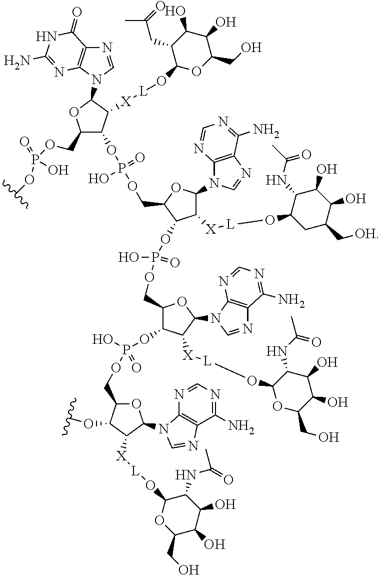

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is stable. Examples are shown below for a loop comprising from 5′ to 3′ the nucleotides GAAA, in which GalNAc moieties are attached to 3 or 4 nucleotides of the loop using an acetal linker. Such a loop may be present, for example, at positions 27-30 of the any one of the sense strands listed in Tables 4 and 5. In the chemical formula,

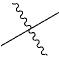

is an attachment point to the oligonucleotide strand:

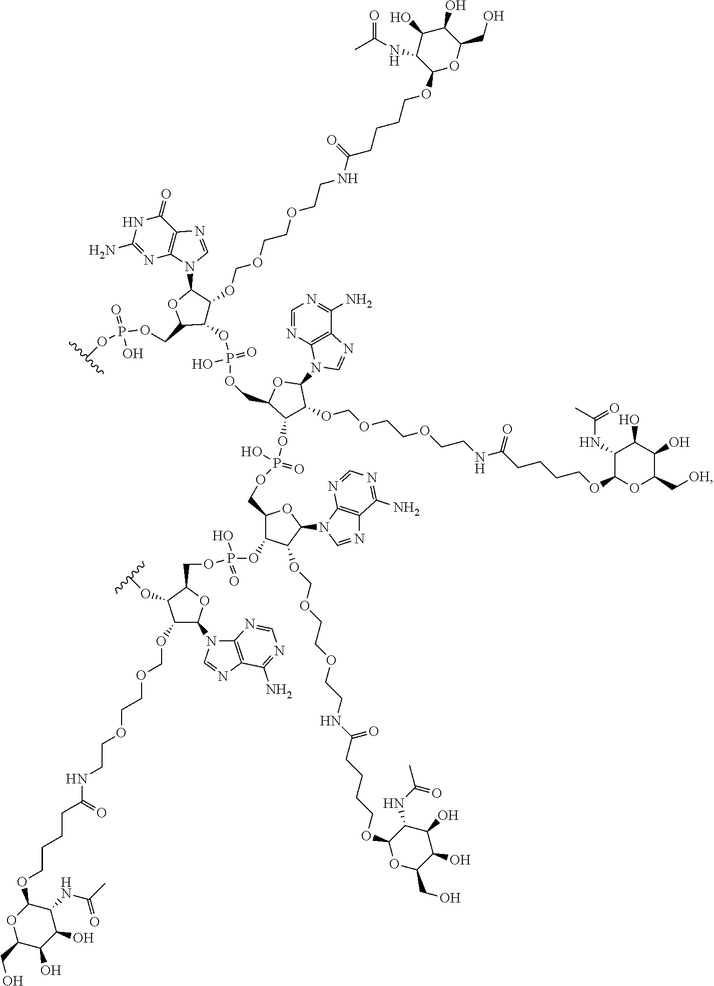

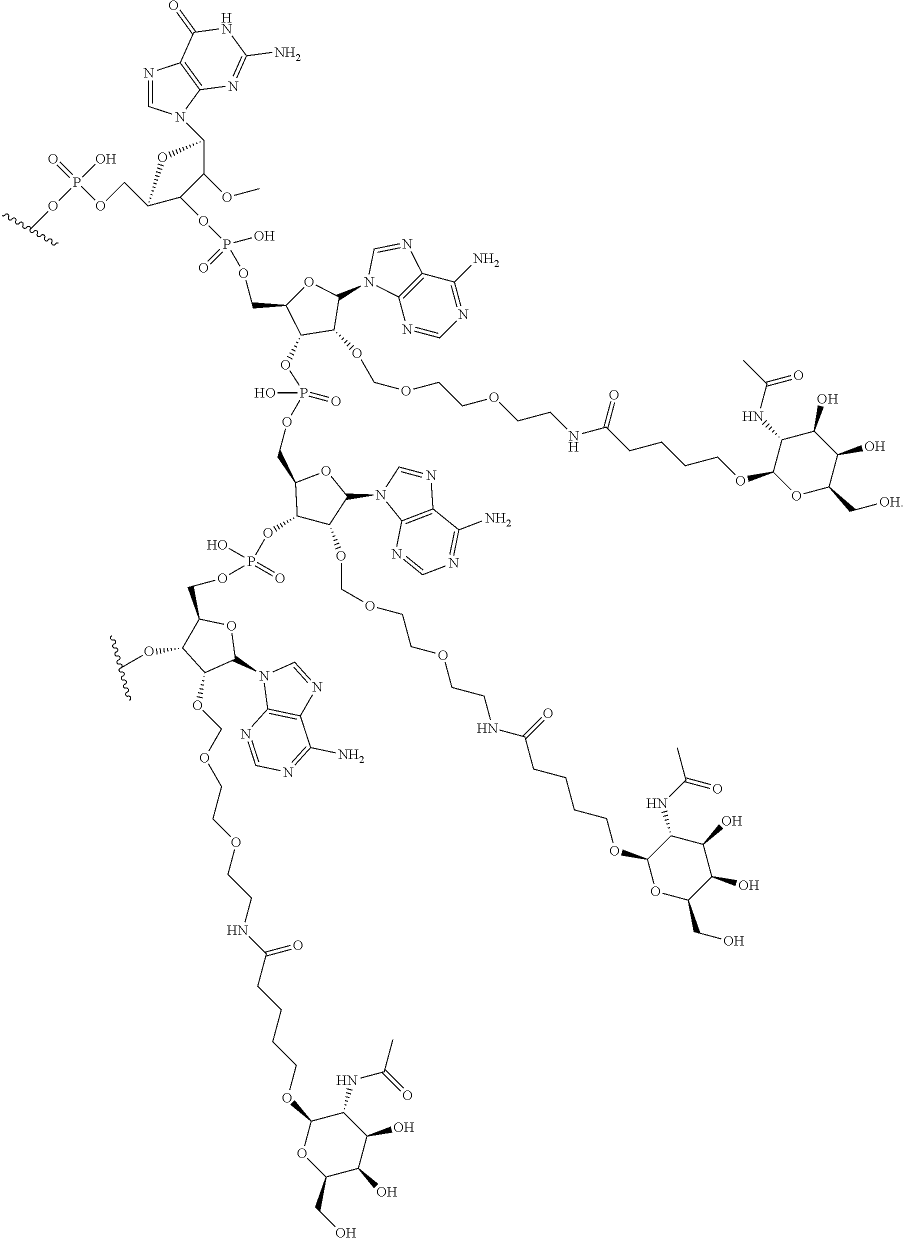

As mentioned, various appropriate methods or chemistry synthetic techniques (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in Intl. Patent Application Publication No. WO 2016/100401. In some embodiments, the linker is a labile linker. However, in other embodiments, the linker is a stable linker.

In some embodiments, a duplex extension (e.g., of up to 3, 4, 5, or 6 bp in length) is provided between a targeting ligand (e.g., a GalNAc moiety) and a RNAi oligonucleotide. In some embodiments, the oligonucleotides herein do not have a GalNAc conjugated thereto.

In some embodiments, the sense and antisense strands of the oligonucleotide comprise nucleotides sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
s) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
W) SEQ ID NOs: 791 and 826, respectively;
x) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively; and
ii) SEQ ID NOs: 803 and 838, respectively, wherein the oligonucleotide comprises at least one GalNAc moiety conjugated to a nucleotide.

In some embodiments, the sense and antisense strands of the oligonucleotide comprise nucleotides sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively; and
j) SEQ ID NOs: 803 and 838, respectively, wherein the oligonucleotide comprises at least one GalNAc moiety conjugated to a nucleotide.

In some embodiments, the sense and antisense strands of the oligonucleotide comprise nucleotides sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively; and
f) SEQ ID NOs: 803 and 838, respectively, wherein the oligonucleotide comprises at least one GalNAc moiety conjugated to a nucleotide.

### Lipid Conjugation

In some embodiments, one or more lipid moieties are conjugated to a 5′ terminal nucleotide of a sense strand. In some embodiments, one or more lipid moieties are conjugated to an adenine nucleotide. In some embodiments, one or more lipid moieties are conjugated to a guanine nucleotide. In some embodiments, one or more lipid moieties are conjugated to a cytosine nucleotide. In some embodiments, one or more lipid moieties are conjugated to a thymine nucleotide. In some embodiments, one or more lipid moieties are conjugated to a uracil nucleotide.

In some embodiments, the lipid moiety is a hydrocarbon chain. In some embodiments, the hydrocarbon chain is saturated. In some embodiments, the hydrocarbon chain is unsaturated. In some embodiments, the hydrocarbon chain is branched. In some embodiments, the hydrocarbon chain is straight. In some embodiments, the lipid moiety is a C₈-C₃₀ hydrocarbon chain. In some embodiments, the lipid moiety is a C₈:0, C₁₀:0, C₁₁:0, C₁₂:0, C₁₄:0, C₁₆:0, C₁₇:0, C₁₈:0, C₁₈:1, C₁₈:2, C₂₂:5, C₂₂:0, C₂₄:0, C₂₆:0, C₂₂:6, C₂₄:1, diacyl C₁₆:0 or diacyl C₁₈:1. In some embodiments, the lipid moiety is a C₁₆ hydrocarbon chain. In some embodiments, the C₁₆ hydrocarbon chain is represented as:

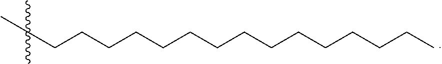 [SMILES: *CCCCCCCCCCCCCCC]

In some embodiments, the sense strand is 20-22 nucleotides in length and the lipid moiety is a hydrocarbon chain that is conjugated to the 5′ terminal nucleotide of the sense strand. In some embodiments, the sense strand is 20-22 nucleotides in length and the hydrocarbon chain is conjugated to the 5′ terminal nucleotide of the sense strand. In some embodiments, the sense strand is 20-22 nucleotides in length and a C₁₄-C₂₂ hydrocarbon chain is conjugated to the 5′ terminal nucleotide of the sense strand. In some embodiments, the sense strand is 20-22 nucleotides in length and a C₁₆ hydrocarbon chain is conjugated to the 5′ terminal nucleotide of the sense strand. In some embodiments, the sense strand is 20 nucleotides in length and the lipid moiety is conjugated to the 5′ terminal nucleotide of the sense strand. In some embodiments, the sense strand is 20 nucleotides in length and the lipid moiety is a hydrocarbon chain is conjugated to the 5′ terminal nucleotide of the sense strand. In some embodiments, the sense strand is 20 nucleotides in length and a C₁₄-C₂₂ hydrocarbon chain is conjugated to the 5′ terminal nucleotide of the sense strand. In some embodiments, the sense strand is 20 nucleotides in length and a C₁₆ hydrocarbon chain is conjugated to the 5′ terminal nucleotide of the sense strand.

In some embodiments, the oligonucleotide comprises (i) a sense strand of 20-22 nucleotides in length; (ii) an antisense strand comprising a 3′ overhang sequence of one or more nucleotides in length; (iii) a blunt end comprising the 3′ end of the sense strand; and (iv) a lipid moiety conjugated to the 5′ terminal nucleotide of the sense strand. In some embodiments, the oligonucleotide comprises (i) a sense strand of 20-22 nucleotides in length; (ii) an antisense strand comprising a 3′ overhang sequence of one or more nucleotides in length; (iii) a blunt end comprising the 3′ end of the sense strand; and (iv) a hydrocarbon chain conjugated to the 5′ terminal nucleotide of the sense strand. In some embodiments, the oligonucleotide comprises (i) a sense strand of 20-22 nucleotides in length; (ii) an antisense strand comprising a 3′ overhang sequence of one or more nucleotides in length; (iii) a blunt end comprising the 3′ end of the sense strand; and (iv) a C₁₄-C₂₂ hydrocarbon chain conjugated to the 5′ terminal nucleotide of the sense strand. In some embodiments, the oligonucleotide comprises (i) a sense strand of 20-22 nucleotides in length; (ii) an antisense strand comprising a 3′ overhang sequence of one or more nucleotides in length; (iii) a blunt end comprising the 3′ end of the sense strand; and (iv) a C₁₆ hydrocarbon chain conjugated to the 5′ terminal nucleotide of the sense strand.

In some embodiments, the oligonucleotide comprises (i) a sense strand of 20 nucleotides in length; (ii) an antisense strand of 22 nucleotides in length comprising a 3′ overhang sequence of two nucleotides in length; (iii) a blunt end comprising the 3′ end of the sense strand; and (iv) a lipid moiety conjugated to the 5′ terminal nucleotide of the sense strand. In some embodiments, the oligonucleotide comprises (i) a sense strand of 20 nucleotides in length; (ii) an antisense strand of 22 nucleotides in length comprising a 3′ overhang sequence of two nucleotides in length; (iii) a blunt end comprising the 3′ end of the sense strand; and (iv) a hydrocarbon chain conjugated to the 5′ terminal nucleotide of the sense strand. In some embodiments, the oligonucleotide comprises (i) a sense strand of 20 nucleotides in length; (ii) an antisense strand of 22 nucleotides in length comprising a 3′ overhang sequence of two nucleotides in length; (iii) a blunt end comprising the 3′ end of the sense strand; and (iv) a C₁₄-C₂₂ hydrocarbon chain conjugated to the 5′ terminal nucleotide of the sense strand. In some embodiments, the oligonucleotide comprises (i) a sense strand of 20 nucleotides in length; (ii) an antisense strand of 22 nucleotides in length comprising a 3′ overhang sequence of two nucleotides in length; (iii) a blunt end comprising the 3′ end of the sense strand; and (iv) a C₁₆ hydrocarbon chain conjugated to the 5′ terminal nucleotide of the sense strand.

In some embodiments, the oligonucleotide comprises (i) an antisense strand of 19-30 nucleotides comprising a region of complementarity to a MAPT mRNA target sequence selected from SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, and 1124; (ii) a sense strand of 19-25 nucleotides that forms a duplex region with the antisense strand; and (iii) a lipid moiety conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1061, 1108, 1119, 1120, 1124, 1130, 1065, 1095, 1096, and 1102; (ii) a sense strand of 19-25 nucleotides that forms a duplex region with the antisense strand; and (iii) a lipid moiety conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1130, 1095, 1096, 1119, 1120, and 1124; (ii) a sense strand of 19-25 nucleotides that forms a duplex region with the antisense strand; and (iii) a lipid moiety conjugated to the 5′ terminal nucleotide of the sense strand.

In some embodiments, the oligonucleotide comprises (i) an antisense strand of 19-30 nucleotides comprising a region of complementarity to a MAPT mRNA target sequence selected from SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, and 1124; (ii) a sense strand of 19-25 nucleotides that forms a duplex region with the antisense strand; and (iii) a hydrocarbon chain conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1061, 1108, 1119, 1120, 1124, 1130, 1065, 1095, 1096, and 1102; (ii) a sense strand of 19-25 nucleotides that forms a duplex region with the antisense strand; and (iii) a hydrocarbon chain conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1130, 1095, 1096, 1119, 1120, and 1124; (ii) a sense strand of 19-25 nucleotides that forms a duplex region with the antisense strand; and (iii) a hydrocarbon chain conjugated to the 5′ terminal nucleotide of the sense strand.

In some embodiments, the oligonucleotide comprises (i) an antisense strand of 19-30 nucleotides comprising a region of complementarity to a MAPT mRNA target sequence selected from SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, and 1124; (ii) a sense strand of 19-25 nucleotides that forms a duplex region with the antisense strand; and (iii) a C₁₄-C₂₂ hydrocarbon chain conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1061, 1108, 1119, 1120, 1124, 1130, 1065, 1095, 1096, and 1102; (ii) a sense strand of 19-25 nucleotides that forms a duplex region with the antisense strand; and (iii) a C₁₄-C₂₂ hydrocarbon chain conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1130, 1095, 1096, 1119, 1120, and 1124; (ii) a sense strand of 19-25 nucleotides that forms a duplex region with the antisense strand; and (iii) a C₁₄-C₂₂ hydrocarbon chain conjugated to the 5′ terminal nucleotide of the sense strand.

In some embodiments, the oligonucleotide comprises (i) an antisense strand of 19-30 nucleotides comprising a region of complementarity to a MAPT mRNA target sequence selected from SEQ ID NOs: 1125, 1127, 1130, 1019, 1031, 1044, 1064, 1065, 1067, 1083, 915, 1095, 1096, 1102, 1110, 923, 925, 1025, 1039, 1049, 1061, 1070, 1072, 1075, 1081, 1108, 1111, 1114, 1119, 1120, 1121, 1122, 1123, and 1124; (ii) a sense strand of 19-25 nucleotides that forms a duplex region with the antisense strand; and (iii) a C₁₆ hydrocarbon chain conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1061, 1108, 1119, 1120, 1124, 1130, 1065, 1095, 1096, and 1102; (ii) a sense strand of 19-25 nucleotides that forms a duplex region with the antisense strand; and (iii) a C₁₆ hydrocarbon chain conjugated to the 5′ terminal nucleotide of the sense strand. In some embodiments, the oligonucleotide comprises (i) an antisense strand of 19-30 nucleotides comprising a region of complementarity to a MAPT mRNA target sequence selected from SEQ ID NOs: 1130, 1095, 1096, 1119, 1120, and 1124; (ii) a sense strand of 19-25 nucleotides that forms a duplex region with the antisense strand; and (iii) a C₁₆ hydrocarbon chain conjugated to the 5′ terminal nucleotide of the sense strand.

In some embodiments, the oligonucleotide comprises a sense strand comprising the nucleotide sequence of SEQ ID NO: 1681 and an antisense strand comprising the nucleotide sequence of SEQ ID NO: 815, wherein the sense strand comprises a lipid moiety conjugated to the 5′ terminal nucleotide of the sense strand. In some embodiments, the oligonucleotide comprises a sense strand comprising the nucleotide sequence of SEQ ID NO: 1681 and an antisense strand comprising the nucleotide sequence of SEQ ID NO: 815, wherein the sense strand comprises a hydrocarbon chain conjugated to the 5′ terminal nucleotide of the sense strand. In some embodiments, the oligonucleotide comprises a sense strand comprising the nucleotide sequence of SEQ ID NO: 1681 and an antisense strand comprising the nucleotide sequence of SEQ ID NO: 815, wherein the sense strand comprises a C₁₄-C₂₂ hydrocarbon chain conjugated to the 5′ terminal nucleotide of the sense strand. In some embodiments, the oligonucleotide comprises a sense strand comprising the nucleotide sequence of SEQ ID NO: 1681 and an antisense strand comprising the nucleotide sequence of SEQ ID NO: 815, wherein the sense strand comprises a C₁₆ hydrocarbon chain conjugated to the 5′ terminal nucleotide of the sense strand.

### Exemplary MAPT-Targeting RNAi Oligonucleotides

In some embodiments, the MAPT-targeting RNAi oligonucleotide for reducing MAPT gene expression provided by the current disclosure comprises a sense strand and an antisense strand, wherein all nucleotides comprising the sense strand and the antisense strand are modified, wherein the antisense strand comprises a region of complementarity to a MAPT mRNA target sequence of any one of SEQ ID NOs: 912-1295, and wherein the region of complementarity is at least 15 contiguous nucleotides in length. In some embodiments, the 5′ terminal nucleotide of the antisense strand comprises 4′-O-monomethylphosphonate-2′-O-methyluridine [MePhosphonate-40-mU], as described herein. In some embodiments, the 5′ terminal nucleotide of the antisense strand comprises a phosphorothioate linkage. In some embodiments, the antisense strand and the sense strand comprise one or more 2′-F- and 2′-OMe-modified nucleotides and at least one phosphorothioate linkage. In some embodiments, the antisense strand comprises 4 phosphorothioate linkages and the sense strand comprises 1 phosphorothioate linkage. In some embodiments, the antisense strand comprises 5 phosphorothioate linkages and the sense strand comprises 1 phosphorothioate linkage.

In some embodiments, an oligonucleotide (e.g., a RNAi oligonucleotide) comprises a sense strand having a sequence of any one of SEQ ID NOs: 912-1295 and an antisense strand comprising a complementary sequence selected from SEQ ID NOs: 1296-1679.

In some embodiments, the oligonucleotide comprises a sense strand having a sequence of any one of SEQ ID NOs: 1-384 and an antisense strand comprising a complementary sequence selected from SEQ ID NOs: 385-768.

In some embodiments, the oligonucleotide comprises a sense strand having a sequence of any one of SEQ ID NOs: 769-803 and an antisense strand comprising a complementary sequence selected from SEQ ID NOs: 804-838.

In some embodiments, the oligonucleotide comprises a sense strand having a sequence of any one of SEQ ID NOs: 769-803 and 1681, and an antisense strand comprising a complementary sequence selected from SEQ ID NOs: 804-838.

In some embodiments, the oligonucleotide comprises a sense strand having a sequence of any one of SEQ ID NOs: 839-873 and an antisense strand comprising a complementary sequence selected from SEQ ID NOs: 874-908.

In some embodiments, the oligonucleotide comprises a sense strand having a sequence of any one of SEQ ID NOs: 839-873 and 1681, and an antisense strand comprising a complementary sequence selected from SEQ ID NOs: 874-908.

In some embodiments, an oligonucleotide (e.g., a RNAi oligonucleotide) for reducing MAPT gene expression comprises:
a sense strand of 36 nucleotides comprising a 2′-F-modified nucleotide at positions 3, 5, 8, 10, 12, 13, 15, and 17; a 2′-OMe-modified nucleotide at positions 1, 2, 4, 6, 7, 9, 11, 14, 16, 18-27, and 31-36; a GalNAc-conjugated nucleotide at position 28, 29, and 30; and a phosphorothioate linkage between positions 1 and 2; and
an antisense strand of 22 nucleotides comprising a 2′-F-modified nucleotide at positions 2, 3, 4, 5, 7, 10, 14, 16, and 19; a 2′-OMe-modified nucleotide at positions 1, 6, 8, 9, 11, 12, 13, 15, 17, 18, and 20-22; a phosphorothioate linkage between positions 1 and 2, positions 2 and 3, positions 20 and 21, and positions 21 and 22; and a 5′ terminal nucleotide at position 1 comprising a 4′-phosphate analog, optionally wherein the 5′ terminal nucleotide comprises 4′-O-monomethylphosphonate-2′-O-methyluridine [MePhosphonate-40-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraL, wherein positions 21 and 22 of the antisense strand comprise an overhang, and wherein the sense strand and antisense strands comprise nucleotide sequences selected from the group consisting of:
a) SEQ ID NOs: 769 and 804, respectively;
b) SEQ ID NOs: 770 and 805, respectively;
c) SEQ ID NOs: 771 and 806, respectively;
d) SEQ ID NOs: 772 and 807, respectively;
e) SEQ ID NOs: 773 and 808, respectively;
f) SEQ ID NOs: 774 and 809, respectively;
g) SEQ ID NOs: 775 and 810, respectively;
h) SEQ ID NOs: 776 and 811, respectively;
i) SEQ ID NOs: 777 and 812, respectively;
j) SEQ ID NOs: 778 and 813, respectively;
k) SEQ ID NOs: 779 and 814, respectively;
l) SEQ ID NOs: 780 and 815, respectively;
m) SEQ ID NOs: 781 and 816, respectively;
n) SEQ ID NOs: 782 and 817, respectively;
o) SEQ ID NOs: 783 and 818, respectively;
p) SEQ ID NOs: 784 and 819, respectively;
q) SEQ ID NOs: 785 and 820, respectively;
r) SEQ ID NOs: 786 and 821, respectively;
S) SEQ ID NOs: 787 and 822, respectively;
t) SEQ ID NOs: 788 and 823, respectively;
u) SEQ ID NOs: 789 and 824, respectively;
v) SEQ ID NOs: 790 and 825, respectively;
W) SEQ ID NOs: 791 and 826, respectively;
X) SEQ ID NOs: 792 and 827, respectively;
y) SEQ ID NOs: 793 and 828, respectively;
z) SEQ ID NOs: 794 and 829, respectively;
aa) SEQ ID NOs: 795 and 830, respectively;
bb) SEQ ID NOs: 796 and 831, respectively;
cc) SEQ ID NOs: 797 and 832, respectively;
dd) SEQ ID NOs: 798 and 833, respectively;
ee) SEQ ID NOs: 799 and 834, respectively;
ff) SEQ ID NOs: 800 and 835, respectively;
gg) SEQ ID NOs: 801 and 836, respectively;
hh) SEQ ID NOs: 802 and 837, respectively; and
ii) SEQ ID NOs: 803 and 838, respectively.

In some embodiments, an oligonucleotide (e.g., and RNAi oligonucleotide) for reducing MAPT gene expression comprises:
a sense strand of 36 nucleotides comprising a 2′-F-modified nucleotide at positions 3, 5, 8, 10, 12, 13, 15, and 17; a 2′-OMe-modified nucleotide at positions 1, 2, 4, 6, 7, 9, 11, 14, 16, 18-27, and 31-36; a GalNAc-conjugated nucleotide at position 28, 29, and 30; and a phosphorothioate linkage between positions 1 and 2; and an antisense strand of 22 nucleotides comprising a 2′-F-modified nucleotide at positions 2, 3, 4, 5, 7, 10, 14, 16, and 19; a 2′-OMe-modified nucleotide at positions 1, 6, 8, 9, 11, 12, 13, 15, 17, 18, and 20-22; a phosphorothioate linkage between positions 1 and 2, positions 2 and 3, positions 20 and 21, and positions 21 and 22; and a 5′ terminal nucleotide at position 1 comprising a 4′-phosphate analog, optionally wherein the 5′ terminal nucleotide comprises 4′-O-monomethylphosphonate-2′-O-methyluridine [MePhosphonate-40-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
a) SEQ ID NOs: 771 and 806, respectively;
b) SEQ ID NOs: 776 and 811, respectively;
c) SEQ ID NOs: 780 and 815, respectively;
d) SEQ ID NOs: 781 and 816, respectively;
e) SEQ ID NOs: 782 and 817, respectively;
f) SEQ ID NOs: 790 and 825, respectively;
g) SEQ ID NOs: 795 and 830, respectively;
h) SEQ ID NOs: 798 and 833, respectively;
i) SEQ ID NOs: 799 and 834, respectively; and
j) SEQ ID NOs: 803 and 838, respectively.

In some embodiments, an oligonucleotide (e.g., a RNAi oligonucleotide) for reducing MAPT gene expression comprises:
a sense strand of 36 nucleotides comprising a 2′-F-modified nucleotide at positions 3, 5, 8, 10, 12, 13, 15, and 17; a 2′-OMe-modified nucleotide at positions 1, 2, 4, 6, 7, 9, 11, 14, 16, 18-27, and 31-36; a GalNAc-conjugated nucleotide at position 28, 29, and 30; and a phosphorothioate linkage between positions 1 and 2; and
an antisense strand of 22 nucleotides comprising a 2′-F-modified nucleotide at positions 2, 3, 4, 5, 7, 10, 14, 16, and 19; a 2′-OMe-modified nucleotide at positions 1, 6, 8, 9, 11, 12, 13, 15, 17, 18, and 20-22; a phosphorothioate linkage between positions 1 and 2, positions 2 and 3, positions 20 and 21, and positions 21 and 22; and a 5′ terminal nucleotide at position 1 comprising a 4′-phosphate analog, optionally wherein the 5′ terminal nucleotide comprises 4′-O-monomethylphosphonate-2′-O-methyluridine [MePhosphonate-40-mU]; wherein positions 1-20 of the antisense strand form a duplex region with positions 1-20 of the sense strand, wherein positions 21-36 of the sense strand form a stem-loop, wherein positions 27-30 form the loop of the stem-loop, optionally wherein positions 27-30 comprise a tetraloop, wherein positions 21 and 22 of the antisense strand comprise an overhang, and wherein the sense strand and antisense strands comprise nucleotide sequences selected from the group consisting of:
a) SEQ ID NOs: 771 and 806, respectively;
b) SEQ ID NOs: 780 and 815, respectively;
c) SEQ ID NOs: 781 and 816, respectively;
d) SEQ ID NOs: 798 and 833, respectively;
e) SEQ ID NOs: 799 and 834, respectively; and
f) SEQ ID NOs: 803 and 838, respectively.

In some embodiments, an oligonucleotide (e.g., a RNAi oligonucleotide) for reducing MAPT gene expression comprises:
a sense strand of 20 nucleotides comprising a 2′-F-modified nucleotide at positions 3, 5, 8, 10, 12, 13, 15, and 17; a 2′-OMe-modified nucleotide at positions 2, 4, 6, 7, 9, 11, 14, 16, and 18-20; a C₁₆ hydrocarbon chain conjugated to a nucleotide at position 1; and a phosphorothioate linkage between positions 1 and 2, between positions 18 and 19, and between positions 19 and 20; and
an antisense strand of 22 nucleotides comprising a 2′-F-modified nucleotide at positions 2, 3, 4, 5, 7, 10, 14, 16, and 19; a 2′-OMe modified-nucleotide at positions 1, 6, 8, 9, 11, 12, 13, 15, 17, 18, and 20-22; a phosphorothioate linkage between positions 1 and 2, positions 2 and 3, positions 20 and 21, and positions 21 and 22; and a 5′ terminal nucleotide at position 1 comprising a 4′-phosphate analog, optionally wherein the 5′ terminal nucleotide comprises 4′-O-monomethylphosphonate-2′-O-methyluridine [MePhosphonate-40-mU]; wherein positions 1-20 of the antisense strand form a duplex region with positions 1-20 of the sense strand, wherein positions 21 and 22 of the antisense strand comprise an overhang, and wherein the sense strand and antisense strands comprise nucleotide sequences of SEQ ID NOs: 1681 and 815, respectively.

In some embodiments, an oligonucleotide for reducing MAPT gene expression comprises a sense strand comprising the nucleotide sequence as set forth in SEQ ID NO: 771 and an antisense strand comprising the nucleotide sequence as set forth in SEQ ID NO: 806. In some embodiments, an oligonucleotide for reducing MAPT gene expression comprises a sense strand comprising the nucleotide sequence as set forth in SEQ ID NO: 780 and an antisense strand comprising the nucleotide sequence as set forth in SEQ ID NO: 815. In some embodiments, an oligonucleotide for reducing MAPT gene expression comprises a sense strand comprising the nucleotide sequence as set forth in SEQ ID NO: 781 and an antisense strand comprising the nucleotide sequence as set forth in SEQ ID NO: 816. In some embodiments, an oligonucleotide for reducing MAPT gene expression comprises a sense strand comprising the nucleotide sequence as set forth in SEQ ID NO: 798 and an antisense strand comprising the nucleotide sequence as set forth in SEQ ID NO: 833. In some embodiments, an oligonucleotide for reducing MAPT gene expression comprises a sense strand comprising the nucleotide sequence as set forth in SEQ ID NO: 799 and an antisense strand comprising the nucleotide sequence as set forth in SEQ ID NO: 834. In some embodiments, an oligonucleotide for reducing MAPT gene expression comprises a sense strand comprising the nucleotide sequence as set forth in SEQ ID NO: 803 and an antisense strand comprising the nucleotide sequence as set forth in SEQ ID NO: 838. In some embodiments, an oligonucleotide for reducing MAPT gene expression comprises a sense strand comprising the nucleotide sequence as set forth in SEQ ID NO: 1681 and an antisense strand comprising the nucleotide sequence as set forth in SEQ ID NO: 815.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1514; and (ii) a sense strand of 19-50 nucleotides in length comprising a region of complementarity to the antisense strand, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1479; and (ii) a sense strand of 19-50 nucleotides in length comprising a region of complementarity to the antisense strand, wherein the antisense and sense strands are separate strands that form an asymmetric duplex region having an overhang of 1-4 nucleotides at the 3′ terminus of the antisense strand.

In some embodiments, an oligonucleotide for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1480; and (ii) a sense strand of 19-50 nucleotides in length comprising a region of complementarity to the antisense strand, wherein the antisense and sense strands are separate strands that form an asymmetric duplex region having an overhang of 1-4 nucleotides at the 3′ terminus of the antisense strand.

In some embodiments, a MAPT-targeting RNAi oligonucleotide for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1503; and (ii) a sense strand of 19-50 nucleotides in length comprising a region of complementarity to the antisense strand, wherein the antisense and sense strands are separate strands that form an asymmetric duplex region having an overhang of 1-4 nucleotides at the 3′ terminus of the antisense strand.

In some embodiments an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1504; and (ii) a sense strand of 19-50 nucleotides in length comprising a region of complementarity to the antisense strand, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1508; and (ii) a sense strand of 19-50 nucleotides in length comprising a region of complementarity to the antisense strand, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1514; and (ii) a sense strand of 19-50 nucleotides in length comprising a region of complementarity to the antisense strand and a stem-loop at the 3′ terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1479; and (ii) a sense strand of 19-50 nucleotides in length comprising a region of complementarity to the antisense strand and a stem-loop at the 3′ terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1480; and (ii) a sense strand of 19-50 nucleotides in length comprising a region of complementarity to the antisense strand and a stem-loop at the 3′ terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1503; and (ii) a sense strand of 19-50 nucleotides in length comprising a region of complementarity to the antisense strand and a stem-loop at the 3′ terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1504; and (ii) a sense strand of 19-50 nucleotides in length comprising a region of complementarity to the antisense strand and a stem-loop at the 3′ terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1508; and (ii) a sense strand of 19-50 nucleotides in length comprising a region of complementarity to the antisense strand and a stem-loop at the 3′ terminus, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1479; and (ii) a sense strand of 19-25 nucleotides in length comprising a region of complementarity to the antisense strand, wherein the oligonucleotide comprises a blunt end comprising the 3′ end of the sense strand, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1514; and (ii) a sense strand of 19-50 nucleotides in length comprising a region of complementarity to the antisense strand, wherein the region of complementarity to the antisense strand is set forth in SEQ ID NO: 1130, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1479; and (ii) a sense strand of 19-50 nucleotides in length comprising a region of complementarity to the antisense strand, wherein the region of complementarity to the antisense strand is set forth in SEQ ID NO: 1095, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1480; and (ii) a sense strand of 19-50 nucleotides in length comprising a region of complementarity to the antisense strand, wherein the region of complementarity to the antisense strand is set forth in SEQ ID NO: 1096, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1503; and (ii) a sense strand of 19-50 nucleotides in length comprising a region of complementarity to the antisense strand, wherein the region of complementarity to the antisense strand is set forth in SEQ ID NO: 1119, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1504; and (ii) a sense strand of 19-50 nucleotides in length comprising a region of complementarity to the antisense strand, wherein the region of complementarity to the antisense strand is set forth in SEQ ID NO: 1120, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1508; and (ii) a sense strand of 19-50 nucleotides in length comprising a region of complementarity to the antisense strand, wherein the region of complementarity to the antisense strand is set forth in SEQ ID NO: 1124, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1514; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 1130, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1479; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 1095, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1480; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 1096, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1503; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 1119, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1504; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 1120, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1508; and (ii) a sense strand of 19-50 nucleotides in length comprising a region of complementarity to the antisense strand and a stem-loop at the 3′ terminus, wherein the region of complementarity to the antisense strand is set forth in SEQ ID NO: 1124, wherein the stem-loop is set forth as S1-L-S2, wherein S1 is complementary to S2 and wherein L forms a loop between S1 and S2 of 3 to 5 nucleotides in length, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises (i) an antisense strand of 19-30 nucleotides in length, wherein the antisense strand comprises a nucleotide sequence comprising a region of complementarity to a MAPT mRNA target sequence, wherein the region of complementarity is set forth in SEQ ID NO: 1479; and (ii) a sense strand of 19-25 nucleotides in length comprising a region of complementarity to the antisense strand, wherein the region of complementarity to the antisense strand is set forth in SEQ ID NO: 1095, wherein the oligonucleotide comprises a blunt end comprising the 3′ end of the sense strand, wherein the antisense and sense strands are separate strands that form an asymmetric duplex region having an overhang of 1-4 nucleotides at the 3′ terminus of the antisense strand.

In some embodiments, an oligonucleotide (e.g., a RNAi oligonucleotide) for reducing MAPT gene expression comprises a sense strand and an antisense strand according to:

Sense Strand:
5′-mX-S-mX-fX-mX-fX-mX-mX-fX-mX-fX-mX-fX-fX-mX-fX-
mX-fX-mX-mX-mX-mX-mX-mX-mX-mX-mX-mX-[ademX-GalNAc]-
[ademX-GalNAc]-[ademX-GalNAc]-mX-mX-mX-mX-mX-mX-3′
hybridized to:
Antisense Strand:
5′-[MePhosphonate-40-mX]-S-fX-S-fX-fX-fX-mX-fX-mX-
mX-fX-mX-mX-mX-fX-mX-fX-mX-mX-fX-mX-S-mX-S-mX-3′,
wherein mX = 2′-OMe-modified nucleotide, fX =
2′-F-modified nucleotide, —S— = phosphorothioate
linkage, -= phosphodiester linkage, [MePhosphonate-
40-mX] = 4′-O-monomethylphosphonate-2′-O-
methyl-modified nucleotide, and ademX-GalNAc =
GalNAc attached to a nucleotide.

In some embodiments, an oligonucleotide (e.g., a RNAi oligonucleotide) for reducing MAPT gene expression comprises a sense strand and an antisense strand according to:

Sense Strand:
5′-mX-S-mX-mX-mX-mX-mX-mX-fX-fX-fX-fX-mX-mX-mX-mX-
mX-mX-mX-mX-mX-mX-mX-mX-mX-mX-mX-mX-[ademX-GalNAc]-
[ademX-GalNAc]-[ademX-GalNAc]-mX-mX-mX-mX-mG-mX-3′
hybridized to:
Antisense Strand:
5′-[MePhosphonate-40-mX]-S-fX-S-fX-S-fX-fX-mX-fX-
mX-mX-fX-mX-mX-mX-fX-mX-mX-mX-mX-mX-mX-S-mX-S-mX-
3′, wherein mX = 2′-OMe-modified nucleotide,
fX = 2′-F-modified nucleotide, —S— =
phosphorothioate linkage, - = phosphodiester
linkage, [MePhosphonate-40-mX] = 4′-O-
monomethylphosphonate-2′-O-methyl modified
nucleotide, and adem X-GalNAc = GalNAc attached to
a nucleotide.

In some embodiments, an oligonucleotide (e.g., a RNAi oligonucleotide) for reducing MAPT gene expression comprises a sense strand and an antisense strand according to:

Sense Strand:
5′-[ AdemX-L]-S-mX-fX-mX-fX-mX-mX-fX-mX-fX-mX-fX-
fX-mX-fX-mX-fX-mX- S-mX-S-mX-3′ hybridized to:
Antisense Strand:
5′-[MePhosphonate-40-mX]-S-fX-S-fX-fX-fX-mX-fX-mX-
mX-fX-mX-mX-mX-fX-mX-fX-mX-mX-fX-mX-S-mX-S-mX-3′,
wherein mX = 2′-OMe-modified nucleotide, fX =
2′-F-modified nucleotide, —S— = phosphorothioate
linkage, - = phosphodiester linkage,
[MePhosphonate-40-mX] = 4′-O-
monomethylphosphonate-2′-O-methyl modified
nucleotide, and ademX-L = lipid moiety attached to
a nucleotide.

In some embodiments, an oligonucleotide (e.g., a RNAi oligonucleotide) for reducing MAPT gene expression comprises a sense strand and an antisense strand according to:

Sense Strand:
5′-[ AdemX-C16]-S-mX-fX-mX-fX-mX-mX-fX-mX-fX-mX-fX-
fX-mX-fX-mX-fX-mX-S-mX-S-mX-3′ hybridized to:
Antisense Strand:
5′-[MePhosphonate-40-mX]-S-fX-S-fX-fX-fX-mX-fX-mX-
mX-fX-mX-mX-mX-fX-mX-fX-mX-mX-fX-mX-S-mX-S-mX-3′,
wherein mX = 2′-OMe-modified nucleotide, fX =
2′-F-modified nucleotide, —S— = phosphorothioate
linkage, - = phosphodiester linkage,
[MePhosphonate-40-mX] = 4′-O-
monomethylphosphonate-2′-O-methyl modified
nucleotide, and ademX-C16 = C₁₆ hydrocarbon chain
attached to a nucleotide.

In some embodiments, the current disclosure provides an oligonucleotide (e.g., a RNAi oligonucleotide) for reducing MAPT gene expression, wherein the oligonucleotide comprises a sense strand and an antisense strand comprising nucleotide sequences selected from the group consisting of:
a) SEQ ID NOs: 839 and 874, respectively;
b) SEQ ID NOs: 840 and 875, respectively;
c) SEQ ID NOs: 841 and 876, respectively;
d) SEQ ID NOs: 842 and 877, respectively;
e) SEQ ID NOs: 843 and 878, respectively;
f) SEQ ID NOs: 844 and 879, respectively;
g) SEQ ID NOs: 845 and 880, respectively;
h) SEQ ID NOs: 846 and 881, respectively;
i) SEQ ID NOs: 847 and 882, respectively;
j) SEQ ID NOs: 848 and 883, respectively;
k) SEQ ID NOs: 849 and 884, respectively;
l) SEQ ID NOs: 850 and 885, respectively;
m) SEQ ID NOs: 851 and 886, respectively;
n) SEQ ID NOs: 852 and 887, respectively;
o) SEQ ID NOs: 853 and 888, respectively;
p) SEQ ID NOs: 854 and 889, respectively;
q) SEQ ID NOs: 855 and 890, respectively;
r) SEQ ID NOs: 856 and 891, respectively;
s) SEQ ID NOs: 857 and 892, respectively;
t) SEQ ID NOs: 858 and 893, respectively;
u) SEQ ID NOs: 859 and 894, respectively;
v) SEQ ID NOs: 860 and 895, respectively;
w) SEQ ID NOs: 861 and 896, respectively;
x) SEQ ID NOs: 862 and 897, respectively;
y) SEQ ID NOs: 863 and 898, respectively;
z) SEQ ID NOs: 864 and 899, respectively;
aa) SEQ ID NOs: 865 and 900, respectively;
bb) SEQ ID NOs: 866 and 901, respectively;
cc) SEQ ID NOs: 867 and 902, respectively;
dd) SEQ ID NOs: 868 and 903, respectively;
ee) SEQ ID NOs: 869 and 904, respectively;
ff) SEQ ID NOs: 870 and 905, respectively;
gg) SEQ ID NOs: 871 and 906, respectively;
hh) SEQ ID NOs: 872 and 907, respectively;
ii) SEQ ID NOs: 873 and 908, respectively; and
jj) SEQ ID NOs: 1682 and 885, respectively.

In some embodiments, an oligonucleotide (e.g., a RNAi oligonucleotide) for reducing MAPT gene expression comprises a sense strand and an antisense strand comprising nucleotide sequences selected from the group consisting of:
a) SEQ ID NOs: 860 and 895, respectively;
b) SEQ ID NOs: 865 and 900, respectively;
c) SEQ ID NOs: 868 and 903, respectively;
d) SEQ ID NOs: 869 and 904, respectively;
e) SEQ ID NOs: 873 and 908, respectively;
f) SEQ ID NOs: 841 and 876, respectively;
g) SEQ ID NOs: 846 and 881, respectively;
h) SEQ ID NOs: 850 and 885, respectively;
i) SEQ ID NOs: 851 and 886, respectively;
j) SEQ ID NOs: 852 and 887, respectively; and
k) SEQ ID NOs: 1682 and 885, respectively.

In some embodiments, an oligonucleotide (e.g., a RNAi oligonucleotide) for reducing MAPT gene expression comprises a sense strand and an antisense strand comprising nucleotide sequences selected from the group consisting of:
a) SEQ ID NOs: 841 and 876, respectively;
b) SEQ ID NOs: 850 and 885, respectively;
c) SEQ ID NOs: 851 and 886, respectively;
d) SEQ ID NOs: 868 and 903, respectively;
e) SEQ ID NOs: 869 and 904, respectively;
f) SEQ ID NOs: 873 and 908, respectively; and
g) SEQ ID NOs: 1682 and 885, respectively.

In some embodiments, an oligonucleotide for reducing MAPT gene expression comprises a sense strand comprising the nucleotide sequence as set forth in SEQ ID NO: 841 and an antisense strand comprising the nucleotide sequence as set forth in SEQ ID NO: 876. In some embodiments, an oligonucleotide for reducing MAPT gene expression comprises a sense strand comprising the nucleotide sequence as set forth in SEQ ID NO: 850 and an antisense strand comprising the nucleotide sequence as set forth in SEQ ID NO: 885. In some embodiments, an oligonucleotide for reducing MAPT gene expression comprises a sense strand comprising the nucleotide sequence as set forth in SEQ ID NO: 851 and an antisense strand comprising the nucleotide sequence as set forth in SEQ ID NO: 886. In some embodiments, an oligonucleotide for reducing MAPT gene expression comprises a sense strand comprising the nucleotide sequence as set forth in SEQ ID NO: 868 and an antisense strand comprising the nucleotide sequence as set forth in SEQ ID NO: 903. In some embodiments, an oligonucleotide for reducing MAPT gene expression comprises a sense strand comprising the nucleotide sequence as set forth in SEQ ID NO: 869 and an antisense strand comprising the nucleotide sequence as set forth in SEQ ID NO: 904. In some embodiments, an oligonucleotide for reducing MAPT gene expression comprises a sense strand comprising the nucleotide sequence as set forth in SEQ ID NO: 873 and an antisense strand comprising the nucleotide sequence as set forth in SEQ ID NO: 908. In some embodiments, an oligonucleotide for reducing MAPT gene expression comprises a sense strand comprising the nucleotide sequence as set forth in SEQ ID NO: 1682 and an antisense strand comprising the nucleotide sequence as set forth in SEQ ID NO: 885.

### Formulations

Various formulations have been developed to facilitate oligonucleotide use. For example, oligonucleotides (e.g., RNAi oligonucleotides) can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides reduce MAPT gene expression. Such compositions can be suitably formulated such that when administered to a subject, either into the immediate environment of a target cell or systemically, a sufficient portion of the oligonucleotides enter the cell to reduce MAPT gene expression. Any variety of suitable oligonucleotide formulations can be used to deliver oligonucleotides for the reduction of MAPT gene expression as disclosed herein. In some embodiments, an oligonucleotide is formulated in buffer solutions such as phosphate buffered saline solutions, liposomes, micellar structures, and capsids. In some embodiments, an oligonucleotide is formulated in buffer solutions such as phosphate buffered saline solutions.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine, can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions. In some embodiments, an oligonucleotide is not formulated with a component to facilitate transfection into cells.

Accordingly, in some embodiments, a formulation comprises a lipid nanoparticle. In some embodiments, an excipient comprises a liposome, a lipid, a lipid complex, a microsphere, a microparticle, a nanosphere or a nanoparticle, or may be otherwise formulated for administration to the cells, tissues, organs, or body of a subject in need thereof (see, e.g., Remington: THE SCIENCE AND PRACTICE OF PHARMACY, 22nd edition, Pharmaceutical Press, 2013).

In some embodiments, the formulations herein comprise an excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, an excipient in a composition comprising any one of the oligonucleotides described herein may be a lyoprotectant (e.g., mannitol, lactose, polyethylene glycol, or polyvinylpyrrolidone) or a collapse temperature modifier (e.g., dextran, Ficoll™, or gelatin).

In some embodiments, a pharmaceutical composition is formulated to be compatible with its intended route of administration. Examples of routes of administration include parenteral (e.g., intravenous, intramuscular, intraperitoneal, intradermal, subcutaneous), oral (e.g., inhalation), transdermal (e.g., topical), transmucosal, and rectal administration.

In some embodiments, a pharmaceutical composition is formulated for administration into the central nervous system. In some embodiments, a pharmaceutical composition is formulated for administration into the cerebral spinal fluid. In some embodiments, a pharmaceutical composition is formulated for administration to the spinal cord. In some embodiments, a pharmaceutical composition is formulated for intrathecal administration. In some embodiments, a pharmaceutical composition is formulated for administration to the brain. In some embodiments, a pharmaceutical composition is formulated for intracerebroventricular administration. In some embodiments, a pharmaceutical composition is formulated for the brain stem. In some embodiments, a pharmaceutical composition is formulated for intracisternal magna administration.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF), or phosphate buffered saline (PBS). The carrier can be a solvent or dispersion medium containing, for example, water, ethanol, polyol (e.g., glycerol, propylene glycol, liquid polyethylene glycol, and the like), and suitable mixtures thereof. In many cases, it will be preferable to include isotonic agents, for example, sugars, polyalcohols such as mannitol, sorbitol, and/or sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., a RNAi oligonucleotide for reducing MAPT gene expression) or more, although the percentage of the active ingredient(s) may be between about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Use

### Reducing MAPT Gene Expression

In some embodiments, the disclosure provides methods for contacting or delivering to a cell or population of cells an effective amount of any of the oligonucleotides (e.g., RNAi oligonucleotides) herein to reduce MAPT gene expression. In some embodiments, a reduction of MAPT gene expression is determined by measuring a reduction in the amount or level of MAPT mRNA, Tau protein, or Tau activity in a cell. The methods include those described herein and known to one of ordinary skill in the art.

In some embodiments, the disclosure provides methods for reducing MAPT gene expression in the CNS. In some embodiments, the CNS comprises the brain and spinal cord. In some embodiments, MAPT gene expression is reduced in at least one region of the brain. In some embodiments, regions of the brain cervical spinal cord, thoracic spinal cord, lumbar spinal cord, frontal cortex, temporal cortex, cerebellum, midbrain, occipital cortex, parietal cortex, hippocampus, caudate nucleus, thalamus, and brainstem. In some embodiments, MAPT gene expression is reduced in at least one region of the spinal cord. In some embodiments, regions of the spinal cord include the cervical spinal cord, thoracic spinal cord, and lumbar spinal cord. In some embodiments, MAPT gene expression is reduced in at least one region of the brain and at least one region of the spinal cord. In some embodiments, MAPT gene expression is reduced in at least one of the cervical spinal cord, thoracic spinal cord, lumbar spinal cord, frontal cortex, temporal cortex, cerebellum, midbrain, occipital cortex, parietal cortex, hippocampus, caudate nucleus, thalamus, brainstem, motor cortex, globus pallidus, midbrain tegmentum, substantia nigra, pons, cerebellar white matter, and cerebellar dentate nucleus. In some embodiments, MAPT gene expression is reduced in at least one of the lumbar spinal cord, thoracic spinal cord, and cervical spinal cord. In some embodiments, MAPT gene expression is reduced in tissue of the brain and/or spinal cord associated with Alzheimer's disease. In some embodiments, tissue associated with AD includes, but is not limited to, prefrontal cortex, motor cortex, temporal cortex, parietal cortex, and hippocampus. In some embodiments, MAPT gene expression is reduced in tissue of the brain and/or spinal cord associated with progressive supranuclear palsy. In some embodiments, tissue associated with AD includes, but is not limited to caudate nucleus, globus pallidus, thalamus, midbrain tegmentum, substantia nigra, pons, cerebellar white matter, cerebellar dentate nucleus, medulla, cervical spinal cord, thoracic spinal cord, and lumbar spinal cord.

In some embodiments, MAPT gene expression is reduced for about 1 week to about 12 weeks after administration of an oligonucleotide described herein. In some embodiments, MAPT gene expression is reduced for 1, 2, 3, 4, 5, 6, 7, 8, 9, 10, 11, or 12 weeks after administration of an oligonucleotide described herein. In some embodiments, MAPT expression is reduced for about 1 to about 4 months after administration of an oligonucleotide described herein. In some embodiments, MAPT expression is reduced for about 1 to about 6 months after administration of an oligonucleotide described herein. In some embodiments, MAPT gene expression is reduced for 1, 2, 3, or 4 months after administration of an oligonucleotide described herein. In some embodiments, MAPT gene expression is reduced for 1, 2, 3 4, 5, or 6 months after administration of an oligonucleotide described herein. In some embodiments, MAPT gene expression is reduced for about 7 to about 91 days after administration of an oligonucleotide described herein. In some embodiments, MAPT gene expression is reduced for 7, 14, 21, 28, 35, 42, 49, 56, 63, 70, 77, 84, or 91 days after administration of an oligonucleotide described herein.

In some embodiments, MAPT gene expression is reduced in at least one region of the brain and/or at least one region of the spinal cord for about 1 to about 12 weeks after administration of an oligonucleotide described herein. In some embodiments, MAPT gene expression is reduced in at least one region of the brain and/or at least one region of the spinal cord for 1, 2, 3, 4, 5, 6, 7, 8, 9, 10, 11, or 12 weeks after administration of an oligonucleotide described herein. In some embodiments, MAPT gene expression is reduced in at least one region of the brain and/or at least one region of the spinal cord for about 1 to about 4 months after administration of an oligonucleotide described herein. In some embodiments, MAPT gene expression is reduced in at least one region of the brain and/or at least one region of the spinal cord for about 1 to about 6 months after administration of an oligonucleotide described herein. In some embodiments, MAPT gene expression is reduced in at least one region of the brain and/or at least one region of the spinal cord for 1, 2, 3, or 4 months after administration of an oligonucleotide described herein. In some embodiments, MAPT gene expression is reduced in at least one region of the brain and/or at least one region of the spinal cord for 1, 2, 3 4, 5, or 6 months after administration of an oligonucleotide described herein. In some embodiments, MAPT gene expression is reduced in at least one region of the brain and/or at least one region of the spinal cord for about 7 to about 91 days after administration of an oligonucleotide described herein. In some embodiments, MAPT gene expression is reduced in at least one region of the brain and/or at least one region of the spinal cord for 7, 14, 21, 28, 35, 42, 49, 56, 63, 70, 77, 84, or 91 days after administration of an oligonucleotide described herein.

Methods provided herein are useful in any appropriate cell type. In some embodiments, a cell is any cell that expresses MAPT mRNA (e.g., oligodendrocyte). In some embodiments, the cell is a primary cell obtained from a subject. In some embodiments, the primary cell has undergone a limited number of passages such that the cell substantially maintains is natural phenotypic properties. In some embodiments, a cell to which the oligonucleotide is delivered is ex vivo or in vitro (i.e., can be delivered to a cell in culture or to an organism in which the cell resides).

In some embodiments, the oligonucleotides disclosed herein are delivered to a cell or population of cells using a nucleic acid delivery method known in the art including, but not limited to, injection of a solution or pharmaceutical composition containing the oligonucleotide, bombardment by particles covered by the oligonucleotide, exposing the cell or population of cells to a solution containing the oligonucleotide, or electroporation of cell membranes in the presence of the oligonucleotide. Other methods known in the art for delivering oligonucleotides to cells may be used, such as lipid-mediated carrier transport, chemical-mediated transport, and cationic liposome transfection such as calcium phosphate, and others.

In some embodiments, reduction of MAPT gene expression is determined by an assay or technique that evaluates one or more molecules, properties or characteristics of a cell or population of cells associated with MAPT gene expression, or by an assay or technique that evaluates molecules that are directly indicative of MAPT gene expression in a cell or population of cells (e.g., MAPT mRNA or Tau protein). In some embodiments, the extent to which an oligonucleotide reduces MAPT gene expression is evaluated by comparing MAPT gene expression in a cell or population of cells contacted with the oligonucleotide to a control cell or population of cells (e.g., a cell or population of cells not contacted with the oligonucleotide or contacted with a control oligonucleotide). In some embodiments, a control amount or level of MAPT gene expression in a control cell or population of cells is predetermined, such that the control amount or level need not be measured in every instance the assay or technique is performed. The predetermined level or value can take a variety of forms. In some embodiments, a predetermined level or value can be single cut-off value, such as a median or mean.

In some embodiments, contacting or delivering an oligonucleotide to a cell or a population of cells results in a reduction in MAPT gene expression. In some embodiments, the reduction in MAPT gene expression is relative to a control amount or level of MAPT gene expression in cell or population of cells not contacted with the oligonucleotide or contacted with a control oligonucleotide. In some embodiments, the reduction in MAPT gene expression is about 1% or lower, about 5% or lower, about 10% or lower, about 15% or lower, about 20% or lower, about 25% or lower, about 30% or lower, about 35% or lower, about 40% or lower, about 45% or lower, about 50% or lower, about 55% or lower, about 60% or lower, about 70% or lower, about 80% or lower, or about 90% or lower relative to a control amount or level of MAPT gene expression. In some embodiments, the control amount or level of MAPT gene expression is an amount or level of MAPT mRNA and/or Tau protein in a cell or population of cells that has not been contacted with an oligonucleotide herein. In some embodiments, the effect of delivery of an oligonucleotide to a cell or population of cells according to a method herein is assessed after any finite period or amount of time (e.g., minutes, hours, days, weeks, months). For example, in some embodiments, MAPT gene expression is determined in a cell or population of cells at least about 4 hours, about 8 hours, about 12 hours, about 18 hours, about 24 hours; or at least about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 35 days, about 42 days, about 49 days, about 56 days, about 63 days, about 70 days, about 77 days, about 84 days, or more after contacting or delivering the oligonucleotide to the cell or population of cells. In some embodiments, MAPT gene expression is determined in a cell or population of cells at least about 1 month, about 2 months, about 3 months, about 4 months, about 5 months, about 6 months, or more after contacting or delivering the oligonucleotide to the cell or population of cells.

In some embodiments, the oligonucleotide is delivered in the form of a transgene that is engineered to express in a cell the oligonucleotide or strands comprising the oligonucleotide (e.g., its sense and antisense strands). In some embodiments, the oligonucleotide is delivered using a transgene engineered to express any oligonucleotide disclosed herein. Transgenes may be delivered using viral vectors (e.g., adenovirus, retrovirus, vaccinia virus, poxvirus, adeno-associated virus, or herpes simplex virus) or non-viral vectors (e.g., plasmids or synthetic mRNAs). In some embodiments, transgenes can be injected directly to a subject.

### Treatment Methods

The disclosure also provides oligonucleotides (e.g., RNAi oligonucleotides) for use, or adaptable for use, to treat a subject (e.g., a human having a disease, disorder, or condition associated with MAPT gene expression) that would benefit from reducing MAPT gene expression. In some aspects, the disclosure provides oligonucleotides for use, or adapted for use, to treat a subject having a disease, disorder, or condition associated with MAPT gene expression. The disclosure also provides oligonucleotides for use, or adaptable for use, in the manufacture of a medicament or pharmaceutical composition for treating a disease, disorder, or condition associated with MAPT gene expression. In some embodiments, the oligonucleotides for use, or adaptable for use, target MAPT mRNA and reduce MAPT gene expression (e.g., via the RNAi pathway). In some embodiments, the oligonucleotides for use, or adaptable for use, target MAPT mRNA and reduce the amount or level of MAPT mRNA, Tau protein, and/or Tau activity.

In addition, in some embodiments of the methods herein, a subject having a disease, disorder or condition associated with MAPT gene expression or is predisposed to the same is selected for treatment with an oligonucleotide (e.g., a ds oligonucleotide) herein. In some embodiments, the method comprises selecting an individual having a marker (e.g., a biomarker) for a disease, disorder, or condition associated with MAPT gene expression, or predisposed to the same, such as, but not limited to, MAPT mRNA, Tau protein, or a combination thereof. Likewise, and as detailed below, some embodiments of the methods provided by the disclosure include steps such as measuring or obtaining a baseline value for a marker of MAPT gene expression (e.g., Tau protein or Tau activity), and then comparing such obtained value to one or more other baseline values or values obtained after the subject is administered the oligonucleotide to assess the effectiveness of treatment.

The disclosure also provides methods of treating a subject having, suspected of having, or at risk of developing a disease, disorder, or condition associated with MAPT gene expression with an oligonucleotide provided herein. In some aspects, the disclosure provides methods of treating or attenuating the onset or progression of a disease, disorder, or condition associated with MAPT gene expression using the oligonucleotides provided herein. In other aspects, the disclosure provides methods to achieve one or more therapeutic benefits in a subject having a disease, disorder, or condition associated with MAPT gene expression using the oligonucleotides provided herein. In some embodiments of the methods herein, the subject is treated by administering a therapeutically effective amount of any one or more of the oligonucleotides provided herein. In some embodiments, treatment comprises reducing MAPT gene expression. In some embodiments, the subject is treated therapeutically. In some embodiments, the subject is treated prophylactically.

In some embodiments of the methods herein, an oligonucleotide (e.g., a RNAi oligonucleotide), or a pharmaceutical composition comprising the oligonucleotide, is administered to a subject having a disease, disorder, or condition associated with MAPT gene expression such that MAPT gene expression is reduced in the subject, thereby treating the subject. In some embodiments, an amount or level of MAPT mRNA is reduced in the subject. In some embodiments, an amount or level of Tau protein is reduced in the subject.

In some embodiments of the methods herein, the oligonucleotide, or a pharmaceutical composition comprising the oligonucleotide, is administered to a subject having a disease, disorder, or condition associated with MAPT gene expression such that MAPT gene expression is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to MAPT gene expression prior to administration of the oligonucleotide or pharmaceutical composition. In some embodiments of the methods herein, the oligonucleotide, or the pharmaceutical composition comprising the oligonucleotide, is administered to a subject having a disease, disorder, or condition associated with MAPT gene expression such that MAPT gene expression is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to MAPT gene expression prior to administration of the oligonucleotide or pharmaceutical composition. In some embodiments, MAPT gene expression is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to MAPT gene expression in a subject (e.g., a reference or control subject) not receiving the oligonucleotide or pharmaceutical composition or receiving a control oligonucleotide, pharmaceutical composition or treatment. In some embodiments, MAPT gene expression is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to MAPT gene expression in a subject (e.g., a reference or control subject) not receiving the oligonucleotide or pharmaceutical composition or receiving a control oligonucleotide, pharmaceutical composition or treatment.

In some embodiments of the methods herein, the oligonucleotide or the pharmaceutical composition is administered to a subject having a disease, disorder, or condition associated with MAPT gene expression such that an amount or level of MAPT mRNA is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to the amount or level of MAPT mRNA prior to administration of the oligonucleotide or pharmaceutical composition. In some embodiments of the methods, the oligonucleotide or the pharmaceutical composition is administered to a subject having a disease, disorder, or condition associated with MAPT gene expression such that an amount or level of MAPT mRNA is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to the amount or level of MAPT mRNA prior to administration of the oligonucleotide or pharmaceutical composition. In some embodiments, an amount or level of MAPT mRNA is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of MAPT mRNA in a subject (e.g., a reference or control subject) not receiving the oligonucleotide or pharmaceutical composition or receiving a control oligonucleotide, pharmaceutical composition or treatment. In some embodiments, an amount or level of MAPT mRNA is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to an amount or level of MAPT mRNA in a subject (e.g., a reference or control subject) not receiving the oligonucleotide or pharmaceutical composition or receiving a control oligonucleotide, pharmaceutical composition or treatment.

In some embodiments of the methods, the oligonucleotide or the pharmaceutical composition is administered to a subject having a disease, disorder, or condition associated with MAPT gene expression such that an amount or level of Tau protein is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to the amount or level of Tau protein prior to administration of the oligonucleotide or pharmaceutical composition. In some embodiments of the methods herein, an oligonucleotide herein, or a pharmaceutical composition comprising the oligonucleotide, is administered to a subject having a disease, disorder, or condition associated with MAPT gene expression such that an amount or level of Tau protein is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to the amount or level of Tau protein prior to administration of the oligonucleotide or pharmaceutical composition. In some embodiments, an amount or level of Tau protein is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of Tau protein in a subject (e.g., a reference or control subject) not receiving the oligonucleotide or pharmaceutical composition or receiving a control oligonucleotide, pharmaceutical composition or treatment. In some embodiments, an amount or level of Tau protein is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to an amount or level of Tau protein in a subject (e.g., a reference or control subject) not receiving the oligonucleotide or pharmaceutical composition or receiving a control oligonucleotide, pharmaceutical composition or treatment.

In some embodiments of the methods, the oligonucleotide or the pharmaceutical composition is administered to a subject having a disease, disorder, or condition associated with MAPT gene expression such that an amount or level of Tau activity is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to the amount or level of Tau activity prior to administration of the oligonucleotide or pharmaceutical composition. In some embodiments, the oligonucleotide or the pharmaceutical composition is administered to a subject having a disease, disorder, or condition associated with MAPT gene expression such that an amount or level of Tau activity is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to the amount or level of Tau activity prior to administration of the oligonucleotide or pharmaceutical composition. In some embodiments, an amount or level of Tau activity is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% when compared to an amount or level of Tau activity in a subject (e.g., a reference or control subject) not receiving the oligonucleotide or pharmaceutical composition or receiving a control oligonucleotide, pharmaceutical composition or treatment. In some embodiments, an amount or level of Tau activity is reduced in the subject by at least about 30%, about 35%, about 40%, about 45%, about 50%, about 55%, about 60%, about 65%, about 70%, about 75%, about 80%, about 85%, about 90%, about 95%, about 99%, or greater than 99% for about 1 week to about 12 weeks, about 1 month to about 6 months, or about 7 days to about 91 days when compared to an amount or level of Tau activity in a subject (e.g., a reference or control subject) not receiving the oligonucleotide or pharmaceutical composition or receiving a control oligonucleotide, pharmaceutical composition or treatment.

Suitable methods for determining MAPT gene expression, an amount or level of MAPT mRNA, an amount or level of Tau protein, and/or an amount or level of Tau activity, in the subject, or in a sample from the subject, are known in the art. Further, the Examples set forth herein illustrate exemplary methods for determining MAPT gene expression.

In some embodiments, MAPT gene expression, an amount or level of MAPT mRNA, an amount or level of Tau protein, an amount or level of Tau activity, or any combination thereof, is reduced in a cell (e.g., an oligodendrocyte), a population or a group of cells (e.g., an organoid), an organ (e.g., frontal cortex), blood or a fraction thereof (e.g., plasma), a tissue (e.g., brain tissue), a sample (e.g., a brain biopsy sample), or any other biological material obtained or isolated from the subject. In some embodiments, MAPT gene expression, an amount or level of MAPT mRNA, an amount or level of Tau protein, an amount or level of Tau activity, or any combination thereof, is reduced in more than one type of cell (e.g., an oligodendrocyte and one or more other type(s) of cell), more than one groups of cells, more than one organ (e.g., brain and one or more other organ(s)), more than one fraction of blood (e.g., plasma and one or more other blood fraction(s)), more than one type of tissue (e.g., brain tissue and one or more other type(s) of tissue), more than one type of sample (e.g., a brain biopsy sample and one or more other type(s) of biopsy sample) obtained or isolated from the subject. In some embodiments, MAPT gene expression, an amount or level of MAPT mRNA, an amount or level of Tau protein, an amount or level of Tau activity, or any combination thereof is reduced in one or more of the cervical spinal cord, thoracic spinal cord, lumbar spinal cord, frontal cortex, temporal cortex, cerebellum, midbrain, occipital cortex, parietal cortex, hippocampus, caudate nucleus, thalamus, brainstem, motor cortex, globus pallidus, midbrain tegmentum, substantia nigra, pons, cerebellar white matter, and cerebellar dentate nucleus. In some embodiments, MAPT gene expression, an amount or level of MAPT mRNA, an amount or level of Tau protein, an amount or level of Tau activity, or any combination thereof is reduced in tissue of the brain and/or spinal cord associated with AD. In some embodiments, tissue associated with AD includes, but is not limited to, prefrontal cortex, motor cortex, temporal cortex, parietal cortex, and hippocampus. In some embodiments, MAPT gene expression, an amount or level of MAPT mRNA, an amount or level of Tau protein, an amount or level of Tau activity, or any combination thereof is reduced in tissue of the brain and/or spinal cord associated with PSP. In some embodiments, tissue associated with AD includes, but is not limited to caudate nucleus, globus pallidus, thalamus, midbrain tegmentum, substantia nigra, pons, cerebellar white matter, cerebellar dentate nucleus, medulla, cervical spinal cord, thoracic spinal cord, and lumbar spinal cord.

Examples of a disease, disorder, or condition associated with MAPT gene expression include, but are not limited to, AD, FTD, PD, PSP, and Tau protein associated diseases (e.g., primary age-related tauopathy, chronic traumatic encephalopathy, corticobasal degeneration, lytico-bodig disease, ganglioglioma, meningioangiomatosis, postencephalitic parkinsonism, and subacute sclerosing panencephalitis), which have aberrant MAPT gene expression that results in pathology of these diseases. Over 50 missense, silencing, and intronic mutations are known in MAPT (Ghetti et al. (2015) NEUROPATHOL. APPL. NEUROBIOL. 41:24-46) that lead to these diseases.

Because of their high specificity, the oligonucleotides herein (e.g., RNAi oligonucleotides) specifically target mRNAs of target genes of cells, tissue(s), or organ(s) (e.g., brain). In preventing disease, the target gene may be one that is required for initiation or maintenance of the disease or that has been identified as being associated with a higher risk of contracting the disease. In treating disease, the oligonucleotide can be brought into contact with the cells, tissue(s), or organ(s) (e.g., brain) exhibiting or responsible for mediating the disease. For example, an oligonucleotide substantially identical to all or part of a wild-type (i.e., native) or mutated gene associated with a disorder or condition associated with MAPT gene expression may be brought into contact with or introduced into a cell or tissue type of interest such as an oligodendrocyte or other brain cell.

In some embodiments, the target gene may be a target gene from any mammal, such as a human. Any gene may be silenced according to the method described herein.

Methods described herein are typically involve administering to a subject a therapeutically effective amount of an oligonucleotide (e.g., a RNAi oligonucleotide), that is, an amount capable of producing a desirable therapeutic result. A therapeutically acceptable amount may be an amount that can therapeutically treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

In some embodiments, a subject is administered any one of the compositions herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intraosseous infusion, intramuscular injection, intracerebral injection, intracerebroventricular injection, or intrathecal), topically (e.g., epicutaneous, inhalational, via eye drops, or through a mucous membrane), or by direct injection into a target organ (e.g., the brain of a subject). Typically, the oligonucleotides are administered intravenously or subcutaneously. In some embodiments, the oligonucleotides are administered to the cerebral spinal fluid. In some embodiments, the oligonucleotides described herein are administered intrathecally. In some embodiments, the oligonucleotides are administered intracerebroventricularly. In some embodiments, the oligonucleotides are administered by intracisternal magna injection.

As a non-limiting set of examples, the oligonucleotides would typically be administered quarterly (once every three months), bi-monthly (once every two months), monthly or weekly. For example, the oligonucleotides may be administered every week or at intervals of two, or three weeks. Alternatively, the oligonucleotides may be administered daily. In some embodiments, a subject is administered one or more loading doses of the oligonucleotide followed by one or more maintenance doses of the oligonucleotide.

In some embodiments, the subject to be treated is a human or NHP or other mammalian subject. Other exemplary subjects include domesticated animals such as dogs and cats; livestock such as horses, cattle, pigs, sheep, goats, and chickens; and animals such as mice, rats, guinea pigs, and hamsters.

### Kits

In some embodiments, the disclosure provides a kit comprising an oligonucleotide herein (e.g., a RNAi oligonucleotide), and instructions for use. In some embodiments, the kit comprises the oligonucleotide and a package insert containing instructions for use of the kit and/or any component thereof. In some embodiments, the kit comprises, in a suitable container, the oligonucleotide, one or more controls, and various buffers, reagents, enzymes and other standard ingredients well known in the art. In some embodiments, the container comprises at least one vial, well, test tube, flask, bottle, syringe, or other container means, into which the oligonucleotide is placed, and in some instances, suitably aliquoted. In some embodiments where an additional component is provided, the kit contains additional containers into which this component is placed. The kits can also include a means for containing the oligonucleotide and any other reagent in close confinement for commercial sale. Such containers may include injection or blow-molded plastic containers into which the desired vials are retained. Containers and/or kits can include labeling with instructions for use and/or warnings.

In some embodiments, the kit comprises the oligonucleotide and a pharmaceutically acceptable carrier, or a pharmaceutical composition comprising the oligonucleotide and instructions for treating or delaying progression of a disease, disorder or condition associated with MAPT gene expression in a subject in need thereof.

In some embodiments, the kit comprises the oligonucleotide and a pharmaceutically acceptable carrier or a pharmaceutical composition comprising the oligonucleotide, and instructions for administering the oligonucleotide or pharmaceutical composition to the cerebral spinal fluid to reduce MAPT gene expression in at least one region of the brain and/or at least one region of the spinal cord in a subject in need thereof.

### Definitions

As used herein, “approximately” or “about,” as applied to one or more values of interest, refers to a value that is similar to a stated reference value. In certain embodiments, “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

As used herein, “administer,” “administering,” “administration” and the like refers to providing a substance (e.g., an oligonucleotide) to a subject in a manner that is pharmacologically useful (e.g., to treat a condition in the subject).

As used herein, “asialoglycoprotein receptor” or “ASGPR” refers to a bipartite C-type lectin formed by a major 48 kDa subunit (ASGPR-1) and minor 40 kDa subunit (ASGPR-2). ASGPR is primarily expressed on the sinusoidal surface of hepatocyte cells and has a major role in binding, internalizing and subsequent clearing of circulating glycoproteins that contain terminal galactose or GalNAc residues (asialoglycoproteins).

As used herein, “attenuate,” “attenuating,” “attenuation” and the like refers to reducing or effectively halting. As a non-limiting example, one or more of the treatments herein may reduce or effectively halt the onset or progression of a disease associated with MAPT gene expression (e.g., Tau-associated diseases) in a subject. This attenuation may be exemplified by, for example, a decrease in one or more aspects (e.g., symptoms, tissue characteristics, and cellular, inflammatory or immunological activity, etc.) of a disease associated with MAPT gene expression (e.g., Tau-associated diseases), no detectable progression (worsening) of one or more aspects of the disease, or no detectable aspects of the disease in a subject when they might otherwise be expected.

As used herein, “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have regions of multiple nucleotides that are complementary with each other to form regions of complementarity, as described herein.

As used herein, “deoxyribonucleotide” refers to a nucleotide having a hydrogen in place of a hydroxyl at the 2′ position of its pentose sugar when compared with a ribonucleotide. A modified deoxyribonucleotide is a deoxyribonucleotide having one or more modifications or substitutions of atoms other than at the 2′ position, including modifications or substitutions in or of the sugar, phosphate group or base.

As used herein, “double-stranded oligonucleotide” or “ds oligonucleotide” refers to an oligonucleotide that is substantially in a duplex form. In some embodiments, the complementary base-pairing of duplex region(s) of a ds oligonucleotide is formed between antiparallel sequences of nucleotides of covalently separate nucleic acid strands. In some embodiments, complementary base-pairing of duplex region(s) of a ds oligonucleotide is formed between antiparallel sequences of nucleotides of nucleic acid strands that are covalently linked. In some embodiments, complementary base-pairing of duplex region(s) of a ds oligonucleotide is formed from single nucleic acid strand that is folded (e.g., via a hairpin) to provide complementary antiparallel sequences of nucleotides that base pair together. In some embodiments, a ds oligonucleotide comprises two covalently separate nucleic acid strands that are fully duplexed with one another. However, in some embodiments, a ds oligonucleotide comprises two covalently separate nucleic acid strands that are partially duplexed (e.g., having overhangs at one or both ends). In some embodiments, a ds oligonucleotide comprises antiparallel sequence of nucleotides that are partially complementary, and thus, may have one or more mismatches, which may include internal mismatches or end mismatches.

As used herein, “duplex,” in reference to nucleic acids (e.g., oligonucleotides), refers to a structure formed through complementary base pairing of two antiparallel sequences of nucleotides.

As used herein, “excipient” refers to a non-therapeutic agent that may be included in a composition, for example, to provide or contribute to a desired consistency or stabilizing effect.

As used herein, “labile linker” refers to a linker that can be cleaved (e.g., by acidic pH). A “fairly stable linker” refers to a linker that cannot be cleaved.

As used herein, “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

As used herein, “modified internucleotide linkage” refers to an internucleotide linkage having one or more chemical modifications when compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, “modified nucleotide” refers to a nucleotide having one or more chemical modifications when compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modification in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc.

As used herein, “nicked tetraloop structure” refers to a structure of an RNAi oligonucleotide that is characterized by separate sense (passenger) and antisense (guide) strands, in which the sense strand has a region of complementarity with the antisense strand, and in which at least one of the strands, generally the sense strand, has a tetraL configured to stabilize an adjacent stem region formed within the at least one strand.

As used herein, “oligonucleotide” refers to a short nucleic acid (e.g., less than about 100 nucleotides in length). An oligonucleotide may be ss or ds. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or ss siRNA. In some embodiments, a ds oligonucleotide is an RNAi oligonucleotide.

As used herein, “overhang” refers to terminal non-base pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a ds oligonucleotide. In certain embodiments, the overhang is a 3′ or 5′ overhang on the antisense strand or sense strand of a ds oligonucleotides.

As used herein, “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′-phosphate analog contains a phosphatase-resistant linkage. Examples of phosphate analogs include, but are not limited to, 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′ terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., U.S. Provisional Patent Application Nos. 62/383,207 (filed on 2 Sep. 2016) and 62/393,401 (filed on 12 Sep. 2016). Other modifications have been developed for the 5′ end of oligonucleotides (see, e.g., Intl. Patent Application Publication No. WO 2011/133871; U.S. Pat. No. 8,927,513; and Prakash et al. (2015) NUCLEIC ACIDS RES. 43:2993-3011).

As used herein, “MAPT” refers to Microtubule-Associated Protein Tau. The MAPT transcript undergoes several types of alternative splicing to produce different mRNA species and Tau proteins. There are six known Tau isoforms produced by the splicing of MAPT mRNA. MAPT gene expression is found primarily in the axons of neurons in the CNS. Tau protein interacts with tubulin to generate microtubules which are involved in several cellular processes. The MAPT mRNA encoding wild-type human Tau protein is set forth in SEQ ID NO: 909. The MAPT mRNA encoding mouse Tau protein is set forth in SEQ ID NO: 910. The MAPT mRNA encoding monkey Tau protein is set forth in SEQ ID NO: 911. One of skill in the art, however, understands that additional examples of MAPT mRNA sequences are readily available using publicly available databases such as, for example, GenBank and UniProt.

As used herein, “reduced expression” of a gene (e.g., MAPT) refers to a decrease in the amount or level of RNA transcript (e.g., MAPT mRNA) or protein encoded by the gene and/or a decrease in the amount or level of activity of the gene in a cell, a population of cells, a sample or a subject, when compared to an appropriate reference (e.g., a reference cell, population of cells, sample, or subject). For example, the act of contacting a cell with an oligonucleotide herein (e.g., an oligonucleotide comprising an antisense strand having a nucleotide sequence that is complementary to a nucleotide sequence comprising MAPT mRNA) may result in a decrease in the amount or level of MAPT mRNA, Tau protein, and/or Tau activity (e.g., via inactivation and/or degradation of MAPT mRNA by the RNAi pathway) when compared to a cell that is not treated with the ds oligonucleotide. Similarly, and as used herein, “reducing expression” refers to an act that results in reduced expression of a gene (e.g., MAPT).

As used herein, “reduction of MAPT gene expression” refers to a decrease in the amount or level of MAPT mRNA, Tau protein, and/or Tau activity in a cell, a population of cells, a sample, or a subject when compared to an appropriate reference (e.g., a reference cell, population of cells, sample, or subject).

As used herein, “region of complementarity” refers to a sequence of nucleotides of a nucleic acid (e.g., a ds oligonucleotide) that is sufficiently complementary to an antiparallel sequence of nucleotides to permit hybridization between the two sequences of nucleotides under appropriate hybridization conditions (e.g., in a phosphate buffer, in a cell, etc.). In some embodiments, an oligonucleotide herein comprises a targeting sequence having a region of complementary to a mRNA target sequence.

As used herein, “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, “RNAi oligonucleotide” refers to either (a) a ds oligonucleotide having a sense strand (passenger) and antisense strand (guide), in which the antisense strand or part of the antisense strand is used by the Argonaute 2 (Ago2) endonuclease in the cleavage of a target mRNA (e.g., MAPT mRNA) or (b) a ss oligonucleotide having a single antisense strand, where that antisense strand (or part of that antisense strand) is used by the Ago2 endonuclease in the cleavage of a target mRNA (e.g., MAPT mRNA).

As used herein, “strand” refers to a single, contiguous sequence of nucleotides linked together through internucleotide linkages (e.g., phosphodiester linkages or phosphorothioate linkages). In some embodiments, a strand has two free ends (e.g., a 5′ end and a 3′ end).

As used herein, “subject” means any mammal, including mice, rabbits, and humans. In one embodiment, the subject is a human or NHP. Moreover, “individual” or “patient” may be used interchangeably with “subject.”

As used herein, “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

As used herein, “targeting ligand” refers to a molecule (e.g., a carbohydrate, amino sugar, cholesterol, or polypeptide) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, “tetraloop” or “tetraL” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraL can confer a Tₘ of at least about 50° C., at least about 55° C., at least about 56° C., at least about 58° C., at least about 60° C., at least about 65° C. or at least about 75° C. in 10 mM NaHPO₄ to a hairpin comprising a duplex of at least 2 base pairs (bp) in length. In some embodiments, a tetraL may stabilize a bp in an adjacent stem duplex by stacking interactions. In addition, interactions among the nucleotides in a tetraL include, but are not limited to, non-Watson-Crick base pairing, stacking interactions, hydrogen bonding and contact interactions (Cheong et al. (1990) NATURE 346:680-682; Heus & Pardi (1991) SCIENCE 253:191-94). In some embodiments, a tetraL comprises or consists of 3 to 6 nucleotides and is typically 4 to 5 nucleotides. In certain embodiments, a tetraL comprises or consists of 3, 4, 5, or 6 nucleotides, which may or may not be modified (e.g., which may or may not be conjugated to a targeting moiety). In certain embodiments, a tetraL comprises or consists of 3, 4, 5, or 6 nucleotides, which may or may not be modified (e.g., which may or may not be conjugated to a targeting ligand). In one embodiment, a tetraL consists of 4 nucleotides. Any nucleotide may be used in the tetraloop and standard IUPAC-IUB symbols for such nucleotides may be used as described in Cornish-Bowden (1985) NUCLEIC ACIDS RES. 13:3021-30. For example, the letter “N” may be used to mean that any base may be in that position, the letter “R” may be used to show that A (adenine) or G (guanine) may be in that position, and “B” may be used to show that C (cytosine), G (guanine), T (thymine) or U (uracil) may be in that position. Examples of tetraLs include the UNCG family of tetraLs (e.g., UUCG), the GNRA family of tetraLs (e.g., GAAA), and the CUUG tetraloop (Woese et al. (1990) PROC. NATL. ACAD. SCI. USA 87:8467-71; Antao et al. (1991) NUCLEIC ACIDS RES. 19:5901-05). Examples of DNA tetraLs include the d(GNNA) family of tetraLs (e.g., d(GTTA), the d(GNRA)) family of tetraLs, the d(GNAB) family of tetraLs, the d(CNNG) family of tetraLs, and the d(TNCG) family of tetraLs (e.g., d(TTCG)). See, e.g., Nakano et al. (2002) BIOCHEM. 41:4281-92; Shinji et al. (2000) NIPPON KAGAKKAI KOEN YOKOSHU 78:731. In some embodiments, the tetraloop is contained within a nicked tetraL structure.

As used herein, “treat” or “treating” refers to the act of providing care to a subject in need thereof, for example, by administering a therapeutic agent (e.g., an oligonucleotide herein) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom, or contributing factor of a condition (e.g., disease or disorder) experienced by a subject.

### EXAMPLES

The following non-limiting examples are offered for purposes of illustration, not limitation.

### Example 1: Preparation of RNAi Oligonucleotides

### Oligonucleotide Synthesis and Purification

The oligonucleotides (RNAi oligonucleotides) described in the foregoing Examples are chemically synthesized using methods described herein. Generally, RNAi oligonucleotides are synthesized using solid phase oligonucleotide synthesis methods as described for 19-23mer siRNAs (see, e.g., Scaringe et al. (1990) NUCLEIC ACIDS RES. 18:5433-5441 and Usman et al. (1987) J. AM. CHEM. SOC. 109:7845-45; see also, U.S. Pat. Nos. 5,804,683; 5,831,071; 5,998,203; 6,008,400; 6,111,086; 6,117,657; 6,353,098; 6,362,323; 6,437,117 and 6,469,158) in addition to using known phosphoramidite synthesis (see, e.g. Hughes & Ellington (2017) COLD SPRING HARB. PERSPECT. BIOL. 9 (1): a023812; Beaucage & Caruthers (1981) TETRAHEDRON LETT. 22:1859-62). dsRNAi oligonucleotides have a 19mer core sequence were formatted into constructs having a 25mer sense strand and a 27mer antisense strand to allow for processing by the RNAi machinery. The 19mer core sequence is complementary to a region in the MAPT mRNA.

Individual RNA strands were synthesized and HPLC purified according to standard methods (Integrated DNA Technologies). For example, RNA oligonucleotides were synthesized using solid phase phosphoramidite chemistry, deprotected, and desalted on NAP-5 columns (Amersham Pharmacia Biotech) using standard techniques (Damha & Olgivie (1993) METHODS MOL. BIOL. 20:81-114; Wincott et al. (1995) NUCLEIC ACIDS RES. 23:2677-84). The oligomers were purified using ion-exchange high performance liquid chromatography (IE-HPLC) on an Amersham Source 15Q column (1.0 cm×25 cm; Amersham Pharmacia Biotech) using a 15 min step-linear gradient. The gradient varied from 90:10 Buffers A:B to 52:48 Buffers A:B, where Buffer A is 100 mM Tris pH 8.5 and Buffer B is 100 mM Tris pH 8.5, 1 M NaCl. Samples were monitored at 260 nm and peaks corresponding to the full-length oligonucleotide species were collected, pooled, desalted on NAP-5 columns, and lyophilized.

The purity of each oligomer was determined by capillary electrophoresis (CE) on a Beckman PACE 5000 (Beckman Coulter, Inc.). The CE capillaries have a 100 μm inner diameter and contain ssDNA 100R Gel (Beckman-Coulter). Typically, about 0.6 nmole of oligonucleotide was injected into a capillary, run in an electric field of 444 V/cm, and was detected by UV absorbance at 260 nm. Denaturing Tris-Borate-7 M-urea running buffer was purchased from Beckman-Coulter. Oligoribonucleotides were obtained that were at least 90% pure as assessed by CE for use in experiments described below. Compound identity was verified by matrix-assisted laser desorption ionization time-of-flight (MALDI-TOF) mass spectroscopy on a Voyager DE™ Biospectometry Work Station (Applied Biosystems) following the manufacturer's recommended protocol. Relative molecular masses of all oligomers were obtained, often within 0.2% of expected molecular mass.

### Preparation of Duplexes

ss RNA oligomers were resuspended (e.g., at 100 μM concentration) in duplex buffer consisting of 100 mM potassium acetate, 30 mM HEPES, pH 7.5. Complementary sense and antisense strands were mixed in equal molar amounts to yield a final solution of, for example, 50 μM duplex. Samples were heated to 100° C. for 5 min in RNA buffer (IDT) and were allowed to cool to room temperature before use. The RNAi oligonucleotides were stored at −20° C. ss RNA oligomers were stored lyophilized or in nuclease-free water at −80° C.

### Example 2: Generation of MAPT-Targeting ds RNAi Oligonucleotides

Identification of MAPT mRNA Target Sequences

To generate MAPT-targeting RNAi oligonucleotides, a computer-based algorithm was used to computationally identify MAPT mRNA target sequences suitable for assaying inhibition of MAPT gene expression by the RNAi pathway. The algorithm provided RNAi oligonucleotide antisense (guide) strand sequences each having a region of complementarity to a suitable MAPT mRNA target sequence of human (Hs) or murine (Mm) mRNA (e.g., SEQ ID NOs: 909 and 910, respectively; Table 1). Due to sequence conservation across species, some of the MAPT mRNA target sequences identified for human MAPT mRNA are homologous to the corresponding MAPT mRNA target sequence of murine (mM) MAPT mRNA (SEQ ID NO: 910; Table 1) and/or monkey (Mf) MAPT mRNA (SEQ ID NO: 911; Table 1). MAPT-targeting RNAi oligonucleotides comprising a region of complementarity to homologous MAPT mRNA target sequences with nucleotide sequence similarity are predicted to have the ability to target homologous MAPT mRNAs (e.g., human and monkey MAPT mRNAs).

TABLE 1
Exemplary Human, Monkey, and
Mouse MAPT mRNA Sequences.

| Species | GenBank Ref Seq # | SEQ ID NO |
|---|---|---|
| Human (Hs) | NM_001123066.3 | 909 |
| Mouse (Mm) | NM_001038609.02 | 910 |
| Cynomolgus monkey (Mf) | XM_005584531.2 | 911 |

RNAi oligonucleotides (formatted as DsiRNA oligonucleotides) were generated as described in Example 1 for evaluation in vitro. Each DsiRNA was generated with the same modification pattern, and each with a unique guide strand having a region of complementarity to a MAPT target sequence identified by the algorithm. Modifications for the sense and antisense DsiRNA included the following (X-any nucleotide; m-2′-OMe-modified nucleotide; r-ribosyl-modified nucleotide):

Sense Strand:
rXmXrXmXrXrXrXrXrXrXrXrXrXmXrXmXrXrXrXrXrXrXrXXX
Anti-sense Strand:
mXmXmXmXrXrXrXrXrXrXmXrXmXrXrXrXrXrXrXrXrXrXmXrXmX
mXmX

### In Vitro Cell-Based Assays

The ability of each of the modified DsiRNA in Table 2 to reduce MAPT mRNA was measured using in vitro cell-based assays. Briefly, human T98G cells (glioblastoma cell line) expressing endogenous human MAPT gene were transfected with each of the DsiRNAs listed in Table 2 at 1 nM in separate wells of a multi-well cell-culture plate. Cells were maintained for 24 hours following transfection with the modified DsiRNA, and then the amount of remaining MAPT mRNA from the transfected cells was determined using TAQMAN®-based qPCR assays. Two qPCR assays, a 3′ assay (Forward; GAA GAT TGG GTC CCT GGA (SEQ ID NO: 1683), Reverse; TGT CTT GGC TTT GGC GTT (SEQ ID NO: 1684), Probe; 5′-6FAM-CGG AAG GTC/ZEN/AGC TTG TGG GTT TCA (SEQ ID NO: 1685); and a 5′ assay (Forward; CAC CAC AGC CAC CTT CTC (SEQ ID NO: 1686), Reverse; CTT CCA TCA CTT CGA ACT CCT (SEQ ID NO: 1687), Probe; 5′-6FAM-CGT CCT CGC/ZEN/CTC TGT CGA CTA (SEQ ID NO: 1688) were used to determine MAPT mRNA levels as measured using PCR probes conjugated to 6-carboxy-fluorescein (FAM). Primer pairs were assayed for % remaining mRNA as shown in Table 2. DsiRNAs resulting in less than or equal to 10% MAPT mRNA remaining in DsiRNA-transfected cells when compared to mock-transfected cells were considered DsiRNA “hits.” The T98G cell-based assay evaluating the ability of the DsiRNAs listed in Table 2 to inhibit MAPT gene expression identified several candidate DsiRNAs.

Taken together, these results show that DsiRNAs designed to target human MAPT mRNA inhibit MAPT gene expression in cells, as determined by a reduced amount of MAPT mRNA in DsiRNA-transfected cells relative to control cells. These results demonstrate that the nucleotide sequences comprising the DsiRNA are useful for generating RNAi oligonucleotides to inhibit MAPT gene expression. Further, these results demonstrate that multiple MAPT mRNA target sequences are suitable for the RNAi-mediated inhibition of MAPT gene expression.

TABLE 2
In Vitro Screening Results.

| | | Anti- | | | | | | |
|---|---|---|---|---|---|---|---|---|
| | Sense | sense | | | | | | |
| | strand | strand | | | HsMAPT- | | HsMAPT- | |
| | SEQ | SEQ | 1 nM; T98G Cells | | 5′/SFRS9-F569 | | 3′/HPRT1-F517 | |
| | ID | ID | average % | | % | | % | |
| Construct | NO | NO | remaining | average SD | remaining | SEM | remaining | SEM |
| MAPT- | 1 | 385 | 95.01 | 25.6510056 | 76.872 | 12.015 | 113.148 | 14.537 |
| 2141 | | | | | | | | |
| MAPT- | 2 | 386 | 45.0915 | 14.8556064 | 34.587 | 3.412 | 55.596 | 5.956 |
| 2142 | | | | | | | | |
| MAPT- | 3 | 387 | 49.649 | 1.54149278 | 48.559 | 4.694 | 50.739 | 7.759 |
| 2303 | | | | | | | | |
| MAPT- | 4 | 388 | 14.9465 | 2.79802153 | 16.925 | 2.294 | 12.968 | 2.03 |
| 2347 | | | | | | | | |
| MAPT- | 5 | 389 | 36.102 | 6.40497322 | 40.631 | 12.81 | 31.573 | 8.578 |
| 2349 | | | | | | | | |
| MAPT- | 6 | 390 | 40.698 | 0.85701342 | 40.092 | 7.14 | 41.304 | 6.668 |
| 2350 | | | | | | | | |
| MAPT- | 7 | 391 | 34.233 | 1.88514668 | 35.566 | 7.156 | 32.9 | 12.834 |
| 2351 | | | | | | | | |
| MAPT- | 8 | 392 | 18.5755 | 5.96161727 | 22.791 | 7.452 | 14.36 | 4.732 |
| 2352 | | | | | | | | |
| MAPT- | 9 | 393 | 48.36 | 11.8822224 | 39.958 | 4.633 | 56.762 | 6.089 |
| 2353 | | | | | | | | |
| MAPT- | 10 | 394 | 24.094 | 3.67271262 | 26.691 | 5.688 | 21.497 | 6.358 |
| 2354 | | | | | | | | |
| MAPT- | 11 | 395 | 18.7485 | 3.90252233 | 21.508 | 3.374 | 15.989 | 2.482 |
| 2355 | | | | | | | | |
| MAPT- | 12 | 396 | 14.572 | 3.86645988 | 17.306 | 5.142 | 11.838 | 2.977 |
| 2459 | | | | | | | | |
| MAPT- | 13 | 397 | 13.041 | 4.14647416 | 15.973 | 2.64 | 10.109 | 1.577 |
| 2460 | | | | | | | | |
| MAPT- | 14 | 398 | 16.537 | 3.28097546 | 14.217 | 6.702 | 18.857 | 2.975 |
| 2461 | | | | | | | | |
| MAPT- | 15 | 399 | 21.8695 | 2.158797 | 23.396 | 6.242 | 20.343 | 6.622 |
| 2462 | | | | | | | | |
| MAPT- | 16 | 400 | 36.615 | 1.28127749 | 35.709 | 11 | 37.521 | 8.285 |
| 2463 | | | | | | | | |
| MAPT- | 17 | 401 | 24.302 | 4.31335137 | 21.252 | 4.336 | 27.352 | 3.33 |
| 2464 | | | | | | | | |
| MAPT- | 18 | 402 | 57.3185 | 24.0494087 | 40.313 | 6.657 | 74.324 | 8.436 |
| 2465 | | | | | | | | |
| MAPT- | 19 | 403 | 43.9035 | 1.65109433 | 42.736 | 8.925 | 45.071 | 6.344 |
| 2466 | | | | | | | | |
| MAPT- | 20 | 404 | 42.107 | 27.9957717 | 22.311 | 4.822 | 61.903 | 9.046 |
| 2467 | | | | | | | | |
| MAPT- | 21 | 405 | 38.319 | 5.58472936 | 34.37 | 10.991 | 42.268 | 9.76 |
| 2495 | | | | | | | | |
| MAPT- | 22 | 406 | 24.6465 | 2.92105811 | 26.712 | 4.387 | 22.581 | 6.794 |
| 2496 | | | | | | | | |
| MAPT- | 23 | 407 | 82.684 | 13.0956176 | 73.424 | 15.054 | 91.944 | 13.074 |
| 3686 | | | | | | | | |
| MAPT- | 24 | 408 | 55.0335 | 15.0691526 | 65.689 | 6.468 | 44.378 | 10.238 |
| 3687 | | | | | | | | |
| MAPT- | 25 | 409 | 87.266 | 1.77200959 | 88.519 | 15.089 | 86.013 | 15.763 |
| 3688 | | | | | | | | |
| MAPT- | 26 | 410 | 44.8115 | 4.63932759 | 48.092 | 12.584 | 41.531 | 10.617 |
| 3691 | | | | | | | | |
| MAPT- | 27 | 411 | 42.868 | 1.70412734 | 41.663 | 6.449 | 44.073 | 5.791 |
| 3692 | | | | | | | | |
| MAPT- | 28 | 412 | 49.581 | 11.071878 | 41.752 | 5.802 | 57.41 | 8.211 |
| 3693 | | | | | | | | |
| MAPT- | 29 | 413 | 47.3565 | 11.3016877 | 39.365 | 3.71 | 55.348 | 6.574 |
| 4534 | | | | | | | | |
| MAPT- | 30 | 414 | 56.0275 | 17.8608102 | 43.398 | 5.409 | 68.657 | 9.992 |
| 4535 | | | | | | | | |
| MAPT- | 31 | 415 | 62.025 | 10.3096169 | 54.735 | 7.123 | 69.315 | 12.404 |
| 4536 | | | | | | | | |
| MAPT- | 32 | 416 | 38.464 | 6.65811745 | 43.172 | 7.528 | 33.756 | 11.817 |
| 4537 | | | | | | | | |
| MAPT- | 33 | 417 | 44.9465 | 13.5743289 | 35.348 | 11.081 | 54.545 | 9.032 |
| 4538 | | | | | | | | |
| MAPT- | 34 | 418 | 38.0325 | 1.6058395 | 36.897 | 10.035 | 39.168 | 10.49 |
| 4566 | | | | | | | | |
| MAPT- | 35 | 419 | 40.872 | 8.78650886 | 34.659 | 6.539 | 47.085 | 6.222 |
| 4567 | | | | | | | | |
| MAPT- | 36 | 420 | 39.4745 | 0.77428193 | 40.022 | 6.35 | 38.927 | 3.497 |
| 4568 | | | | | | | | |
| MAPT- | 37 | 421 | 44.188 | 6.29183614 | 39.739 | 5.336 | 48.637 | 6.198 |
| 4569 | | | | | | | | |
| MAPT- | 38 | 422 | 52.8135 | 22.3947789 | 36.978 | 7.769 | 68.649 | 12.743 |
| 4570 | | | | | | | | |
| MAPT- | 39 | 423 | 50.4355 | 6.61639815 | 45.757 | 6.03 | 55.114 | 10.233 |
| 4571 | | | | | | | | |
| MAPT- | 40 | 424 | 51.2905 | 5.50765472 | 47.396 | 8.8 | 55.185 | 7.514 |
| 4572 | | | | | | | | |
| MAPT- | 41 | 425 | 41.6435 | 3.91100761 | 44.409 | 11.822 | 38.878 | 5.955 |
| 4573 | | | | | | | | |
| MAPT- | 42 | 426 | 44.6415 | 3.87140963 | 47.379 | 14.765 | 41.904 | 17.629 |
| 4574 | | | | | | | | |
| MAPT- | 43 | 427 | 43.044 | 4.46184379 | 39.889 | 4.461 | 46.199 | 6.498 |
| 4575 | | | | | | | | |
| MAPT- | 44 | 428 | 35.613 | 5.81100353 | 31.504 | 2.99 | 39.722 | 6.824 |
| 4576 | | | | | | | | |
| MAPT- | 45 | 429 | 32.979 | 3.62462936 | 35.542 | 4.01 | 30.416 | 7.004 |
| 4577 | | | | | | | | |
| MAPT- | 46 | 430 | 59.0835 | 11.5053344 | 50.948 | 8.8 | 67.219 | 11.491 |
| 4578 | | | | | | | | |
| MAPT- | 47 | 431 | 44.268 | 11.4565441 | 36.167 | 4.845 | 52.369 | 5.65 |
| 4579 | | | | | | | | |
| MAPT- | 48 | 432 | 57.2225 | 22.8713688 | 41.05 | 10.952 | 73.395 | 8.852 |
| 4580 | | | | | | | | |
| MAPT- | 49 | 433 | 96.059 | 3.22440692 | 93.779 | 15.189 | 98.339 | 15.945 |
| 4605 | | | | | | | | |
| MAPT- | 50 | 434 | 57.348 | 2.49467272 | 59.112 | 13.383 | 55.584 | 9.058 |
| 4606 | | | | | | | | |
| MAPT- | 51 | 435 | 67.9825 | 13.5799857 | 58.38 | 10.161 | 77.585 | 9.466 |
| 4607 | | | | | | | | |
| MAPT- | 52 | 436 | 35.004 | 14.4589195 | 45.228 | 5.369 | 24.78 | 7.968 |
| 4608 | | | | | | | | |
| MAPT- | 53 | 437 | 52.854 | 7.33552575 | 58.041 | 9.122 | 47.667 | 6.181 |
| 4609 | | | | | | | | |
| MAPT- | 54 | 438 | 56.244 | 5.2764308 | 52.513 | 8.718 | 59.975 | 11.85 |
| 4610 | | | | | | | | |
| MAPT- | 55 | 439 | 60.552 | 13.7489843 | 70.274 | 19.308 | 50.83 | 9.512 |
| 4611 | | | | | | | | |
| MAPT- | 56 | 440 | 44.801 | 10.820148 | 37.15 | 9.793 | 52.452 | 16.948 |
| 4612 | | | | | | | | |
| MAPT- | 57 | 441 | 55.2605 | 12.1374879 | 46.678 | 13.942 | 63.843 | 4.47 |
| 4613 | | | | | | | | |
| MAPT- | 58 | 442 | 51.7385 | 24.0451661 | 68.741 | 10.458 | 34.736 | 6.591 |
| 4614 | | | | | | | | |
| MAPT- | 59 | 443 | 47.4125 | 3.24915566 | 45.115 | 14.811 | 49.71 | 8.998 |
| 5969 | | | | | | | | |
| MAPT- | 60 | 444 | 44.3725 | 3.58998113 | 41.834 | 5.855 | 46.911 | 3.033 |
| 5970 | | | | | | | | |
| MAPT- | 61 | 445 | 63.7505 | 23.6336299 | 47.039 | 6.803 | 80.462 | 9.959 |
| 5971 | | | | | | | | |
| MAPT- | 62 | 446 | 49.5005 | 23.8089924 | 32.665 | 4.895 | 66.336 | 6.085 |
| 5972 | | | | | | | | |
| MAPT- | 63 | 447 | 52.079 | 7.14884956 | 57.134 | 10.719 | 47.024 | 8.743 |
| 5973 | | | | | | | | |
| MAPT- | 64 | 448 | 44.9585 | 12.1686006 | 36.354 | 12.432 | 53.563 | 8.866 |
| 5974 | | | | | | | | |
| MAPT- | 65 | 449 | 45.1555 | 0.32456201 | 45.385 | 9.699 | 44.926 | 10.859 |
| 5975 | | | | | | | | |
| MAPT- | 66 | 450 | 43.056 | 1.32511811 | 43.993 | 18.502 | 42.119 | 5.098 |
| 5976 | | | | | | | | |
| MAPT- | 67 | 451 | 47.3185 | 1.16319066 | 48.141 | 6.434 | 46.496 | 7.198 |
| 5977 | | | | | | | | |
| MAPT- | 68 | 452 | 58.9385 | 3.69887557 | 56.323 | 8.866 | 61.554 | 8.153 |
| 5978 | | | | | | | | |
| MAPT- | 69 | 453 | 90.251 | 49.5568717 | 55.209 | 23.067 | 125.293 | 41.072 |
| 5979 | | | | | | | | |
| MAPT- | 70 | 454 | 93.5025 | 1.75433192 | 92.262 | 15.249 | 94.743 | 12.851 |
| 5980 | | | | | | | | |
| MAPT- | 71 | 455 | 73.614 | 15.0401612 | 62.979 | 9.331 | 84.249 | 10.224 |
| 5981 | | | | | | | | |
| MAPT- | 72 | 456 | 52.7605 | 4.23627673 | 55.756 | 7.219 | 49.765 | 6.897 |
| 5982 | | | | | | | | |
| MAPT- | 73 | 457 | 52.0305 | 13.3805816 | 61.492 | 16.873 | 42.569 | 15.962 |
| 5983 | | | | | | | | |
| MAPT- | 74 | 458 | 39.226 | 13.1267303 | 29.944 | 8.671 | 48.508 | 18.496 |
| 5984 | | | | | | | | |
| MAPT- | 75 | 459 | 47.514 | 0.09475231 | 47.581 | 10.955 | 47.447 | 15.502 |
| 5985 | | | | | | | | |
| MAPT- | 76 | 460 | 56.8945 | 5.52321107 | 52.989 | 23.098 | 60.8 | 21.716 |
| 6662 | | | | | | | | |
| MAPT- | 77 | 461 | 50.6 | 16.8984379 | 38.651 | 7.897 | 62.549 | 14.03 |
| 6663 | | | | | | | | |
| MAPT- | 78 | 462 | 59.894 | 1.37461558 | 60.866 | 17.793 | 58.922 | 20.062 |
| 6664 | | | | | | | | |
| MAPT- | 79 | 463 | 45.514 | 6.31022092 | 49.976 | 19.816 | 41.052 | 16.434 |
| 6665 | | | | | | | | |
| MAPT- | 80 | 464 | 49.963 | 1.58391919 | 51.083 | 13.407 | 48.843 | 10.693 |
| 6800 | | | | | | | | |
| MAPT- | 81 | 465 | 78.6545 | 21.2761359 | 63.61 | 10.347 | 93.699 | 12.651 |
| 6801 | | | | | | | | |
| MAPT- | 82 | 466 | 36.959 | 2.8468119 | 34.946 | 11.719 | 38.972 | 6.592 |
| 6802 | | | | | | | | |
| MAPT- | 83 | 467 | 60.1565 | 7.48896792 | 54.861 | 6.209 | 65.452 | 7.341 |
| 6803 | | | | | | | | |
| MAPT- | 84 | 468 | 58.1295 | 25.573931 | 40.046 | 7.718 | 76.213 | 14.881 |
| 6804 | | | | | | | | |
| MAPT- | 85 | 469 | 40.6695 | 7.0180348 | 35.707 | 8.81 | 45.632 | 6.173 |
| 6805 | | | | | | | | |
| MAPT- | 86 | 470 | 49.47 | 9.29421153 | 42.898 | 13.633 | 56.042 | 8.573 |
| 6806 | | | | | | | | |
| MAPT- | 87 | 471 | 44.6315 | 14.4172002 | 34.437 | 12.547 | 54.826 | 14.094 |
| 6807 | | | | | | | | |
| MAPT- | 88 | 472 | 47.8265 | 1.73311872 | 46.601 | 15.127 | 49.052 | 9.845 |
| 6808 | | | | | | | | |
| MAPT- | 89 | 473 | 100.4455 | 12.3199214 | 91.734 | 32.653 | 109.157 | 62.617 |
| 6809 | | | | | | | | |
| MAPT- | 90 | 474 | 38.2355 | 5.25168206 | 34.522 | 8.787 | 41.949 | 19.197 |
| 6810 | | | | | | | | |
| MAPT- | 91 | 475 | 64.862 | 2.72801796 | 66.791 | 6.959 | 62.933 | 10.212 |
| 6811 | | | | | | | | |
| MAPT- | 92 | 476 | 78.776 | 32.6782328 | 55.669 | 16.433 | 101.883 | 16.548 |
| 6812 | | | | | | | | |
| MAPT- | 93 | 477 | 67.15 | 7.39068008 | 61.924 | 11.954 | 72.376 | 13.616 |
| 6813 | | | | | | | | |
| MAPT- | 94 | 478 | 44.55 | 0.562857 | 44.152 | 9.142 | 44.948 | 5.743 |
| 6814 | | | | | | | | |
| MAPT- | 95 | 479 | 72.563 | 12.5695301 | 63.675 | 13.171 | 81.451 | 15.732 |
| 6815 | | | | | | | | |
| MAPT- | 96 | 480 | 42.662 | 7.06258253 | 37.668 | 7.799 | 47.656 | 9.971 |
| 6816 | | | | | | | | |
| MAPT- | 97 | 481 | 10.801 | 0.99843478 | 11.507 | 5.145 | 10.095 | 2.3 |
| 363 | | | | | | | | |
| MAPT- | 98 | 482 | 17.493 | 1.23319423 | 18.365 | 8.672 | 16.621 | 3.855 |
| 364 | | | | | | | | |
| MAPT- | 99 | 483 | 49.6115 | 18.8917719 | 36.253 | 12.86 | 62.97 | 12.877 |
| 365 | | | | | | | | |
| MAPT- | 100 | 484 | 60.2915 | 5.79898271 | 64.392 | 9.598 | 56.191 | 8.387 |
| 367 | | | | | | | | |
| MAPT- | 101 | 485 | 18.496 | 5.14349473 | 14.859 | 2.908 | 22.133 | 5.121 |
| 369 | | | | | | | | |
| MAPT- | 102 | 486 | 54.2605 | 7.24855161 | 49.135 | 6.979 | 59.386 | 8.929 |
| 374 | | | | | | | | |
| MAPT- | 103 | 487 | 29.862 | 11.7125167 | 38.144 | 8.293 | 21.58 | 5.252 |
| 395 | | | | | | | | |
| MAPT- | 104 | 488 | 34.402 | 16.4246763 | 22.788 | 7.436 | 46.016 | 11.445 |
| 400 | | | | | | | | |
| MAPT- | 105 | 489 | 17.7005 | 6.73802052 | 12.936 | 4.852 | 22.465 | 4.318 |
| 443 | | | | | | | | |
| MAPT- | 106 | 490 | 62.8045 | 3.40047651 | 65.209 | 13.424 | 60.4 | 5.794 |
| 688 | | | | | | | | |
| MAPT- | 107 | 491 | 26.4395 | 3.02853834 | 28.581 | 4.295 | 24.298 | 6.236 |
| 689 | | | | | | | | |
| MAPT- | 108 | 492 | 14.6445 | 4.81327586 | 11.241 | 2.659 | 18.048 | 2.561 |
| 690 | | | | | | | | |
| MAPT- | 109 | 493 | 63.3205 | 15.4368481 | 52.405 | 6.513 | 74.236 | 8.881 |
| 693 | | | | | | | | |
| MAPT- | 110 | 494 | 116.4165 | 9.93838581 | 123.444 | 17.981 | 109.389 | 37.256 |
| 695 | | | | | | | | |
| MAPT- | 111 | 495 | 31.593 | 2.7322606 | 33.525 | 9.92 | 29.661 | 8.245 |
| 696 | | | | | | | | |
| MAPT- | 112 | 496 | 26.2465 | 9.29491864 | 19.674 | 5.59 | 32.819 | 7.787 |
| 1475 | | | | | | | | |
| MAPT- | 113 | 497 | 30.5425 | 1.07975205 | 29.779 | 5.507 | 31.306 | 6.256 |
| 1476 | | | | | | | | |
| MAPT- | 114 | 498 | 13.127 | 0.9588368 | 13.805 | 1.935 | 12.449 | 2.983 |
| 1479 | | | | | | | | |
| MAPT- | 115 | 499 | 47.5765 | 5.71554411 | 43.535 | 9.405 | 51.618 | 7.164 |
| 1480 | | | | | | | | |
| MAPT- | 116 | 500 | 37.489 | 3.0349023 | 39.635 | 3.962 | 35.343 | 5.214 |
| 1481 | | | | | | | | |
| MAPT- | 117 | 501 | 24.0215 | 8.67690731 | 17.886 | 3.238 | 30.157 | 3.712 |
| 1484 | | | | | | | | |
| MAPT- | 118 | 502 | 55.319 | 27.0807755 | 36.17 | 8.411 | 74.468 | 21.392 |
| 1485 | | | | | | | | |
| MAPT- | 119 | 503 | 77.784 | 20.2685088 | 63.452 | 9.291 | 92.116 | 8.346 |
| 1492 | | | | | | | | |
| MAPT- | 120 | 504 | 15.4475 | 3.58432427 | 12.913 | 5.176 | 17.982 | 3.997 |
| 1494 | | | | | | | | |
| MAPT- | 121 | 505 | 18.204 | 1.04793225 | 18.945 | 3.15 | 17.463 | 3.766 |
| 1495 | | | | | | | | |
| MAPT- | 122 | 506 | 20.014 | 2.4607316 | 18.274 | 6.805 | 21.754 | 5.32 |
| 1498 | | | | | | | | |
| MAPT- | 123 | 507 | 65.7465 | 24.832883 | 48.187 | 11.539 | 83.306 | 19.091 |
| 1499 | | | | | | | | |
| MAPT- | 124 | 508 | 18.62 | 5.80393246 | 14.516 | 4.376 | 22.724 | 5.504 |
| 1500 | | | | | | | | |
| MAPT- | 125 | 509 | 34.775 | 5.02187236 | 38.326 | 10.536 | 31.224 | 4.366 |
| 1502 | | | | | | | | |
| MAPT- | 126 | 510 | 31.277 | 11.1058191 | 39.13 | 11.069 | 23.424 | 3.92 |
| 1503 | | | | | | | | |
| MAPT- | 127 | 511 | 24.627 | 3.87211673 | 27.365 | 4.52 | 21.889 | 2.089 |
| 1504 | | | | | | | | |
| MAPT- | 128 | 512 | 10.3425 | 2.81074946 | 12.33 | 1.998 | 8.355 | 2.686 |
| 1505 | | | | | | | | |
| MAPT- | 129 | 513 | 57.631 | 0.21354625 | 57.48 | 23.99 | 57.782 | 17.417 |
| 1506 | | | | | | | | |
| MAPT- | 130 | 514 | 32.112 | 3.45068109 | 34.552 | 9.62 | 29.672 | 4.255 |
| 1507 | | | | | | | | |
| MAPT- | 131 | 515 | 27.841 | 5.67099639 | 31.851 | 8.28 | 23.831 | 6.196 |
| 1508 | | | | | | | | |
| MAPT- | 132 | 516 | 38.7 | 4.51699812 | 41.894 | 7.785 | 35.506 | 5.674 |
| 1509 | | | | | | | | |
| MAPT- | 133 | 517 | 15.758 | 4.39961839 | 18.869 | 4.717 | 12.647 | 3.372 |
| 1733 | | | | | | | | |
| MAPT- | 134 | 518 | 78.742 | 9.03116781 | 85.128 | 12.726 | 72.356 | 9.03 |
| 1796 | | | | | | | | |
| MAPT- | 135 | 519 | 77.3505 | 24.4199327 | 94.618 | 20.422 | 60.083 | 13.368 |
| 1835 | | | | | | | | |
| MAPT- | 136 | 520 | 24.699 | 5.16329372 | 28.35 | 13.873 | 21.048 | 4.701 |
| 1912 | | | | | | | | |
| MAPT- | 137 | 521 | 18.8405 | 9.74463855 | 25.731 | 6.678 | 11.95 | 1.689 |
| 2094 | | | | | | | | |
| MAPT- | 138 | 522 | 13.6925 | 1.29471252 | 14.608 | 3.403 | 12.777 | 2.067 |
| 2096 | | | | | | | | |
| MAPT- | 139 | 523 | 21.9025 | 4.07081374 | 19.024 | 4.924 | 24.781 | 3.696 |
| 2097 | | | | | | | | |
| MAPT- | 140 | 524 | 17.656 | 9.87828173 | 10.671 | 3.205 | 24.641 | 2.915 |
| 2098 | | | | | | | | |
| MAPT- | 141 | 525 | 22.912 | 10.0098036 | 29.99 | 6.919 | 15.834 | 5.794 |
| 2105 | | | | | | | | |
| MAPT- | 142 | 526 | 16.6545 | 1.53937146 | 17.743 | 3.658 | 15.566 | 3.854 |
| 2106 | | | | | | | | |
| MAPT- | 143 | 527 | 33.657 | 2.05343809 | 35.109 | 4.469 | 32.205 | 5.491 |
| 2107 | | | | | | | | |
| MAPT- | 144 | 528 | 27.0645 | 15.2940126 | 37.879 | 8.018 | 16.25 | 2.844 |
| 2108 | | | | | | | | |
| MAPT- | 145 | 529 | 18.066 | 0.36769553 | 18.326 | 6.375 | 17.806 | 4.395 |
| 2109 | | | | | | | | |
| MAPT- | 146 | 530 | 70.417 | 5.66958217 | 66.408 | 16.607 | 74.426 | 11.191 |
| 2117 | | | | | | | | |
| MAPT- | 147 | 531 | 30.333 | 0.4794184 | 30.672 | 6.394 | 29.994 | 3.926 |
| 2136 | | | | | | | | |
| MAPT- | 148 | 532 | 30.2155 | 0.61023315 | 29.784 | 5.066 | 30.647 | 4.74 |
| 2137 | | | | | | | | |
| MAPT- | 149 | 533 | 22.638 | 2.83974083 | 20.63 | 4.29 | 24.646 | 6.881 |
| 2269 | | | | | | | | |
| MAPT- | 150 | 534 | 12.216 | 1.87241876 | 10.892 | 3.111 | 13.54 | 3.021 |
| 2270 | | | | | | | | |
| MAPT- | 151 | 535 | 31.5845 | 14.4270997 | 21.383 | 9.46 | 41.786 | 5.402 |
| 2271 | | | | | | | | |
| MAPT- | 152 | 536 | 27.5985 | 12.4514433 | 36.403 | 7.932 | 18.794 | 4.51 |
| 2272 | | | | | | | | |
| MAPT- | 153 | 537 | 14.2115 | 3.25764094 | 11.908 | 3.525 | 16.515 | 2.483 |
| 2273 | | | | | | | | |
| MAPT- | 154 | 538 | 13.829 | 4.75317178 | 10.468 | 4.009 | 17.19 | 2.488 |
| 2274 | | | | | | | | |
| MAPT- | 155 | 539 | 19.6215 | 0.85772053 | 19.015 | 6.27 | 20.228 | 3.6 |
| 2275 | | | | | | | | |
| MAPT- | 156 | 540 | 13.741 | 6.74155605 | 8.974 | 4.323 | 18.508 | 4.472 |
| 2276 | | | | | | | | |
| MAPT- | 157 | 541 | 35.06 | 2.40133463 | 33.362 | 8.636 | 36.758 | 4.222 |
| 2277 | | | | | | | | |
| MAPT- | 158 | 542 | 62.9755 | 7.38007348 | 57.757 | 15.426 | 68.194 | 14.479 |
| 2278 | | | | | | | | |
| MAPT- | 159 | 543 | 12.3635 | 0.0629325 | 12.408 | 6.106 | 12.319 | 4.131 |
| 2279 | | | | | | | | |
| MAPT- | 160 | 544 | 22.4485 | 4.5672027 | 19.219 | 6.284 | 25.678 | 6.587 |
| 2280 | | | | | | | | |
| MAPT- | 161 | 545 | 11.262 | 3.82544769 | 13.967 | 4.235 | 8.557 | 4.509 |
| 2281 | | | | | | | | |
| MAPT- | 162 | 546 | 22.766 | 10.3704281 | 30.099 | 11.793 | 15.433 | 2.563 |
| 2282 | | | | | | | | |
| MAPT- | 163 | 547 | 22.093 | 2.21748687 | 23.661 | 9.459 | 20.525 | 5.485 |
| 2283 | | | | | | | | |
| MAPT- | 164 | 548 | 9.3375 | 0.48578236 | 9.681 | 3.741 | 8.994 | 2.065 |
| 2284 | | | | | | | | |
| MAPT- | 165 | 549 | 43.909 | 5.46452121 | 40.045 | 5.708 | 47.773 | 11.236 |
| 2286 | | | | | | | | |
| MAPT- | 166 | 550 | 44.999 | 0.15839192 | 45.111 | 3.984 | 44.887 | 11.572 |
| 2288 | | | | | | | | |
| MAPT- | 167 | 551 | 16.7085 | 2.74710984 | 18.651 | 6.81 | 14.766 | 5.374 |
| 2289 | | | | | | | | |
| MAPT- | 168 | 552 | 20.7095 | 1.76423142 | 19.462 | 4.227 | 21.957 | 4.191 |
| 2291 | | | | | | | | |
| MAPT- | 169 | 553 | 36.6055 | 15.5033162 | 47.568 | 9.742 | 25.643 | 7.144 |
| 2294 | | | | | | | | |
| MAPT- | 170 | 554 | 12.62 | 3.60624458 | 15.17 | 3.604 | 10.07 | 3.636 |
| 2299 | | | | | | | | |
| MAPT- | 171 | 555 | 17.296 | 0.32385491 | 17.525 | 5.091 | 17.067 | 4.382 |
| 2300 | | | | | | | | |
| MAPT- | 172 | 556 | 14.4115 | 2.7838794 | 12.443 | 3.439 | 16.38 | 5.47 |
| 2301 | | | | | | | | |
| MAPT- | 173 | 557 | 39.5035 | 12.6225632 | 48.429 | 8.232 | 30.578 | 4.345 |
| 2308 | | | | | | | | |
| MAPT- | 174 | 558 | 36.121 | 1.50755166 | 35.055 | 9.878 | 37.187 | 6.948 |
| 2316 | | | | | | | | |
| MAPT- | 175 | 559 | 23.2925 | 4.71145248 | 19.961 | 7.977 | 26.624 | 5.453 |
| 2317 | | | | | | | | |
| MAPT- | 176 | 560 | 46.479 | 18.4795286 | 33.412 | 7.15 | 59.546 | 16.236 |
| 2319 | | | | | | | | |
| MAPT- | 177 | 561 | 34.5325 | 16.1340554 | 45.941 | 18.317 | 23.124 | 5.385 |
| 2320 | | | | | | | | |
| MAPT- | 178 | 562 | 49.3475 | 9.8747462 | 56.33 | 10.684 | 42.365 | 6.267 |
| 2322 | | | | | | | | |
| MAPT- | 179 | 563 | 37.4055 | 9.99071171 | 44.47 | 7.337 | 30.341 | 7.651 |
| 2323 | | | | | | | | |
| MAPT- | 180 | 564 | 29.453 | 6.20981175 | 25.062 | 3.998 | 33.844 | 9.822 |
| 2324 | | | | | | | | |
| MAPT- | 181 | 565 | 40.7595 | 0.49709607 | 41.111 | 8.995 | 40.408 | 14.253 |
| 2326 | | | | | | | | |
| MAPT- | 182 | 566 | 53.6645 | 1.63129534 | 54.818 | 11.735 | 52.511 | 11.097 |
| 2330 | | | | | | | | |
| MAPT- | 183 | 567 | 41.555 | 10.4071976 | 34.196 | 6.582 | 48.914 | 10.912 |
| 2356 | | | | | | | | |
| MAPT- | 184 | 568 | 16.16 | 7.5702852 | 21.513 | 1.827 | 10.807 | 3.58 |
| 2357 | | | | | | | | |
| MAPT- | 185 | 569 | 15.9115 | 0.34718943 | 16.157 | 5.317 | 15.666 | 2.953 |
| 2358 | | | | | | | | |
| MAPT- | 186 | 570 | 38.779 | 13.5198817 | 48.339 | 12.534 | 29.219 | 3.728 |
| 2359 | | | | | | | | |
| MAPT- | 187 | 571 | 56.6595 | 2.41759808 | 58.369 | 9.583 | 54.95 | 7.626 |
| 2360 | | | | | | | | |
| MAPT- | 188 | 572 | 19.9675 | 15.8893965 | 31.203 | 7.037 | 8.732 | 1.814 |
| 2361 | | | | | | | | |
| MAPT- | 189 | 573 | 25.7945 | 4.27021785 | 28.814 | 11.436 | 22.775 | 6.312 |
| 2362 | | | | | | | | |
| MAPT- | 190 | 574 | 29.327 | 6.8094383 | 34.142 | 13.111 | 24.512 | 10.071 |
| 2363 | | | | | | | | |
| MAPT- | 191 | 575 | 13.174 | 2.66154992 | 11.292 | 3.193 | 15.056 | 3.026 |
| 2364 | | | | | | | | |
| MAPT- | 192 | 576 | 22.5375 | 0.18314066 | 22.408 | 4.494 | 22.667 | 3.198 |
| 2365 | | | | | | | | |
| MAPT- | 193 | 577 | 10.775 | 5.70918015 | 14.812 | 6.29 | 6.738 | 2.466 |
| 2372 | | | | | | | | |
| MAPT- | 194 | 578 | 14.297 | 0.80468752 | 13.728 | 3.586 | 14.866 | 3.452 |
| 2373 | | | | | | | | |
| MAPT- | 195 | 579 | 11.664 | 0.88529769 | 11.038 | 4.433 | 12.29 | 2.181 |
| 2374 | | | | | | | | |
| MAPT- | 196 | 580 | 19.954 | 1.3449171 | 20.905 | 3.625 | 19.003 | 5.119 |
| 2375 | | | | | | | | |
| MAPT- | 197 | 581 | 11.6305 | 7.16228459 | 6.566 | 2.913 | 16.695 | 5.49 |
| 2376 | | | | | | | | |
| MAPT- | 198 | 582 | 17.774 | 4.86630887 | 14.333 | 4.695 | 21.215 | 5.419 |
| 2377 | | | | | | | | |
| MAPT- | 199 | 583 | 15.912 | 6.94096016 | 11.004 | 7.297 | 20.82 | 6.571 |
| 2378 | | | | | | | | |
| MAPT- | 200 | 584 | 10.8575 | 3.47825826 | 8.398 | 3.267 | 13.317 | 2.091 |
| 2379 | | | | | | | | |
| MAPT- | 201 | 585 | 54.1995 | 16.1213275 | 42.8 | 16.849 | 65.599 | 12.511 |
| 2380 | | | | | | | | |
| MAPT- | 202 | 586 | 23.8745 | 5.39027499 | 20.063 | 5.735 | 27.686 | 4.702 |
| 2381 | | | | | | | | |
| MAPT- | 203 | 587 | 6.365 | 0.30547013 | 6.581 | 2.357 | 6.149 | 2.123 |
| 2382 | | | | | | | | |
| MAPT- | 204 | 588 | 6.377 | 0.34931075 | 6.13 | 2.062 | 6.624 | 1.482 |
| 2390 | | | | | | | | |
| MAPT- | 205 | 589 | 12.498 | 2.13121984 | 10.991 | 5.358 | 14.005 | 4.476 |
| 2391 | | | | | | | | |
| MAPT- | 206 | 590 | 66.506 | 38.9842111 | 38.94 | 9.142 | 94.072 | 16.71 |
| 2414 | | | | | | | | |
| MAPT- | 207 | 591 | 101.6285 | 16.4791235 | 89.976 | 32.779 | 113.281 | 40.057 |
| 2448 | | | | | | | | |
| MAPT- | 208 | 592 | 6.118 | 1.94171522 | 7.491 | 2.885 | 4.745 | 2.142 |
| 2449 | | | | | | | | |
| MAPT- | 209 | 593 | 5.428 | 0.44264885 | 5.741 | 1.379 | 5.115 | 0.708 |
| 2450 | | | | | | | | |
| MAPT- | 210 | 594 | 9.3195 | 2.04141728 | 7.876 | 3.092 | 10.763 | 3.026 |
| 2451 | | | | | | | | |
| MAPT- | 211 | 595 | 12.4495 | 0.23688077 | 12.282 | 3.89 | 12.617 | 3.728 |
| 2452 | | | | | | | | |
| MAPT- | 212 | 596 | 6.363 | 2.26981277 | 4.758 | 1.495 | 7.968 | 1.487 |
| 2453 | | | | | | | | |
| MAPT- | 213 | 597 | 7.1635 | 0.37264527 | 7.427 | 3.314 | 6.9 | 1.49 |
| 2454 | | | | | | | | |
| MAPT- | 214 | 598 | 5.641 | 0.69437886 | 6.132 | 1.582 | 5.15 | 2.739 |
| 2456 | | | | | | | | |
| MAPT- | 215 | 599 | 16.472 | 7.58584155 | 11.108 | 2.63 | 21.836 | 5.922 |
| 2457 | | | | | | | | |
| MAPT- | 216 | 600 | 12.6805 | 3.12329065 | 14.889 | 6.56 | 10.472 | 3.719 |
| 2567 | | | | | | | | |
| MAPT- | 217 | 601 | 64.8695 | 33.2927086 | 41.328 | 16.719 | 88.411 | 33.918 |
| 2598 | | | | | | | | |
| MAPT- | 218 | 602 | 68.409 | 37.9249651 | 41.592 | 8.109 | 95.226 | 13.823 |
| 2657 | | | | | | | | |
| MAPT- | 219 | 603 | 11.61 | 0.04384062 | 11.579 | 8.192 | 11.641 | 11.451 |
| 2723 | | | | | | | | |
| MAPT- | 220 | 604 | 233.808 | | 233.808 | 161.639 | | |
| 2724 | | | | | | | | |
| MAPT- | 221 | 605 | 1302.476 | | 1302.476 | 848.106 | | |
| 2726 | | | | | | | | |
| MAPT- | 222 | 606 | 10.97 | 5.81100353 | 6.861 | 1.876 | 15.079 | 4.091 |
| 2784 | | | | | | | | |
| MAPT- | 223 | 607 | 40.636 | 1.52876486 | 39.555 | 13.364 | 41.717 | 9.834 |
| 2963 | | | | | | | | |
| MAPT- | 224 | 608 | 67.4985 | 25.5979726 | 49.398 | 18.559 | 85.599 | 30.988 |
| 3110 | | | | | | | | |
| MAPT- | 225 | 609 | 34.0185 | 10.2622407 | 26.762 | 7.333 | 41.275 | 9.007 |
| 3114 | | | | | | | | |
| MAPT- | 226 | 610 | 23.677 | 5.0883404 | 20.079 | 5.942 | 27.275 | 7.786 |
| 3116 | | | | | | | | |
| MAPT- | 227 | 611 | 17.6195 | 5.74948524 | 21.685 | 7.03 | 13.554 | 4.395 |
| 3118 | | | | | | | | |
| MAPT- | 228 | 612 | 33.388 | 17.8502036 | 20.766 | 7.711 | 46.01 | 13.684 |
| 3158 | | | | | | | | |
| MAPT- | 229 | 613 | 108.324 | 38.5344911 | 135.572 | 60.571 | 81.076 | 13.918 |
| 3503 | | | | | | | | |
| MAPT- | 230 | 614 | 39.759 | 3.12258355 | 41.967 | 10.832 | 37.551 | 6.071 |
| 3589 | | | | | | | | |
| MAPT- | 231 | 615 | 204.4415 | 111.869243 | 125.338 | 69.843 | 283.545 | 162.626 |
| 3591 | | | | | | | | |
| MAPT- | 232 | 616 | 136.1045 | 32.3423571 | 113.235 | 30.524 | 158.974 | 46.677 |
| 3592 | | | | | | | | |
| MAPT- | 233 | 617 | 63.914 | 14.1619346 | 53.9 | 10.02 | 73.928 | 12.246 |
| 3593 | | | | | | | | |
| MAPT- | 234 | 618 | 80.7265 | 46.8465314 | 47.601 | 28.291 | 113.852 | 56.081 |
| 3594 | | | | | | | | |
| MAPT- | 235 | 619 | 33.518 | 3.51997756 | 31.029 | 3.774 | 36.007 | 4.987 |
| 3595 | | | | | | | | |
| MAPT- | 236 | 620 | 50.5415 | 9.44623949 | 57.221 | 22.189 | 43.862 | 7.353 |
| 3596 | | | | | | | | |
| MAPT- | 237 | 621 | 207.694 | 70.014885 | 158.186 | 64.728 | 257.202 | 178.898 |
| 3597 | | | | | | | | |
| MAPT- | 238 | 622 | 52.1005 | 3.0865211 | 49.918 | 5.249 | 54.283 | 7.413 |
| 3598 | | | | | | | | |
| MAPT- | 239 | 623 | 364.1355 | 362.407075 | 620.396 | 189.266 | 107.875 | 48.489 |
| 3599 | | | | | | | | |
| MAPT- | 240 | 624 | 100.4525 | 30.5364063 | 122.045 | 56.065 | 78.86 | 42.186 |
| 3600 | | | | | | | | |
| MAPT- | 241 | 625 | 47.233 | 12.1297097 | 38.656 | 5.821 | 55.81 | 7.764 |
| 3601 | | | | | | | | |
| MAPT- | 242 | 626 | 43.078 | 9.10894956 | 36.637 | 5.569 | 49.519 | 8.21 |
| 3602 | | | | | | | | |
| MAPT- | 243 | 627 | 46.1485 | 9.83797665 | 39.192 | 6.61 | 53.105 | 14.334 |
| 3603 | | | | | | | | |
| MAPT- | 244 | 628 | 38.537 | 6.97490129 | 43.469 | 6.803 | 33.605 | 12.721 |
| 3605 | | | | | | | | |
| MAPT- | 245 | 629 | 52.929 | 12.1099107 | 44.366 | 6.167 | 61.492 | 10.195 |
| 3607 | | | | | | | | |
| MAPT- | 246 | 630 | 61.4085 | 31.4839294 | 39.146 | 5.412 | 83.671 | 11.371 |
| 3609 | | | | | | | | |
| MAPT- | 247 | 631 | 53.294 | 26.6211561 | 34.47 | 5.122 | 72.118 | 6.918 |
| 3610 | | | | | | | | |
| MAPT- | 248 | 632 | 72.7675 | 16.9755125 | 60.764 | 8.013 | 84.771 | 12.398 |
| 3677 | | | | | | | | |
| MAPT- | 249 | 633 | 44.9005 | 19.9778879 | 30.774 | 5.138 | 59.027 | 10.698 |
| 3678 | | | | | | | | |
| MAPT- | 250 | 634 | 72.05 | 31.5638325 | 49.731 | 5.218 | 94.369 | 10.439 |
| 3679 | | | | | | | | |
| MAPT- | 251 | 635 | 58.9445 | 1.90565278 | 60.292 | 9.168 | 57.597 | 15.808 |
| 3680 | | | | | | | | |
| MAPT- | 252 | 636 | 60.937 | 9.03682466 | 54.547 | 8.505 | 67.327 | 20.084 |
| 3958 | | | | | | | | |
| MAPT- | 253 | 637 | 76.6655 | 34.4580206 | 52.3 | 5.769 | 101.031 | 26.056 |
| 3959 | | | | | | | | |
| MAPT- | 254 | 638 | 75.305 | 39.354735 | 47.477 | 5.803 | 103.133 | 19.335 |
| 3960 | | | | | | | | |
| MAPT- | 255 | 639 | 45.3385 | 7.61483293 | 39.954 | 10.197 | 50.723 | 5.643 |
| 3961 | | | | | | | | |
| MAPT- | 256 | 640 | 72.0995 | 2.20688026 | 70.539 | 7.456 | 73.66 | 8.937 |
| 3965 | | | | | | | | |
| MAPT- | 257 | 641 | 74.1615 | 12.9549033 | 65.001 | 14.647 | 83.322 | 16.913 |
| 3970 | | | | | | | | |
| MAPT- | 258 | 642 | 77.359 | 47.5543452 | 43.733 | 6.924 | 110.985 | 17.09 |
| 4146 | | | | | | | | |
| MAPT- | 259 | 643 | 42.89 | 0.51194531 | 43.252 | 6.788 | 42.528 | 13.112 |
| 4474 | | | | | | | | |
| MAPT- | 260 | 644 | 66.3895 | 44.1764962 | 35.152 | 5.684 | 97.627 | 19.713 |
| 4475 | | | | | | | | |
| MAPT- | 261 | 645 | 68.927 | 35.8870834 | 43.551 | 6.949 | 94.303 | 12.932 |
| 4477 | | | | | | | | |
| MAPT- | 262 | 646 | 50.6895 | 22.0624387 | 35.089 | 7.887 | 66.29 | 21.359 |
| 4478 | | | | | | | | |
| MAPT- | 263 | 647 | 40.2915 | 16.0223326 | 51.621 | 8.132 | 28.962 | 6.75 |
| 4479 | | | | | | | | |
| MAPT- | 264 | 648 | 62.963 | 18.7227734 | 76.202 | 15.622 | 49.724 | 17.779 |
| 4480 | | | | | | | | |
| MAPT- | 265 | 649 | 62.488 | 35.7329341 | 37.221 | 8.557 | 87.755 | 19.687 |
| 4481 | | | | | | | | |
| MAPT- | 266 | 650 | 96.4665 | 98.6859437 | 26.685 | 10.537 | 166.248 | 71.368 |
| 4482 | | | | | | | | |
| MAPT- | 267 | 651 | 67.466 | 56.8216867 | 27.287 | 9.898 | 107.645 | 19.981 |
| 4485 | | | | | | | | |
| MAPT- | 268 | 652 | 64.4675 | 54.6770319 | 25.805 | 8.223 | 103.13 | 13.135 |
| 4486 | | | | | | | | |
| MAPT- | 269 | 653 | 50.712 | 44.6028815 | 19.173 | 5.038 | 82.251 | 13.252 |
| 4532 | | | | | | | | |
| MAPT- | 270 | 654 | 56.2795 | 28.997742 | 35.775 | 6.797 | 76.784 | 17.19 |
| 4533 | | | | | | | | |
| MAPT- | 271 | 655 | 84.0985 | 66.4744014 | 37.094 | 8.676 | 131.103 | 23.594 |
| 4539 | | | | | | | | |
| MAPT- | 272 | 656 | 56.93 | 25.0980481 | 39.183 | 14.927 | 74.677 | 14.251 |
| 4540 | | | | | | | | |
| MAPT- | 273 | 657 | 42.061 | 23.7969716 | 25.234 | 4.689 | 58.888 | 6.414 |
| 4541 | | | | | | | | |
| MAPT- | 274 | 658 | 37.3465 | 16.4183124 | 25.737 | 2.55 | 48.956 | 6.301 |
| 4543 | | | | | | | | |
| MAPT- | 275 | 659 | 49.8975 | 30.8022785 | 28.117 | 6.319 | 71.678 | 14.003 |
| 4544 | | | | | | | | |
| MAPT- | 276 | 660 | 52.179 | 17.3524004 | 39.909 | 9.44 | 64.449 | 17.044 |
| 4545 | | | | | | | | |
| MAPT- | 277 | 661 | 34.6085 | 5.14420183 | 30.971 | 6.568 | 38.246 | 10.405 |
| 4546 | | | | | | | | |
| MAPT- | 278 | 662 | 38.51 | 12.7293363 | 29.509 | 9.311 | 47.511 | 5.308 |
| 4547 | | | | | | | | |
| MAPT- | 279 | 663 | 46.233 | 8.19678181 | 52.029 | 9.095 | 40.437 | 10.11 |
| 4548 | | | | | | | | |
| MAPT- | 280 | 664 | 46.195 | 5.19440641 | 49.868 | 9.773 | 42.522 | 9.731 |
| 4549 | | | | | | | | |
| MAPT- | 281 | 665 | 64.53 | 30.5625693 | 42.919 | 7.626 | 86.141 | 16.456 |
| 4550 | | | | | | | | |
| MAPT- | 282 | 666 | 48.566 | 4.18890057 | 45.604 | 10.327 | 51.528 | 13.065 |
| 4551 | | | | | | | | |
| MAPT- | 283 | 667 | 38.5625 | 36.1041651 | 13.033 | 5.295 | 64.092 | 10.666 |
| 4552 | | | | | | | | |
| MAPT- | 284 | 668 | 58.8795 | 40.1375022 | 30.498 | 5.099 | 87.261 | 14.948 |
| 4554 | | | | | | | | |
| MAPT- | 285 | 669 | 60.179 | 22.017891 | 44.61 | 13.332 | 75.748 | 6.493 |
| 4556 | | | | | | | | |
| MAPT- | 286 | 670 | 44.634 | 7.32704047 | 39.453 | 7.608 | 49.815 | 7.645 |
| 4557 | | | | | | | | |
| MAPT- | 287 | 671 | 44.4695 | 15.0436968 | 55.107 | 13.83 | 33.832 | 4.355 |
| 4558 | | | | | | | | |
| MAPT- | 288 | 672 | 62.3795 | 19.7912117 | 76.374 | 17.486 | 48.385 | 7.434 |
| 4559 | | | | | | | | |
| MAPT- | 289 | 673 | 48.0585 | 20.8518719 | 33.314 | 11.514 | 62.803 | 7.869 |
| 4560 | | | | | | | | |
| MAPT- | 290 | 674 | 45.0945 | 0.04030509 | 45.123 | 15.353 | 45.066 | 13.72 |
| 4561 | | | | | | | | |
| MAPT- | 291 | 675 | 31.412 | 1.63341666 | 32.567 | 5.823 | 30.257 | 5.108 |
| 4562 | | | | | | | | |
| MAPT- | 292 | 676 | 40.812 | 16.5957962 | 29.077 | 4.004 | 52.547 | 13.653 |
| 4563 | | | | | | | | |
| MAPT- | 293 | 677 | 51.221 | 23.9878904 | 34.259 | 9.457 | 68.183 | 17.349 |
| 4564 | | | | | | | | |
| MAPT- | 294 | 678 | 50.634 | 4.53396868 | 47.428 | 11.37 | 53.84 | 9.461 |
| 4615 | | | | | | | | |
| MAPT- | 295 | 679 | 50.985 | 5.68372431 | 55.004 | 11.45 | 46.966 | 8.038 |
| 4616 | | | | | | | | |
| MAPT- | 296 | 680 | 55.1025 | 21.5702924 | 70.355 | 20.557 | 39.85 | 5.507 |
| 4617 | | | | | | | | |
| MAPT- | 297 | 681 | 48.908 | 3.70665375 | 46.287 | 11.781 | 51.529 | 19.126 |
| 4618 | | | | | | | | |
| MAPT- | 298 | 682 | 38.3115 | 20.5859997 | 23.755 | 5.348 | 52.868 | 12.438 |
| 4619 | | | | | | | | |
| MAPT- | 299 | 683 | 34.8815 | 12.0597062 | 26.354 | 7.457 | 43.409 | 13.598 |
| 4620 | | | | | | | | |
| MAPT- | 300 | 684 | 50.155 | 18.3437641 | 37.184 | 8.204 | 63.126 | 8.376 |
| 4621 | | | | | | | | |
| MAPT- | 301 | 685 | 32.6225 | 1.30178358 | 33.543 | 5.593 | 31.702 | 6.676 |
| 4622 | | | | | | | | |
| MAPT- | 302 | 686 | 45.0005 | 13.5983705 | 35.385 | 8.943 | 54.616 | 7.648 |
| 4623 | | | | | | | | |
| MAPT- | 303 | 687 | 48.5845 | 6.76347636 | 43.802 | 5.103 | 53.367 | 7.374 |
| 4625 | | | | | | | | |
| MAPT- | 304 | 688 | 54.916 | 15.3710872 | 65.785 | 13.194 | 44.047 | 13.218 |
| 4627 | | | | | | | | |
| MAPT- | 305 | 689 | 35.7 | 2.4508321 | 33.967 | 6.77 | 37.433 | 7.946 |
| 4628 | | | | | | | | |
| MAPT- | 306 | 690 | 31.7615 | 4.28718841 | 28.73 | 7.064 | 34.793 | 10.844 |
| 4629 | | | | | | | | |
| MAPT- | 307 | 691 | 26.347 | 10.6235723 | 18.835 | 4.239 | 33.859 | 12.366 |
| 4630 | | | | | | | | |
| MAPT- | 308 | 692 | 39.4495 | 7.60917607 | 34.069 | 5.742 | 44.83 | 10.263 |
| 4632 | | | | | | | | |
| MAPT- | 309 | 693 | 61.3835 | 14.1074874 | 51.408 | 15.912 | 71.359 | 22.804 |
| 4633 | | | | | | | | |
| MAPT- | 310 | 694 | 52.238 | 12.4252804 | 43.452 | 10.809 | 61.024 | 10.061 |
| 4825 | | | | | | | | |
| MAPT- | 311 | 695 | 48.2045 | 2.66508546 | 46.32 | 8.016 | 50.089 | 16.64 |
| 4828 | | | | | | | | |
| MAPT- | 312 | 696 | 89.127 | 35.3779665 | 114.143 | 29.128 | 64.111 | 16.557 |
| 5682 | | | | | | | | |
| MAPT- | 313 | 697 | 46.249 | 12.6359982 | 37.314 | 6.879 | 55.184 | 14.344 |
| 5958 | | | | | | | | |
| MAPT- | 314 | 698 | 43.587 | 19.7523208 | 29.62 | 8.048 | 57.554 | 13.293 |
| 5959 | | | | | | | | |
| MAPT- | 315 | 699 | 40.428 | 6.24658131 | 36.011 | 10.381 | 44.845 | 4.108 |
| 5961 | | | | | | | | |
| MAPT- | 316 | 700 | 38.295 | 2.01808275 | 36.868 | 5.593 | 39.722 | 10.772 |
| 5963 | | | | | | | | |
| MAPT- | 317 | 701 | 52.7395 | 16.4678098 | 41.095 | 8.774 | 64.384 | 7.757 |
| 5964 | | | | | | | | |
| MAPT- | 318 | 702 | 45.2395 | 21.7541401 | 29.857 | 7.021 | 60.622 | 8.921 |
| 5965 | | | | | | | | |
| MAPT- | 319 | 703 | 51.0255 | 9.61877354 | 44.224 | 6.395 | 57.827 | 9.074 |
| 5966 | | | | | | | | |
| MAPT- | 320 | 704 | 56.756 | 19.2488608 | 43.145 | 10.168 | 70.367 | 13.456 |
| 5967 | | | | | | | | |
| MAPT- | 321 | 705 | 49.755 | 14.8365145 | 60.246 | 12.788 | 39.264 | 9.459 |
| 5968 | | | | | | | | |
| MAPT- | 322 | 706 | 30.953 | 2.88358145 | 32.992 | 7.492 | 28.914 | 6.891 |
| 6006 | | | | | | | | |
| MAPT- | 323 | 707 | 46.1245 | 14.5968053 | 35.803 | 7.552 | 56.446 | 6.025 |
| 6007 | | | | | | | | |
| MAPT- | 324 | 708 | 52.218 | 3.03065966 | 54.361 | 9.738 | 50.075 | 7.532 |
| 6008 | | | | | | | | |
| MAPT- | 325 | 709 | 76.4995 | 42.195183 | 106.336 | 19.189 | 46.663 | 11.419 |
| 6009 | | | | | | | | |
| MAPT- | 326 | 710 | 58.729 | 23.2892689 | 42.261 | 7.547 | 75.197 | 5.272 |
| 6010 | | | | | | | | |
| MAPT- | 327 | 711 | 52.692 | 4.01212388 | 49.855 | 12.113 | 55.529 | 14.5 |
| 6011 | | | | | | | | |
| MAPT- | 328 | 712 | 63.9645 | 0.68377226 | 64.448 | 15.339 | 63.481 | 10.773 |
| 6012 | | | | | | | | |
| MAPT- | 329 | 713 | 53.8995 | 17.2314852 | 66.084 | 23.254 | 41.715 | 10.372 |
| 6013 | | | | | | | | |
| MAPT- | 330 | 714 | 33.9865 | 1.58886894 | 32.863 | 4.884 | 35.11 | 8.916 |
| 6014 | | | | | | | | |
| MAPT- | 331 | 715 | 47.801 | 25.4077609 | 29.835 | 8.764 | 65.767 | 6.147 |
| 6015 | | | | | | | | |
| MAPT- | 332 | 716 | 46.9375 | 0.02192031 | 46.953 | 10.206 | 46.922 | 11.089 |
| 6017 | | | | | | | | |
| MAPT- | 333 | 717 | 54.6625 | 9.43492578 | 61.334 | 13.818 | 47.991 | 12.587 |
| 6119 | | | | | | | | |
| MAPT- | 334 | 718 | 64.7155 | 0.73468395 | 65.235 | 6.382 | 64.196 | 10.199 |
| 6628 | | | | | | | | |
| MAPT- | 335 | 719 | 50.0975 | 11.687768 | 58.362 | 10.929 | 41.833 | 14.095 |
| 6629 | | | | | | | | |
| MAPT- | 336 | 720 | 81.9435 | 12.0568777 | 73.418 | 19.87 | 90.469 | 17.638 |
| 6631 | | | | | | | | |
| MAPT- | 337 | 721 | 33.822 | 12.4677068 | 25.006 | 8.472 | 42.638 | 12.985 |
| 6672 | | | | | | | | |
| MAPT- | 338 | 722 | 24.959 | 10.8512607 | 17.286 | 2.982 | 32.632 | 9.031 |
| 6731 | | | | | | | | |
| MAPT- | 339 | 723 | 38.0145 | 13.7228213 | 28.311 | 11.443 | 47.718 | 15.906 |
| 6732 | | | | | | | | |
| MAPT- | 340 | 724 | 34.031 | 28.3691241 | 13.971 | 3.303 | 54.091 | 13.69 |
| 6738 | | | | | | | | |
| MAPT- | 341 | 725 | 38.316 | 29.593833 | 17.39 | 2.841 | 59.242 | 14.056 |
| 6739 | | | | | | | | |
| MAPT- | 342 | 726 | 34.866 | 13.8847488 | 25.048 | 11.345 | 44.684 | 6.187 |
| 6740 | | | | | | | | |
| MAPT- | 343 | 727 | 34.507 | 11.2825958 | 26.529 | 7.507 | 42.485 | 10.487 |
| 6741 | | | | | | | | |
| MAPT- | 344 | 728 | 62.1435 | 29.7882874 | 41.08 | 12.738 | 83.207 | 17.359 |
| 6742 | | | | | | | | |
| MAPT- | 345 | 729 | 29.6205 | 23.3790715 | 13.089 | 2.767 | 46.152 | 5.76 |
| 6743 | | | | | | | | |
| MAPT- | 346 | 730 | 26.926 | 10.1229407 | 19.768 | 4.146 | 34.084 | 5.015 |
| 6745 | | | | | | | | |
| MAPT- | 347 | 731 | 60.0265 | 3.40471915 | 57.619 | 11.694 | 62.434 | 11.211 |
| 6748 | | | | | | | | |
| MAPT- | 348 | 732 | 50.6395 | 36.3502383 | 24.936 | 4.104 | 76.343 | 16.107 |
| 6749 | | | | | | | | |
| MAPT- | 349 | 733 | 44.856 | 3.86080303 | 47.586 | 12.024 | 42.126 | 13.817 |
| 6750 | | | | | | | | |
| MAPT- | 350 | 734 | 78.4055 | 17.0730932 | 66.333 | 7.978 | 90.478 | 12.547 |
| 6751 | | | | | | | | |
| MAPT- | 351 | 735 | 50.173 | 15.0231907 | 60.796 | 7.566 | 39.55 | 9.59 |
| 6752 | | | | | | | | |
| MAPT- | 352 | 736 | 71.371 | 11.5413969 | 63.21 | 16.035 | 79.532 | 21.212 |
| 6753 | | | | | | | | |
| MAPT- | 353 | 737 | 36.8455 | 3.50654253 | 34.366 | 6.737 | 39.325 | 9.505 |
| 6754 | | | | | | | | |
| MAPT- | 354 | 738 | 49.7045 | 24.223357 | 66.833 | 10.306 | 32.576 | 5.734 |
| 6755 | | | | | | | | |
| MAPT- | 355 | 739 | 44.4755 | 32.1599235 | 21.735 | 7.721 | 67.216 | 9.314 |
| 6756 | | | | | | | | |
| MAPT- | 356 | 740 | 33.5075 | 4.63225652 | 30.232 | 9.588 | 36.783 | 6.407 |
| 6757 | | | | | | | | |
| MAPT- | 357 | 741 | 31.353 | 12.2753737 | 22.673 | 6.04 | 40.033 | 9.561 |
| 6758 | | | | | | | | |
| MAPT- | 358 | 742 | 41.85 | 0.46669048 | 41.52 | 10.866 | 42.18 | 7.164 |
| 6759 | | | | | | | | |
| MAPT- | 359 | 743 | 32.4735 | 18.9158135 | 45.849 | 11.71 | 19.098 | 3.864 |
| 6760 | | | | | | | | |
| MAPT- | 360 | 744 | 45.049 | 19.3718974 | 31.351 | 10.31 | 58.747 | 11.659 |
| 6761 | | | | | | | | |
| MAPT- | 361 | 745 | 38.174 | 8.13031377 | 43.923 | 15.429 | 32.425 | 4.765 |
| 6762 | | | | | | | | |
| MAPT- | 362 | 746 | 53.7735 | 2.06828733 | 52.311 | 15.07 | 55.236 | 8.063 |
| 6763 | | | | | | | | |
| MAPT- | 363 | 747 | 53.1035 | 33.2686669 | 29.579 | 9.81 | 76.628 | 17.226 |
| 6764 | | | | | | | | |
| MAPT- | 364 | 748 | 52.8995 | 16.5795327 | 41.176 | 10.853 | 64.623 | 15.24 |
| 6765 | | | | | | | | |
| MAPT- | 365 | 749 | 49.2605 | 4.00293149 | 52.091 | 10.614 | 46.43 | 16.518 |
| 6766 | | | | | | | | |
| MAPT- | 366 | 750 | 71.423 | 0.85701342 | 72.029 | 26.702 | 70.817 | 16.417 |
| 6767 | | | | | | | | |
| MAPT- | 367 | 751 | 55.4255 | 9.05308812 | 61.827 | 22.465 | 49.024 | 12.369 |
| 6768 | | | | | | | | |
| MAPT- | 368 | 752 | 72.08 | 0.29839906 | 71.869 | 16.441 | 72.291 | 37.177 |
| 6769 | | | | | | | | |
| MAPT- | 369 | 753 | 49.619 | 14.4377063 | 59.828 | 20.675 | 39.41 | 13.235 |
| 6772 | | | | | | | | |
| MAPT- | 370 | 754 | 48.624 | 11.5583674 | 56.797 | 12.783 | 40.451 | 6.391 |
| 6773 | | | | | | | | |
| MAPT- | 371 | 755 | 52.4405 | 31.8855661 | 29.894 | 6.706 | 74.987 | 5.573 |
| 6774 | | | | | | | | |
| MAPT- | 372 | 756 | 28.415 | 6.30456406 | 32.873 | 8.748 | 23.957 | 5.955 |
| 6775 | | | | | | | | |
| MAPT- | 373 | 757 | 39.772 | 4.84509566 | 43.198 | 3.707 | 36.346 | 9.384 |
| 6777 | | | | | | | | |
| MAPT- | 374 | 758 | 50.719 | 5.85342993 | 54.858 | 14.257 | 46.58 | 14.17 |
| 6778 | | | | | | | | |
| MAPT- | 375 | 759 | 35.5865 | 10.7459018 | 43.185 | 6.174 | 27.988 | 3.65 |
| 6779 | | | | | | | | |
| MAPT- | 376 | 760 | 71.501 | 11.9614183 | 79.959 | 23.953 | 63.043 | 14.252 |
| 6780 | | | | | | | | |
| MAPT- | 377 | 761 | 43.875 | 9.50210093 | 37.156 | 6.446 | 50.594 | 8.933 |
| 6781 | | | | | | | | |
| MAPT- | 378 | 762 | 43.4265 | 3.49381461 | 40.956 | 13.993 | 45.897 | 6.518 |
| 6789 | | | | | | | | |
| MAPT- | 379 | 763 | 46.25 | 28.0028427 | 26.449 | 10.778 | 66.051 | 11.602 |
| 6792 | | | | | | | | |
| MAPT- | 380 | 764 | 33.324 | 13.8762635 | 43.136 | 13.663 | 23.512 | 6.983 |
| 6793 | | | | | | | | |
| MAPT- | 381 | 765 | 33.747 | 11.8765655 | 42.145 | 13.155 | 25.349 | 9.4 |
| 6795 | | | | | | | | |
| MAPT- | 382 | 766 | 65.7615 | 8.10556503 | 71.493 | 9.454 | 60.03 | 12.964 |
| 6796 | | | | | | | | |
| MAPT- | 383 | 767 | 55.439 | 15.7302975 | 66.562 | 17.934 | 44.316 | 16.313 |
| 6797 | | | | | | | | |
| MAPT- | 384 | 768 | 50.3055 | 4.55023214 | 47.088 | 13.033 | 53.523 | 13.441 |
| 6798 | | | | | | | | |

### Example 3: GalNAc-Conjugated MAPT RNAi Oligonucleotides Inhibit Human MAPT mRNA Expression In Vivo

The in vitro screening assays in Example 2 validated the ability of MAPT-targeting oligonucleotides to knockdown target mRNA. To further evaluate the ability of MAPT RNAi oligonucleotides to inhibit MAPT mRNA expression, GalNAc-conjugated MAPT-targeting oligonucleotides were generated to confirm knockdown in vivo.

Specifically, a subset of the DsiRNAs identified in Example 2 were used to generate corresponding ds RNAi oligonucleotides comprising a nicked tetraloop GalNAc-conjugated structure (referred to herein as “GalNAc-conjugated MAPT oligonucleotides” or “GalNAc-MAPT oligonucleotides”) having a 36-mer sense strand and a 22-mer antisense strand (Tables 4 and 5). Further, the nucleotide sequences comprising the sense strand and antisense strand have a distinct pattern of modified nucleotides and phosphorothioate linkages. Three of the nucleotides comprising the tetraL were each conjugated to a GalNAc moiety (CAS #14131-60-3). The benchmark control (MAPT-2460) has a different modification pattern than the remaining oligonucleotides. The modification patterns are illustrated below:

Sense Strand:
5′-X-S-mX-fX-mX-fX-mX-mX-fX-mX-fX-mX-fX-fX-mX-fX-
mX-fX-mX-mX-mX-mX-mX-mX-mX-mX-mX-mX-[ademX-
GalNAc]-[ademX-GalNAc]-[ademX-GalNAc]-mX-mX-mX-mX-
mX-mX-3′ hybridized to:
Antisense Strand:
5′-[MePhosphonate-4O-mX]-S-fX-S-fX-fX-fX-mX-fX-mX-
mX-fX-mX-mX-mX-fX-mX-fX-mX-mX-fX-mX-S-mX-S-mX-3′.
(Modification key: Table 3).

Or, represented as:

Sense Strand:
[mXs][mX][fX][mX][fX][mX][mX][fX][mX][fX][mX][fX]
[fX][mX][fX][mX][fX][mX][mX][X][mX][mX][X][mX][mX]
[mX][mX][ademX-GalNAc][ademX-GalNAc][ademX-GalNAc]
[mX][mX][mX][mX][mX][mX] hybridized to:
Antisense Strand:
[MePhosphonate-4O-mXs][fXs][fX][fX][fX][mX][fX]
[mX][mX][fX][mX][mX][mX][fX][mX][fX][mX][mX][fX]
[mXs][mXs][mX].
(Modification key: Table 3).

### Benchmark Modification Pattern

Sense Strand:
5′-mX-S-mX-mX-mX-mX-mX-mX-fX-fX-fX-fX-mX-mX-mX-mX-
mX-mX-mX-mX-mX-mX-mX-mX-mX-mX-mX-mX-[ademX-
GalNAc]-[ademX-GalNAc]-[ademX-GalNAc]-mX-mX-mX-mX-
mX-mX-3′ hybridized to:
Antisense Strand:
5′-[MePhosphonate-4O-mX]-S-fX-S-fX-S-fX-fX-mX-fX-
mX-mX-fX-mX-mX-mX-fX-mX-mX-mX-mX-mX-mX-S-mX-S-mX-
3′.
(Modification key: Table 3).

Or, represented as:

Sense Strand:
[mXs][mX][mX][mX][mX][mX][mX][fX][fX][fX][fX][mX]
[mX][mX][mX][mX][mX][mX][mX][mX][mX][mX][mX][mX]
[mX][mX][mX][ademX-GalNAc][ademX-GalNAc][ademX-
GalNAc][mX][mX][mX][mX][mX][X] hybridized to:
Antisense Strand:
[MePhosphonate-4O-mXs][fXs][fX][fX][fX][mX][fX][mX]
[mX][fX][mX][mX][mX][fX][mX][mX][mX][mX][mX][mXs]
[mXs][mX].
(Modification key: Table 3).

TABLE 3
Key for Modification Patterns.

| | |
|---|---|
| Symbol | Modification/linkage |

Key 1

| | |
|---|---|
| mX | 2′-OMe-modified nucleotide |
| fX | 2′-F-modified nucleotide |
| -S- | phosphorothioate linkage |
| - | phosphodiester linkage |
| [MePhosphonate- | 4′-O-monomethylphosphonate-2′-O-methyl modified |
| 4O-mX] | nucleotide |
| ademX-GalNAc | GalNAc attached to a nucleotide |
| ademX-C16 | C₁₆ hydrocarbon chain attached to a nucleotide |

Key 2

| | |
|---|---|
| [mXs] | 2′-OMe-modified nucleotide with a phosphorothioate |
| | linkage to the neighboring nucleotide |
| [fXs] | 2′-F-modified nucleotide with a phosphorothioate |
| | linkage to the neighboring nucleotide |
| [mX] | 2′-OMe-modified nucleotide with phosphodiester |
| | linkages to neighboring nucleotides |
| [fX] | 2′-F-modified nucleotide with phosphodiester linkages |
| | to neighboring nucleotides |
| [ademXs-C16] | C₁₆ hydrocarbon chain attached to a nucleotide with |
| | phosphodiester linkages to neighboring nucleotides |

The GalNAc-conjugated MAPT-targeting oligonucleotides were used in an HDI model to confirm the ability of the RNAi oligonucleotides to knockdown MAPT gene expression in vivo. The GalNAc-conjugated MAPT-targeting oligonucleotides listed in Tables 4 and 5 were evaluated in mice engineered to transiently express human MAPT mRNA in hepatocytes of the mouse liver. Briefly, 6-8-week-old female CD-1 mice (n=4-5) were subcutaneously administered the indicated GalNAc-conjugated MAPT-targeting oligonucleotides at a dose of 3 mg/kg formulated in PBS. A control group of mice (n=5) were administered only PBS. Four days later (96 hours), the mice were HDI with a DNA plasmid encoding the full human MAPT gene (SEQ ID NO: 909) (10 μg) under control of a ubiquitous cytomegalovirus (CMV) promoter sequence. One day after introduction of the DNA plasmid, liver samples from HDI mice were collected. Total RNA derived from these HDI mice were subjected to qRT-PCR analysis to determine human MAPT mRNA levels as described in Example 2. The values were normalized for transfection efficiency using the NeoR gene included on the DNA plasmid. Benchmark controls (MAPT-2460) were used to confirm successful knock-down.

TABLE 4
GalNAc-Conjugated Human MAPT-Targeting RNAi
Oligonucleotides for HDI Screen (Set I).

| RNAi | SEQ ID NO | SEQ ID NO | SEQ ID NO | SEQ ID NO |
|---|---|---|---|---|
| Oligonucleo- | (Sense) | (Antisense) | (Sense) | (Antisense) |
| tide | Unmodified | | Modified | |
| MAPT-1479 | 787 | 822 | 857 | 892 |
| MAPT-1505 | 788 | 823 | 858 | 893 |
| MAPT-2096 | 789 | 824 | 859 | 894 |
| MAPT-2270 | 790 | 825 | 860 | 895 |
| MAPT-2279 | 791 | 826 | 861 | 896 |
| MAPT-2281 | 792 | 827 | 862 | 897 |
| MAPT-2284 | 793 | 828 | 863 | 898 |
| MAPT-2299 | 794 | 829 | 864 | 899 |
| MAPT-2376 | 795 | 830 | 865 | 900 |
| MAPT-2379 | 796 | 831 | 866 | 901 |
| MAPT-2382 | 797 | 832 | 867 | 902 |
| MAPT-2449 | 798 | 833 | 868 | 903 |
| MAPT-2450 | 799 | 834 | 869 | 904 |
| MAPT-2451 | 800 | 835 | 870 | 905 |
| MAPT-2452 | 801 | 836 | 871 | 906 |
| MAPT-2453 | 802 | 837 | 872 | 907 |
| MAPT-2454 | 803 | 838 | 873 | 908 |
| MAPT-2460 | 786 | 821 | 856 | 891 |

TABLE 5
GalNAc-Conjugated Human MAPT-Targeting RNAi
Oligonucleotides for HDI Screen (Set II).

| RNAi | SEQ ID NO | SEQ ID NO | SEQ ID NO | SEQ ID NO |
|---|---|---|---|---|
| Oligonucleo- | (Sense) | (Antisense) | (Sense) | (Antisense) |
| tide | Unmodified | | Modified | |
| MAPT-2456 | 769 | 804 | 839 | 874 |
| MAPT-2567 | 770 | 805 | 840 | 875 |
| MAPT-2723 | 771 | 806 | 841 | 876 |
| MAPT-0690 | 772 | 807 | 842 | 877 |
| MAPT-1494 | 773 | 808 | 843 | 878 |
| MAPT-1733 | 774 | 809 | 844 | 879 |
| MAPT-2273 | 775 | 810 | 845 | 880 |
| MAPT-2274 | 776 | 811 | 846 | 881 |
| MAPT-2276 | 777 | 812 | 847 | 882 |
| MAPT-2301 | 778 | 813 | 848 | 883 |
| MAPT-2347 | 779 | 814 | 849 | 884 |
| MAPT-2357 | 780 | 815 | 850 | 885 |
| MAPT-2358 | 781 | 816 | 851 | 886 |
| MAPT-2364 | 782 | 817 | 852 | 887 |
| MAPT-2378 | 783 | 818 | 853 | 888 |
| MAPT-2459 | 784 | 819 | 854 | 889 |
| MAPT-2461 | 785 | 820 | 855 | 890 |
| MAPT-2460 | 786 | 821 | 856 | 891 |

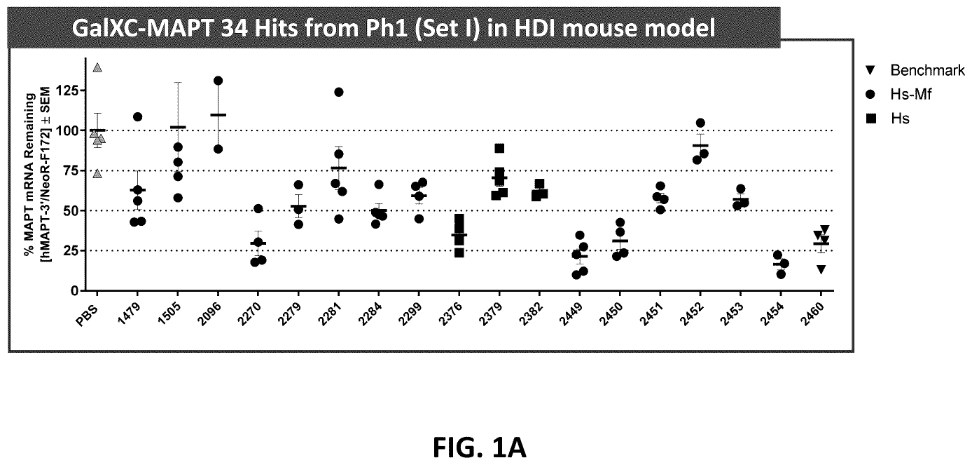

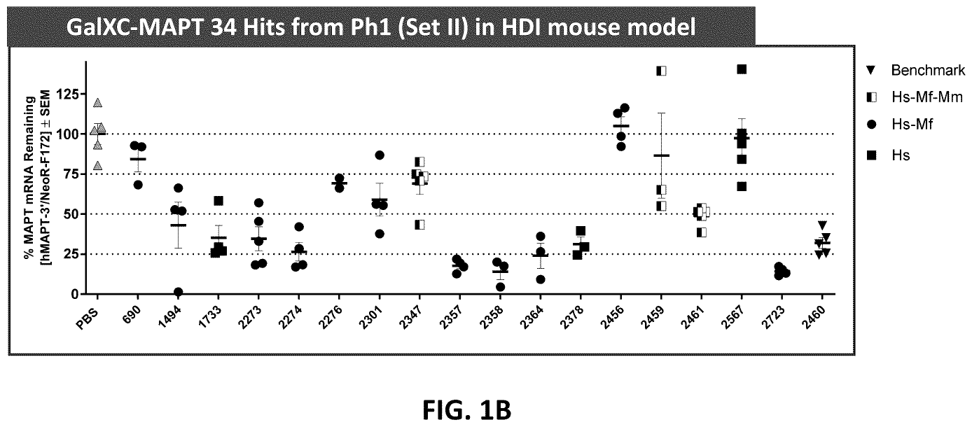

The results in FIGS. 1A and 1B demonstrate that GalNAc-conjugated MAPT-targeting oligonucleotides (as shown in Tables 4 and 5, respectively) designed to target human MAPT mRNA successfully inhibited human MAPT mRNA expression in HDI mice, as determined by a reduction in the amount of human MAPT mRNA expression in liver samples from HDI mice treated with GalNAc-conjugated MAPT-targeting oligonucleotides relative to control HDI mice treated with only PBS.

### Example 4: GalNAc-Conjugated MAPT-Targeting RNAi Oligonucleotides Inhibit Human MAPT Gene Expression in a Dose-Dependent Manner

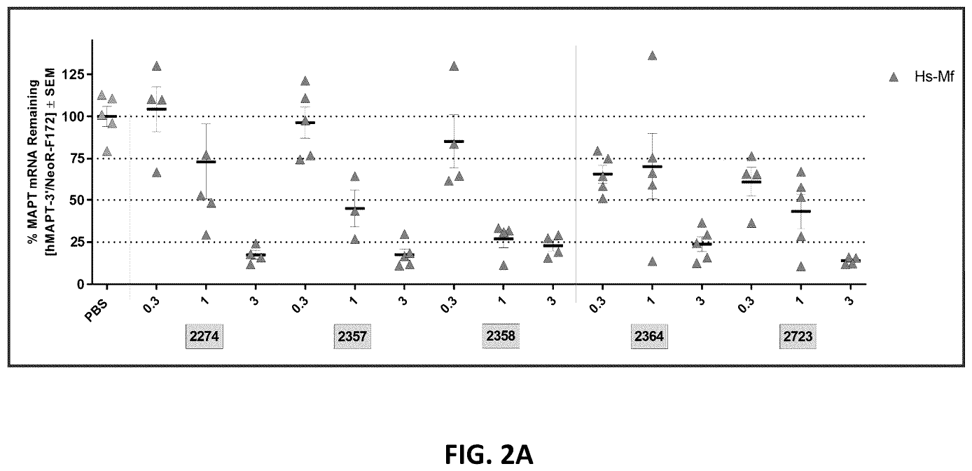

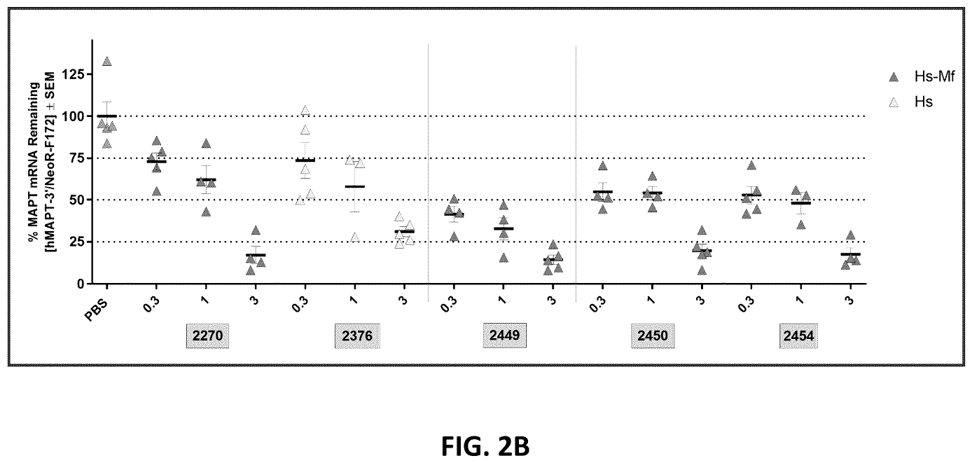

To further evaluate the ability of GalNAc-conjugated MAPT-targeting RNAi oligonucleotides to inhibit MAPT gene expression, a dose response study was carried out. Specifically, in separate treatment groups, selected GalNAc-conjugated MAPT-targeting RNAi oligonucleotides (Tables 6 and 7) were formulated in PBS and were administered to CD-1 mice at doses of 0.3 mg/kg, 1 mg/kg, or 3 mg/kg subcutaneously. As described in Example 3, a human MAPT DNA expression plasmid was administered to the mice 4 days post-oligonucleotide dosing, and livers were collected 20 hours later for qRT-PCR analysis. As shown in FIGS. 2A and 2B, all of the GalNAc-conjugated MAPT-targeting RNAi oligonucleotides tested inhibited human MAPT gene expression in a dose-dependent manner. Potent GalNAc-conjugated MAPT-targeting oligonucleotides (i.e., MAPT-2449, MAPT-2357, MAPT-2450, MAPT-2358, MAPT-2454, and MAPT-2723) reduced MAPT mRNA by around 50% or more at 1 mg/kg and even further at 3 mg/kg. These constructs were selected for further studies in NHPs.

TABLE 6
GalNAc-Conjugated Human MAPT-Targeting RNAi
Oligonucleotides for Dose Screen (Set I).

| RNAi | SEQ ID NO | SEQ ID NO | SEQ ID NO | SEQ ID NO |
|---|---|---|---|---|
| Oligonucleo- | (Sense) | (Antisense) | (Sense) | (Antisense) |
| tide | Unmodified | | Modified | |
| MAPT-2270 | 790 | 825 | 860 | 895 |
| MAPT-2376 | 795 | 830 | 865 | 900 |
| MAPT-2449 | 798 | 833 | 868 | 903 |
| MAPT-2450 | 799 | 834 | 869 | 904 |
| MAPT-2454 | 803 | 838 | 873 | 908 |

TABLE 7
GalNAc-Conjugated Human MAPT-Targeting RNAi
Oligonucleotides for Dose Screen (Set II).

| RNAi | SEQ ID NO | SEQ ID NO | SEQ ID NO | SEQ ID NO |
|---|---|---|---|---|
| Oligonucleo- | (Sense) | (Antisense) | (Sense) | (Antisense) |
| tide | Unmodified | | Modified | |
| MAPT-2723 | 771 | 806 | 841 | 876 |
| MAPT-2274 | 776 | 811 | 846 | 881 |
| MAPT-2357 | 780 | 815 | 850 | 885 |
| MAPT-2358 | 781 | 816 | 851 | 886 |
| MAPT-2364 | 782 | 817 | 852 | 887 |

### Example 5: RNAi Oligonucleotide Inhibition of MAPT Gene Expression in NHP CNS

Effective GalNAc-conjugated MAPT-targeting oligonucleotides identified in the HDI mouse studies were assayed for inhibition in NHP. Specifically, GalNAc-conjugated MAPT-targeting oligonucleotides listed in Table 8 were evaluated in non-naïve cynomolgus monkeys (Macaca fascicularis; Mf). Each cohort contained 4 female subjects weighing 2.6-4.3 kg. The GalNAc-conjugated MAPT-targeting oligonucleotides were administered at a dose of 50 mg in 1.6 mL of artificial cerebrospinal fluid (aCSF) on study days 0 and 7 via intra cisterna magna (i.e.m.) injection.

TABLE 8
GalNAc-Conjugated MAPT-Targeting RNAi
Oligonucleotides for NHP Study.

| | | SEQ | SEQ | SEQ | SEQ |
|---|---|---|---|---|---|
| RNAi | | ID NO | ID NO | ID NO | ID NO |
| Oligonucleo- | Alternate | (Sense) | (Antisense) | (Sense) | (Antisense) |
| tide | name | Unmodified | | Modified | |
| MAPT-2723 | DCR 214 | 771 | 806 | 841 | 876 |
| MAPT-2357 | DCR 211 | 780 | 815 | 850 | 885 |
| MAPT-2358 | DCR 212 | 781 | 816 | 851 | 886 |
| MAPT-2449 | DCR 207 | 798 | 833 | 868 | 903 |
| MAPT-2450 | DCR 208 | 799 | 834 | 869 | 904 |
| MAPT-2454 | DCR 209 | 803 | 838 | 873 | 908 |

On study day 14, CNS tissue was collected and subjected to qRT-PCR analysis to measure MAPT mRNA in oligonucleotide-treated monkeys relative to those treated with a comparable volume of aCSF. To normalize the data, the measurements were made relative to the reference gene, RPL23. The following SYBR assays purchased from Integrated DNA Technologies were used to evaluate gene expressions:

Forward:
AGGACAGAGTGCAGTCGAAGATC;
Reverse:
AGGTCAGCTTGTGGGTTTCAA;
and
Probe:
CACCCATGTCCCTGGCGGAGG.

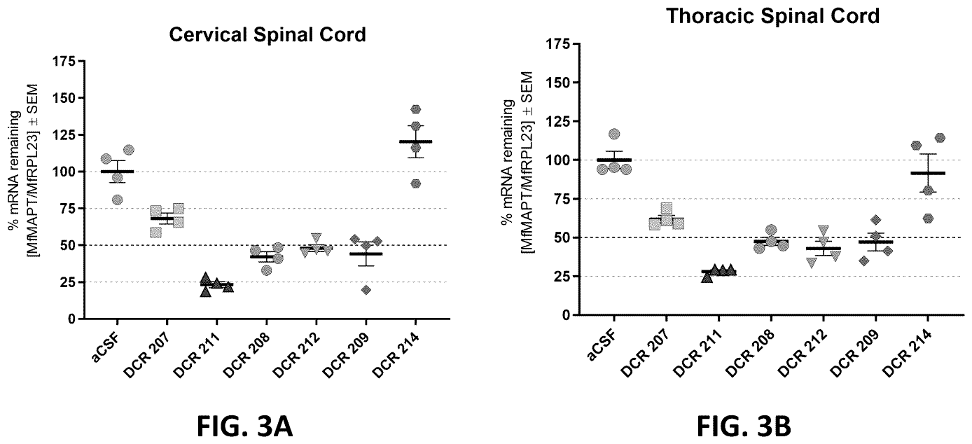

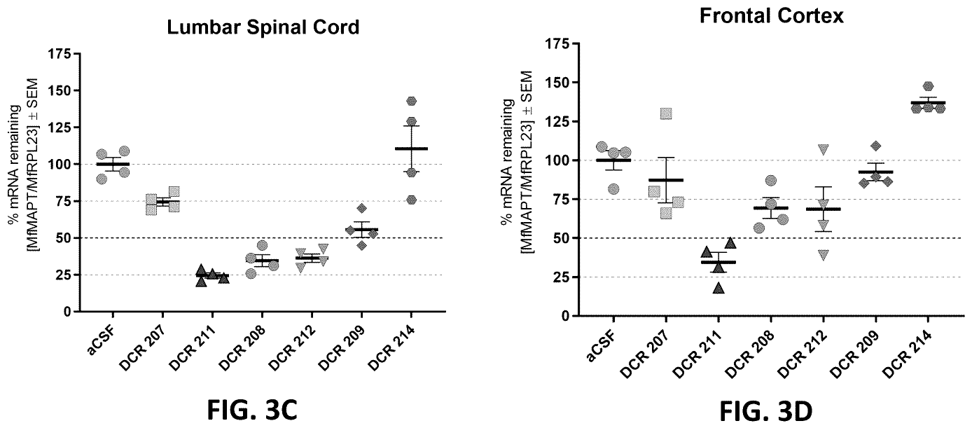

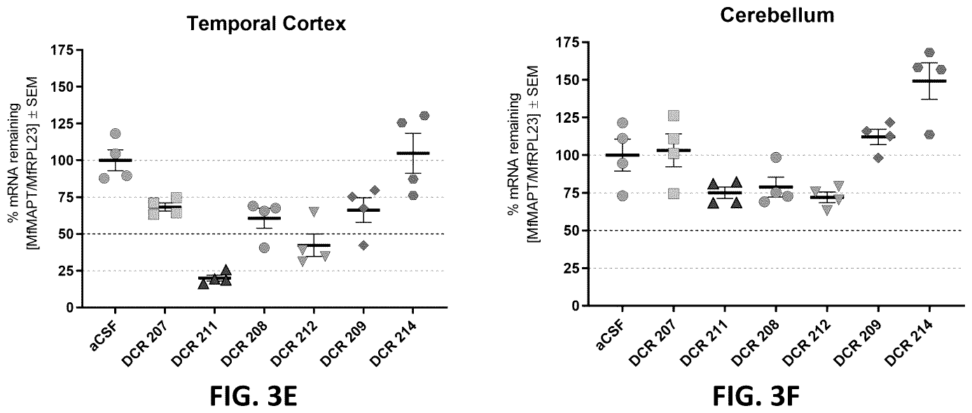

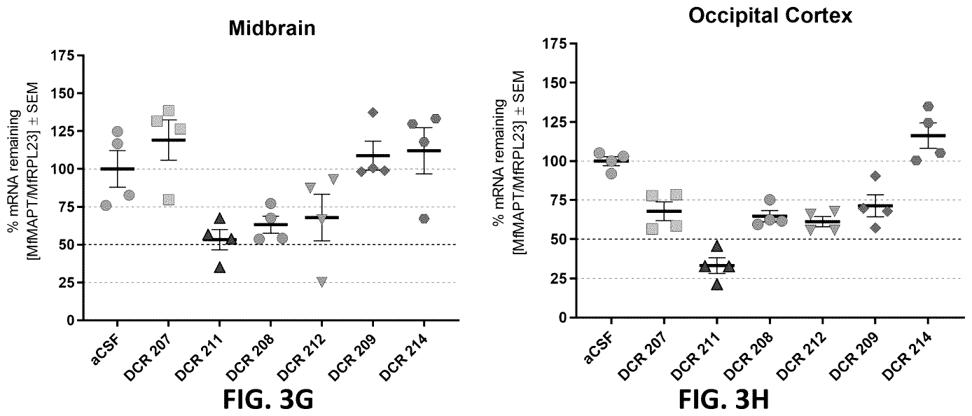

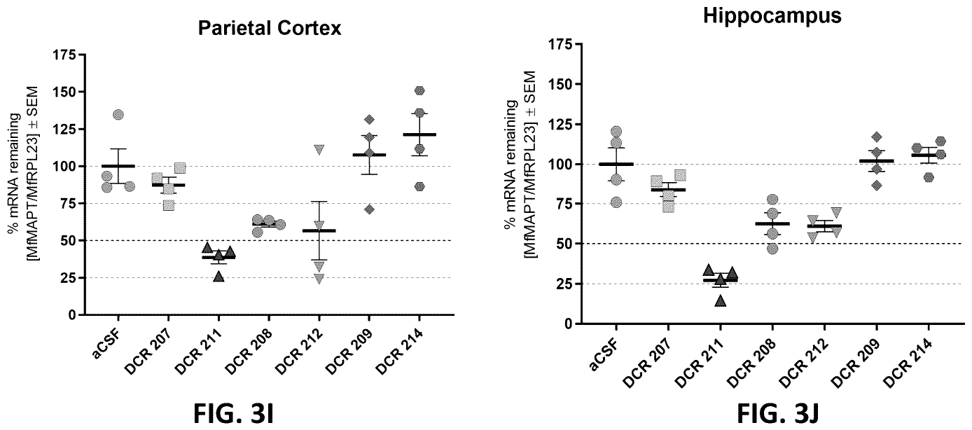

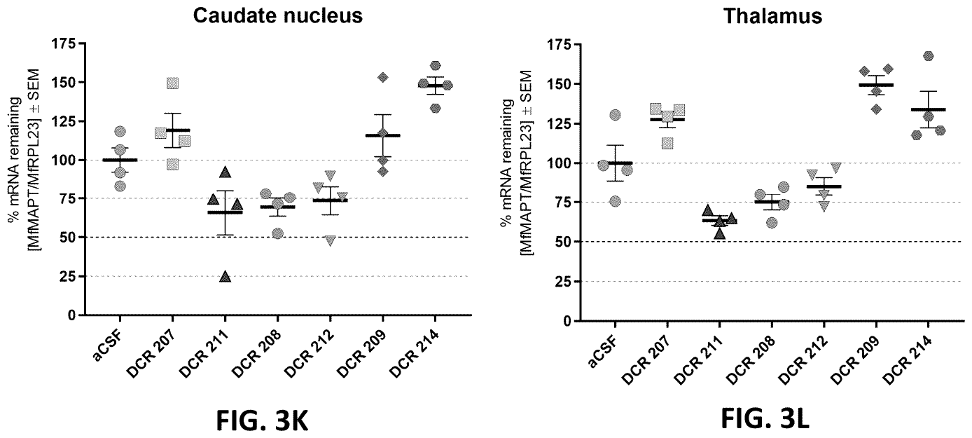

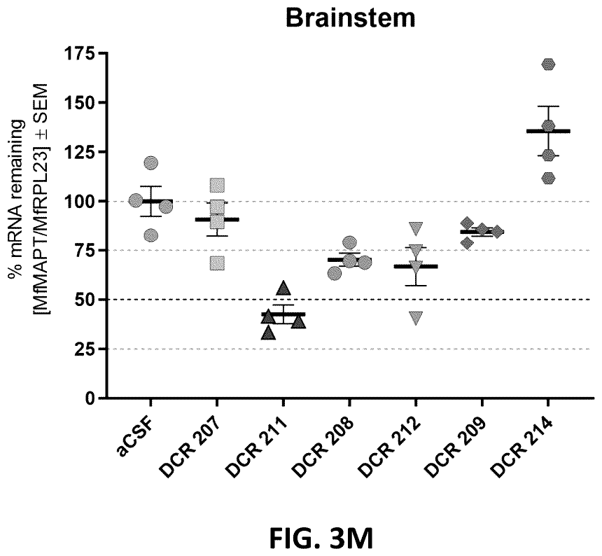

As shown in FIGS. 3A-3M (Day 14), treating NHPs with the GalNAc-conjugated MAPT-targeting oligonucleotides inhibited MAPT gene expression in several regions of the CNS, as determined by a reduced amount of MAPT mRNA in brain samples from oligonucleotide-treated NHPs relative to NHPs treated with aCSF. Several GalNAc-conjugated MAPT-targeting oligonucleotides reduced MAPT gene expression throughout the CNS. MAPT-2357 (DCR 211) was particularly potent in the cervical spinal cord, thoracic spinal cord, lumbar spinal cord, frontal cortex, temporal cortex, occipital cortex, and brain stem. These results demonstrate that treating NHPs with the GalNAc-conjugated MAPT-targeting oligonucleotides reduces the amount of MAPT mRNA in the CNS.

### Example 6: Lipid Conjugation of MAPT-Targeting Oligonucleotides Reduces Expression in NHP CNS

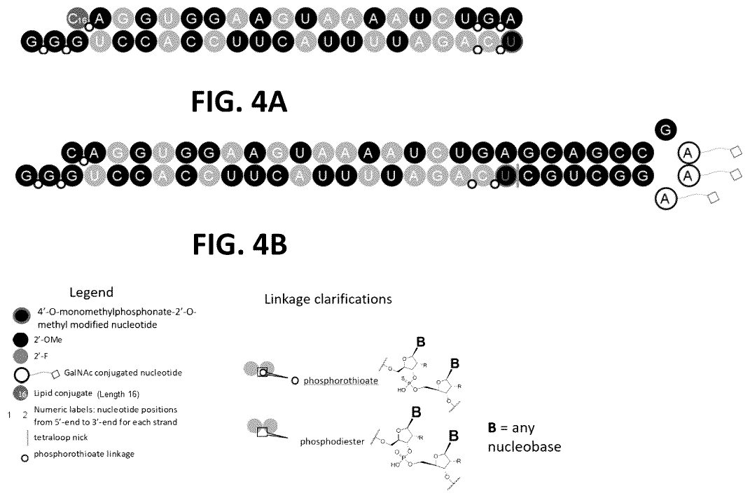

To further investigate the efficacy of oligonucleotides targeting MAPT, a lipid-conjugated oligonucleotide was assessed in NHP compared to a GalNAc-conjugated oligonucleotide. Specifically, the GalNAc-conjugated MAPT-2357 (DCR 211) described in Example 3, having a 36-mer sense strand and 22-mer antisense strand (SEQ ID NOs: 850 and 885, respectively) was compared to a lipid-conjugated MAPT-2357 (DCR 211), having a 20-mer sense strand and 22-mer antisense strand (SEQ ID NOs: 1682 and 885, respectively). FIGS. 4A-4B show the chemical modification patterns of each oligonucleotide, and the chemical modification pattern of the lipid-conjugated oligonucleotide is provided below:

Sense Strand:
5′-[ademX-C16]-S-mX-fX-mX-fX-mX-mX-fX-mX-fX-mX-fX-
fX-mX-fX-mX-fX-mX-S-mX-S-mX-3′ hybridized to:
Antisense Strand:
5′-[MePhosphonate-4O-mX]-S-fX-S-fX-fX-fX-mX-fX-mX-
mX-fX-mX-mX-mX-fX-mX-fX-mX-mX-fX-mX-S-mX-S-mX-3′.
(Modification key: Table 3).

Or, represented as:

Sense Strand:
[ademXs-C16][mX][fX][mX][fX][mX][mX][fX][mX][fX]
[mX][fX][fX][mX][fX][mX][fX][mXs][mXs][mX]
hybridized to:
Antisense Strand:
[MePhosphonate-4O-mXs][fXs][fX][fX][fX][mX][fX]
[mX][mX][fX][mX][mX][mX][fX][mX][fX][mX][mX][fX]
[mXs][mXs][mX]
(Modification key: Table 3).

### Lipid Conjugation

Conjugation of a lipid moiety to the MAPT-targeting oligonucleotide was carried out using phosphoramidite synthesis as shown below.

### Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy) methoxy) ethoxy) ethan-1-ammonium formate (1-6)

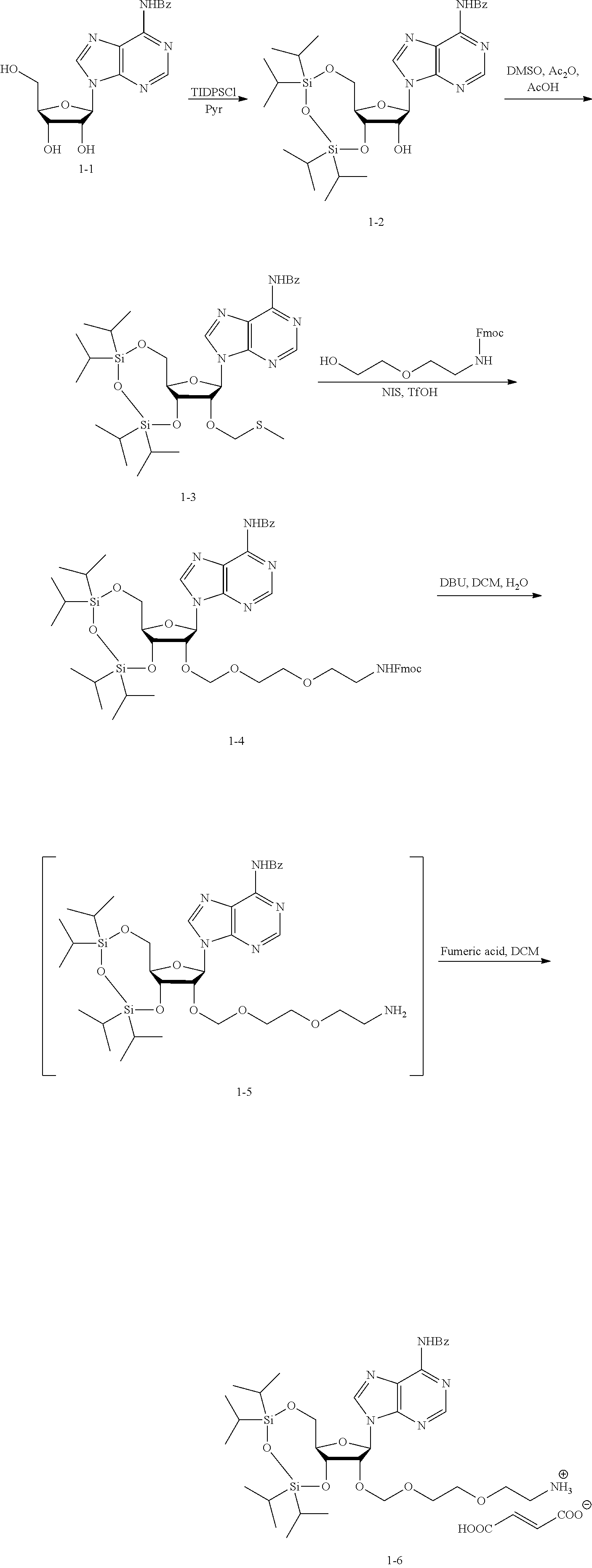

A solution of compound 1-1 (25.00 g, 67.38 mmol) in 20 mL of dimethylformamide (DMF) was treated with pyridine (11 mL, 134.67 mmol) and tetraisopropyldisiloxane dichloride (22.63 mL, 70.75 mmol) at 10° C. The resulting mixture was stirred at 25° C. for 3 hours and quenched with 20% citric acid (50 mL). The aqueous layer was extracted with ethyl acetate (EtOAc; 3λ50 mL) and the combined organic layers were concentrated in vacuo. The crude residue was recrystallized from a mixture of methyl tert-butyl ether (MTBE) and n-heptane (1:15, 320 mL) to afford compound 1-2 (37.20 g, 90%) as a white oily solid.

A solution of compound 1-2 (37.00 g, 60.33 mmol) in 20 mL of DMSO was treated with acetic acid (AcOH; 20 mL, 317.20 mmol) and Ac₂O (15 mL, 156.68 mmol). The mixture was stirred at 25° C. for 15 h. The reaction was diluted with EtOAc (100 mL) and quenched with sat. potassium carbonate (K₂CO₃; 50 mL). The aqueous layer was extracted with EtOAc (3×50 mL). The combined organic layers were concentrated and recrystallized with acetonitrile (can; 30 mL) to afford compound 1-3 (15.65 g, 38.4%) as a white solid.

A solution of compound 1-3 (20.00 g, 29.72 mmol) in 120 mL of dichloromethane (DCM) was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and trifluoromethanesulfonic acid (TfOH; 5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. Sodium bicarbonate (NaHCO₃; 2λ100 mL), sat. sodium sulfite (Na₂SO₃; 2λ100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with 1,8-diazabicyclo[5.4.0]undec-7-ene (DBU; 7.00 mL, 45.08 mmol) at 5° C. The mixture was stirred at 5-25° C. for 1 hour. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 hour, concentrated, and recrystallized from a mixture of MTBE and DCM (5:1) to afford compound 1-6 (14.74 g, 62.9%) as a white solid: ¹H NMR (400 MHz, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl(2-cyanoethyl)diisopropylphosphoramidite (2-4a to 2-4e)

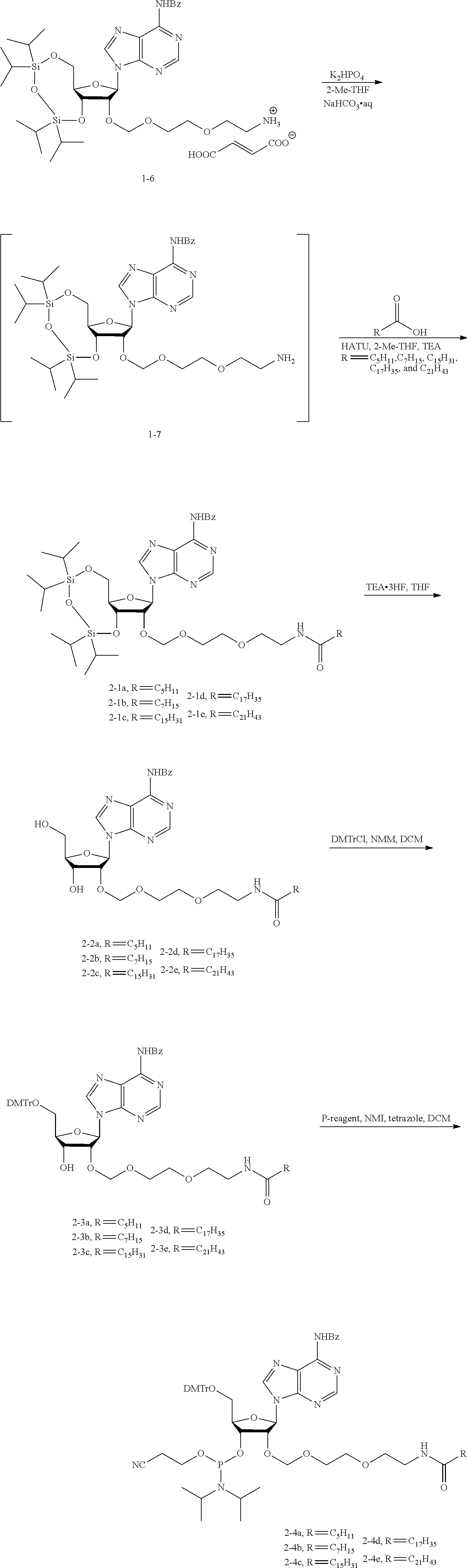

A solution of compound 1-6 (50.00 g, 59.01 mmol) in 150 mL of 2-methyltetrahydrofuran was washed with ice cold aqueous dipotassium hydrogen phosphate (K₂HPO₄; 6%, 100 mL) and brine (20%, 2λ100 mL). The organic layer was separated and treated with hexanoic acid (10.33 mL, 82.61 mmol), 1-[bis(dimethylamino)methylene]-1H-1,2,3-triazolo[4,5-b]pyridinium 3-oxide hexafluorophosphate (HATU; 33.66 g, 88.52 mmol), and 4-dimethylaminopyridine (DMAP; 10.81 g, 147.52 mmol) at 0° C. The resulting mixture was warmed to 25° C. and stirred for 1 hour. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of tetrahydrofuran (THF) was treated with triethylamine trihydrofluoride (20.61 mL, 126.58 mmol) dropwise at 10° C. The mixture was warmed to 25° C. and stirred for 2 hours. The reaction was concentrated, dissolved in DCM (100 mL), and washed with sat. NaHCO₃ (5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 hours and quenched with sat. NaHCO₃ (50 mL). The organic layer was separated, washed with water, and concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise, and the resulting mixture was stirred at 25° C. for 4 hours. The reaction was quenched with water (15 mL), and the aqueous layer was extracted with DCM (3×50 mL). The combined organic layers were washed with sat. NaHCO₃ (50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHz, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHz, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

Lipid-conjugated blunt-ended oligonucleotides described herein were synthesized using a standard procedure known in the literature for oligo synthesis on a synthesizer using amidite chemistry.

### NHP Study

NHPs (n=4) were intrathecally administered 37.5 mg lipid-conjugated or 45 mg GalNAc-conjugated MAPT-2357 (DCR 211) via lumbar infusion at L1 (see Table 9). Artificial cerebral spinal fluid (aCSF) was used as a control.

TABLE 9
Conjugated MAPTRNAi Oligonucleotides for NHP Study.

| | | | SEQ ID NO | SEQ ID NO | SEQ ID NO | SEQ ID NO |
|---|---|---|---|---|---|---|
| | Alternate | | (Sense) | (Antisense) | (Sense) | (Antisense) |
| RNAi Oligo | Name | Conjugate | Unmodified | | Modified | |
| MAPT-2357 | DCR 211 | C₁₆ | 1681 | 815 | 1682 | 885 |
| MAPT-2357 | DCR 211 | GalNAc | 780 | 815 | 850 | 885 |

28 days after administration, CNS tissue was collected to determine the concentration of the oligonucleotide and the level of MAPT gene expression. AD is a chronic neurodegenerative disease characterized by a progressive decline in cognitive abilities such as memory, thinking, language, and learning; whereas, PSP is a less common brain disorder characterized by deterioration in brain regions responsible for movement, coordination, and eventually cognition. Accordingly, CNS tissues associated with AD or PSP were analyzed separately.

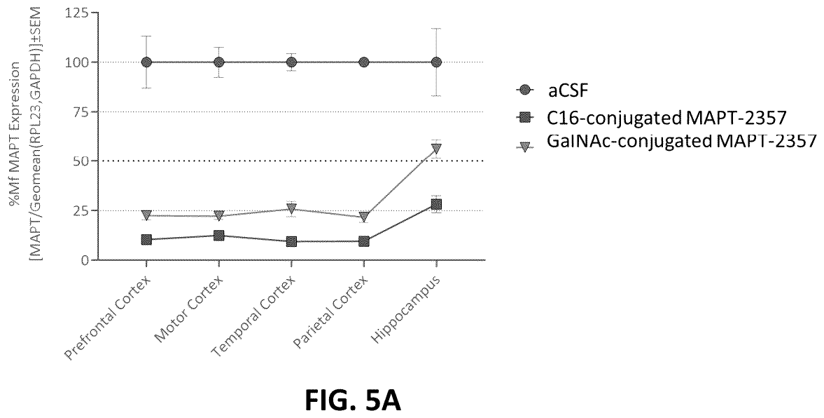

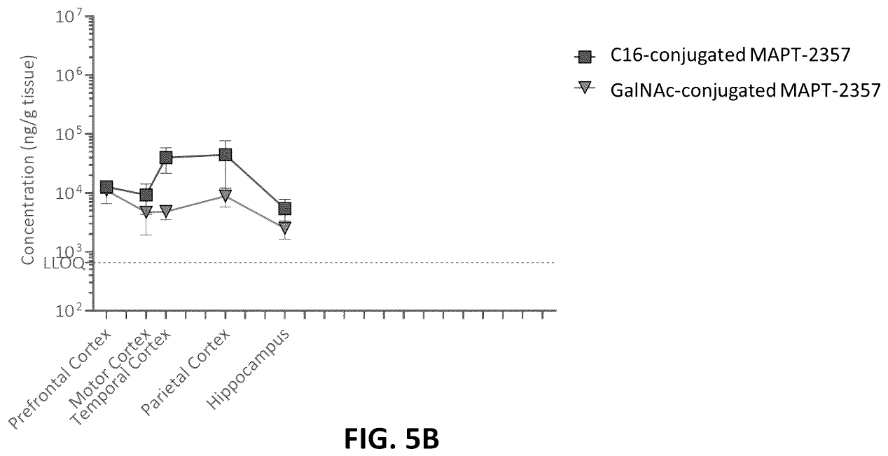

As shown in FIG. 5A, MAPT gene expression was reduced in tissues associated with AD, including the prefrontal cortex, motor cortex, temporal cortex, parietal cortex, and hippocampus, with both lipid-conjugated and GalNAc-conjugated MAPT-2357. Lipid conjugation resulted in a higher reduction of MAPT gene expression compared to GalNAc conjugation. MAPT gene expression was determined as described in the above Examples. FIG. 5B shows a higher concentration of lipid-conjugated MAPT-2357 in the same tissues compared to GalNAc-conjugated MAPT-2357. These results indicate lipid-conjugated MAPT-targeting oligonucleotides have enhanced potency even at a reduced dose compared to GalNAc-conjugated MAPT-targeting oligonucleotides across tissues associated with AD.

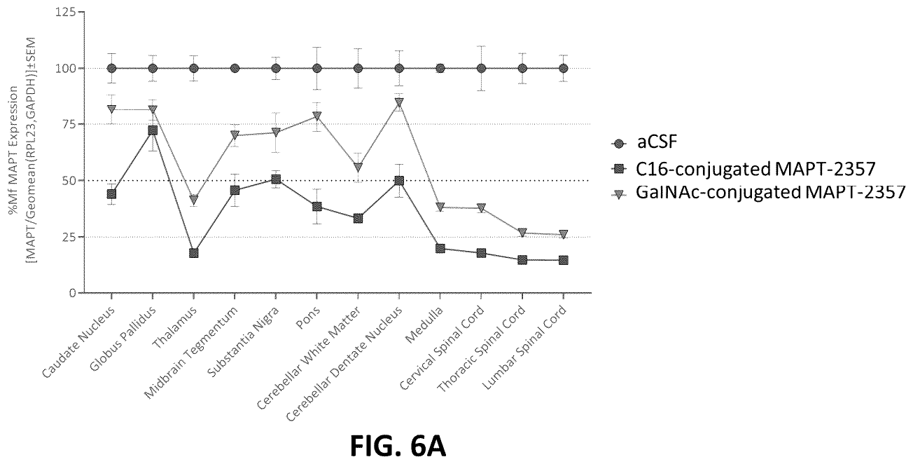

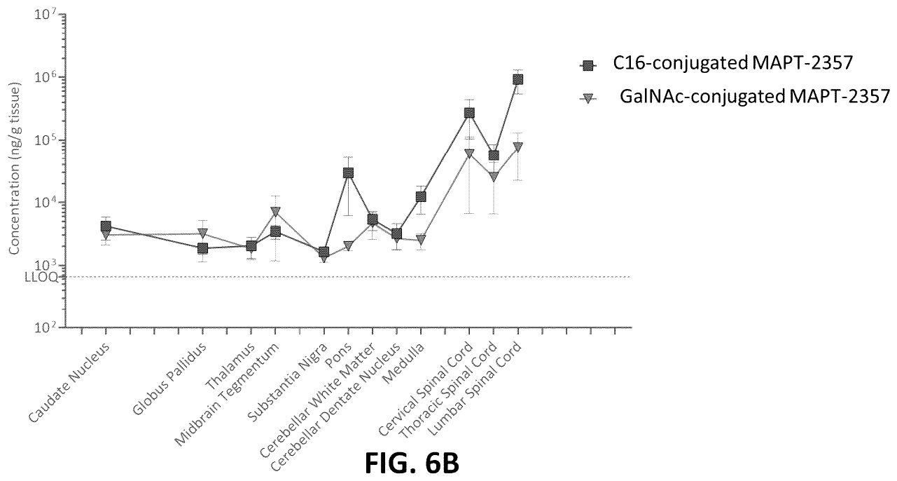

As shown in FIG. 6A, MAPT gene expression was reduced in tissues associated with PSP, including the caudate nucleus, thalamus, midbrain tegmentum, substantia nigra, pons, cerebellar white matter, cerebellar dentate nucleus, medulla, cervical spinal cord, thoracic spinal cord, and lumbar spinal cord with both lipid-conjugated and GalNAc-conjugated MAPT-2357. Lipid-conjugation resulted in a higher reduction of MAPT gene expression compared to GalNAc-conjugation. MAPT gene expression was determined as described in the above Examples. FIG. 6B shows a higher concentration of lipid-conjugated MAPT-2357 in the same tissues compared to GalNAc-conjugated MAPT-2357. These results indicate lipid-conjugated MAPT-targeting oligonucleotides have enhanced potency even at a reduced dose compared to GalNAc-conjugated MAPT-targeting oligonucleotides across tissues associated with PSP.

### SEQUENCE LISTING

The following nucleic and/or amino acid sequences are referred to in the disclosure and are provided below for reference.

| | | Species | | |
|---|---|---|---|---|
| | | and | | SEQ ID |
| Construct | | location | Sequence | NO |
| MAPT- | 25 mer | 2141-2218- | GAGAACCUGAAGCACCAGCAGGGAG | 1 |
| 2141 | sense | 966 (Hs- | | |
| | strand | Mf-Mm) | | |
| MAPT- | 25 mer | 2142-2219- | AGAACCUGAAGCACCAGCCAGGAGG | 2 |
| 2142 | sense | 967 (Hs- | | |
| | strand | Mf-Mm) | | |
| MAPT- | 25 mer | 2303-2380- | GUGACCUCCAAGUGUGGCUAAUUAG | 3 |
| 2303 | sense | 1128 (Hs- | | |
| | strand | Mf-Mm) | | |
| MAPT- | 25 mer | 2347-2424- | AGGAGGUGGCCAGGUGGAAAUAAAA | 4 |
| 2347 | sense | 1172 (Hs- | | |
| | strand | Mf-Mm) | | |
| MAPT- | 25 mer | 2349-2426- | GAGGUGGCCAGGUGGAAGUAAAATC | 5 |
| 2349 | sense | 1174 (Hs- | | |
| | strand | Mf-Mm) | | |
| MAPT- | 25 mer | 2350-2427- | AGGUGGCCAGGUGGAAGUAAAAUCT | 6 |
| 2350 | sense | 1175 (Hs- | | |
| | strand | Mf-Mm) | | |
| MAPT- | 25 mer | 2351-2428- | GGUGGCCAGGUGGAAGUAAAAUCTG | 7 |
| 2351 | sense | 1176 (Hs- | | |
| | strand | Mf-Mm) | | |
| MAPT- | 25 mer | 2352-2429- | GUGGCCAGGUGGAAGUAAAAUCUGA | 8 |
| 2352 | sense | 1177 (Hs- | | |
| | strand | Mf-Mm) | | |
| MAPT- | 25 mer | 2353-2430- | UGGCCAGGUGGAAGUAAAAACUGAG | 9 |
| 2353 | sense | 1178 (Hs- | | |
| | strand | Mf-Mm) | | |
| MAPT- | 25 mer | 2354-2431- | GGCCAGGUGGAAGUAAAAUAUGAGA | 10 |
| 2354 | sense | 1179 (Hs- | | |
| | strand | Mf-Mm) | | |
| MAPT- | 25 mer | 2355-2432- | GCCAGGUGGAAGUAAAAUCAGAGAA | 11 |
| 2355 | sense | 1180 (Hs- | | |
| | strand | Mf-Mm) | | |
| MAPT- | 25 mer | 2459-2536- | AAGAUUGAAACCCACAAGCAGACCT | 12 |
| 2459 | sense | 1284 (Hs- | | |
| | strand | Mf-Mm) | | |
| MAPT- | 25 mer | 2460-2537- | AGAUUGAAACCCACAAGCUAACCTT | 13 |
| 2460 | sense | 1285 (Hs- | | |
| | strand | Mf-Mm) | | |
| MAPT- | 25 mer | 2461-2538- | GAUUGAAACCCACAAGCUGACCUTC | 14 |
| 2461 | sense | 1286 (Hs- | | |
| | strand | Mf-Mm) | | |
| MAPT- | 25 mer | 2462-2539- | AUUGAAACCCACAAGCUGAACUUCC | 15 |
| 2462 | sense | 1287 (Hs- | | |
| | strand | Mf-Mm) | | |
| MAPT- | 25 mer | 2463-2540- | UUGAAACCCACAAGCUGACAUUCCG | 16 |
| 2463 | sense | 1288 (Hs- | | |
| | strand | Mf-Mm) | | |
| MAPT- | 25 mer | 2464-2541- | UGAAACCCACAAGCUGACCAUCCGC | 17 |
| 2464 | sense | 1289 (Hs- | | |
| | strand | Mf-Mm) | | |
| MAPT- | 25 mer | 2465-2542- | GAAACCCACAAGCUGACCUACCGCG | 18 |
| 2465 | sense | 1290 (Hs- | | |
| | strand | Mf-Mm) | | |
| MAPT- | 25 mer | 2466-2543- | AAACCCACAAGCUGACCUUACGCGA | 19 |
| 2466 | sense | 1291 (Hs- | | |
| | strand | Mf-Mm) | | |
| MAPT- | 25 mer | 2467-2544- | AACCCACAAGCUGACCUUCAGCGAG | 20 |
| 2467 | sense | 1292 (Hs- | | |
| | strand | Mf-Mm) | | |
| MAPT- | 25 mer | 2495-2572- | GCCAAAGCCAAGACAGACCACGGGG | 21 |
| 2495 | sense | 1320 (Hs- | | |
| | strand | Mf-Mm) | | |
| MAPT- | 25 mer | 2496-2573- | CCAAAGCCAAGACAGACCAAGGGGC | 22 |
| 2496 | sense | 1321 (Hs- | | |
| | strand | Mf-Mm) | | |
| MAPT- | 25 mer | 3686-3758- | UCUUUGUAAGGACUUGUGCAUCUTG | 23 |
| 3686 | sense | 2505 (Hs- | | |
| | strand | Mf-Mm) | | |
| MAPT- | 25 mer | 3687-3759- | CUUUGUAAGGACUUGUGCCACUUGG | 24 |
| 3687 | sense | 2506 (Hs- | | |
| | strand | Mf-Mm) | | |
| MAPT- | 25 mer | 3688-3760- | UUUGUAAGGACUUGUGCCUAUUGGG | 25 |
| 3688 | sense | 2507 (Hs- | | |
| | strand | Mf-Mm) | | |
| MAPT- | 25 mer | 3691-3763- | GUAAGGACUUGUGCCUCUUAGGAGA | 26 |
| 3691 | sense | 2510 (Hs- | | |
| | strand | Mf-Mm) | | |
| MAPT- | 25 mer | 3692-3764- | UAAGGACUUGUGCCUCUUGAGAGAC | 27 |
| 3692 | sense | 2511 (Hs- | | |
| | strand | Mf-Mm) | | |
| MAPT- | 25 mer | 3693-3765- | AAGGACUUGUGCCUCUUGGAAGACG | 28 |
| 3693 | sense | 2512 (Hs- | | |
| | strand | Mf-Mm) | | |
| MAPT- | 25 mer | 4534-4605- | GUUGUAGUUGGAUUUGUCUAUUUAT | 29 |
| 4534 | sense | 3332 (Hs- | | |
| | strand | Mf-Mm) | | |
| MAPT- | 25 mer | 4535-4606- | UUGUAGUUGGAUUUGUCUGAUUATG | 30 |
| 4535 | sense | 3333 (Hs- | | |
| | strand | Mf-Mm) | | |
| MAPT- | 25 mer | 4536-4607- | UGUAGUUGGAUUUGUCUGUAUAUGC | 31 |
| 4536 | sense | 3334 (Hs- | | |
| | strand | Mf-Mm) | | |
| MAPT- | 25 mer | 4537-4608- | GUAGUUGGAUUUGUCUGUUAAUGCT | 32 |
| 4537 | sense | 3335 (Hs- | | |
| | strand | Mf-Mm) | | |
| MAPT- | 25 mer | 4538-4609- | UAGUUGGAUUUGUCUGUUUAUGCTT | 33 |
| 4538 | sense | 3336 (Hs- | | |
| | strand | Mf-Mm) | | |
| MAPT- | 25 mer | 4566-4637- | UUCACCAGAGUGACUAUGAAAGUGA | 34 |
| 4566 | sense | 3362 (Hs- | | |
| | strand | Mf-Mm) | | |
| MAPT- | 25 mer | 4567-4638- | UCACCAGAGUGACUAUGAUAGUGAA | 35 |
| 4567 | sense | 3363 (Hs- | | |
| | strand | Mf-Mm) | | |
| MAPT- | 25 mer | 4568-4639- | CACCAGAGUGACUAUGAUAAUGAAA | 36 |
| 4568 | sense | 3364 (Hs- | | |
| | strand | Mf-Mm) | | |
| MAPT- | 25 mer | 4569-4640- | ACCAGAGUGACUAUGAUAGAGAAAA | 37 |
| 4569 | sense | 3365 (Hs- | | |
| | strand | Mf-Mm) | | |
| MAPT- | 25 mer | 4570-4641- | CCAGAGUGACUAUGAUAGUAAAAAG | 38 |
| 4570 | sense | 3366 (Hs- | | |
| | strand | Mf-Mm) | | |
| MAPT- | 25 mer | 4571-4642- | CAGAGUGACUAUGAUAGUGAAAAGA | 39 |
| 4571 | sense | 3367 (Hs- | | |
| | strand | Mf-Mm) | | |
| MAPT- | 25 mer | 4572-4643- | AGAGUGACUAUGAUAGUGAAAAGAA | 40 |
| 4572 | sense | 3368 (Hs- | | |
| | strand | Mf-Mm) | | |
| MAPT- | 25 mer | 4573-4644- | GAGUGACUAUGAUAGUGAAAAGAAA | 41 |
| 4573 | sense | 3369 (Hs- | | |
| | strand | Mf-Mm) | | |
| MAPT- | 25 mer | 4574-4645- | AGUGACUAUGAUAGUGAAAAGAAAA | 42 |
| 4574 | sense | 3370 (Hs- | | |
| | strand | Mf-Mm) | | |
| MAPT- | 25 mer | 4575-4646- | GUGACUAUGAUAGUGAAAAAAAAAA | 43 |
| 4575 | sense | 3371 (Hs- | | |
| | strand | Mf-Mm) | | |
| MAPT- | 25 mer | 4576-4647- | UGACUAUGAUAGUGAAAAGAAAAAA | 44 |
| 4576 | sense | 3372 (Hs- | | |
| | strand | Mf-Mm) | | |
| MAPT- | 25 mer | 4577-4648- | GACUAUGAUAGUGAAAAGAAAAAAA | 45 |
| 4577 | sense | 3373 (Hs- | | |
| | strand | Mf-Mm) | | |
| MAPT- | 25 mer | 4578-4649- | ACUAUGAUAGUGAAAAGAAAAAAAA | 46 |
| 4578 | sense | 3374 (Hs- | | |
| | strand | Mf-Mm) | | |
| MAPT- | 25 mer | 4579-4650- | CUAUGAUAGUGAAAAGAAAAAAAAA | 47 |
| 4579 | sense | 3375 (Hs- | | |
| | strand | Mf-Mm) | | |
| MAPT- | 25 mer | 4580-4651- | UAUGAUAGUGAAAAGAAAAAAAAAA | 48 |
| 4580 | sense | 3376 (Hs- | | |
| | strand | Mf-Mm) | | |
| MAPT- | 25 mer | 4605-4677- | AAAAAAAAGGACGCAUGUAACUUGA | 49 |
| 4605 | sense | 3439 (Hs- | | |
| | strand | Mf-Mm) | | |
| MAPT- | 25 mer | 4606-4678- | AAAAAAAGGACGCAUGUAUAUUGAA | 50 |
| 4606 | sense | 3440 (Hs- | | |
| | strand | Mf-Mm) | | |
| MAPT- | 25 mer | 4607-4679- | AAAAAAGGACGCAUGUAUCAUGAAA | 51 |
| 4607 | sense | 3441 (Hs- | | |
| | strand | Mf-Mm) | | |
| MAPT- | 25 mer | 4608-4680- | AAAAAGGACGCAUGUAUCUAGAAAT | 52 |
| 4608 | sense | 3442 (Hs- | | |
| | strand | Mf-Mm) | | |
| MAPT- | 25 mer | 4609-4681- | AAAAGGACGCAUGUAUCUUAAAATG | 53 |
| 4609 | sense | 3443 (Hs- | | |
| | strand | Mf-Mm) | | |
| MAPT- | 25 mer | 4610-4682- | AAAGGACGCAUGUAUCUUGAAAUGC | 54 |
| 4610 | sense | 3444 (Hs- | | |
| | strand | Mf-Mm) | | |
| MAPT- | 25 mer | 4611-4683- | AAGGACGCAUGUAUCUUGAAAUGCT | 55 |
| 4611 | sense | 3445 (Hs- | | |
| | strand | Mf-Mm) | | |
| MAPT- | 25 mer | 4612-4684- | AGGACGCAUGUAUCUUGAAAUGCTT | 56 |
| 4612 | sense | 3446 (Hs- | | |
| | strand | Mf-Mm) | | |
| MAPT- | 25 mer | 4613-4685- | GGACGCAUGUAUCUUGAAAAGCUTG | 57 |
| 4613 | sense | 3447 (Hs- | | |
| | strand | Mf-Mm) | | |
| MAPT- | 25 mer | 4614-4686- | GACGCAUGUAUCUUGAAAUACUUGT | 58 |
| 4614 | sense | 3448 (Hs- | | |
| | strand | Mf-Mm) | | |
| MAPT- | 25 mer | 5969-6024- | UCACUUUAUCAAUAGUUCCAUUUAA | 59 |
| 5969 | sense | 4540 (Hs- | | |
| | strand | Mf-Mm) | | |
| MAPT- | 25 mer | 5970-6025- | CACUUUAUCAAUAGUUCCAAUUAAA | 60 |
| 5970 | sense | 4541 (Hs- | | |
| | strand | Mf-Mm) | | |
| MAPT- | 25 mer | 5971-6026- | ACUUUAUCAAUAGUUCCAUAUAAAT | 61 |
| 5971 | sense | 4542 (Hs- | | |
| | strand | Mf-Mm) | | |
| MAPT- | 25 mer | 5972-6027- | CUUUAUCAAUAGUUCCAUUAAAATT | 62 |
| 5972 | sense | 4543 (Hs- | | |
| | strand | Mf-Mm) | | |
| MAPT- | 25 mer | 5973-6028- | UUUAUCAAUAGUUCCAUUUAAAUTG | 63 |
| 5973 | sense | 4544 (Hs- | | |
| | strand | Mf-Mm) | | |
| MAPT- | 25 mer | 5974-6029- | UUAUCAAUAGUUCCAUUUAAAUUGA | 64 |
| 5974 | sense | 4545 (Hs- | | |
| | strand | Mf-Mm) | | |
| MAPT- | 25 mer | 5975-6030- | UAUCAAUAGUUCCAUUUAAAUUGAC | 65 |
| 5975 | sense | 4546 (Hs- | | |
| | strand | Mf-Mm) | | |
| MAPT- | 25 mer | 5976-6031- | AUCAAUAGUUCCAUUUAAAAUGACT | 66 |
| 5976 | sense | 4547 (Hs- | | |
| | strand | Mf-Mm) | | |
| MAPT- | 25 mer | 5977-6032- | UCAAUAGUUCCAUUUAAAUAGACTT | 67 |
| 5977 | sense | 4548 (Hs- | | |
| | strand | Mf-Mm) | | |
| MAPT- | 25 mer | 5978-6033- | CAAUAGUUCCAUUUAAAUUAACUTC | 68 |
| 5978 | sense | 4549 (Hs- | | |
| | strand | Mf-Mm) | | |
| MAPT- | 25 mer | 5979-6034- | AAUAGUUCCAUUUAAAUUGACUUCA | 69 |
| 5979 | sense | 4550 (Hs- | | |
| | strand | Mf-Mm) | | |
| MAPT- | 25 mer | 5980-6035- | AUAGUUCCAUUUAAAUUGAAUUCAG | 70 |
| 5980 | sense | 4551 (Hs- | | |
| | strand | Mf-Mm) | | |
| MAPT- | 25 mer | 5981-6036- | UAGUUCCAUUUAAAUUGACAUCAGT | 71 |
| 5981 | sense | 4552 (Hs- | | |
| | strand | Mf-Mm) | | |
| MAPT- | 25 mer | 5982-6037- | AGUUCCAUUUAAAUUGACUACAGTG | 72 |
| 5982 | sense | 4553 (Hs- | | |
| | strand | Mf-Mm) | | |
| MAPT- | 25 mer | 5983-6038- | GUUCCAUUUAAAUUGACUUAAGUGG | 73 |
| 5983 | sense | 4554 (Hs- | | |
| | strand | Mf-Mm) | | |
| MAPT- | 25 mer | 5984-6039- | UUCCAUUUAAAUUGACUUCAGUGGT | 74 |
| 5984 | sense | 4555 (Hs- | | |
| | strand | Mf-Mm) | | |
| MAPT- | 25 mer | 5985-6040- | UCCAUUUAAAUUGACUUCAAUGGTG | 75 |
| 5985 | sense | 4556 (Hs- | | |
| | strand | Mf-Mm) | | |
| MAPT- | 25 mer | 6662-6723- | CUUGCAAGUCCCAUGAUUUAUUCGG | 76 |
| 6662 | sense | 5230 (Hs- | | |
| | strand | Mf-Mm) | | |
| MAPT- | 25 mer | 6663-6724- | UUGCAAGUCCCAUGAUUUCAUCGGT | 77 |
| 6663 | sense | 5231 (Hs- | | |
| | strand | Mf-Mm) | | |
| MAPT- | 25 mer | 6664-6725- | UGCAAGUCCCAUGAUUUCUACGGTA | 78 |
| 6664 | sense | 5232 (Hs- | | |
| | strand | Mf-Mm) | | |
| MAPT- | 25 mer | 6665-6726- | GCAAGUCCCAUGAUUUCUUAGGUAA | 79 |
| 6665 | sense | 5233 (Hs- | | |
| | strand | Mf-Mm) | | |
| MAPT- | 25 mer | 6800-6861- | GUAAAAGUGAAUUUGGAAAAAAAGT | 80 |
| 6800 | sense | 5365 (Hs- | | |
| | strand | Mf-Mm) | | |
| MAPT- | 25 mer | 6801-6862- | UAAAAGUGAAUUUGGAAAUAAAGTT | 81 |
| 6801 | sense | 5366 (Hs- | | |
| | strand | Mf-Mm) | | |
| MAPT- | 25 mer | 6802-6863- | AAAAGUGAAUUUGGAAAUAAAGUTA | 82 |
| 6802 | sense | 5367 (Hs- | | |
| | strand | Mf-Mm) | | |
| MAPT- | 25 mer | 6803-6864- | AAAGUGAAUUUGGAAAUAAAGUUAT | 83 |
| 6803 | sense | 5368 (Hs- | | |
| | strand | Mf-Mm) | | |
| MAPT- | 25 mer | 6804-6865- | AAGUGAAUUUGGAAAUAAAAUUATT | 84 |
| 6804 | sense | 5369 (Hs- | | |
| | strand | Mf-Mm) | | |
| MAPT- | 25 mer | 6805-6866- | AGUGAAUUUGGAAAUAAAGAUAUTA | 85 |
| 6805 | sense | 5370 (Hs- | | |
| | strand | Mf-Mm) | | |
| MAPT- | 25 mer | 6806-6867- | GUGAAUUUGGAAAUAAAGUAAUUAC | 86 |
| 6806 | sense | 5371 (Hs- | | |
| | strand | Mf-Mm) | | |
| MAPT- | 25 mer | 6807-6868- | UGAAUUUGGAAAUAAAGUUAUUACT | 87 |
| 6807 | sense | 5372 (Hs- | | |
| | strand | Mf-Mm) | | |
| MAPT- | 25 mer | 6808-6869- | GAAUUUGGAAAUAAAGUUAAUACTC | 88 |
| 6808 | sense | 5373 (Hs- | | |
| | strand | Mf-Mm) | | |
| MAPT- | 25 mer | 6809-6870- | AAUUUGGAAAUAAAGUUAUAACUCT | 89 |
| 6809 | sense | 5374 (Hs- | | |
| | strand | Mf-Mm) | | |
| MAPT- | 25 mer | 6810-6871- | AUUUGGAAAUAAAGUUAUUACUCTG | 90 |
| 6810 | sense | 5375 (Hs- | | |
| | strand | Mf-Mm) | | |
| MAPT- | 25 mer | 6811-6872- | UUUGGAAAUAAAGUUAUUAAUCUGA | 91 |
| 6811 | sense | 5376 (Hs- | | |
| | strand | Mf-Mm) | | |
| MAPT- | 25 mer | 6812-6873- | UUGGAAAUAAAGUUAUUACACUGAT | 92 |
| 6812 | sense | 5377 (Hs- | | |
| | strand | Mf-Mm) | | |
| MAPT- | 25 mer | 6813-6874- | UGGAAAUAAAGUUAUUACUAUGATT | 93 |
| 6813 | sense | 5378 (Hs- | | |
| | strand | Mf-Mm) | | |
| MAPT- | 25 mer | 6814-6875- | GGAAAUAAAGUUAUUACUCAGAUTA | 94 |
| 6814 | sense | 5379 (Hs- | | |
| | strand | Mf-Mm) | | |
| MAPT- | 25 mer | 6815-6876- | GAAAUAAAGUUAUUACUCUAAUUAA | 95 |
| 6815 | sense | 5380 (Hs- | | |
| | strand | Mf-Mm) | | |
| MAPT- | 25 mer | 6816-6877- | AAAUAAAGUUAUUACUCUGAUUAAA | 96 |
| 6816 | sense | 5381 (Hs- | | |
| | strand | Mf-Mm) | | |
| MAPT- | 25 mer | 363 (Hs) | AGGAGUUCGAAGUGAUGGAAGAUCA | 97 |
| 363 | sense | | | |
| | strand | | | |
| MAPT- | 25 mer | 364 (Hs) | GGAGUUCGAAGUGAUGGAAAAUCAC | 98 |
| 364 | sense | | | |
| | strand | | | |
| MAPT- | 25 mer | 365 (Hs) | GAGUUCGAAGUGAUGGAAGAUCACG | 99 |
| 365 | sense | | | |
| | strand | | | |
| MAPT- | 25 mer | 367 (Hs) | GUUCGAAGUGAUGGAAGAUAACGCT | 100 |
| 367 | sense | | | |
| | strand | | | |
| MAPT- | 25 mer | 369 (Hs) | UCGAAGUGAUGGAAGAUCAAGCUGG | 101 |
| 369 | sense | | | |
| | strand | | | |
| MAPT- | 25 mer | 374-226 | GUGAUGGAAGAUCACGCUGAGACGT | 102 |
| 374 | sense | (Hs-Mf) | | |
| | strand | | | |
| MAPT- | 25 mer | 395-247 | ACGUACGGGUUGGGGGACAAGAAAG | 103 |
| 395 | sense | (Hs-Mf) | | |
| | strand | | | |
| MAPT- | 25 mer | 400-252 | CGGGUUGGGGGACAGGAAAAAUCAG | 104 |
| 400 | sense | (Hs-Mf) | | |
| | strand | | | |
| MAPT- | 25 mer | 443-295 | CAAGACCAAGAGGGUGACAAGGACG | 105 |
| 443 | sense | (Hs-Mf) | | |
| | strand | | | |
| MAPT- | 25 mer | 688-453 | GGAAGACGAAGCUGCUGGUAACGTG | 106 |
| 688 | sense | (Hs-Mf) | | |
| | strand | | | |
| MAPT- | 25 mer | 689-454 | GAAGACGAAGCUGCUGGUCACGUGA | 107 |
| 689 | sense | (Hs-Mf) | | |
| | strand | | | |
| MAPT- | 25 mer | 690-455 | AAGACGAAGCUGCUGGUCAAGUGAC | 108 |
| 690 | sense | (Hs-Mf) | | |
| | strand | | | |
| MAPT- | 25 mer | 693-458 | ACGAAGCUGCUGGUCACGUAACCCA | 109 |
| 693 | sense | (Hs-Mf) | | |
| | strand | | | |
| MAPT- | 25 mer | 695-460 | GAAGCUGCUGGUCACGUGAACCAAG | 110 |
| 695 | sense | (Hs-Mf) | | |
| | strand | | | |
| MAPT- | 25 mer | 696-461 | AAGCUGCUGGUCACGUGACACAAGA | 111 |
| 696 | sense | (Hs-Mf) | | |
| | strand | | | |
| MAPT- | 25 mer | 1475-1552 | CGCAUGGUCAGUAAAAGCAAAGACG | 112 |
| 1475 | sense | (Hs-Mf) | | |
| | strand | | | |
| MAPT- | 25 mer | 1476-1553 | GCAUGGUCAGUAAAAGCAAAGACGG | 113 |
| 1476 | sense | (Hs-Mf) | | |
| | strand | | | |
| MAPT- | 25 mer | 1479-1556 | UGGUCAGUAAAAGCAAAGAAGGGAC | 114 |
| 1479 | sense | (Hs-Mf) | | |
| | strand | | | |
| MAPT- | 25 mer | 1480-1557 | GGUCAGUAAAAGCAAAGACAGGACT | 115 |
| 1480 | sense | (Hs-Mf) | | |
| | strand | | | |
| MAPT- | 25 mer | 1481-1558 | GUCAGUAAAAGCAAAGACGAGACTG | 116 |
| 1481 | sense | (Hs-Mf) | | |
| | strand | | | |
| MAPT- | 25 mer | 1484-1561 | AGUAAAAGCAAAGACGGGAAUGGAA | 117 |
| 1484 | sense | (Hs-Mf) | | |
| | strand | | | |
| MAPT- | 25 mer | 1485-1562 | GUAAAAGCAAAGACGGGACAGGAAG | 118 |
| 1485 | sense | (Hs-Mf) | | |
| | strand | | | |
| MAPT- | 25 mer | 1492-1569 | CAAAGACGGGACUGGAAGCAAUGAC | 119 |
| 1492 | sense | (Hs-Mf) | | |
| | strand | | | |
| MAPT- | 25 mer | 1494-1571 | AAGACGGGACUGGAAGCGAAGACAA | 120 |
| 1494 | sense | (Hs-Mf) | | |
| | strand | | | |
| MAPT- | 25 mer | 1495-1572 | AGACGGGACUGGAAGCGAUAACAAA | 121 |
| 1495 | sense | (Hs-Mf) | | |
| | strand | | | |
| MAPT- | 25 mer | 1498-1575 | CGGGACUGGAAGCGAUGACAAAAAA | 122 |
| 1498 | sense | (Hs-Mf) | | |
| | strand | | | |
| MAPT- | 25 mer | 1499-1576 | GGGACUGGAAGCGAUGACAAAAAAG | 123 |
| 1499 | sense | (Hs-Mf) | | |
| | strand | | | |
| MAPT- | 25 mer | 1500-1577 | GGACUGGAAGCGAUGACAAAAAAGC | 124 |
| 1500 | sense | (Hs-Mf) | | |
| | strand | | | |
| MAPT- | 25 mer | 1502-1579 | ACUGGAAGCGAUGACAAAAAAGCCA | 125 |
| 1502 | sense | (Hs-Mf) | | |
| | strand | | | |
| MAPT- | 25 mer | 1503-1580 | CUGGAAGCGAUGACAAAAAAGCCAA | 126 |
| 1503 | sense | (Hs-Mf) | | |
| | strand | | | |
| MAPT- | 25 mer | 1504-1581 | UGGAAGCGAUGACAAAAAAACCAAG | 127 |
| 1504 | sense | (Hs-Mf) | | |
| | strand | | | |
| MAPT- | 25 mer | 1505-1582 | GGAAGCGAUGACAAAAAAGACAAGA | 128 |
| 1505 | sense | (Hs-Mf) | | |
| | strand | | | |
| MAPT- | 25 mer | 1506-1583 | GAAGCGAUGACAAAAAAGCAAAGAC | 129 |
| 1506 | sense | (Hs-Mf) | | |
| | strand | | | |
| MAPT- | 25 mer | 1507-1584 | AAGCGAUGACAAAAAAGCCAAGACA | 130 |
| 1507 | sense | (Hs-Mf) | | |
| | strand | | | |
| MAPT- | 25 mer | 1508-1585 | AGCGAUGACAAAAAAGCCAAGACAT | 131 |
| 1508 | sense | (Hs-Mf) | | |
| | strand | | | |
| MAPT- | 25 mer | 1509-1586 | GCGAUGACAAAAAAGCCAAAACATC | 132 |
| 1509 | sense | (Hs-Mf) | | |
| | strand | | | |
| MAPT- | 25 mer | 1733 (Hs) | GAUGGUAAAACGAAGAUCGACACAC | 133 |
| 1733 | sense | | | |
| | strand | | | |
| MAPT- | 25 mer | 1796-1873 | AACGCCACCAGGAUUCCAGAAAAAA | 134 |
| 1796 | sense | (Hs-Mf) | | |
| | strand | | | |
| MAPT- | 25 mer | 1835-1912 | AAGACACCACCCAGCUCUGAGACTA | 135 |
| 1835 | sense | (Hs-Mf) | | |
| | strand | | | |
| MAPT- | 25 mer | 1912-1989 | ACCUCCAAAAUCAGGGGAUAGCAGC | 136 |
| 1912 | sense | (Hs-Mf) | | |
| | strand | | | |
| MAPT- | 25 mer | 2094-2171 | UGCCCAUGCCAGACCUGAAAAAUGT | 137 |
| 2094 | sense | (Hs-Mf) | | |
| | strand | | | |
| MAPT- | 25 mer | 2096-2173 | CCCAUGCCAGACCUGAAGAAUGUCA | 138 |
| 2096 | sense | (Hs-Mf) | | |
| | strand | | | |
| MAPT- | 25 mer | 2097-2174 | CCAUGCCAGACCUGAAGAAAGUCAA | 139 |
| 2097 | sense | (Hs-Mf) | | |
| | strand | | | |
| MAPT- | 25 mer | 2098 (Hs) | CAUGCCAGACCUGAAGAAUAUCAAG | 140 |
| 2098 | sense | | | |
| | strand | | | |
| MAPT- | 25 mer | 2105 (Hs) | GACCUGAAGAAUGUCAAGUACAAGA | 141 |
| 2105 | sense | | | |
| | strand | | | |
| MAPT- | 25 mer | 2106 (Hs) | ACCUGAAGAAUGUCAAGUCAAAGAT | 142 |
| 2106 | sense | | | |
| | strand | | | |
| MAPT- | 25 mer | 2107 (Hs) | CCUGAAGAAUGUCAAGUCCAAGATC | 143 |
| 2107 | sense | | | |
| | strand | | | |
| MAPT- | 25 mer | 2108 (Hs) | CUGAAGAAUGUCAAGUCCAAGAUCG | 144 |
| 2108 | sense | | | |
| | strand | | | |
| MAPT- | 25 mer | 2109 (Hs) | UGAAGAAUGUCAAGUCCAAAAUCGG | 145 |
| 2109 | sense | | | |
| | strand | | | |
| MAPT- | 25 mer | 2117-2194 | GUCAAGUCCAAGAUCGGCUACACTG | 146 |
| 2117 | sense | (Hs-Mf) | | |
| | strand | | | |
| MAPT- | 25 mer | 2136 (Hs) | CCACUGAGAACCUGAAGCAACAGCC | 147 |
| 2136 | sense | | | |
| | strand | | | |
| MAPT- | 25 mer | 2137 (Hs) | CACUGAGAACCUGAAGCACAAGCCG | 148 |
| 2137 | sense | | | |
| | strand | | | |
| MAPT- | 25 mer | 2269-2346 | GCAAAUAGUCUACAAACCAAUUGAC | 149 |
| 2269 | sense | (Hs-Mf) | | |
| | strand | | | |
| MAPT- | 25 mer | 2270-2347 | CAAAUAGUCUACAAACCAGAUGACC | 150 |
| 2270 | sense | (Hs-Mf) | | |
| | strand | | | |
| MAPT- | 25 mer | 2271-2348 | AAAUAGUCUACAAACCAGUAGACCT | 151 |
| 2271 | sense | (Hs-Mf) | | |
| | strand | | | |
| MAPT- | 25 mer | 2272-2349 | AAUAGUCUACAAACCAGUUAACCTG | 152 |
| 2272 | sense | (Hs-Mf) | | |
| | strand | | | |
| MAPT- | 25 mer | 2273-2350 | AUAGUCUACAAACCAGUUGACCUGA | 153 |
| 2273 | sense | (Hs-Mf) | | |
| | strand | | | |
| MAPT- | 25 mer | 2274-2351 | UAGUCUACAAACCAGUUGAACUGAG | 154 |
| 2274 | sense | (Hs-Mf) | | |
| | strand | | | |
| MAPT- | 25 mer | 2275-2352 | AGUCUACAAACCAGUUGACAUGAGC | 155 |
| 2275 | sense | (Hs-Mf) | | |
| | strand | | | |
| MAPT- | 25 mer | 2276-2353 | GUCUACAAACCAGUUGACCAGAGCA | 156 |
| 2276 | sense | (Hs-Mf) | | |
| | strand | | | |
| MAPT- | 25 mer | 2277-2354 | UCUACAAACCAGUUGACCUAAGCAA | 157 |
| 2277 | sense | (Hs-Mf) | | |
| | strand | | | |
| MAPT- | 25 mer | 2278-2355 | CUACAAACCAGUUGACCUGAGCAAG | 158 |
| 2278 | sense | (Hs-Mf) | | |
| | strand | | | |
| MAPT- | 25 mer | 2279-2356 | UACAAACCAGUUGACCUGAACAAGG | 159 |
| 2279 | sense | (Hs-Mf) | | |
| | strand | | | |
| MAPT- | 25 mer | 2280-2357 | ACAAACCAGUUGACCUGAGAAAGGT | 160 |
| 2280 | sense | (Hs-Mf) | | |
| | strand | | | |
| MAPT- | 25 mer | 2281-2358 | CAAACCAGUUGACCUGAGCAAGGTG | 16 |
| 2281 | sense | (Hs-Mf) | | |
| | strand | | | |
| MAPT- | 25 mer | 2282-2359 | AAACCAGUUGACCUGAGCAAGGUGA | 162 |
| 2282 | sense | (Hs-Mf) | | |
| | strand | | | |
| MAPT- | 25 mer | 2283-2360 | AACCAGUUGACCUGAGCAAAGUGAC | 163 |
| 2283 | sense | (Hs-Mf) | | |
| | strand | | | |
| MAPT- | 25 mer | 2284-2361 | ACCAGUUGACCUGAGCAAGAUGACC | 164 |
| 2284 | sense | (Hs-Mf) | | |
| | strand | | | |
| MAPT- | 25 mer | 2286-2363 | CAGUUGACCUGAGCAAGGUAACCTC | 165 |
| 2286 | sense | (Hs-Mf) | | |
| | strand | | | |
| MAPT- | 25 mer | 2288-2365 | GUUGACCUGAGCAAGGUGAACUCCA | 166 |
| 2288 | sense | (Hs-Mf) | | |
| | strand | | | |
| MAPT- | 25 mer | 2289-2366 | UUGACCUGAGCAAGGUGACAUCCAA | 167 |
| 2289 | sense | (Hs-Mf) | | |
| | strand | | | |
| MAPT- | 25 mer | 2291-2368 | GACCUGAGCAAGGUGACCUACAAGT | 168 |
| 2291 | sense | (Hs-Mf) | | |
| | strand | | | |
| MAPT- | 25 mer | 2294-2371 | CUGAGCAAGGUGACCUCCAAGUGTG | 169 |
| 2294 | sense | (Hs-Mf) | | |
| | strand | | | |
| MAPT- | 25 mer | 2299-2376 | CAAGGUGACCUCCAAGUGUAGCUCA | 170 |
| 2299 | sense | (Hs-Mf) | | |
| | strand | | | |
| MAPT- | 25 mer | 2300-2377 | AAGGUGACCUCCAAGUGUGACUCAT | 171 |
| 2300 | sense | (Hs-Mf) | | |
| | strand | | | |
| MAPT- | 25 mer | 2301-2378 | AGGUGACCUCCAAGUGUGGAUCATT | 172 |
| 2301 | sense | (Hs-Mf) | | |
| | strand | | | |
| MAPT- | 25 mer | 2308-2385 | CUCCAAGUGUGGCUCAUUAAGCAAC | 173 |
| 2308 | sense | (Hs-Mf) | | |
| | strand | | | |
| MAPT- | 25 mer | 2316-2393 | GUGGCUCAUUAGGCAACAUACAUCA | 174 |
| 2316 | sense | (Hs-Mf) | | |
| | strand | | | |
| MAPT- | 25 mer | 2317-2394 | UGGCUCAUUAGGCAACAUCAAUCAT | 175 |
| 2317 | sense | (Hs-Mf) | | |
| | strand | | | |
| MAPT- | 25 mer | 2319-2396 | GCUCAUUAGGCAACAUCCAACAUAA | 176 |
| 2319 | sense | (Hs-Mf) | | |
| | strand | | | |
| MAPT- | 25 mer | 2320-2397 | CUCAUUAGGCAACAUCCAUAAUAAA | 177 |
| 2320 | sense | (Hs-Mf) | | |
| | strand | | | |
| MAPT- | 25 mer | 2322-2399 | CAUUAGGCAACAUCCAUCAAAAACC | 178 |
| 2322 | sense | (Hs-Mf) | | |
| | strand | | | |
| MAPT- | 25 mer | 2323-2400 | AUUAGGCAACAUCCAUCAUAAACCA | 179 |
| 2323 | sense | (Hs-Mf) | | |
| | strand | | | |
| MAPT- | 25 mer | 2324-2401 | UUAGGCAACAUCCAUCAUAAACCAG | 180 |
| 2324 | sense | (Hs-Mf) | | |
| | strand | | | |
| MAPT- | 25 mer | 2326-2403 | AGGCAACAUCCAUCAUAAAACAGGA | 181 |
| 2326 | sense | (Hs-Mf) | | |
| | strand | | | |
| MAPT- | 25 mer | 2330-2407 | AACAUCCAUCAUAAACCAGAAGGTG | 182 |
| 2330 | sense | (Hs-Mf) | | |
| | strand | | | |
| MAPT- | 25 mer | 2356-2433 | CCAGGUGGAAGUAAAAUCUAAGAAG | 183 |
| 2356 | sense | (Hs-Mf) | | |
| | strand | | | |
| MAPT- | 25 mer | 2357-2434 | CAGGUGGAAGUAAAAUCUGAGAAGC | 184 |
| 2357 | sense | (Hs-Mf) | | |
| | strand | | | |
| MAPT- | 25 mer | 2358-2435 | AGGUGGAAGUAAAAUCUGAAAAGCT | 185 |
| 2358 | sense | (Hs-Mf) | | |
| | strand | | | |
| MAPT- | 25 mer | 2359-2436 | GGUGGAAGUAAAAUCUGAGAAGCTT | 186 |
| 2359 | sense | (Hs-Mf) | | |
| | strand | | | |
| MAPT- | 25 mer | 2360-2437 | GUGGAAGUAAAAUCUGAGAAGCUTG | 187 |
| 2360 | sense | (Hs-Mf) | | |
| | strand | | | |
| MAPT- | 25 mer | 2361-2438 | UGGAAGUAAAAUCUGAGAAACUUGA | 188 |
| 2361 | sense | (Hs-Mf) | | |
| | strand | | | |
| MAPT- | 25 mer | 2362-2439 | GGAAGUAAAAUCUGAGAAGAUUGAC | 189 |
| 2362 | sense | (Hs-Mf) | | |
| | strand | | | |
| MAPT- | 25 mer | 2363-2440 | GAAGUAAAAUCUGAGAAGCAUGACT | 190 |
| 2363 | sense | (Hs-Mf) | | |
| | strand | | | |
| MAPT- | 25 mer | 2364-2441 | AAGUAAAAUCUGAGAAGCUAGACTT | 191 |
| 2364 | sense | (Hs-Mf) | | |
| | strand | | | |
| MAPT- | 25 mer | 2365 (Hs) | AGUAAAAUCUGAGAAGCUUAACUTC | 192 |
| 2365 | sense | | | |
| | strand | | | |
| MAPT- | 25 mer | 2372 (Hs) | UCUGAGAAGCUUGACUUCAAGGACA | 193 |
| 2372 | sense | | | |
| | strand | | | |
| MAPT- | 25 mer | 2373 (Hs) | CUGAGAAGCUUGACUUCAAAGACAG | 194 |
| 2373 | sense | | | |
| | strand | | | |
| MAPT- | 25 mer | 2374 (Hs) | UGAGAAGCUUGACUUCAAGAACAGA | 195 |
| 2374 | sense | | | |
| | strand | | | |
| MAPT- | 25 mer | 2375 (Hs) | GAGAAGCUUGACUUCAAGGACAGAG | 196 |
| 2375 | sense | | | |
| | strand | | | |
| MAPT- | 25 mer | 2376 (Hs) | AGAAGCUUGACUUCAAGGAAAGAGT | 197 |
| 2376 | sense | | | |
| | strand | | | |
| MAPT- | 25 mer | 2377 (Hs) | GAAGCUUGACUUCAAGGACAGAGTC | 198 |
| 2377 | sense | | | |
| | strand | | | |
| MAPT- | 25 mer | 2378 (Hs) | AAGCUUGACUUCAAGGACAAAGUCC | 199 |
| 2378 | sense | | | |
| | strand | | | |
| MAPT- | 25 mer | 2379 (Hs) | AGCUUGACUUCAAGGACAGAGUCCA | 200 |
| 2379 | sense | | | |
| | strand | | | |
| MAPT- | 25 mer | 2380 (Hs) | GCUUGACUUCAAGGACAGAAUCCAG | 201 |
| 2380 | sense | | | |
| | strand | | | |
| MAPT- | 25 mer | 2381 (Hs) | CUUGACUUCAAGGACAGAGACCAGT | 202 |
| 2381 | sense | | | |
| | strand | | | |
| MAPT- | 25 mer | 2382 (Hs) | UUGACUUCAAGGACAGAGUACAGTC | 203 |
| 2382 | sense | | | |
| | strand | | | |
| MAPT- | 25 mer | 2390 (Hs) | AAGGACAGAGUCCAGUCGAAGAUTG | 204 |
| 2390 | sense | | | |
| | strand | | | |
| MAPT- | 25 mer | 2391 (Hs) | AGGACAGAGUCCAGUCGAAAAUUGG | 205 |
| 2391 | sense | | | |
| | strand | | | |
| MAPT- | 25 mer | 2414-2491 | GGGUCCCUGGACAAUAUCAACCACG | 206 |
| 2414 | sense | (Hs-Mf) | | |
| | strand | | | |
| MAPT- | 25 mer | 2448-2525 | GAGGAAAUAAAAAGAUUGAAACCCA | 207 |
| 2448 | sense | (Hs-Mf) | | |
| | strand | | | |
| MAPT- | 25 mer | 2449-2526 | AGGAAAUAAAAAGAUUGAAACCCAC | 208 |
| 2449 | sense | (Hs-Mf) | | |
| | strand | | | |
| MAPT- | 25 mer | 2450-2527 | GGAAAUAAAAAGAUUGAAAACCACA | 209 |
| 2450 | sense | (Hs-Mf) | | |
| | strand | | | |
| MAPT- | 25 mer | 2451-2528 | GAAAUAAAAAGAUUGAAACACACAA | 210 |
| 2451 | sense | (Hs-Mf) | | |
| | strand | | | |
| MAPT- | 25 mer | 2452-2529 | AAAUAAAAAGAUUGAAACCAACAAG | 211 |
| 2452 | sense | (Hs-Mf) | | |
| | strand | | | |
| MAPT- | 25 mer | 2453-2530 | AAUAAAAAGAUUGAAACCCACAAGC | 212 |
| 2453 | sense | (Hs-Mf) | | |
| | strand | | | |
| MAPT- | 25 mer | 2454-2531 | AUAAAAAGAUUGAAACCCAAAAGCT | 213 |
| 2454 | sense | (Hs-Mf) | | |
| | strand | | | |
| MAPT- | 25 mer | 2456-2533 | AAAAAGAUUGAAACCCACAAGCUGA | 214 |
| 2456 | sense | (Hs-Mf) | | |
| | strand | | | |
| MAPT- | 25 mer | 2457-2534 | AAAAGAUUGAAACCCACAAACUGAC | 215 |
| 2457 | sense | (Hs-Mf) | | |
| | strand | | | |
| MAPT- | 25 mer | 2567 (Hs) | CGGCAUCUCAGCAAUGUCUACUCCA | 216 |
| 2567 | sense | | | |
| | strand | | | |
| MAPT- | 25 mer | 2598-2675 | GCAUCGACAUGGUAGACUCACCCCA | 217 |
| 2598 | sense | (Hs-Mf) | | |
| | strand | | | |
| MAPT- | 25 mer | 2657-2734 | CUGGCCAAGCAGGGUUUGUAAUCAG | 218 |
| 2657 | sense | (Hs-Mf) | | |
| | strand | | | |
| MAPT- | 25 mer | 2723-2800 | AGAGUGUGGAAAAAAAAAGAAUAAT | 219 |
| 2723 | sense | (Hs-Mf) | | |
| | strand | | | |
| MAPT- | 25 mer | 2724-2801 | GAGUGUGGAAAAAAAAAGAAUAATG | 220 |
| 2724 | sense | (Hs-Mf) | | |
| | strand | | | |
| MAPT- | 25 mer | 2726-2803 | GUGUGGAAAAAAAAAGAAUAAUGAC | 221 |
| 2726 | sense | (Hs-Mf) | | |
| | strand | | | |
| MAPT- | 25 mer | 2784-2860- | GCAGUUCGGUUAAUUGGUUAAUCAC | 222 |
| 2784 | sense | 1 mismatch | | |
| | strand | (Hs-Mf) | | |
| MAPT- | 25 mer | 2963-3039 | GGCAAUUCCUUUUGAUUCUAUUUTC | 223 |
| 2963 | sense | (Hs-Mf) | | |
| | strand | | | |
| MAPT- | 25 mer | 3110-3186 | AGCAACAAAGGAUUUGAAAAUUGGT | 224 |
| 3110 | sense | (Hs-Mf) | | |
| | strand | | | |
| MAPT- | 25 mer | 3114-3190 | ACAAAGGAUUUGAAACUUGAUGUGT | 225 |
| 3114 | sense | (Hs-Mf) | | |
| | strand | | | |
| MAPT- | 25 mer | 3116-3192 | AAAGGAUUUGAAACUUGGUAUGUTC | 226 |
| 3116 | sense | (Hs-Mf) | | |
| | strand | | | |
| MAPT- | 25 mer | 3118-3194 | AGGAUUUGAAACUUGGUGUAUUCGT | 227 |
| 3118 | sense | (Hs-Mf) | | |
| | strand | | | |
| MAPT- | 25 mer | 3158-3234 | CGAUGUCAACCUUGUGUGAAUGUGA | 228 |
| 3158 | sense | (Hs-Mf) | | |
| | strand | | | |
| MAPT- | 25 mer | 3503-3576 | AAAGACUGACCUUGAUGUCAUGAGA | 229 |
| 3503 | sense | (Hs-Mf) | | |
| | strand | | | |
| MAPT- | 25 mer | 3589-3661 | CUCCACAGAAACCCUGUUUAAUUGA | 230 |
| 3589 | sense | (Hs-Mf) | | |
| | strand | | | |
| MAPT- | 25 mer | 3591-3663 | CCACAGAAACCCUGUUUUAAUGAGT | 231 |
| 3591 | sense | (Hs-Mf) | | |
| | strand | | | |
| MAPT- | 25 mer | 3592-3664 | CACAGAAACCCUGUUUUAUAGAGTT | 232 |
| 3592 | sense | (Hs-Mf) | | |
| | strand | | | |
| MAPT- | 25 mer | 3593-3665 | ACAGAAACCCUGUUUUAUUAAGUTC | 233 |
| 3593 | sense | (Hs-Mf) | | |
| | strand | | | |
| MAPT- | 25 mer | 3594-3666 | CAGAAACCCUGUUUUAUUGAGUUCT | 234 |
| 3594 | sense | (Hs-Mf) | | |
| | strand | | | |
| MAPT- | 25 mer | 3595-3667 | AGAAACCCUGUUUUAUUGAAUUCTG | 235 |
| 3595 | sense | (Hs-Mf) | | |
| | strand | | | |
| MAPT- | 25 mer | 3596-3668 | GAAACCCUGUUUUAUUGAGAUCUGA | 236 |
| 3596 | sense | (Hs-Mf) | | |
| | strand | | | |
| MAPT- | 25 mer | 3597-3669 | AAACCCUGUUUUAUUGAGUACUGAA | 237 |
| 3597 | sense | (Hs-Mf) | | |
| | strand | | | |
| MAPT- | 25 mer | 3598-3670 | AACCCUGUUUUAUUGAGUUAUGAAG | 238 |
| 3598 | sense | (Hs-Mf) | | |
| | strand | | | |
| MAPT- | 25 mer | 3599-3671 | ACCCUGUUUUAUUGAGUUCAGAAGG | 239 |
| 3599 | sense | (Hs-Mf) | | |
| | strand | | | |
| MAPT- | 25 mer | 3600-3672 | CCCUGUUUUAUUGAGUUCUAAAGGT | 240 |
| 3600 | sense | (Hs-Mf) | | |
| | strand | | | |
| MAPT- | 25 mer | 3601-3673 | CCUGUUUUAUUGAGUUCUGAAGGTT | 241 |
| 3601 | sense | (Hs-Mf) | | |
| | strand | | | |
| MAPT- | 25 mer | 3602-3674 | CUGUUUUAUUGAGUUCUGAAGGUTG | 242 |
| 3602 | sense | (Hs-Mf) | | |
| | strand | | | |
| MAPT- | 25 mer | 3603-3675 | UGUUUUAUUGAGUUCUGAAAGUUGG | 243 |
| 3603 | sense | (Hs-Mf) | | |
| | strand | | | |
| MAPT- | 25 mer | 3605-3677 | UUUUAUUGAGUUCUGAAGGAUGGAA | 244 |
| 3605 | sense | (Hs-Mf) | | |
| | strand | | | |
| MAPT- | 25 mer | 3607-3679 | UUAUUGAGUUCUGAAGGUUAGAACT | 245 |
| 3607 | sense | (Hs-Mf) | | |
| | strand | | | |
| MAPT- | 25 mer | 3609-3681 | AUUGAGUUCUGAAGGUUGGAACUGC | 246 |
| 3609 | sense | (Hs-Mf) | | |
| | strand | | | |
| MAPT- | 25 mer | 3610-3682 | UUGAGUUCUGAAGGUUGGAACUGCT | 247 |
| 3610 | sense | (Hs-Mf) | | |
| | strand | | | |
| MAPT- | 25 mer | 3677-3749 | AACCAGUUCUCUUUGUAAGAACUTG | 248 |
| 3677 | sense | (Hs-Mf) | | |
| | strand | | | |
| MAPT- | 25 mer | 3678-3750 | ACCAGUUCUCUUUGUAAGGACUUGT | 249 |
| 3678 | sense | (Hs-Mf) | | |
| | strand | | | |
| MAPT- | 25 mer | 3679-3751 | CCAGUUCUCUUUGUAAGGAAUUGTG | 250 |
| 3679 | sense | (Hs-Mf) | | |
| | strand | | | |
| MAPT- | 25 mer | 3680-3752 | CAGUUCUCUUUGUAAGGACAUGUGC | 251 |
| 3680 | sense | (Hs-Mf) | | |
| | strand | | | |
| MAPT- | 25 mer | 3958-4030 | CUACUCCAUACUGAGGGUGAAAUTA | 252 |
| 3958 | sense | (Hs-Mf) | | |
| | strand | | | |
| MAPT- | 25 mer | 3959-4031 | UACUCCAUACUGAGGGUGAAAUUAA | 253 |
| 3959 | sense | (Hs-Mf) | | |
| | strand | | | |
| MAPT- | 25 mer | 3960-4032 | ACUCCAUACUGAGGGUGAAAUUAAG | 254 |
| 3960 | sense | (Hs-Mf) | | |
| | strand | | | |
| MAPT- | 25 mer | 3961-4033 | CUCCAUACUGAGGGUGAAAAUAAGG | 255 |
| 3961 | sense | (Hs-Mf) | | |
| | strand | | | |
| MAPT- | 25 mer | 3965-4037 | AUACUGAGGGUGAAAUUAAAGGAAG | 256 |
| 3965 | sense | (Hs-Mf) | | |
| | strand | | | |
| MAPT- | 25 mer | 3970-4042 | GAGGGUGAAAUUAAGGGAAAGCAAA | 257 |
| 3970 | sense | (Hs-Mf) | | |
| | strand | | | |
| MAPT- | 25 mer | 4146-4218 | GGUGUUUCUGCCUUGUUGAAAUGGA | 258 |
| 4146 | sense | (Hs-Mf) | | |
| | strand | | | |
| MAPT- | 25 mer | 4474-4545 | CUGGAGCAGCUGAACAUAUACAUAG | 259 |
| 4474 | sense | (Hs-Mf) | | |
| | strand | | | |
| MAPT- | 25 mer | 4475-4546 | UGGAGCAGCUGAACAUAUAAAUAGA | 260 |
| 4475 | sense | (Hs-Mf) | | |
| | strand | | | |
| MAPT- | 25 mer | 4477-4548 | GAGCAGCUGAACAUAUACAAAGATG | 261 |
| 4477 | sense | (Hs-Mf) | | |
| | strand | | | |
| MAPT- | 25 mer | 4478-4549 | AGCAGCUGAACAUAUACAUAGAUGT | 262 |
| 4478 | sense | (Hs-Mf) | | |
| | strand | | | |
| MAPT- | 25 mer | 4479-4550 | GCAGCUGAACAUAUACAUAAAUGTT | 263 |
| 4479 | sense | (Hs-Mf) | | |
| | strand | | | |
| MAPT- | 25 mer | 4480-4551 | CAGCUGAACAUAUACAUAGAUGUTG | 264 |
| 4480 | sense | (Hs-Mf) | | |
| | strand | | | |
| MAPT- | 25 mer | 4481-4552 | AGCUGAACAUAUACAUAGAAGUUGC | 265 |
| 4481 | sense | (Hs-Mf) | | |
| | strand | | | |
| MAPT- | 25 mer | 4482-4553 | GCUGAACAUAUACAUAGAUAUUGCC | 266 |
| 4482 | sense | (Hs-Mf) | | |
| | strand | | | |
| MAPT- | 25 mer | 4485-4556 | GAACAUAUACAUAGAUGUUACCCTG | 267 |
| 4485 | sense | (Hs-Mf) | | |
| | strand | | | |
| MAPT- | 25 mer | 4486-4557 | AACAUAUACAUAGAUGUUGACCUGC | 268 |
| 4486 | sense | (Hs-Mf) | | |
| | strand | | | |
| MAPT- | 25 mer | 4532 (Hs) | GAGUUGUAGUUGGAUUUGUAUGUTT | 269 |
| 4532 | sense | | | |
| | strand | | | |
| MAPT- | 25 mer | 4533 (Hs) | AGUUGUAGUUGGAUUUGUCAGUUTA | 270 |
| 4533 | sense | | | |
| | strand | | | |
| MAPT- | 25 mer | 4539-4610 | AGUUGGAUUUGUCUGUUUAAGCUTG | 271 |
| 4539 | sense | (Hs-Mf) | | |
| | strand | | | |
| MAPT- | 25 mer | 4540-4611 | GUUGGAUUUGUCUGUUUAUACUUGG | 272 |
| 4540 | sense | (Hs-Mf) | | |
| | strand | | | |
| MAPT- | 25 mer | 4541-4612 | UUGGAUUUGUCUGUUUAUGAUUGGA | 273 |
| 4541 | sense | (Hs-Mf) | | |
| | strand | | | |
| MAPT- | 25 mer | 4543-4614 | GGAUUUGUCUGUUUAUGCUAGGATT | 274 |
| 4543 | sense | (Hs-Mf) | | |
| | strand | | | |
| MAPT- | 25 mer | 4544-4615 | GAUUUGUCUGUUUAUGCUUAGAUTC | 275 |
| 4544 | sense | (Hs-Mf) | | |
| | strand | | | |
| MAPT- | 25 mer | 4545-4616 | AUUUGUCUGUUUAUGCUUGAAUUCA | 276 |
| 4545 | sense | (Hs-Mf) | | |
| | strand | | | |
| MAPT- | 25 mer | 4546-4617 | UUUGUCUGUUUAUGCUUGGAUUCAC | 277 |
| 4546 | sense | (Hs-Mf) | | |
| | strand | | | |
| MAPT- | 25 mer | 4547-4618 | UUGUCUGUUUAUGCUUGGAAUCACC | 278 |
| 4547 | sense | (Hs-Mf) | | |
| | strand | | | |
| MAPT- | 25 mer | 4548-4619 | UGUCUGUUUAUGCUUGGAUACACCA | 279 |
| 4548 | sense | (Hs-Mf) | | |
| | strand | | | |
| MAPT- | 25 mer | 4549-4620 | GUCUGUUUAUGCUUGGAUUAACCAG | 280 |
| 4549 | sense | (Hs-Mf) | | |
| | strand | | | |
| MAPT- | 25 mer | 4550-4621 | UCUGUUUAUGCUUGGAUUCACCAGA | 281 |
| 4550 | sense | (Hs-Mf) | | |
| | strand | | | |
| MAPT- | 25 mer | 4551-4622 | CUGUUUAUGCUUGGAUUCAACAGAG | 282 |
| 4551 | sense | (Hs-Mf) | | |
| | strand | | | |
| MAPT- | 25 mer | 4552-4623 | UGUUUAUGCUUGGAUUCACAAGAGT | 283 |
| 4552 | sense | (Hs-Mf) | | |
| | strand | | | |
| MAPT- | 25 mer | 4554-4625 | UUUAUGCUUGGAUUCACCAAAGUGA | 284 |
| 4554 | sense | (Hs-Mf) | | |
| | strand | | | |
| MAPT- | 25 mer | 4556-4627 | UAUGCUUGGAUUCACCAGAAUGACT | 285 |
| 4556 | sense | (Hs-Mf) | | |
| | strand | | | |
| MAPT- | 25 mer | 4557-4628 | AUGCUUGGAUUCACCAGAGAGACTA | 286 |
| 4557 | sense | (Hs-Mf) | | |
| | strand | | | |
| MAPT- | 25 mer | 4558-4629 | UGCUUGGAUUCACCAGAGUAACUAT | 287 |
| 4558 | sense | (Hs-Mf) | | |
| | strand | | | |
| MAPT- | 25 mer | 4559-4630 | GCUUGGAUUCACCAGAGUGACUATG | 288 |
| 4559 | sense | (Hs-Mf) | | |
| | strand | | | |
| MAPT- | 25 mer | 4560-4631 | CUUGGAUUCACCAGAGUGAAUAUGA | 289 |
| 4560 | sense | (Hs-Mf) | | |
| | strand | | | |
| MAPT- | 25 mer | 4561-4632 | UUGGAUUCACCAGAGUGACAAUGAT | 290 |
| 4561 | sense | (Hs-Mf) | | |
| | strand | | | |
| MAPT- | 25 mer | 4562-4633 | UGGAUUCACCAGAGUGACUAUGATA | 291 |
| 4562 | sense | (Hs-Mf) | | |
| | strand | | | |
| MAPT- | 25 mer | 4563-4634 | GGAUUCACCAGAGUGACUAAGAUAG | 292 |
| 4563 | sense | (Hs-Mf) | | |
| | strand | | | |
| MAPT- | 25 mer | 4564-4635 | GAUUCACCAGAGUGACUAUAAUAGT | 293 |
| 4564 | sense | (Hs-Mf) | | |
| | strand | | | |
| MAPT- | 25 mer | 4615-4687 | ACGCAUGUAUCUUGAAAUGAUUGTA | 294 |
| 4615 | sense | (Hs-Mf) | | |
| | strand | | | |
| MAPT- | 25 mer | 4616-4688 | CGCAUGUAUCUUGAAAUGCAUGUAA | 295 |
| 4616 | sense | (Hs-Mf) | | |
| | strand | | | |
| MAPT- | 25 mer | 4617-4689 | GCAUGUAUCUUGAAAUGCUAGUAAA | 296 |
| 4617 | sense | (Hs-Mf) | | |
| | strand | | | |
| MAPT- | 25 mer | 4618-4690 | CAUGUAUCUUGAAAUGCUUAUAAAG | 297 |
| 4618 | sense | (Hs-Mf) | | |
| | strand | | | |
| MAPT- | 25 mer | 4619-4691 | AUGUAUCUUGAAAUGCUUGAAAAGA | 298 |
| 4619 | sense | (Hs-Mf) | | |
| | strand | | | |
| MAPT- | 25 mer | 4620-4692 | UGUAUCUUGAAAUGCUUGUAAAGAG | 299 |
| 4620 | sense | (Hs-Mf) | | |
| | strand | | | |
| MAPT- | 25 mer | 4621-4693 | GUAUCUUGAAAUGCUUGUAAAGAGG | 300 |
| 4621 | sense | (Hs-Mf) | | |
| | strand | | | |
| MAPT- | 25 mer | 4622-4694 | UAUCUUGAAAUGCUUGUAAAGAGGT | 301 |
| 4622 | sense | (Hs-Mf) | | |
| | strand | | | |
| MAPT- | 25 mer | 4623-4695 | AUCUUGAAAUGCUUGUAAAAAGGTT | 302 |
| 4623 | sense | (Hs-Mf) | | |
| | strand | | | |
| MAPT- | 25 mer | 4625-4697 | CUUGAAAUGCUUGUAAAGAAGUUTC | 303 |
| 4625 | sense | (Hs-Mf) | | |
| | strand | | | |
| MAPT- | 25 mer | 4627-4699 | UGAAAUGCUUGUAAAGAGGAUUCTA | 304 |
| 4627 | sense | (Hs-Mf) | | |
| | strand | | | |
| MAPT- | 25 mer | 4628-4700 | GAAAUGCUUGUAAAGAGGUAUCUAA | 305 |
| 4628 | sense | (Hs-Mf) | | |
| | strand | | | |
| MAPT- | 25 mer | 4629-4701 | AAAUGCUUGUAAAGAGGUUACUAAC | 306 |
| 4629 | sense | (Hs-Mf) | | |
| | strand | | | |
| MAPT- | 25 mer | 4630-4702 | AAUGCUUGUAAAGAGGUUUAUAACC | 307 |
| 4630 | sense | (Hs-Mf) | | |
| | strand | | | |
| MAPT- | 25 mer | 4632-4704 | UGCUUGUAAAGAGGUUUCUAACCCA | 308 |
| 4632 | sense | (Hs-Mf) | | |
| | strand | | | |
| MAPT- | 25 mer | 4633-4705 | GCUUGUAAAGAGGUUUCUAACCCAC | 309 |
| 4633 | sense | (Hs-Mf) | | |
| | strand | | | |
| MAPT- | 25 mer | 4825-4897 | ACAGGAUUAGGACUGAAGCAAUGAT | 310 |
| 4825 | sense | (Hs-Mf) | | |
| | strand | | | |
| MAPT- | 25 mer | 4828-4900 | GGAUUAGGACUGAAGCGAUAAUGTC | 311 |
| 4828 | sense | (Hs-Mf) | | |
| | strand | | | |
| MAPT- | 25 mer | 5682-5743 | GAAGUUCUUGUGCCCUGCUAUUCAG | 312 |
| 5682 | sense | (Hs-Mf) | | |
| | strand | | | |
| MAPT- | 25 mer | 5958 (Hs) | AAGCUGCUGACUCACUUUAACAATA | 313 |
| 5958 | sense | | | |
| | strand | | | |
| MAPT- | 25 mer | 5959 (Hs) | AGCUGCUGACUCACUUUAUAAAUAG | 314 |
| 5959 | sense | | | |
| | strand | | | |
| MAPT- | 25 mer | 5961 (Hs) | CUGCUGACUCACUUUAUCAAUAGTT | 315 |
| 5961 | sense | | | |
| | strand | | | |
| MAPT- | 25 mer | 5963 (Hs) | GCUGACUCACUUUAUCAAUAGUUCC | 316 |
| 5963 | sense | | | |
| | strand | | | |
| MAPT- | 25 mer | 5964 (Hs) | CUGACUCACUUUAUCAAUAAUUCCA | 317 |
| 5964 | sense | | | |
| | strand | | | |
| MAPT- | 25 mer | 5965 (Hs) | UGACUCACUUUAUCAAUAGUUCCAU | 318 |
| 5965 | sense | | | |
| | strand | | | |
| MAPT- | 25 mer | 5966-6021 | GACUCACUUUAUCAAUAGUACCATT | 319 |
| 5966 | sense | (Hs-Mf) | | |
| | strand | | | |
| MAPT- | 25 mer | 5967-6022 | ACUCACUUUAUCAAUAGUUACAUTT | 320 |
| 5967 | sense | (Hs-Mf) | | |
| | strand | | | |
| MAPT- | 25 mer | 5968-6023 | CUCACUUUAUCAAUAGUUCAAUUTA | 321 |
| 5968 | sense | (Hs-Mf) | | |
| | strand | | | |
| MAPT- | 25 mer | 6006-6061 | GGUGAGACUGUAUCCUGUUAGCUAT | 322 |
| 6006 | sense | (Hs-Mf) | | |
| | strand | | | |
| MAPT- | 25 mer | 6007-6062 | GUGAGACUGUAUCCUGUUUACUATT | 323 |
| 6007 | sense | (Hs-Mf) | | |
| | strand | | | |
| MAPT- | 25 mer | 6008-6063 | UGAGACUGUAUCCUGUUUGAUAUTG | 324 |
| 6008 | sense | (Hs-Mf) | | |
| | strand | | | |
| MAPT- | 25 mer | 6009-6064 | GAGACUGUAUCCUGUUUGCAAUUGC | 325 |
| 6009 | sense | (Hs-Mf) | | |
| | strand | | | |
| MAPT- | 25 mer | 6010-6065 | AGACUGUAUCCUGUUUGCUAUUGCT | 326 |
| 6010 | sense | (Hs-Mf) | | |
| | strand | | | |
| MAPT- | 25 mer | 6011-6066 | GACUGUAUCCUGUUUGCUAAUGCTT | 327 |
| 6011 | sense | (Hs-Mf) | | |
| | strand | | | |
| MAPT- | 25 mer | 6012-6067 | ACUGUAUCCUGUUUGCUAUAGCUTG | 328 |
| 6012 | sense | (Hs-Mf) | | |
| | strand | | | |
| MAPT- | 25 mer | 6013-6068 | CUGUAUCCUGUUUGCUAUUACUUGT | 329 |
| 6013 | sense | (Hs-Mf) | | |
| | strand | | | |
| MAPT- | 25 mer | 6014-6069 | UGUAUCCUGUUUGCUAUUGAUUGTT | 330 |
| 6014 | sense | (Hs-Mf) | | |
| | strand | | | |
| MAPT- | 25 mer | 6015-6070 | GUAUCCUGUUUGCUAUUGCAUGUTG | 331 |
| 6015 | sense | (Hs-Mf) | | |
| | strand | | | |
| MAPT- | 25 mer | 6017-6072 | AUCCUGUUUGCUAUUGCUUAUUGTG | 332 |
| 6017 | sense | (Hs-Mf) | | |
| | strand | | | |
| MAPT- | 25 mer | 6119-6174 | GCCUCGUAACCCUUUUCAUAAUUTC | 333 |
| 6119 | sense | (Hs-Mf) | | |
| | strand | | | |
| MAPT- | 25 mer | 6628-6689 | GAGUUUGCCAUGUUGAGCAAGACTA | 334 |
| 6628 | sense | (Hs-Mf) | | |
| | strand | | | |
| MAPT- | 25 mer | 6629-6690 | AGUUUGCCAUGUUGAGCAGAACUAT | 335 |
| 6629 | sense | (Hs-Mf) | | |
| | strand | | | |
| MAPT- | 25 mer | 6631-6692 | UUUGCCAUGUUGAGCAGGAAUAUTT | 336 |
| 6631 | sense | (Hs-Mf) | | |
| | strand | | | |
| MAPT- | 25 mer | 6672-6733 | CCAUGAUUUCUUCGGUAAUACUGAG | 337 |
| 6672 | sense | (Hs-Mf) | | |
| | strand | | | |
| MAPT- | 25 mer | 6731 (Hs) | GCUUUCUGUCUGUGAAUGUAUAUAT | 338 |
| 6731 | sense | | | |
| | strand | | | |
| MAPT- | 25 mer | 6732 (Hs) | CUUUCUGUCUGUGAAUGUCAAUATA | 339 |
| 6732 | sense | | | |
| | strand | | | |
| MAPT- | 25 mer | 6738-6799 | GUCUGUGAAUGUCUAUAUAAUGUAT | 340 |
| 6738 | sense | (Hs-Mf) | | |
| | strand | | | |
| MAPT- | 25 mer | 6739-6800 | UCUGUGAAUGUCUAUAUAGAGUATT | 341 |
| 6739 | sense | (Hs-Mf) | | |
| | strand | | | |
| MAPT- | 25 mer | 6740-6801 | CUGUGAAUGUCUAUAUAGUAUAUTG | 342 |
| 6740 | sense | (Hs-Mf) | | |
| | strand | | | |
| MAPT- | 25 mer | 6741-6802 | UGUGAAUGUCUAUAUAGUGAAUUGT | 343 |
| 6741 | sense | (Hs-Mf) | | |
| | strand | | | |
| MAPT- | 25 mer | 6742-6803 | GUGAAUGUCUAUAUAGUGUAUUGTG | 344 |
| 6742 | sense | (Hs-Mf) | | |
| | strand | | | |
| MAPT- | 25 mer | 6743-6804 | UGAAUGUCUAUAUAGUGUAAUGUGT | 345 |
| 6743 | sense | (Hs-Mf) | | |
| | strand | | | |
| MAPT- | 25 mer | 6745-6806 | AAUGUCUAUAUAGUGUAUUAUGUGT | 346 |
| 6745 | sense | (Hs-Mf) | | |
| | strand | | | |
| MAPT- | 25 mer | 6748-6809 | GUCUAUAUAGUGUAUUGUGAGUUTT | 347 |
| 6748 | sense | (Hs-Mf) | | |
| | strand | | | |
| MAPT- | 25 mer | 6749-6810 | UCUAUAUAGUGUAUUGUGUAUUUTA | 348 |
| 6749 | sense | (Hs-Mf) | | |
| | strand | | | |
| MAPT- | 25 mer | 6750-6811 | CUAUAUAGUGUAUUGUGUGAUUUAA | 349 |
| 6750 | sense | (Hs-Mf) | | |
| | strand | | | |
| MAPT- | 25 mer | 6751-6812 | UAUAUAGUGUAUUGUGUGUAUUAAC | 350 |
| 6751 | sense | (Hs-Mf) | | |
| | strand | | | |
| MAPT- | 25 mer | 6752-6813 | AUAUAGUGUAUUGUGUGUUAUAACA | 351 |
| 6752 | sense | (Hs-Mf) | | |
| | strand | | | |
| MAPT- | 25 mer | 6753-6814 | UAUAGUGUAUUGUGUGUUUAAACAA | 352 |
| 6753 | sense | (Hs-Mf) | | |
| | strand | | | |
| MAPT- | 25 mer | 6754-6815 | AUAGUGUAUUGUGUGUUUUAACAAA | 353 |
| 6754 | sense | (Hs-Mf) | | |
| | strand | | | |
| MAPT- | 25 mer | 6755-6816 | UAGUGUAUUGUGUGUUUUAACAAAT | 354 |
| 6755 | sense | (Hs-Mf) | | |
| | strand | | | |
| MAPT- | 25 mer | 6756-6817 | AGUGUAUUGUGUGUUUUAAAAAATG | 355 |
| 6756 | sense | (Hs-Mf) | | |
| | strand | | | |
| MAPT- | 25 mer | 6757-6818 | GUGUAUUGUGUGUUUUAACAAAUGA | 356 |
| 6757 | sense | (Hs-Mf) | | |
| | strand | | | |
| MAPT- | 25 mer | 6758-6819 | UGUAUUGUGUGUUUUAACAAAUGAT | 357 |
| 6758 | sense | (Hs-Mf) | | |
| | strand | | | |
| MAPT- | 25 mer | 6759-6820 | GUAUUGUGUGUUUUAACAAAUGATT | 358 |
| 6759 | sense | (Hs-Mf) | | |
| | strand | | | |
| MAPT- | 25 mer | 6760-6821 | UAUUGUGUGUUUUAACAAAAGAUTT | 359 |
| 6760 | sense | (Hs-Mf) | | |
| | strand | | | |
| MAPT- | 25 mer | 6761-6822 | AUUGUGUGUUUUAACAAAUAAUUTA | 360 |
| 6761 | sense | (Hs-Mf) | | |
| | strand | | | |
| MAPT- | 25 mer | 6762-6823 | UUGUGUGUUUUAACAAAUGAUUUAC | 361 |
| 6762 | sense | (Hs-Mf) | | |
| | strand | | | |
| MAPT- | 25 mer | 6763-6824 | UGUGUGUUUUAACAAAUGAAUUACA | 362 |
| 6763 | sense | (Hs-Mf) | | |
| | strand | | | |
| MAPT- | 25 mer | 6764-6825 | GUGUGUUUUAACAAAUGAUAUACAC | 363 |
| 6764 | sense | (Hs-Mf) | | |
| | strand | | | |
| MAPT- | 25 mer | 6765-6826 | UGUGUUUUAACAAAUGAUUAACACT | 364 |
| 6765 | sense | (Hs-Mf) | | |
| | strand | | | |
| MAPT- | 25 mer | 6766-6827 | GUGUUUUAACAAAUGAUUUACACTG | 365 |
| 6766 | sense | (Hs-Mf) | | |
| | strand | | | |
| MAPT- | 25 mer | 6767-6828 | UGUUUUAACAAAUGAUUUAAACUGA | 366 |
| 6767 | sense | (Hs-Mf) | | |
| | strand | | | |
| MAPT- | 25 mer | 6768-6829 | GUUUUAACAAAUGAUUUACACUGAC | 367 |
| 6768 | sense | (Hs-Mf) | | |
| | strand | | | |
| MAPT- | 25 mer | 6769-6830 | UUUUAACAAAUGAUUUACAAUGACT | 368 |
| 6769 | sense | (Hs-Mf) | | |
| | strand | | | |
| MAPT- | 25 mer | 6772-6833 | UAACAAAUGAUUUACACUGACUGTT | 369 |
| 6772 | sense | (Hs-Mf) | | |
| | strand | | | |
| MAPT- | 25 mer | 6773-6834 | AACAAAUGAUUUACACUGAAUGUTG | 370 |
| 6773 | sense | (Hs-Mf) | | |
| | strand | | | |
| MAPT- | 25 mer | 6774-6835 | ACAAAUGAUUUACACUGACAGUUGC | 371 |
| 6774 | sense | (Hs-Mf) | | |
| | strand | | | |
| MAPT- | 25 mer | 6775-6836 | CAAAUGAUUUACACUGACUAUUGCT | 372 |
| 6775 | sense | (Hs-Mf) | | |
| | strand | | | |
| MAPT- | 25 mer | 6777-6838 | AAUGAUUUACACUGACUGUAGCUGT | 373 |
| 6777 | sense | (Hs-Mf) | | |
| | strand | | | |
| MAPT- | 25 mer | 6778-6839 | AUGAUUUACACUGACUGUUACUGTA | 374 |
| 6778 | sense | (Hs-Mf) | | |
| | strand | | | |
| MAPT- | 25 mer | 6779-6840 | UGAUUUACACUGACUGUUGAUGUAA | 375 |
| 6779 | sense | (Hs-Mf) | | |
| | strand | | | |
| MAPT- | 25 mer | 6780-6841 | GAUUUACACUGACUGUUGCAGUAAA | 376 |
| 6780 | sense | (Hs-Mf) | | |
| | strand | | | |
| MAPT- | 25 mer | 6781 (Hs) | AUUUACACUGACUGUUGCUAUAAAA | 377 |
| 6781 | sense | | | |
| | strand | | | |
| MAPT- | 25 mer | 6789 (Hs) | UGACUGUUGCUGUAAAAGUAAAUTT | 378 |
| 6789 | sense | | | |
| | strand | | | |
| MAPT- | 25 mer | 6792 (Hs) | CUGUUGCUGUAAAAGUGAAAUUGGA | 379 |
| 6792 | sense | | | |
| | strand | | | |
| MAPT- | 25 mer | 6793 (Hs) | UGUUGCUGUAAAAGUGAAUAUGGAA | 380 |
| 6793 | sense | | | |
| | strand | | | |
| MAPT- | 25 mer | 6795 (Hs) | UUGCUGUAAAAGUGAAUUUAGAAAT | 381 |
| 6795 | sense | | | |
| | strand | | | |
| MAPT- | 25 mer | 6796 (Hs) | UGCUGUAAAAGUGAAUUUGAAAATA | 382 |
| 6796 | sense | | | |
| | strand | | | |
| MAPT- | 25 mer | 6797 (Hs) | GCUGUAAAAGUGAAUUUGGAAAUAA | 383 |
| 6797 | sense | | | |
| | strand | | | |
| MAPT- | 25 mer | 6798 (Hs) | CUGUAAAAGUGAAUUUGGAAAUAAA | 384 |
| 6798 | sense | | | |
| | strand | | | |
| MAPT- | 27 mer | 2141-2218- | CUCCCUGCUGGUGCUUCAGGUUCUCAG | 385 |
| 2141 | antisense | 966 (Hs- | | |
| | strand | Mf-Mm) | | |
| MAPT- | 27 mer | 2142-2219- | CCUCCUGGCUGGUGCUUCAGGUUCUCA | 386 |
| 2142 | antisense | 967 (Hs- | | |
| | strand | Mf-Mm) | | |
| MAPT- | 27 mer | 2303-2380- | CUAAUUAGCCACACUUGGAGGUCACCU | 387 |
| 2303 | antisense | 1128 (Hs- | | |
| | strand | Mf-Mm) | | |
| MAPT- | 27 mer | 2347-2424- | UUUUAUUUCCACCUGGCCACCUCCUGG | 388 |
| 2347 | antisense | 1172 (Hs- | | |
| | strand | Mf-Mm) | | |
| MAPT- | 27 mer | 2349-2426- | GAUUUUACUUCCACCUGGCCACCUCCU | 389 |
| 2349 | antisense | 1174 (Hs- | | |
| | strand | Mf-Mm) | | |
| MAPT- | 27 mer | 2350-2427- | AGAUUUUACUUCCACCUGGCCACCUCC | 390 |
| 2350 | antisense | 1175 (Hs- | | |
| | strand | Mf-Mm) | | |
| MAPT- | 27 mer | 2351-2428- | CAGAUUUUACUUCCACCUGGCCACCUC | 391 |
| 2351 | antisense | 1176 (Hs- | | |
| | strand | Mf-Mm) | | |
| MAPT- | 27 mer | 2352-2429- | UCAGAUUUUACUUCCACCUGGCCACCU | 392 |
| 2352 | antisense | 1177 (Hs- | | |
| | strand | Mf-Mm) | | |
| MAPT- | 27 mer | 2353-2430- | CUCAGUUUUUACUUCCACCUGGCCACC | 393 |
| 2353 | antisense | 1178 (Hs- | | |
| | strand | Mf-Mm) | | |
| MAPT- | 27 mer | 2354-2431- | UCUCAUAUUUUACUUCCACCUGGCCAC | 394 |
| 2354 | antisense | 1179 (Hs- | | |
| | strand | Mf-Mm) | | |
| MAPT- | 27 mer | 2355-2432- | UUCUCUGAUUUUACUUCCACCUGGCCA | 395 |
| 2355 | antisense | 1180 (Hs- | | |
| | strand | Mf-Mm) | | |
| MAPT- | 27 mer | 2459-2536- | AGGUCUGCUUGUGGGUUUCAAUCUUUU | 396 |
| 2459 | antisense | 1284 (Hs- | | |
| | strand | Mf-Mm) | | |
| MAPT- | 27 mer | 2460-2537- | AAGGUUAGCUUGUGGGUUUCAAUCUUU | 397 |
| 2460 | antisense | 1285 (Hs- | | |
| | strand | Mf-Mm) | | |
| MAPT- | 27 mer | 2461-2538- | GAAGGUCAGCUUGUGGGUUUCAAUCUU | 398 |
| 2461 | antisense | 1286 (Hs- | | |
| | strand | Mf-Mm) | | |
| MAPT- | 27 mer | 2462-2539- | GGAAGUUCAGCUUGUGGGUUUCAAUCU | 399 |
| 2462 | antisense | 1287 (Hs- | | |
| | strand | Mf-Mm) | | |
| MAPT- | 27 mer | 2463-2540- | CGGAAUGUCAGCUUGUGGGUUUCAAUC | 400 |
| 2463 | antisense | 1288 (Hs- | | |
| | strand | Mf-Mm) | | |
| MAPT- | 27 mer | 2464-2541- | GCGGAUGGUCAGCUUGUGGGUUUCAAU | 401 |
| 2464 | antisense | 1289 (Hs- | | |
| | strand | Mf-Mm) | | |
| MAPT- | 27 mer | 2465-2542- | CGCGGUAGGUCAGCUUGUGGGUUUCAA | 402 |
| 2465 | antisense | 1290 (Hs- | | |
| | strand | Mf-Mm) | | |
| MAPT- | 27 mer | 2466-2543- | UCGCGUAAGGUCAGCUUGUGGGUUUCA | 403 |
| 2466 | antisense | 1291 (Hs- | | |
| | strand | Mf-Mm) | | |
| MAPT- | 27 mer | 2467-2544- | CUCGCUGAAGGUCAGCUUGUGGGUUUC | 404 |
| 2467 | antisense | 1292 (Hs- | | |
| | strand | Mf-Mm) | | |
| MAPT- | 27 mer | 2495-2572- | CCCCGUGGUCUGUCUUGGCUUUGGCGU | 405 |
| 2495 | antisense | 1320 (Hs- | | |
| | strand | Mf-Mm) | | |
| MAPT- | 27 mer | 2496-2573- | GCCCCUUGGUCUGUCUUGGCUUUGGCG | 406 |
| 2496 | antisense | 1321 (Hs- | | |
| | strand | Mf-Mm) | | |
| MAPT- | 27 mer | 3686-3758- | CAAGAUGCACAAGUCCUUACAAAGAGA | 407 |
| 3686 | antisense | 2505 (Hs- | | |
| | strand | Mf-Mm) | | |
| MAPT- | 27 mer | 3687-3759- | CCAAGUGGCACAAGUCCUUACAAAGAG | 408 |
| 3687 | antisense | 2506 (Hs- | | |
| | strand | Mf-Mm) | | |
| MAPT- | 27 mer | 3688-3760- | CCCAAUAGGCACAAGUCCUUACAAAGA | 409 |
| 3688 | antisense | 2507 (Hs- | | |
| | strand | Mf-Mm) | | |
| MAPT- | 27 mer | 3691-3763- | UCUCCUAAGAGGCACAAGUCCUUACAA | 410 |
| 3691 | antisense | 2510 (Hs- | | |
| | strand | Mf-Mm) | | |
| MAPT- | 27 mer | 3692-3764- | GUCUCUCAAGAGGCACAAGUCCUUACA | 411 |
| 3692 | antisense | 2511 (Hs- | | |
| | strand | Mf-Mm) | | |
| MAPT- | 27 mer | 3693-3765- | CGUCUUCCAAGAGGCACAAGUCCUUAC | 412 |
| 3693 | antisense | 2512 (Hs- | | |
| | strand | Mf-Mm) | | |
| MAPT- | 27 mer | 4534-4605- | AUAAAUAGACAAAUCCAACUACAACUC | 413 |
| 4534 | antisense | 3332 (Hs- | | |
| | strand | Mf-Mm) | | |
| MAPT- | 27 mer | 4535-4606- | CAUAAUCAGACAAAUCCAACUACAACU | 414 |
| 4535 | antisense | 3333 (Hs- | | |
| | strand | Mf-Mm) | | |
| MAPT- | 27 mer | 4536-4607- | GCAUAUACAGACAAAUCCAACUACAAC | 415 |
| 4536 | antisense | 3334 (Hs- | | |
| | strand | Mf-Mm) | | |
| MAPT- | 27 mer | 4537-4608- | AGCAUUAACAGACAAAUCCAACUACAA | 416 |
| 4537 | antisense | 3335 (Hs- | | |
| | strand | Mf-Mm) | | |
| MAPT- | 27 mer | 4538-4609- | AAGCAUAAACAGACAAAUCCAACUACA | 417 |
| 4538 | antisense | 3336 (Hs- | | |
| | strand | Mf-Mm) | | |
| MAPT- | 27 mer | 4566-4637- | UCACUUUCAUAGUCACUCUGGUGAAUC | 418 |
| 4566 | antisense | 3362 (Hs- | | |
| | strand | Mf-Mm) | | |
| MAPT- | 27 mer | 4567-4638- | UUCACUAUCAUAGUCACUCUGGUGAAU | 419 |
| 4567 | antisense | 3363 (Hs- | | |
| | strand | Mf-Mm) | | |
| MAPT- | 27 mer | 4568-4639- | UUUCAUUAUCAUAGUCACUCUGGUGAA | 420 |
| 4568 | antisense | 3364 (Hs- | | |
| | strand | Mf-Mm) | | |
| MAPT- | 27 mer | 4569-4640- | UUUUCUCUAUCAUAGUCACUCUGGUGA | 421 |
| 4569 | antisense | 3365 (Hs- | | |
| | strand | Mf-Mm) | | |
| MAPT- | 27 mer | 4570-4641- | CUUUUUACUAUCAUAGUCACUCUGGUG | 422 |
| 4570 | antisense | 3366 (Hs- | | |
| | strand | Mf-Mm) | | |
| MAPT- | 27 mer | 4571-4642- | UCUUUUCACUAUCAUAGUCACUCUGGU | 423 |
| 4571 | antisense | 3367 (Hs- | | |
| | strand | Mf-Mm) | | |
| MAPT- | 27 mer | 4572-4643- | UUCUUUUCACUAUCAUAGUCACUCUGG | 424 |
| 4572 | antisense | 3368 (Hs- | | |
| | strand | Mf-Mm) | | |
| MAPT- | 27 mer | 4573-4644- | UUUCUUUUCACUAUCAUAGUCACUCUG | 425 |
| 4573 | antisense | 3369 (Hs- | | |
| | strand | Mf-Mm) | | |
| MAPT- | 27 mer | 4574-4645- | UUUUCUUUUCACUAUCAUAGUCACUCU | 426 |
| 4574 | antisense | 3370 (Hs- | | |
| | strand | Mf-Mm) | | |
| MAPT- | 27 mer | 4575-4646- | UUUUUUUUUUCACUAUCAUAGUCACUC | 427 |
| 4575 | antisense | 3371 (Hs- | | |
| | strand | Mf-Mm) | | |
| MAPT- | 27 mer | 4576-4647- | UUUUUUCUUUUCACUAUCAUAGUCACU | 428 |
| 4576 | antisense | 3372 (Hs- | | |
| | strand | Mf-Mm) | | |
| MAPT- | 27 mer | 4577-4648- | UUUUUUUCUUUUCACUAUCAUAGUCAC | 429 |
| 4577 | antisense | 3373 (Hs- | | |
| | strand | Mf-Mm) | | |
| MAPT- | 27 mer | 4578-4649- | UUUUUUUUCUUUUCACUAUCAUAGUCA | 430 |
| 4578 | antisense | 3374 (Hs- | | |
| | strand | Mf-Mm) | | |
| MAPT- | 27 mer | 4579-4650- | UUUUUUUUUCUUUUCACUAUCAUAGUC | 431 |
| 4579 | antisense | 3375 (Hs- | | |
| | strand | Mf-Mm) | | |
| MAPT- | 27 mer | 4580-4651- | UUUUUUUUUUCUUUUCACUAUCAUAGU | 432 |
| 4580 | antisense | 3376 (Hs- | | |
| | strand | Mf-Mm) | | |
| MAPT- | 27 mer | 4605-4677- | UCAAGUUACAUGCGUCCUUUUUUUUUU | 433 |
| 4605 | antisense | 3439 (Hs- | | |
| | strand | Mf-Mm) | | |
| MAPT- | 27 mer | 4606-4678- | UUCAAUAUACAUGCGUCCUUUUUUUUU | 434 |
| 4606 | antisense | 3440 (Hs- | | |
| | strand | Mf-Mm) | | |
| MAPT- | 27 mer | 4607-4679- | UUUCAUGAUACAUGCGUCCUUUUUUUU | 435 |
| 4607 | antisense | 3441 (Hs- | | |
| | strand | Mf-Mm) | | |
| MAPT- | 27 mer | 4608-4680- | AUUUCUAGAUACAUGCGUCCUUUUUUU | 436 |
| 4608 | antisense | 3442 (Hs- | | |
| | strand | Mf-Mm) | | |
| MAPT- | 27 mer | 4609-4681- | CAUUUUAAGAUACAUGCGUCCUUUUUU | 437 |
| 4609 | antisense | 3443 (Hs- | | |
| | strand | Mf-Mm) | | |
| MAPT- | 27 mer | 4610-4682- | GCAUUUCAAGAUACAUGCGUCCUUUUU | 438 |
| 4610 | antisense | 3444 (Hs- | | |
| | strand | Mf-Mm) | | |
| MAPT- | 27 mer | 4611-4683- | AGCAUUUCAAGAUACAUGCGUCCUUUU | 439 |
| 4611 | antisense | 3445 (Hs- | | |
| | strand | Mf-Mm) | | |
| MAPT- | 27 mer | 4612-4684- | AAGCAUUUCAAGAUACAUGCGUCCUUU | 440 |
| 4612 | antisense | 3446 (Hs- | | |
| | strand | Mf-Mm) | | |
| MAPT- | 27 mer | 4613-4685- | CAAGCUUUUCAAGAUACAUGCGUCCUU | 441 |
| 4613 | antisense | 3447 (Hs- | | |
| | strand | Mf-Mm) | | |
| MAPT- | 27 mer | 4614-4686- | ACAAGUAUUUCAAGAUACAUGCGUCCU | 442 |
| 4614 | antisense | 3448 (Hs- | | |
| | strand | Mf-Mm) | | |
| MAPT- | 27 mer | 5969-6024- | UUAAAUGGAACUAUUGAUAAAGUGAGU | 443 |
| 5969 | antisense | 4540 (Hs- | | |
| | strand | Mf-Mm) | | |
| MAPT- | 27 mer | 5970-6025- | UUUAAUUGGAACUAUUGAUAAAGUGAG | 444 |
| 5970 | antisense | 4541 (Hs- | | |
| | strand | Mf-Mm) | | |
| MAPT- | 27 mer | 5971-6026- | AUUUAUAUGGAACUAUUGAUAAAGUGA | 445 |
| 5971 | antisense | 4542 (Hs- | | |
| | strand | Mf-Mm) | | |
| MAPT- | 27 mer | 5972-6027- | AAUUUUAAUGGAACUAUUGAUAAAGUG | 446 |
| 5972 | antisense | 4543 (Hs- | | |
| | strand | Mf-Mm) | | |
| MAPT- | 27 mer | 5973-6028- | CAAUUUAAAUGGAACUAUUGAUAAAGU | 447 |
| 5973 | antisense | 4544 (Hs- | | |
| | strand | Mf-Mm) | | |
| MAPT- | 27 mer | 5974-6029- | UCAAUUUAAAUGGAACUAUUGAUAAAG | 448 |
| 5974 | antisense | 4545 (Hs- | | |
| | strand | Mf-Mm) | | |
| MAPT- | 27 mer | 5975-6030- | GUCAAUUUAAAUGGAACUAUUGAUAAA | 449 |
| 5975 | antisense | 4546 (Hs- | | |
| | strand | Mf-Mm) | | |
| MAPT- | 27 mer | 5976-6031- | AGUCAUUUUAAAUGGAACUAUUGAUAA | 450 |
| 5976 | antisense | 4547 (Hs- | | |
| | strand | Mf-Mm) | | |
| MAPT- | 27 mer | 5977-6032- | AAGUCUAUUUAAAUGGAACUAUUGAUA | 451 |
| 5977 | antisense | 4548 (Hs- | | |
| | strand | Mf-Mm) | | |
| MAPT- | 27 mer | 5978-6033- | GAAGUUAAUUUAAAUGGAACUAUUGAU | 452 |
| 5978 | antisense | 4549 (Hs- | | |
| | strand | Mf-Mm) | | |
| MAPT- | 27 mer | 5979-6034- | UGAAGUCAAUUUAAAUGGAACUAUUGA | 453 |
| 5979 | antisense | 4550 (Hs- | | |
| | strand | Mf-Mm) | | |
| MAPT- | 27 mer | 5980-6035- | CUGAAUUCAAUUUAAAUGGAACUAUUG | 454 |
| 5980 | antisense | 4551 (Hs- | | |
| | strand | Mf-Mm) | | |
| MAPT- | 27 mer | 5981-6036- | ACUGAUGUCAAUUUAAAUGGAACUAUU | 455 |
| 5981 | antisense | 4552 (Hs- | | |
| | strand | Mf-Mm) | | |
| MAPT- | 27 mer | 5982-6037- | CACUGUAGUCAAUUUAAAUGGAACUAU | 456 |
| 5982 | antisense | 4553 (Hs- | | |
| | strand | Mf-Mm) | | |
| MAPT- | 27 mer | 5983-6038- | CCACUUAAGUCAAUUUAAAUGGAACUA | 457 |
| 5983 | antisense | 4554 (Hs- | | |
| | strand | Mf-Mm) | | |
| MAPT- | 27 mer | 5984-6039- | ACCACUGAAGUCAAUUUAAAUGGAACU | 458 |
| 5984 | antisense | 4555 (Hs- | | |
| | strand | Mf-Mm) | | |
| MAPT- | 27 mer | 5985-6040- | CACCAUUGAAGUCAAUUUAAAUGGAAC | 459 |
| 5985 | antisense | 4556 (Hs- | | |
| | strand | Mf-Mm) | | |
| MAPT- | 27 mer | 6662-6723- | CCGAAUAAAUCAUGGGACUUGCAAGUG | 460 |
| 6662 | antisense | 5230 (Hs- | | |
| | strand | Mf-Mm) | | |
| MAPT- | 27 mer | 6663-6724- | ACCGAUGAAAUCAUGGGACUUGCAAGU | 461 |
| 6663 | antisense | 5231 (Hs- | | |
| | strand | Mf-Mm) | | |
| MAPT- | 27 mer | 6664-6725- | UACCGUAGAAAUCAUGGGACUUGCAAG | 462 |
| 6664 | antisense | 5232 (Hs- | | |
| | strand | Mf-Mm) | | |
| MAPT- | 27 mer | 6665-6726- | UUACCUAAGAAAUCAUGGGACUUGCAA | 463 |
| 6665 | antisense | 5233 (Hs- | | |
| | strand | Mf-Mm) | | |
| MAPT- | 27 mer | 6800-6861- | ACUUUUUUUCCAAAUUCACUUUUACAG | 464 |
| 6800 | antisense | 5365 (Hs- | | |
| | strand | Mf-Mm) | | |
| MAPT- | 27 mer | 6801-6862- | AACUUUAUUUCCAAAUUCACUUUUACA | 465 |
| 6801 | antisense | 5366 (Hs- | | |
| | strand | Mf-Mm) | | |
| MAPT- | 27 mer | 6802-6863- | UAACUUUAUUUCCAAAUUCACUUUUAC | 466 |
| 6802 | antisense | 5367 (Hs- | | |
| | strand | Mf-Mm) | | |
| MAPT- | 27 mer | 6803-6864- | AUAACUUUAUUUCCAAAUUCACUUUUA | 467 |
| 6803 | antisense | 5368 (Hs- | | |
| | strand | Mf-Mm) | | |
| MAPT- | 27 mer | 6804-6865- | AAUAAUUUUAUUUCCAAAUUCACUUUU | 468 |
| 6804 | antisense | 5369 (Hs- | | |
| | strand | Mf-Mm) | | |
| MAPT- | 27 mer | 6805-6866- | UAAUAUCUUUAUUUCCAAAUUCACUUU | 469 |
| 6805 | antisense | 5370 (Hs- | | |
| | strand | Mf-Mm) | | |
| MAPT- | 27 mer | 6806-6867- | GUAAUUACUUUAUUUCCAAAUUCACUU | 470 |
| 6806 | antisense | 5371 (Hs- | | |
| | strand | Mf-Mm) | | |
| MAPT- | 27 mer | 6807-6868- | AGUAAUAACUUUAUUUCCAAAUUCACU | 471 |
| 6807 | antisense | 5372 (Hs- | | |
| | strand | Mf-Mm) | | |
| MAPT- | 27 mer | 6808-6869- | GAGUAUUAACUUUAUUUCCAAAUUCAC | 472 |
| 6808 | antisense | 5373 (Hs- | | |
| | strand | Mf-Mm) | | |
| MAPT- | 27 mer | 6809-6870- | AGAGUUAUAACUUUAUUUCCAAAUUCA | 473 |
| 6809 | antisense | 5374 (Hs- | | |
| | strand | Mf-Mm) | | |
| MAPT- | 27 mer | 6810-6871- | CAGAGUAAUAACUUUAUUUCCAAAUUC | 474 |
| 6810 | antisense | 5375 (Hs- | | |
| | strand | Mf-Mm) | | |
| MAPT- | 27 mer | 6811-6872- | UCAGAUUAAUAACUUUAUUUCCAAAUU | 475 |
| 6811 | antisense | 5376 (Hs- | | |
| | strand | Mf-Mm) | | |
| MAPT- | 27 mer | 6812-6873- | AUCAGUGUAAUAACUUUAUUUCCAAAU | 476 |
| 6812 | antisense | 5377 (Hs- | | |
| | strand | Mf-Mm) | | |
| MAPT- | 27 mer | 6813-6874- | AAUCAUAGUAAUAACUUUAUUUCCAAA | 477 |
| 6813 | antisense | 5378 (Hs- | | |
| | strand | Mf-Mm) | | |
| MAPT- | 27 mer | 6814-6875- | UAAUCUGAGUAAUAACUUUAUUUCCAA | 478 |
| 6814 | antisense | 5379 (Hs- | | |
| | strand | Mf-Mm) | | |
| MAPT- | 27 mer | 6815-6876- | UUAAUUAGAGUAAUAACUUUAUUUCCA | 479 |
| 6815 | antisense | 5380 (Hs- | | |
| | strand | Mf-Mm) | | |
| MAPT- | 27 mer | 6816-6877- | UUUAAUCAGAGUAAUAACUUUAUUUCC | 480 |
| 6816 | antisense | 5381 (Hs- | | |
| | strand | Mf-Mm) | | |
| MAPT- | 27 mer | 363 (Hs) | UGAUCUUCCAUCACUUCGAACUCCUGG | 481 |
| 363 | antisense | | | |
| | strand | | | |
| MAPT- | 27 mer | 364 (Hs) | GUGAUUUUCCAUCACUUCGAACUCCUG | 482 |
| 364 | antisense | | | |
| | strand | | | |
| MAPT- | 27 mer | 365 (Hs) | CGUGAUCUUCCAUCACUUCGAACUCCU | 483 |
| 365 | antisense | | | |
| | strand | | | |
| MAPT- | 27 mer | 367 (Hs) | AGCGUUAUCUUCCAUCACUUCGAACUC | 484 |
| 367 | antisense | | | |
| | strand | | | |
| MAPT- | 27 mer | 369 (Hs) | CCAGCUUGAUCUUCCAUCACUUCGAAC | 485 |
| 369 | antisense | | | |
| | strand | | | |
| MAPT- | 27 mer | 374-226 | ACGUCUCAGCGUGAUCUUCCAUCACUU | 486 |
| 374 | antisense | (Hs-Mf) | | |
| | strand | | | |
| MAPT- | 27 mer | 395-247 | CUUUCUUGUCCCCCAACCCGUACGUCC | 487 |
| 395 | antisense | (Hs-Mf) | | |
| | strand | | | |
| MAPT- | 27 mer | 400-252 | CUGAUUUUUCCUGUCCCCCAACCCGUA | 488 |
| 400 | antisense | (Hs-Mf) | | |
| | strand | | | |
| MAPT- | 27 mer | 443-295 | CGUCCUUGUCACCCUCUUGGUCUUGGU | 489 |
| 443 | antisense | (Hs-Mf) | | |
| | strand | | | |
| MAPT- | 27 mer | 688-453 | CACGUUACCAGCAGCUUCGUCUUCCAG | 490 |
| 688 | antisense | (Hs-Mf) | | |
| | strand | | | |
| MAPT- | 27 mer | 689-454 | UCACGUGACCAGCAGCUUCGUCUUCCA | 491 |
| 689 | antisense | (Hs-Mf) | | |
| | strand | | | |
| MAPT- | 27 mer | 690-455 | GUCACUUGACCAGCAGCUUCGUCUUCC | 492 |
| 690 | antisense | (Hs-Mf) | | |
| | strand | | | |
| MAPT- | 27 mer | 693-458 | UGGGUUACGUGACCAGCAGCUUCGUCU | 493 |
| 693 | antisense | (Hs-Mf) | | |
| | strand | | | |
| MAPT- | 27 mer | 695-460 | CUUGGUUCACGUGACCAGCAGCUUCGU | 494 |
| 695 | antisense | (Hs-Mf) | | |
| | strand | | | |
| MAPT- | 27 mer | 696-461 | UCUUGUGUCACGUGACCAGCAGCUUCG | 495 |
| 696 | antisense | (Hs-Mf) | | |
| | strand | | | |
| MAPT- | 27 mer | 1475-1552 | CGUCUUUGCUUUUACUGACCAUGCGAG | 496 |
| 1475 | antisense | (Hs-Mf) | | |
| | strand | | | |
| MAPT- | 27 mer | 1476-1553 | CCGUCUUUGCUUUUACUGACCAUGCGA | 497 |
| 1476 | antisense | (Hs-Mf) | | |
| | strand | | | |
| MAPT- | 27 mer | 1479-1556 | GUCCCUUCUUUGCUUUUACUGACCAUG | 498 |
| 1479 | antisense | (Hs-Mf) | | |
| | strand | | | |
| MAPT- | 27 mer | 1480-1557 | AGUCCUGUCUUUGCUUUUACUGACCAU | 499 |
| 1480 | antisense | (Hs-Mf) | | |
| | strand | | | |
| MAPT- | 27 mer | 1481-1558 | CAGUCUCGUCUUUGCUUUUACUGACCA | 500 |
| 1481 | antisense | (Hs-Mf) | | |
| | strand | | | |
| MAPT- | 27 mer | 1484-1561 | UUCCAUUCCCGUCUUUGCUUUUACUGA | 501 |
| 1484 | antisense | (Hs-Mf) | | |
| | strand | | | |
| MAPT- | 27 mer | 1485-1562 | CUUCCUGUCCCGUCUUUGCUUUUACUG | 502 |
| 1485 | antisense | (Hs-Mf) | | |
| | strand | | | |
| MAPT- | 27 mer | 1492-1569 | GUCAUUGCUUCCAGUCCCGUCUUUGCU | 503 |
| 1492 | antisense | (Hs-Mf) | | |
| | strand | | | |
| MAPT- | 27 mer | 1494-1571 | UUGUCUUCGCUUCCAGUCCCGUCUUUG | 504 |
| 1494 | antisense | (Hs-Mf) | | |
| | strand | | | |
| MAPT- | 27 mer | 1495-1572 | UUUGUUAUCGCUUCCAGUCCCGUCUUU | 505 |
| 1495 | antisense | (Hs-Mf) | | |
| | strand | | | |
| MAPT- | 27 mer | 1498-1575 | UUUUUUGUCAUCGCUUCCAGUCCCGUC | 506 |
| 1498 | antisense | (Hs-Mf) | | |
| | strand | | | |
| MAPT- | 27 mer | 1499-1576 | CUUUUUUGUCAUCGCUUCCAGUCCCGU | 507 |
| 1499 | antisense | (Hs-Mf) | | |
| | strand | | | |
| MAPT- | 27 mer | 1500-1577 | GCUUUUUUGUCAUCGCUUCCAGUCCCG | 508 |
| 1500 | antisense | (Hs-Mf) | | |
| | strand | | | |
| MAPT- | 27 mer | 1502-1579 | UGGCUUUUUUGUCAUCGCUUCCAGUCC | 509 |
| 1502 | antisense | (Hs-Mf) | | |
| | strand | | | |
| MAPT- | 27 mer | 1503-1580 | UUGGCUUUUUUGUCAUCGCUUCCAGUC | 510 |
| 1503 | antisense | (Hs-Mf) | | |
| | strand | | | |
| MAPT- | 27 mer | 1504-1581 | CUUGGUUUUUUUGUCAUCGCUUCCAGU | 511 |
| 1504 | antisense | (Hs-Mf) | | |
| | strand | | | |
| MAPT- | 27 mer | 1505-1582 | UCUUGUCUUUUUUGUCAUCGCUUCCAG | 512 |
| 1505 | antisense | (Hs-Mf) | | |
| | strand | | | |
| MAPT- | 27 mer | 1506-1583 | GUCUUUGCUUUUUUGUCAUCGCUUCCA | 513 |
| 1506 | antisense | (Hs-Mf) | | |
| | strand | | | |
| MAPT- | 27 mer | 1507-1584 | UGUCUUGGCUUUUUUGUCAUCGCUUCC | 514 |
| 1507 | antisense | (Hs-Mf) | | |
| | strand | | | |
| MAPT- | 27 mer | 1508-1585 | AUGUCUUGGCUUUUUUGUCAUCGCUUC | 515 |
| 1508 | antisense | (Hs-Mf) | | |
| | strand | | | |
| MAPT- | 27 mer | 1509-1586 | GAUGUUUUGGCUUUUUUGUCAUCGCUU | 516 |
| 1509 | antisense | (Hs-Mf) | | |
| | strand | | | |
| MAPT- | 27 mer | 1733 (Hs) | GUGUGUCGAUCUUCGUUUUACCAUCAG | 517 |
| 1733 | antisense | | | |
| | strand | | | |
| MAPT- | 27 mer | 1796-1873 | UUUUUUCUGGAAUCCUGGUGGCGUUGG | 518 |
| 1796 | antisense | (Hs-Mf) | | |
| | strand | | | |
| MAPT- | 27 mer | 1835-1912 | UAGUCUCAGAGCUGGGUGGUGUCUUUG | 519 |
| 1835 | antisense | (Hs-Mf) | | |
| | strand | | | |
| MAPT- | 27 mer | 1912-1989 | GCUGCUAUCCCCUGAUUUUGGAGGUUC | 520 |
| 1912 | antisense | (Hs-Mf) | | |
| | strand | | | |
| MAPT- | 27 mer | 2094-2171 | ACAUUUUUCAGGUCUGGCAUGGGCACG | 521 |
| 2094 | antisense | (Hs-Mf) | | |
| | strand | | | |
| MAPT- | 27 mer | 2096-2173 | UGACAUUCUUCAGGUCUGGCAUGGGCA | 522 |
| 2096 | antisense | (Hs-Mf) | | |
| | strand | | | |
| MAPT- | 27 mer | 2097-2174 | UUGACUUUCUUCAGGUCUGGCAUGGGC | 523 |
| 2097 | antisense | (Hs-Mf) | | |
| | strand | | | |
| MAPT- | 27 mer | 2098 (Hs) | CUUGAUAUUCUUCAGGUCUGGCAUGGG | 524 |
| 2098 | antisense | | | |
| | strand | | | |
| MAPT- | 27 mer | 2105 (Hs) | UCUUGUACUUGACAUUCUUCAGGUCUG | 525 |
| 2105 | antisense | | | |
| | strand | | | |
| MAPT- | 27 mer | 2106 (Hs) | AUCUUUGACUUGACAUUCUUCAGGUCU | 526 |
| 2106 | antisense | | | |
| | strand | | | |
| MAPT- | 27 mer | 2107 (Hs) | GAUCUUGGACUUGACAUUCUUCAGGUC | 527 |
| 2107 | antisense | | | |
| | strand | | | |
| MAPT- | 27 mer | 2108 (Hs) | CGAUCUUGGACUUGACAUUCUUCAGGU | 528 |
| 2108 | antisense | | | |
| | strand | | | |
| MAPT- | 27 mer | 2109 (Hs) | CCGAUUUUGGACUUGACAUUCUUCAGG | 529 |
| 2109 | antisense | | | |
| | strand | | | |
| MAPT- | 27 mer | 2117-2194 | CAGUGUAGCCGAUCUUGGACUUGACAU | 530 |
| 2117 | antisense | (Hs-Mf) | | |
| | strand | | | |
| MAPT- | 27 mer | 2136 (Hs) | GGCUGUUGCUUCAGGUUCUCAGUGGAG | 531 |
| 2136 | antisense | | | |
| | strand | | | |
| MAPT- | 27 mer | 2137 (Hs) | CGGCUUGUGCUUCAGGUUCUCAGUGGA | 532 |
| 2137 | antisense | | | |
| | strand | | | |
| MAPT- | 27 mer | 2269-2346 | GUCAAUUGGUUUGUAGACUAUUUGCAC | 533 |
| 2269 | antisense | (Hs-Mf) | | |
| | strand | | | |
| MAPT- | 27 mer | 2270-2347 | GGUCAUCUGGUUUGUAGACUAUUUGCA | 534 |
| 2270 | antisense | (Hs-Mf) | | |
| | strand | | | |
| MAPT- | 27 mer | 2271-2348 | AGGUCUACUGGUUUGUAGACUAUUUGC | 535 |
| 2271 | antisense | (Hs-Mf) | | |
| | strand | | | |
| MAPT- | 27 mer | 2272-2349 | CAGGUUAACUGGUUUGUAGACUAUUUG | 536 |
| 2272 | antisense | (Hs-Mf) | | |
| | strand | | | |
| MAPT- | 27 mer | 2273-2350 | UCAGGUCAACUGGUUUGUAGACUAUUU | 537 |
| 2273 | antisense | (Hs-Mf) | | |
| | strand | | | |
| MAPT- | 27 mer | 2274-2351 | CUCAGUUCAACUGGUUUGUAGACUAUU | 538 |
| 2274 | antisense | (Hs-Mf) | | |
| | strand | | | |
| MAPT- | 27 mer | 2275-2352 | GCUCAUGUCAACUGGUUUGUAGACUAU | 539 |
| 2275 | antisense | (Hs-Mf) | | |
| | strand | | | |
| MAPT- | 27 mer | 2276-2353 | UGCUCUGGUCAACUGGUUUGUAGACUA | 540 |
| 2276 | antisense | (Hs-Mf) | | |
| | strand | | | |
| MAPT- | 27 mer | 2277-2354 | UUGCUUAGGUCAACUGGUUUGUAGACU | 541 |
| 2277 | antisense | (Hs-Mf) | | |
| | strand | | | |
| MAPT- | 27 mer | 2278-2355 | CUUGCUCAGGUCAACUGGUUUGUAGAC | 542 |
| 2278 | antisense | (Hs-Mf) | | |
| | strand | | | |
| MAPT- | 27 mer | 2279-2356 | CCUUGUUCAGGUCAACUGGUUUGUAGA | 543 |
| 2279 | antisense | (Hs-Mf) | | |
| | strand | | | |
| MAPT- | 27 mer | 2280-2357 | ACCUUUCUCAGGUCAACUGGUUUGUAG | 544 |
| 2280 | antisense | (Hs-Mf) | | |
| | strand | | | |
| MAPT- | 27 mer | 2281-2358 | CACCUUGCUCAGGUCAACUGGUUUGUA | 545 |
| 2281 | antisense | (Hs-Mf) | | |
| | strand | | | |
| MAPT- | 27 mer | 2282-2359 | UCACCUUGCUCAGGUCAACUGGUUUGU | 546 |
| 2282 | antisense | (Hs-Mf) | | |
| | strand | | | |
| MAPT- | 27 mer | 2283-2360 | GUCACUUUGCUCAGGUCAACUGGUUUG | 547 |
| 2283 | antisense | (Hs-Mf) | | |
| | strand | | | |
| MAPT- | 27 mer | 2284-2361 | GGUCAUCUUGCUCAGGUCAACUGGUUU | 548 |
| 2284 | antisense | (Hs-Mf) | | |
| | strand | | | |
| MAPT- | 27 mer | 2286-2363 | GAGGUUACCUUGCUCAGGUCAACUGGU | 549 |
| 2286 | antisense | (Hs-Mf) | | |
| | strand | | | |
| MAPT- | 27 mer | 2288-2365 | UGGAGUUCACCUUGCUCAGGUCAACUG | 550 |
| 2288 | antisense | (Hs-Mf) | | |
| | strand | | | |
| MAPT- | 27 mer | 2289-2366 | UUGGAUGUCACCUUGCUCAGGUCAACU | 551 |
| 2289 | antisense | (Hs-Mf) | | |
| | strand | | | |
| MAPT- | 27 mer | 2291-2368 | ACUUGUAGGUCACCUUGCUCAGGUCAA | 552 |
| 2291 | antisense | (Hs-Mf) | | |
| | strand | | | |
| MAPT- | 27 mer | 2294-2371 | CACACUUGGAGGUCACCUUGCUCAGGU | 553 |
| 2294 | antisense | (Hs-Mf) | | |
| | strand | | | |
| MAPT- | 27 mer | 2299-2376 | UGAGCUACACUUGGAGGUCACCUUGCU | 554 |
| 2299 | antisense | (Hs-Mf) | | |
| | strand | | | |
| MAPT- | 27 mer | 2300-2377 | AUGAGUCACACUUGGAGGUCACCUUGC | 555 |
| 2300 | antisense | (Hs-Mf) | | |
| | strand | | | |
| MAPT- | 27 mer | 2301-2378 | AAUGAUCCACACUUGGAGGUCACCUUG | 556 |
| 2301 | antisense | (Hs-Mf) | | |
| | strand | | | |
| MAPT- | 27 mer | 2308-2385 | GUUGCUUAAUGAGCCACACUUGGAGGU | 557 |
| 2308 | antisense | (Hs-Mf) | | |
| | strand | | | |
| MAPT- | 27 mer | 2316-2393 | UGAUGUAUGUUGCCUAAUGAGCCACAC | 558 |
| 2316 | antisense | (Hs-Mf) | | |
| | strand | | | |
| MAPT- | 27 mer | 2317-2394 | AUGAUUGAUGUUGCCUAAUGAGCCACA | 559 |
| 2317 | antisense | (Hs-Mf) | | |
| | strand | | | |
| MAPT- | 27 mer | 2319-2396 | UUAUGUUGGAUGUUGCCUAAUGAGCCA | 560 |
| 2319 | antisense | (Hs-Mf) | | |
| | strand | | | |
| MAPT- | 27 mer | 2320-2397 | UUUAUUAUGGAUGUUGCCUAAUGAGCC | 561 |
| 2320 | antisense | (Hs-Mf) | | |
| | strand | | | |
| MAPT- | 27 mer | 2322-2399 | GGUUUUUGAUGGAUGUUGCCUAAUGAG | 562 |
| 2322 | antisense | (Hs-Mf) | | |
| | strand | | | |
| MAPT- | 27 mer | 2323-2400 | UGGUUUAUGAUGGAUGUUGCCUAAUGA | 563 |
| 2323 | antisense | (Hs-Mf) | | |
| | strand | | | |
| MAPT- | 27 mer | 2324-2401 | CUGGUUUAUGAUGGAUGUUGCCUAAUG | 564 |
| 2324 | antisense | (Hs-Mf) | | |
| | strand | | | |
| MAPT- | 27 mer | 2326-2403 | UCCUGUUUUAUGAUGGAUGUUGCCUAA | 565 |
| 2326 | antisense | (Hs-Mf) | | |
| | strand | | | |
| MAPT- | 27 mer | 2330-2407 | CACCUUCUGGUUUAUGAUGGAUGUUGC | 566 |
| 2330 | antisense | (Hs-Mf) | | |
| | strand | | | |
| MAPT- | 27 mer | 2356-2433 | CUUCUUAGAUUUUACUUCCACCUGGCC | 567 |
| 2356 | antisense | (Hs-Mf) | | |
| | strand | | | |
| MAPT- | 27 mer | 2357-2434 | GCUUCUCAGAUUUUACUUCCACCUGGC | 568 |
| 2357 | antisense | (Hs-Mf) | | |
| | strand | | | |
| MAPT- | 27 mer | 2358-2435 | AGCUUUUCAGAUUUUACUUCCACCUGG | 569 |
| 2358 | antisense | (Hs-Mf) | | |
| | strand | | | |
| MAPT- | 27 mer | 2359-2436 | AAGCUUCUCAGAUUUUACUUCCACCUG | 570 |
| 2359 | antisense | (Hs-Mf) | | |
| | strand | | | |
| MAPT- | 27 mer | 2360-2437 | CAAGCUUCUCAGAUUUUACUUCCACCU | 571 |
| 2360 | antisense | (Hs-Mf) | | |
| | strand | | | |
| MAPT- | 27 mer | 2361-2438 | UCAAGUUUCUCAGAUUUUACUUCCACC | 572 |
| 2361 | antisense | (Hs-Mf) | | |
| | strand | | | |
| MAPT- | 27 mer | 2362-2439 | GUCAAUCUUCUCAGAUUUUACUUCCAC | 573 |
| 2362 | antisense | (Hs-Mf) | | |
| | strand | | | |
| MAPT- | 27 mer | 2363-2440 | AGUCAUGCUUCUCAGAUUUUACUUCCA | 574 |
| 2363 | antisense | (Hs-Mf) | | |
| | strand | | | |
| MAPT- | 27 mer | 2364-2441 | AAGUCUAGCUUCUCAGAUUUUACUUCC | 575 |
| 2364 | antisense | (Hs-Mf) | | |
| | strand | | | |
| MAPT- | 27 mer | 2365 (Hs) | GAAGUUAAGCUUCUCAGAUUUUACUUC | 576 |
| 2365 | antisense | | | |
| | strand | | | |
| MAPT- | 27 mer | 2372 (Hs) | UGUCCUUGAAGUCAAGCUUCUCAGAUU | 577 |
| 2372 | antisense | | | |
| | strand | | | |
| MAPT- | 27 mer | 2373 (Hs) | CUGUCUUUGAAGUCAAGCUUCUCAGAU | 578 |
| 2373 | antisense | | | |
| | strand | | | |
| MAPT- | 27 mer | 2374 (Hs) | UCUGUUCUUGAAGUCAAGCUUCUCAGA | 579 |
| 2374 | antisense | | | |
| | strand | | | |
| MAPT- | 27 mer | 2375 (Hs) | CUCUGUCCUUGAAGUCAAGCUUCUCAG | 580 |
| 2375 | antisense | | | |
| | strand | | | |
| MAPT- | 27 mer | 2376 (Hs) | ACUCUUUCCUUGAAGUCAAGCUUCUCA | 581 |
| 2376 | antisense | | | |
| | strand | | | |
| MAPT- | 27 mer | 2377 (Hs) | GACUCUGUCCUUGAAGUCAAGCUUCUC | 582 |
| 2377 | antisense | | | |
| | strand | | | |
| MAPT- | 27 mer | 2378 (Hs) | GGACUUUGUCCUUGAAGUCAAGCUUCU | 583 |
| 2378 | antisense | | | |
| | strand | | | |
| MAPT- | 27 mer | 2379 (Hs) | UGGACUCUGUCCUUGAAGUCAAGCUUC | 584 |
| 2379 | antisense | | | |
| | strand | | | |
| MAPT- | 27 mer | 2380 (Hs) | CUGGAUUCUGUCCUUGAAGUCAAGCUU | 585 |
| 2380 | antisense | | | |
| | strand | | | |
| MAPT- | 27 mer | 2381 (Hs) | ACUGGUCUCUGUCCUUGAAGUCAAGCU | 586 |
| 2381 | antisense | | | |
| | strand | | | |
| MAPT- | 27 mer | 2382 (Hs) | GACUGUACUCUGUCCUUGAAGUCAAGC | 587 |
| 2382 | antisense | | | |
| | strand | | | |
| MAPT- | 27 mer | 2390 (Hs) | CAAUCUUCGACUGGACUCUGUCCUUGA | 588 |
| 2390 | antisense | | | |
| | strand | | | |
| MAPT- | 27 mer | 2391 (Hs) | CCAAUUUUCGACUGGACUCUGUCCUUG | 589 |
| 2391 | antisense | | | |
| | strand | | | |
| MAPT- | 27 mer | 2414-2491 | CGUGGUUGAUAUUGUCCAGGGACCCAA | 590 |
| 2414 | antisense | (Hs-Mf) | | |
| | strand | | | |
| MAPT- | 27 mer | 2448-2525 | UGGGUUUCAAUCUUUUUAUUUCCUCCG | 591 |
| 2448 | antisense | (Hs-Mf) | | |
| | strand | | | |
| MAPT- | 27 mer | 2449-2526 | GUGGGUUUCAAUCUUUUUAUUUCCUCC | 592 |
| 2449 | antisense | (Hs-Mf) | | |
| | strand | | | |
| MAPT- | 27 mer | 2450-2527 | UGUGGUUUUCAAUCUUUUUAUUUCCUC | 593 |
| 2450 | antisense | (Hs-Mf) | | |
| | strand | | | |
| MAPT- | 27 mer | 2451-2528 | UUGUGUGUUUCAAUCUUUUUAUUUCCU | 594 |
| 2451 | antisense | (Hs-Mf) | | |
| | strand | | | |
| MAPT- | 27 mer | 2452-2529 | CUUGUUGGUUUCAAUCUUUUUAUUUCC | 595 |
| 2452 | antisense | (Hs-Mf) | | |
| | strand | | | |
| MAPT- | 27 mer | 2453-2530 | GCUUGUGGGUUUCAAUCUUUUUAUUUC | 596 |
| 2453 | antisense | (Hs-Mf) | | |
| | strand | | | |
| MAPT- | 27 mer | 2454-2531 | AGCUUUUGGGUUUCAAUCUUUUUAUUU | 597 |
| 2454 | antisense | (Hs-Mf) | | |
| | strand | | | |
| MAPT- | 27 mer | 2456-2533 | UCAGCUUGUGGGUUUCAAUCUUUUUAU | 598 |
| 2456 | antisense | (Hs-Mf) | | |
| | strand | | | |
| MAPT- | 27 mer | 2457-2534 | GUCAGUUUGUGGGUUUCAAUCUUUUUA | 599 |
| 2457 | antisense | (Hs-Mf) | | |
| | strand | | | |
| MAPT- | 27 mer | 2567 (Hs) | UGGAGUAGACAUUGCUGAGAUGCCGUG | 600 |
| 2567 | antisense | | | |
| | strand | | | |
| MAPT- | 27 mer | 2598-2675 | UGGGGUGAGUCUACCAUGUCGAUGCUG | 601 |
| 2598 | antisense | (Hs-Mf) | | |
| | strand | | | |
| MAPT- | 27 mer | 2657-2734 | CUGAUUACAAACCCUGCUUGGCCAGGG | 602 |
| 2657 | antisense | (Hs-Mf) | | |
| | strand | | | |
| MAPT- | 27 mer | 2723-2800 | AUUAUUCUUUUUUUUUCCACACUCUCU | 603 |
| 2723 | antisense | (Hs-Mf) | | |
| | strand | | | |
| MAPT- | 27 mer | 2724-2801 | CAUUAUUCUUUUUUUUUCCACACUCUC | 604 |
| 2724 | antisense | (Hs-Mf) | | |
| | strand | | | |
| MAPT- | 27 mer | 2726-2803 | GUCAUUAUUCUUUUUUUUUCCACACUC | 605 |
| 2726 | antisense | (Hs-Mf) | | |
| | strand | | | |
| MAPT- | 27 mer | 2784-2860- | GUGAUUAACCAAUUAACCGAACUGCGA | 606 |
| 2784 | antisense | 1 mismatch | | |
| | strand | (Hs-Mf) | | |
| MAPT- | 27 mer | 2963-3039 | GAAAAUAGAAUCAAAAGGAAUUGCCUG | 607 |
| 2963 | antisense | (Hs-Mf) | | |
| | strand | | | |
| MAPT- | 27 mer | 3110-3186 | ACCAAUUUUCAAAUCCUUUGUUGCUGC | 608 |
| 3110 | antisense | (Hs-Mf) | | |
| | strand | | | |
| MAPT- | 27 mer | 3114-3190 | ACACAUCAAGUUUCAAAUCCUUUGUUG | 609 |
| 3114 | antisense | (Hs-Mf) | | |
| | strand | | | |
| MAPT- | 27 mer | 3116-3192 | GAACAUACCAAGUUUCAAAUCCUUUGU | 610 |
| 3116 | antisense | (Hs-Mf) | | |
| | strand | | | |
| MAPT- | 27 mer | 3118-3194 | ACGAAUACACCAAGUUUCAAAUCCUUU | 611 |
| 3118 | antisense | (Hs-Mf) | | |
| | strand | | | |
| MAPT- | 27 mer | 3158-3234 | UCACAUUCACACAAGGUUGACAUCGUC | 612 |
| 3158 | antisense | (Hs-Mf) | | |
| | strand | | | |
| MAPT- | 27 mer | 3503-3576 | UCUCAUGACAUCAAGGUCAGUCUUUUC | 613 |
| 3503 | antisense | (Hs-Mf) | | |
| | strand | | | |
| MAPT- | 27 mer | 3589-3661 | UCAAUUAAACAGGGUUUCUGUGGAGCA | 614 |
| 3589 | antisense | (Hs-Mf) | | |
| | strand | | | |
| MAPT- | 27 mer | 3591-3663 | ACUCAUUAAAACAGGGUUUCUGUGGAG | 615 |
| 3591 | antisense | (Hs-Mf) | | |
| | strand | | | |
| MAPT- | 27 mer | 3592-3664 | AACUCUAUAAAACAGGGUUUCUGUGGA | 616 |
| 3592 | antisense | (Hs-Mf) | | |
| | strand | | | |
| MAPT- | 27 mer | 3593-3665 | GAACUUAAUAAAACAGGGUUUCUGUGG | 617 |
| 3593 | antisense | (Hs-Mf) | | |
| | strand | | | |
| MAPT- | 27 mer | 3594-3666 | AGAACUCAAUAAAACAGGGUUUCUGUG | 618 |
| 3594 | antisense | (Hs-Mf) | | |
| | strand | | | |
| MAPT- | 27 mer | 3595-3667 | CAGAAUUCAAUAAAACAGGGUUUCUGU | 619 |
| 3595 | antisense | (Hs-Mf) | | |
| | strand | | | |
| MAPT- | 27 mer | 3596-3668 | UCAGAUCUCAAUAAAACAGGGUUUCUG | 620 |
| 3596 | antisense | (Hs-Mf) | | |
| | strand | | | |
| MAPT- | 27 mer | 3597-3669 | UUCAGUACUCAAUAAAACAGGGUUUCU | 621 |
| 3597 | antisense | (Hs-Mf) | | |
| | strand | | | |
| MAPT- | 27 mer | 3598-3670 | CUUCAUAACUCAAUAAAACAGGGUUUC | 622 |
| 3598 | antisense | (Hs-Mf) | | |
| | strand | | | |
| MAPT- | 27 mer | 3599-3671 | CCUUCUGAACUCAAUAAAACAGGGUUU | 623 |
| 3599 | antisense | (Hs-Mf) | | |
| | strand | | | |
| MAPT- | 27 mer | 3600-3672 | ACCUUUAGAACUCAAUAAAACAGGGUU | 624 |
| 3600 | antisense | (Hs-Mf) | | |
| | strand | | | |
| MAPT- | 27 mer | 3601-3673 | AACCUUCAGAACUCAAUAAAACAGGGU | 625 |
| 3601 | antisense | (Hs-Mf) | | |
| | strand | | | |
| MAPT- | 27 mer | 3602-3674 | CAACCUUCAGAACUCAAUAAAACAGGG | 626 |
| 3602 | antisense | (Hs-Mf) | | |
| | strand | | | |
| MAPT- | 27 mer | 3603-3675 | CCAACUUUCAGAACUCAAUAAAACAGG | 627 |
| 3603 | antisense | (Hs-Mf) | | |
| | strand | | | |
| MAPT- | 27 mer | 3605-3677 | UUCCAUCCUUCAGAACUCAAUAAAACA | 628 |
| 3605 | antisense | (Hs-Mf) | | |
| | strand | | | |
| MAPT- | 27 mer | 3607-3679 | AGUUCUAACCUUCAGAACUCAAUAAAA | 629 |
| 3607 | antisense | (Hs-Mf) | | |
| | strand | | | |
| MAPT- | 27 mer | 3609-3681 | GCAGUUCCAACCUUCAGAACUCAAUAA | 630 |
| 3609 | antisense | (Hs-Mf) | | |
| | strand | | | |
| MAPT- | 27 mer | 3610-3682 | AGCAGUUCCAACCUUCAGAACUCAAUA | 631 |
| 3610 | antisense | (Hs-Mf) | | |
| | strand | | | |
| MAPT- | 27 mer | 3677-3749 | CAAGUUCUUACAAAGAGAACUGGUUAG | 632 |
| 3677 | antisense | (Hs-Mf) | | |
| | strand | | | |
| MAPT- | 27 mer | 3678-3750 | ACAAGUCCUUACAAAGAGAACUGGUUA | 633 |
| 3678 | antisense | (Hs-Mf) | | |
| | strand | | | |
| MAPT- | 27 mer | 3679-3751 | CACAAUUCCUUACAAAGAGAACUGGUU | 634 |
| 3679 | antisense | (Hs-Mf) | | |
| | strand | | | |
| MAPT- | 27 mer | 3680-3752 | GCACAUGUCCUUACAAAGAGAACUGGU | 635 |
| 3680 | antisense | (Hs-Mf) | | |
| | strand | | | |
| MAPT- | 27 mer | 3958-4030 | UAAUUUCACCCUCAGUAUGGAGUAGGU | 636 |
| 3958 | antisense | (Hs-Mf) | | |
| | strand | | | |
| MAPT- | 27 mer | 3959-4031 | UUAAUUUCACCCUCAGUAUGGAGUAGG | 637 |
| 3959 | antisense | (Hs-Mf) | | |
| | strand | | | |
| MAPT- | 27 mer | 3960-4032 | CUUAAUUUCACCCUCAGUAUGGAGUAG | 638 |
| 3960 | antisense | (Hs-Mf) | | |
| | strand | | | |
| MAPT- | 27 mer | 3961-4033 | CCUUAUUUUCACCCUCAGUAUGGAGUA | 639 |
| 3961 | antisense | (Hs-Mf) | | |
| | strand | | | |
| MAPT- | 27 mer | 3965-4037 | CUUCCUUUAAUUUCACCCUCAGUAUGG | 640 |
| 3965 | antisense | (Hs-Mf) | | |
| | strand | | | |
| MAPT- | 27 mer | 3970-4042 | UUUGCUUUCCCUUAAUUUCACCCUCAG | 641 |
| 3970 | antisense | (Hs-Mf) | | |
| | strand | | | |
| MAPT- | 27 mer | 4146-4218 | UCCAUUUCAACAAGGCAGAAACACCUA | 642 |
| 4146 | antisense | (Hs-Mf) | | |
| | strand | | | |
| MAPT- | 27 mer | 4474-4545 | CUAUGUAUAUGUUCAGCUGCUCCAGCA | 643 |
| 4474 | antisense | (Hs-Mf) | | |
| | strand | | | |
| MAPT- | 27 mer | 4475-4546 | UCUAUUUAUAUGUUCAGCUGCUCCAGC | 644 |
| 4475 | antisense | (Hs-Mf) | | |
| | strand | | | |
| MAPT- | 27 mer | 4477-4548 | CAUCUUUGUAUAUGUUCAGCUGCUCCA | 645 |
| 4477 | antisense | (Hs-Mf) | | |
| | strand | | | |
| MAPT- | 27 mer | 4478-4549 | ACAUCUAUGUAUAUGUUCAGCUGCUCC | 646 |
| 4478 | antisense | (Hs-Mf) | | |
| | strand | | | |
| MAPT- | 27 mer | 4479-4550 | AACAUUUAUGUAUAUGUUCAGCUGCUC | 647 |
| 4479 | antisense | (Hs-Mf) | | |
| | strand | | | |
| MAPT- | 27 mer | 4480-4551 | CAACAUCUAUGUAUAUGUUCAGCUGCU | 648 |
| 4480 | antisense | (Hs-Mf) | | |
| | strand | | | |
| MAPT- | 27 mer | 4481-4552 | GCAACUUCUAUGUAUAUGUUCAGCUGC | 649 |
| 4481 | antisense | (Hs-Mf) | | |
| | strand | | | |
| MAPT- | 27 mer | 4482-4553 | GGCAAUAUCUAUGUAUAUGUUCAGCUG | 650 |
| 4482 | antisense | (Hs-Mf) | | |
| | strand | | | |
| MAPT- | 27 mer | 4485-4556 | CAGGGUAACAUCUAUGUAUAUGUUCAG | 651 |
| 4485 | antisense | (Hs-Mf) | | |
| | strand | | | |
| MAPT- | 27 mer | 4486-4557 | GCAGGUCAACAUCUAUGUAUAUGUUCA | 652 |
| 4486 | antisense | (Hs-Mf) | | |
| | strand | | | |
| MAPT- | 27 mer | 4532 (Hs) | AAACAUACAAAUCCAACUACAACUCAA | 653 |
| 4532 | antisense | | | |
| | strand | | | |
| MAPT- | 27 mer | 4533 (Hs) | UAAACUGACAAAUCCAACUACAACUCA | 654 |
| 4533 | antisense | | | |
| | strand | | | |
| MAPT- | 27 mer | 4539-4610 | CAAGCUUAAACAGACAAAUCCAACUAC | 655 |
| 4539 | antisense | (Hs-Mf) | | |
| | strand | | | |
| MAPT- | 27 mer | 4540-4611 | CCAAGUAUAAACAGACAAAUCCAACUA | 656 |
| 4540 | antisense | (Hs-Mf) | | |
| | strand | | | |
| MAPT- | 27 mer | 4541-4612 | UCCAAUCAUAAACAGACAAAUCCAACU | 657 |
| 4541 | antisense | (Hs-Mf) | | |
| | strand | | | |
| MAPT- | 27 mer | 4543-4614 | AAUCCUAGCAUAAACAGACAAAUCCAA | 658 |
| 4543 | antisense | (Hs-Mf) | | |
| | strand | | | |
| MAPT- | 27 mer | 4544-4615 | GAAUCUAAGCAUAAACAGACAAAUCCA | 659 |
| 4544 | antisense | (Hs-Mf) | | |
| | strand | | | |
| MAPT- | 27 mer | 4545-4616 | UGAAUUCAAGCAUAAACAGACAAAUCC | 660 |
| 4545 | antisense | (Hs-Mf) | | |
| | strand | | | |
| MAPT- | 27 mer | 4546-4617 | GUGAAUCCAAGCAUAAACAGACAAAUC | 661 |
| 4546 | antisense | (Hs-Mf) | | |
| | strand | | | |
| MAPT- | 27 mer | 4547-4618 | GGUGAUUCCAAGCAUAAACAGACAAAU | 662 |
| 4547 | antisense | (Hs-Mf) | | |
| | strand | | | |
| MAPT- | 27 mer | 4548-4619 | UGGUGUAUCCAAGCAUAAACAGACAAA | 663 |
| 4548 | antisense | (Hs-Mf) | | |
| | strand | | | |
| MAPT- | 27 mer | 4549-4620 | CUGGUUAAUCCAAGCAUAAACAGACAA | 664 |
| 4549 | antisense | (Hs-Mf) | | |
| | strand | | | |
| MAPT- | 27 mer | 4550-4621 | UCUGGUGAAUCCAAGCAUAAACAGACA | 665 |
| 4550 | antisense | (Hs-Mf) | | |
| | strand | | | |
| MAPT- | 27 mer | 4551-4622 | CUCUGUUGAAUCCAAGCAUAAACAGAC | 666 |
| 4551 | antisense | (Hs-Mf) | | |
| | strand | | | |
| MAPT- | 27 mer | 4552-4623 | ACUCUUGUGAAUCCAAGCAUAAACAGA | 667 |
| 4552 | antisense | (Hs-Mf) | | |
| | strand | | | |
| MAPT- | 27 mer | 4554-4625 | UCACUUUGGUGAAUCCAAGCAUAAACA | 668 |
| 4554 | antisense | (Hs-Mf) | | |
| | strand | | | |
| MAPT- | 27 mer | 4556-4627 | AGUCAUUCUGGUGAAUCCAAGCAUAAA | 669 |
| 4556 | antisense | (Hs-Mf) | | |
| | strand | | | |
| MAPT- | 27 mer | 4557-4628 | UAGUCUCUCUGGUGAAUCCAAGCAUAA | 670 |
| 4557 | antisense | (Hs-Mf) | | |
| | strand | | | |
| MAPT- | 27 mer | 4558-4629 | AUAGUUACUCUGGUGAAUCCAAGCAUA | 671 |
| 4558 | antisense | (Hs-Mf) | | |
| | strand | | | |
| MAPT- | 27 mer | 4559-4630 | CAUAGUCACUCUGGUGAAUCCAAGCAU | 672 |
| 4559 | antisense | (Hs-Mf) | | |
| | strand | | | |
| MAPT- | 27 mer | 4560-4631 | UCAUAUUCACUCUGGUGAAUCCAAGCA | 673 |
| 4560 | antisense | (Hs-Mf) | | |
| | strand | | | |
| MAPT- | 27 mer | 4561-4632 | AUCAUUGUCACUCUGGUGAAUCCAAGC | 674 |
| 4561 | antisense | (Hs-Mf) | | |
| | strand | | | |
| MAPT- | 27 mer | 4562-4633 | UAUCAUAGUCACUCUGGUGAAUCCAAG | 675 |
| 4562 | antisense | (Hs-Mf) | | |
| | strand | | | |
| MAPT- | 27 mer | 4563-4634 | CUAUCUUAGUCACUCUGGUGAAUCCAA | 676 |
| 4563 | antisense | (Hs-Mf) | | |
| | strand | | | |
| MAPT- | 27 mer | 4564-4635 | ACUAUUAUAGUCACUCUGGUGAAUCCA | 677 |
| 4564 | antisense | (Hs-Mf) | | |
| | strand | | | |
| MAPT- | 27 mer | 4615-4687 | UACAAUCAUUUCAAGAUACAUGCGUCC | 678 |
| 4615 | antisense | (Hs-Mf) | | |
| | strand | | | |
| MAPT- | 27 mer | 4616-4688 | UUACAUGCAUUUCAAGAUACAUGCGUC | 679 |
| 4616 | antisense | (Hs-Mf) | | |
| | strand | | | |
| MAPT- | 27 mer | 4617-4689 | UUUACUAGCAUUUCAAGAUACAUGCGU | 680 |
| 4617 | antisense | (Hs-Mf) | | |
| | strand | | | |
| MAPT- | 27 mer | 4618-4690 | CUUUAUAAGCAUUUCAAGAUACAUGCG | 681 |
| 4618 | antisense | (Hs-Mf) | | |
| | strand | | | |
| MAPT- | 27 mer | 4619-4691 | UCUUUUCAAGCAUUUCAAGAUACAUGC | 682 |
| 4619 | antisense | (Hs-Mf) | | |
| | strand | | | |
| MAPT- | 27 mer | 4620-4692 | CUCUUUACAAGCAUUUCAAGAUACAUG | 683 |
| 4620 | antisense | (Hs-Mf) | | |
| | strand | | | |
| MAPT- | 27 mer | 4621-4693 | CCUCUUUACAAGCAUUUCAAGAUACAU | 684 |
| 4621 | antisense | (Hs-Mf) | | |
| | strand | | | |
| MAPT- | 27 mer | 4622-4694 | ACCUCUUUACAAGCAUUUCAAGAUACA | 685 |
| 4622 | antisense | (Hs-Mf) | | |
| | strand | | | |
| MAPT- | 27 mer | 4623-4695 | AACCUUUUUACAAGCAUUUCAAGAUAC | 686 |
| 4623 | antisense | (Hs-Mf) | | |
| | strand | | | |
| MAPT- | 27 mer | 4625-4697 | GAAACUUCUUUACAAGCAUUUCAAGAU | 687 |
| 4625 | antisense | (Hs-Mf) | | |
| | strand | | | |
| MAPT- | 27 mer | 4627-4699 | UAGAAUCCUCUUUACAAGCAUUUCAAG | 688 |
| 4627 | antisense | (Hs-Mf) | | |
| | strand | | | |
| MAPT- | 27 mer | 4628-4700 | UUAGAUACCUCUUUACAAGCAUUUCAA | 689 |
| 4628 | antisense | (Hs-Mf) | | |
| | strand | | | |
| MAPT- | 27 mer | 4629-4701 | GUUAGUAACCUCUUUACAAGCAUUUCA | 690 |
| 4629 | antisense | (Hs-Mf) | | |
| | strand | | | |
| MAPT- | 27 mer | 4630-4702 | GGUUAUAAACCUCUUUACAAGCAUUUC | 691 |
| 4630 | antisense | (Hs-Mf) | | |
| | strand | | | |
| MAPT- | 27 mer | 4632-4704 | UGGGUUAGAAACCUCUUUACAAGCAUU | 692 |
| 4632 | antisense | (Hs-Mf) | | |
| | strand | | | |
| MAPT- | 27 mer | 4633-4705 | GUGGGUUAGAAACCUCUUUACAAGCAU | 693 |
| 4633 | antisense | (Hs-Mf) | | |
| | strand | | | |
| MAPT- | 27 mer | 4825-4897 | AUCAUUGCUUCAGUCCUAAUCCUGUGC | 694 |
| 4825 | antisense | (Hs-Mf) | | |
| | strand | | | |
| MAPT- | 27 mer | 4828-4900 | GACAUUAUCGCUUCAGUCCUAAUCCUG | 695 |
| 4828 | antisense | (Hs-Mf) | | |
| | strand | | | |
| MAPT- | 27 mer | 5682-5743 | CUGAAUAGCAGGGCACAAGAACUUCAG | 696 |
| 5682 | antisense | (Hs-Mf) | | |
| | strand | | | |
| MAPT- | 27 mer | 5958 (Hs) | UAUUGUUAAAGUGAGUCAGCAGCUUGA | 697 |
| 5958 | antisense | | | |
| | strand | | | |
| MAPT- | 27 mer | 5959 (Hs) | CUAUUUAUAAAGUGAGUCAGCAGCUUG | 698 |
| 5959 | antisense | | | |
| | strand | | | |
| MAPT- | 27 mer | 5961 (Hs) | AACUAUUGAUAAAGUGAGUCAGCAGCU | 699 |
| 5961 | antisense | | | |
| | strand | | | |
| MAPT- | 27 mer | 5963 (Hs) | GGAACUAUUGAUAAAGUGAGUCAGCAG | 700 |
| 5963 | antisense | | | |
| | strand | | | |
| MAPT- | 27 mer | 5964 (Hs) | UGGAAUUAUUGAUAAAGUGAGUCAGCA | 701 |
| 5964 | antisense | | | |
| | strand | | | |
| MAPT- | 27 mer | 5965 (Hs) | AUGGAACUAUUGAUAAAGUGAGUCAGC | 702 |
| 5965 | antisense | | | |
| | strand | | | |
| MAPT- | 27 mer | 5966-6021 | AAUGGUACUAUUGAUAAAGUGAGUCAG | 703 |
| 5966 | antisense | (Hs-Mf) | | |
| | strand | | | |
| MAPT- | 27 mer | 5967-6022 | AAAUGUAACUAUUGAUAAAGUGAGUCA | 704 |
| 5967 | antisense | (Hs-Mf) | | |
| | strand | | | |
| MAPT- | 27 mer | 5968-6023 | UAAAUUGAACUAUUGAUAAAGUGAGUC | 705 |
| 5968 | antisense | (Hs-Mf) | | |
| | strand | | | |
| MAPT- | 27 mer | 6006-6061 | AUAGCUAACAGGAUACAGUCUCACCAC | 706 |
| 6006 | antisense | (Hs-Mf) | | |
| | strand | | | |
| MAPT- | 27 mer | 6007-6062 | AAUAGUAAACAGGAUACAGUCUCACCA | 707 |
| 6007 | antisense | (Hs-Mf) | | |
| | strand | | | |
| MAPT- | 27 mer | 6008-6063 | CAAUAUCAAACAGGAUACAGUCUCACC | 708 |
| 6008 | antisense | (Hs-Mf) | | |
| | strand | | | |
| MAPT- | 27 mer | 6009-6064 | GCAAUUGCAAACAGGAUACAGUCUCAC | 709 |
| 6009 | antisense | (Hs-Mf) | | |
| | strand | | | |
| MAPT- | 27 mer | 6010-6065 | AGCAAUAGCAAACAGGAUACAGUCUCA | 710 |
| 6010 | antisense | (Hs-Mf) | | |
| | strand | | | |
| MAPT- | 27 mer | 6011-6066 | AAGCAUUAGCAAACAGGAUACAGUCUC | 711 |
| 6011 | antisense | (Hs-Mf) | | |
| | strand | | | |
| MAPT- | 27 mer | 6012-6067 | CAAGCUAUAGCAAACAGGAUACAGUCU | 712 |
| 6012 | antisense | (Hs-Mf) | | |
| | strand | | | |
| MAPT- | 27 mer | 6013-6068 | ACAAGUAAUAGCAAACAGGAUACAGUC | 713 |
| 6013 | antisense | (Hs-Mf) | | |
| | strand | | | |
| MAPT- | 27 mer | 6014-6069 | AACAAUCAAUAGCAAACAGGAUACAGU | 714 |
| 6014 | antisense | (Hs-Mf) | | |
| | strand | | | |
| MAPT- | 27 mer | 6015-6070 | CAACAUGCAAUAGCAAACAGGAUACAG | 715 |
| 6015 | antisense | (Hs-Mf) | | |
| | strand | | | |
| MAPT- | 27 mer | 6017-6072 | CACAAUAAGCAAUAGCAAACAGGAUAC | 716 |
| 6017 | antisense | (Hs-Mf) | | |
| | strand | | | |
| MAPT- | 27 mer | 6119-6174 | GAAAUUAUGAAAAGGGUUACGAGGCAG | 717 |
| 6119 | antisense | (Hs-Mf) | | |
| | strand | | | |
| MAPT- | 27 mer | 6628-6689 | UAGUCUUGCUCAACAUGGCAAACUCAU | 718 |
| 6628 | antisense | (Hs-Mf) | | |
| | strand | | | |
| MAPT- | 27 mer | 6629-6690 | AUAGUUCUGCUCAACAUGGCAAACUCA | 719 |
| 6629 | antisense | (Hs-Mf) | | |
| | strand | | | |
| MAPT- | 27 mer | 6631-6692 | AAAUAUUCCUGCUCAACAUGGCAAACU | 720 |
| 6631 | antisense | (Hs-Mf) | | |
| | strand | | | |
| MAPT- | 27 mer | 6672-6733 | CUCAGUAUUACCGAAGAAAUCAUGGGA | 721 |
| 6672 | antisense | (Hs-Mf) | | |
| | strand | | | |
| MAPT- | 27 mer | 6731 (Hs) | AUAUAUACAUUCACAGACAGAAAGCUA | 722 |
| 6731 | antisense | | | |
| | strand | | | |
| MAPT- | 27 mer | 6732 (Hs) | UAUAUUGACAUUCACAGACAGAAAGCU | 723 |
| 6732 | antisense | | | |
| | strand | | | |
| MAPT- | 27 mer | 6738-6799 | AUACAUUAUAUAGACAUUCACAGACAG | 724 |
| 6738 | antisense | (Hs-Mf) | | |
| | strand | | | |
| MAPT- | 27 mer | 6739-6800 | AAUACUCUAUAUAGACAUUCACAGACA | 725 |
| 6739 | antisense | (Hs-Mf) | | |
| | strand | | | |
| MAPT- | 27 mer | 6740-6801 | CAAUAUACUAUAUAGACAUUCACAGAC | 726 |
| 6740 | antisense | (Hs-Mf) | | |
| | strand | | | |
| MAPT- | 27 mer | 6741-6802 | ACAAUUCACUAUAUAGACAUUCACAGA | 727 |
| 6741 | antisense | (Hs-Mf) | | |
| | strand | | | |
| MAPT- | 27 mer | 6742-6803 | CACAAUACACUAUAUAGACAUUCACAG | 728 |
| 6742 | antisense | (Hs-Mf) | | |
| | strand | | | |
| MAPT- | 27 mer | 6743-6804 | ACACAUUACACUAUAUAGACAUUCACA | 729 |
| 6743 | antisense | (Hs-Mf) | | |
| | strand | | | |
| MAPT- | 27 mer | 6745-6806 | ACACAUAAUACACUAUAUAGACAUUCA | 730 |
| 6745 | antisense | (Hs-Mf) | | |
| | strand | | | |
| MAPT- | 27 mer | 6748-6809 | AAAACUCACAAUACACUAUAUAGACAU | 731 |
| 6748 | antisense | (Hs-Mf) | | |
| | strand | | | |
| MAPT- | 27 mer | 6749-6810 | UAAAAUACACAAUACACUAUAUAGACA | 732 |
| 6749 | antisense | (Hs-Mf) | | |
| | strand | | | |
| MAPT- | 27 mer | 6750-6811 | UUAAAUCACACAAUACACUAUAUAGAC | 733 |
| 6750 | antisense | (Hs-Mf) | | |
| | strand | | | |
| MAPT- | 27 mer | 6751-6812 | GUUAAUACACACAAUACACUAUAUAGA | 734 |
| 6751 | antisense | (Hs-Mf) | | |
| | strand | | | |
| MAPT- | 27 mer | 6752-6813 | UGUUAUAACACACAAUACACUAUAUAG | 735 |
| 6752 | antisense | (Hs-Mf) | | |
| | strand | | | |
| MAPT- | 27 mer | 6753-6814 | UUGUUUAAACACACAAUACACUAUAUA | 736 |
| 6753 | antisense | (Hs-Mf) | | |
| | strand | | | |
| MAPT- | 27 mer | 6754-6815 | UUUGUUAAAACACACAAUACACUAUAU | 737 |
| 6754 | antisense | (Hs-Mf) | | |
| | strand | | | |
| MAPT- | 27 mer | 6755-6816 | AUUUGUUAAAACACACAAUACACUAUA | 738 |
| 6755 | antisense | (Hs-Mf) | | |
| | strand | | | |
| MAPT- | 27 mer | 6756-6817 | CAUUUUUUAAAACACACAAUACACUAU | 739 |
| 6756 | antisense | (Hs-Mf) | | |
| | strand | | | |
| MAPT- | 27 mer | 6757-6818 | UCAUUUGUUAAAACACACAAUACACUA | 740 |
| 6757 | antisense | (Hs-Mf) | | |
| | strand | | | |
| MAPT- | 27 mer | 6758-6819 | AUCAUUUGUUAAAACACACAAUACACU | 741 |
| 6758 | antisense | (Hs-Mf) | | |
| | strand | | | |
| MAPT- | 27 mer | 6759-6820 | AAUCAUUUGUUAAAACACACAAUACAC | 742 |
| 6759 | antisense | (Hs-Mf) | | |
| | strand | | | |
| MAPT- | 27 mer | 6760-6821 | AAAUCUUUUGUUAAAACACACAAUACA | 743 |
| 6760 | antisense | (Hs-Mf) | | |
| | strand | | | |
| MAPT- | 27 mer | 6761-6822 | UAAAUUAUUUGUUAAAACACACAAUAC | 744 |
| 6761 | antisense | (Hs-Mf) | | |
| | strand | | | |
| MAPT- | 27 mer | 6762-6823 | GUAAAUCAUUUGUUAAAACACACAAUA | 745 |
| 6762 | antisense | (Hs-Mf) | | |
| | strand | | | |
| MAPT- | 27 mer | 6763-6824 | UGUAAUUCAUUUGUUAAAACACACAAU | 746 |
| 6763 | antisense | (Hs-Mf) | | |
| | strand | | | |
| MAPT- | 27 mer | 6764-6825 | GUGUAUAUCAUUUGUUAAAACACACAA | 747 |
| 6764 | antisense | (Hs-Mf) | | |
| | strand | | | |
| MAPT- | 27 mer | 6765-6826 | AGUGUUAAUCAUUUGUUAAAACACACA | 748 |
| 6765 | antisense | (Hs-Mf) | | |
| | strand | | | |
| MAPT- | 27 mer | 6766-6827 | CAGUGUAAAUCAUUUGUUAAAACACAC | 749 |
| 6766 | antisense | (Hs-Mf) | | |
| | strand | | | |
| MAPT- | 27 mer | 6767-6828 | UCAGUUUAAAUCAUUUGUUAAAACACA | 750 |
| 6767 | antisense | (Hs-Mf) | | |
| | strand | | | |
| MAPT- | 27 mer | 6768-6829 | GUCAGUGUAAAUCAUUUGUUAAAACAC | 751 |
| 6768 | antisense | (Hs-Mf) | | |
| | strand | | | |
| MAPT- | 27 mer | 6769-6830 | AGUCAUUGUAAAUCAUUUGUUAAAACA | 752 |
| 6769 | antisense | (Hs-Mf) | | |
| | strand | | | |
| MAPT- | 27 mer | 6772-6833 | AACAGUCAGUGUAAAUCAUUUGUUAAA | 753 |
| 6772 | antisense | (Hs-Mf) | | |
| | strand | | | |
| MAPT- | 27 mer | 6773-6834 | CAACAUUCAGUGUAAAUCAUUUGUUAA | 754 |
| 6773 | antisense | (Hs-Mf) | | |
| | strand | | | |
| MAPT- | 27 mer | 6774-6835 | GCAACUGUCAGUGUAAAUCAUUUGUUA | 755 |
| 6774 | antisense | (Hs-Mf) | | |
| | strand | | | |
| MAPT- | 27 mer | 6775-6836 | AGCAAUAGUCAGUGUAAAUCAUUUGUU | 756 |
| 6775 | antisense | (Hs-Mf) | | |
| | strand | | | |
| MAPT- | 27 mer | 6777-6838 | ACAGCUACAGUCAGUGUAAAUCAUUUG | 757 |
| 6777 | antisense | (Hs-Mf) | | |
| | strand | | | |
| MAPT- | 27 mer | 6778-6839 | UACAGUAACAGUCAGUGUAAAUCAUUU | 758 |
| 6778 | antisense | (Hs-Mf) | | |
| | strand | | | |
| MAPT- | 27 mer | 6779-6840 | UUACAUCAACAGUCAGUGUAAAUCAUU | 759 |
| 6779 | antisense | (Hs-Mf) | | |
| | strand | | | |
| MAPT- | 27 mer | 6780-6841 | UUUACUGCAACAGUCAGUGUAAAUCAU | 760 |
| 6780 | antisense | (Hs-Mf) | | |
| | strand | | | |
| MAPT- | 27 mer | 6781 (Hs) | UUUUAUAGCAACAGUCAGUGUAAAUCA | 761 |
| 6781 | antisense | | | |
| | strand | | | |
| MAPT- | 27 mer | 6789 (Hs) | AAAUUUACUUUUACAGCAACAGUCAGU | 762 |
| 6789 | antisense | | | |
| | strand | | | |
| MAPT- | 27 mer | 6792 (Hs) | UCCAAUUUCACUUUUACAGCAACAGUC | 763 |
| 6792 | antisense | | | |
| | strand | | | |
| MAPT- | 27 mer | 6793 (Hs) | UUCCAUAUUCACUUUUACAGCAACAGU | 764 |
| 6793 | antisense | | | |
| | strand | | | |
| MAPT- | 27 mer | 6795 (Hs) | AUUUCUAAAUUCACUUUUACAGCAACA | 765 |
| 6795 | antisense | | | |
| | strand | | | |
| MAPT- | 27 mer | 6796 (Hs) | UAUUUUCAAAUUCACUUUUACAGCAAC | 766 |
| 6796 | antisense | | | |
| | strand | | | |
| MAPT- | 27 mer | 6797 (Hs) | UUAUUUCCAAAUUCACUUUUACAGCAA | 767 |
| 6797 | antisense | | | |
| | strand | | | |
| MAPT- | 27 mer | 6798 (Hs) | UUUAUUUCCAAAUUCACUUUUACAGCA | 768 |
| 6798 | antisense | | | |
| | strand | | | |
| MAPT- | Unmodified | 2456-2533 | AAAAAGAUUGAAACCCACAAGCAGCCG | 769 |
| 2456 | 36 mer | (Hs-Mf) | AAAGGCUGC | |
| | sense | | | |
| | strand | | | |
| MAPT- | Unmodified | 2567 (Hs) | CGGCAUCUCAGCAAUGUCUAGCAGCCGA | 770 |
| 2567 | 36 mer | | AAGGCUGC | |
| | sense | | | |
| | strand | | | |
| MAPT- | Unmodified | 2723-2800 | AGAGUGUGGAAAAAAAAAGAGCAGCCG | 771 |
| 2723 | 36 mer | (Hs-Mf) | AAAGGCUGC | |
| | sense | | | |
| | strand | | | |
| MAPT- | Unmodified | 690-455 | AAGACGAAGCUGCUGGUCAAGCAGCCG | 772 |
| 0690 | 36 mer | (Hs-Mf) | AAAGGCUGC | |
| | sense | | | |
| | strand | | | |
| MAPT- | Unmodified | 1494-1571 | AAGACGGGACUGGAAGCGAAGCAGCCG | 773 |
| 1494 | 36 mer | (Hs-Mf) | AAAGGCUGC | |
| | sense | | | |
| | strand | | | |
| MAPT- | Unmodified | 1733 (Hs) | GAUGGUAAAACGAAGAUCGAGCAGCCG | 774 |
| 1733 | 36 mer | | AAAGGCUGC | |
| | sense | | | |
| | strand | | | |
| MAPT- | Unmodified | 2273-2350 | AUAGUCUACAAACCAGUUGAGCAGCCG | 775 |
| 2273 | 36 mer | (Hs-Mf) | AAAGGCUGC | |
| | sense | | | |
| | strand | | | |
| MAPT- | Unmodified | 2274-2351 | UAGUCUACAAACCAGUUGAAGCAGCCG | 776 |
| 2274 | 36 mer | (Hs-Mf) | AAAGGCUGC | |
| | sense | | | |
| | strand | | | |
| MAPT- | Unmodified | 2276-2353 | GUCUACAAACCAGUUGACCAGCAGCCGA | 777 |
| 2276 | 36 mer | (Hs-Mf) | AAGGCUGC | |
| | sense | | | |
| | strand | | | |
| MAPT- | Unmodified | 2301-2378 | AGGUGACCUCCAAGUGUGGAGCAGCCG | 778 |
| 2301 | 36 mer | (Hs-Mf) | AAAGGCUGC | |
| | sense | | | |
| | strand | | | |
| MAPT- | Unmodified | 2347-2424- | AGGAGGUGGCCAGGUGGAAAGCAGCCG | 779 |
| 2347 | 36 mer | 1172 (Hs- | AAAGGCUGC | |
| | sense | Mf-Mm) | | |
| | strand | | | |
| MAPT- | Unmodified | 2357-2434 | CAGGUGGAAGUAAAAUCUGAGCAGCCG | 780 |
| 2357 | 36 mer | (Hs-Mf) | AAAGGCUGC | |
| | sense | | | |
| | strand | | | |
| MAPT- | Unmodified | 2358-2435 | AGGUGGAAGUAAAAUCUGAAGCAGCCG | 781 |
| 2358 | 36 mer | (Hs-Mf) | AAAGGCUGC | |
| | sense | | | |
| | strand | | | |
| MAPT- | Unmodified | 2364-2441 | AAGUAAAAUCUGAGAAGCUAGCAGCCG | 782 |
| 2364 | 36 mer | (Hs-Mf) | AAAGGCUGC | |
| | sense | | | |
| | strand | | | |
| MAPT- | Unmodified | 2378 (Hs) | AAGCUUGACUUCAAGGACAAGCAGCCG | 783 |
| 2378 | 36 mer | | AAAGGCUGC | |
| | sense | | | |
| | strand | | | |
| MAPT- | Unmodified | 2459-2536- | AAGAUUGAAACCCACAAGCAGCAGCCG | 784 |
| 2459 | 36 mer | 1284 (Hs- | AAAGGCUGC | |
| | sense | Mf-Mm) | | |
| | strand | | | |
| MAPT- | Unmodified | 2461-2538- | GAUUGAAACCCACAAGCUGAGCAGCCG | 785 |
| 2461 | 36 mer | 1286 (Hs- | AAAGGCUGC | |
| | sense | Mf-Mm) | | |
| | strand | | | |
| MAPT- | Unmodified | 2460-2537- | AGAUUGAAACCCACAAGCUAGCAGCCG | 786 |
| 2460 | 36 mer | 1285 (Hs- | AAAGGCUGC | |
| | sense | Mf-Mm) | | |
| | strand | | | |
| MAPT- | Unmodified | 1479-1556 | UGGUCAGUAAAAGCAAAGAAGCAGCCG | 787 |
| 1479 | 36 mer | (Hs-Mf) | AAAGGCUGC | |
| | sense | | | |
| | strand | | | |
| MAPT- | Unmodified | 1505-1582 | GGAAGCGAUGACAAAAAAGAGCAGCCG | 788 |
| 1505 | 36 mer | (Hs-Mf) | AAAGGCUGC | |
| | sense | | | |
| | strand | | | |
| MAPT- | Unmodified | 2096-2173 | CCCAUGCCAGACCUGAAGAAGCAGCCGA | 789 |
| 2096 | 36 mer | (Hs-Mf) | AAGGCUGC | |
| | sense | | | |
| | strand | | | |
| MAPT- | Unmodified | 2270-2347 | CAAAUAGUCUACAAACCAGAGCAGCCG | 790 |
| 2270 | 36 mer | (Hs-Mf) | AAAGGCUGC | |
| | sense | | | |
| | strand | | | |
| MAPT- | Unmodified | 2279-2356 | UACAAACCAGUUGACCUGAAGCAGCCG | 791 |
| 2279 | 36 mer | (Hs-Mf) | AAAGGCUGC | |
| | sense | | | |
| | strand | | | |
| MAPT- | Unmodified | 2281-2358 | CAAACCAGUUGACCUGAGCAGCAGCCGA | 792 |
| 2281 | 36 mer | (Hs-Mf) | AAGGCUGC | |
| | sense | | | |
| | strand | | | |
| MAPT- | Unmodified | 2284-2361 | ACCAGUUGACCUGAGCAAGAGCAGCCG | 793 |
| 2284 | 36 mer | (Hs-Mf) | AAAGGCUGC | |
| | sense | | | |
| | strand | | | |
| MAPT- | Unmodified | 2299-2376 | CAAGGUGACCUCCAAGUGUAGCAGCCG | 794 |
| 2299 | 36 mer | (Hs-Mf) | AAAGGCUGC | |
| | sense | | | |
| | strand | | | |
| MAPT- | Unmodified | 2376 (Hs) | AGAAGCUUGACUUCAAGGAAGCAGCCG | 795 |
| 2376 | 36 mer | | AAAGGCUGC | |
| | sense | | | |
| | strand | | | |
| MAPT- | Unmodified | 2379 (Hs) | AGCUUGACUUCAAGGACAGAGCAGCCG | 796 |
| 2379 | 36 mer | | AAAGGCUGC | |
| | sense | | | |
| | strand | | | |
| MAPT- | Unmodified | 2382 (Hs) | UUGACUUCAAGGACAGAGUAGCAGCCG | 797 |
| 2382 | 36 mer | | AAAGGCUGC | |
| | sense | | | |
| | strand | | | |
| MAPT- | Unmodified | 2449-2526 | AGGAAAUAAAAAGAUUGAAAGCAGCCG | 798 |
| 2449 | 36 mer | (Hs-Mf) | AAAGGCUGC | |
| | sense | | | |
| | strand | | | |
| MAPT- | Unmodified | 2450-2527 | GGAAAUAAAAAGAUUGAAAAGCAGCCG | 799 |
| 2450 | 36 mer | (Hs-Mf) | AAAGGCUGC | |
| | sense | | | |
| | strand | | | |
| MAPT- | Unmodified | 2451-2528 | GAAAUAAAAAGAUUGAAACAGCAGCCG | 800 |
| 2451 | 36 mer | (Hs-Mf) | AAAGGCUGC | |
| | sense | | | |
| | strand | | | |
| MAPT- | Unmodified | 2452-2529 | AAAUAAAAAGAUUGAAACCAGCAGCCG | 801 |
| 2452 | 36 mer | (Hs-Mf) | AAAGGCUGC | |
| | sense | | | |
| | strand | | | |
| MAPT- | Unmodified | 2453-2530 | AAUAAAAAGAUUGAAACCCAGCAGCCG | 802 |
| 2453 | 36 mer | (Hs-Mf) | AAAGGCUGC | |
| | sense | | | |
| | strand | | | |
| MAPT- | Unmodified | 2454-2531 | AUAAAAAGAUUGAAACCCAAGCAGCCG | 803 |
| 2454 | 36 mer | (Hs-Mf) | AAAGGCUGC | |
| | sense | | | |
| | strand | | | |
| MAPT- | unmodified | 2456-2533 | UUGUGGGUUUCAAUCUUUUUGG | 804 |
| 2456 | 32 mer | (Hs-Mf) | | |
| | antisense | | | |
| | strand | | | |
| MAPT- | unmodified | 2567 (Hs) | UAGACAUUGCUGAGAUGCCGGG | 805 |
| 2567 | 32 mer | | | |
| | antisense | | | |
| | strand | | | |
| MAPT- | unmodified | 2723-2800 | UCUUUUUUUUUCCACACUCUGG | 806 |
| 2723 | 32 mer | (Hs-Mf) | | |
| | antisense | | | |
| | strand | | | |
| MAPT- | unmodified | 690-455 | UUGACCAGCAGCUUCGUCUUGG | 807 |
| 0690 | 32 mer | (Hs-Mf) | | |
| | antisense | | | |
| | strand | | | |
| MAPT- | unmodified | 1494-1571 | UUCGCUUCCAGUCCCGUCUUGG | 808 |
| 1494 | 32 mer | (Hs-Mf) | | |
| | antisense | | | |
| | strand | | | |
| MAPT- | unmodified | 1733 (Hs) | UCGAUCUUCGUUUUACCAUCGG | 809 |
| 1733 | 32 mer | | | |
| | antisense | | | |
| | strand | | | |
| MAPT- | unmodified | 2273-2350 | UCAACUGGUUUGUAGACUAUGG | 810 |
| 2273 | 32 mer | (Hs-Mf) | | |
| | antisense | | | |
| | strand | | | |
| MAPT- | unmodified | 2274-2351 | UUCAACUGGUUUGUAGACUAGG | 811 |
| 2274 | 32 mer | (Hs-Mf) | | |
| | antisense | | | |
| | strand | | | |
| MAPT- | unmodified | 2276-2353 | UGGUCAACUGGUUUGUAGACGG | 812 |
| 2276 | 32 mer | (Hs-Mf) | | |
| | antisense | | | |
| | strand | | | |
| MAPT- | unmodified | 2301-2378 | UCCACACUUGGAGGUCACCUGG | 813 |
| 2301 | 32 mer | (Hs-Mf) | | |
| | antisense | | | |
| | strand | | | |
| MAPT- | unmodified | 2347-2424- | UUUCCACCUGGCCACCUCCUGG | 814 |
| 2347 | 32 mer | 1172 (Hs- | | |
| | antisense | Mf-Mm) | | |
| | strand | | | |
| MAPT- | unmodified | 2357-2434 | UCAGAUUUUACUUCCACCUGGG | 815 |
| 2357 | 32 mer | (Hs-Mf) | | |
| | antisense | | | |
| | strand | | | |
| MAPT- | unmodified | 2358-2435 | UUCAGAUUUUACUUCCACCUGG | 816 |
| 2358 | 32 mer | (Hs-Mf) | | |
| | antisense | | | |
| | strand | | | |
| MAPT- | unmodified | 2364-2441 | UAGCUUCUCAGAUUUUACUUGG | 817 |
| 2364 | 32 mer | (Hs-Mf) | | |
| | antisense | | | |
| | strand | | | |
| MAPT- | unmodified | 2378 (Hs) | UUGUCCUUGAAGUCAAGCUUGG | 818 |
| 2378 | 32 mer | | | |
| | antisense | | | |
| | strand | | | |
| MAPT- | unmodified | 2459-2536- | UGCUUGUGGGUUUCAAUCUUGG | 819 |
| 2459 | 32 mer | 1284 (Hs- | | |
| | antisense | Mf-Mm) | | |
| | strand | | | |
| MAPT- | unmodified | 2461-2538- | UCAGCUUGUGGGUUUCAAUCGG | 820 |
| 2461 | 32 mer | 1286 (Hs- | | |
| | antisense | Mf-Mm) | | |
| | strand | | | |
| MAPT- | unmodified | 2460-2537- | UAGCUUGUGGGUUUCAAUCUGG | 821 |
| 2460 | 32 mer | 1285 (Hs- | | |
| | antisense | Mf-Mm) | | |
| | strand | | | |
| MAPT- | unmodified | 1479-1556 | UUCUUUGCUUUUACUGACCAGG | 822 |
| 1479 | 32 mer | (Hs-Mf) | | |
| | antisense | | | |
| | strand | | | |
| MAPT- | unmodified | 1505-1582 | UCUUUUUUGUCAUCGCUUCCGG | 823 |
| 1505 | 32 mer | (Hs-Mf) | | |
| | antisense | | | |
| | strand | | | |
| MAPT- | unmodified | 2096-2173 | UUCUUCAGGUCUGGCAUGGGGG | 824 |
| 2096 | 32 mer | (Hs-Mf) | | |
| | antisense | | | |
| | strand | | | |
| MAPT- | unmodified | 2270-2347 | UCUGGUUUGUAGACUAUUUGGG | 825 |
| 2270 | 32 mer | (Hs-Mf) | | |
| | antisense | | | |
| | strand | | | |
| MAPT- | unmodified | 2279-2356 | UUCAGGUCAACUGGUUUGUAGG | 826 |
| 2279 | 32 mer | (Hs-Mf) | | |
| | antisense | | | |
| | strand | | | |
| MAPT- | unmodified | 2281-2358 | UGCUCAGGUCAACUGGUUUGGG | 827 |
| 2281 | 32 mer | (Hs-Mf) | | |
| | antisense | | | |
| | strand | | | |
| MAPT- | unmodified | 2284-2361 | UCUUGCUCAGGUCAACUGGUGG | 828 |
| 2284 | 32 mer | (Hs-Mf) | | |
| | antisense | | | |
| | strand | | | |
| MAPT- | unmodified | 2299-2376 | UACACUUGGAGGUCACCUUGGG | 829 |
| 2299 | 32 mer | (Hs-Mf) | | |
| | antisense | | | |
| | strand | | | |
| MAPT- | unmodified | 2376 (Hs) | UUCCUUGAAGUCAAGCUUCUGG | 830 |
| 2376 | 32 mer | | | |
| | antisense | | | |
| | strand | | | |
| MAPT- | unmodified | 2379 (Hs) | UCUGUCCUUGAAGUCAAGCUGG | 831 |
| 2379 | 32 mer | | | |
| | antisense | | | |
| | strand | | | |
| MAPT- | unmodified | 2382 (Hs) | UACUCUGUCCUUGAAGUCAAGG | 832 |
| 2382 | 32 mer | | | |
| | antisense | | | |
| | strand | | | |
| MAPT- | unmodified | 2449-2526 | UUUCAAUCUUUUUAUUUCCUGG | 833 |
| 2449 | 32 mer | (Hs-Mf) | | |
| | antisense | | | |
| | strand | | | |
| MAPT- | unmodified | 2450-2527 | UUUUCAAUCUUUUUAUUUCCGG | 834 |
| 2450 | 32 mer | (Hs-Mf) | | |
| | antisense | | | |
| | strand | | | |
| MAPT- | unmodified | 2451-2528 | UGUUUCAAUCUUUUUAUUUCGG | 835 |
| 2451 | 32 mer | (Hs-Mf) | | |
| | antisense | | | |
| | strand | | | |
| MAPT- | unmodified | 2452-2529 | UGGUUUCAAUCUUUUUAUUUGG | 836 |
| 2452 | 32 mer | (Hs-Mf) | | |
| | antisense | | | |
| | strand | | | |
| MAPT- | unmodified | 2453-2530 | UGGGUUUCAAUCUUUUUAUUGG | 837 |
| 2453 | 32 mer | (Hs-Mf) | | |
| | antisense | | | |
| | strand | | | |
| MAPT- | unmodified | 2454-2531 | UUGGGUUUCAAUCUUUUUAUGG | 838 |
| 2454 | 32 mer | (Hs-Mf) | | |
| | antisense | | | |
| | strand | | | |
| MAPT- | Modified | 2456-2533 | [mAs][mA][fA][mA][fA][mG][mA][fU][mU][fG] | 839 |
| 2456 | 36 mer | (Hs-Mf) | [mA][fA][fA][mC][fC][mC][fA][mC][mA][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2567 (Hs) | [mCs][mG][fG][mC][fA][mU][mC][fU][mC][fA] | 840 |
| 2567 | 36 mer | | [mG][fC][fA][mA][fU][mG][fU][mC][mU][mA] | |
| | sense | | [mG][mC][mA][mG][mC][mC][mG][ademA- | |
| | strand | | GalNAc][ademA-GalNAc][ademA- | |
| | | | GalNAc][mG][G][mC][mU][mG][mC] | |
| MAPT- | Modified | 2723-2800 | [mAs][mG][fA][mG][fU][mG][mU][fG][mG][fA] | 841 |
| 2723 | 36 mer | (Hs-Mf) | [mA][fA][fA][mA][fA][mA][fA][mA][mG] | |
| | sense | | [mA][mG][mC][mA][mG][mC][mC][mG][adem | |
| | strand | | A-GalNAc][ademA-GalNAc][ademA- | |
| | | | GalNAc][mG][mG][mC][mU][mG][mC] | |
| MAPT- | Modified | 690-455 | [mAs][mA][fG][mA][fC][mG][mA][fA][mG][fC] | 842 |
| 0690 | 36 mer | (Hs-Mf) | [mU][fG][fC][mU][fG][mG][fU][mC][mA][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 1494-1571 | [mAs][mA][fG][mA][fC][mG][mG][fG][mA][fC] | 843 |
| 1494 | 36 mer | (Hs-Mf) | [mU][fG][fG][mA][fA][mG][fC][mG][mA] | |
| | sense | | [mA][mG][mC][mA][mG][mC][mC][mA-G][ademA- | |
| | strand | | GalNAc][ademA-GalNAc][ademA- | |
| | | | GalNAc][mG][mG][mC][mU][mG][mC] | |
| MAPT- | Modified | 1733 (Hs) | [mGs][mA][fU][mG][fG][mU][mA][fA][mA][fA] | 844 |
| 1733 | 36 mer | | [mC][fG][fA][mA][fG][mA][fU][mC][mG] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2273-2350 | [mAs][mU][fA][mG][fU][mC][mU][fA][mC][fA] | 845 |
| 2273 | 36 mer | (Hs-Mf) | [mA][fA][fC][mC][fA][mG][fU][mU][mG] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2274-2351 | [mUs][mA][fG][mU][fC][mU][mA][fC][mA][fA] | 846 |
| 2274 | 36 mer | (Hs-Mf) | [mA][fC][fC][mA][fG][mU][fU][mG][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2276-2353 | [mGs][mU][fC][mU][fA][mC][mA][fA][mA][fC] | 847 |
| 2276 | 36 mer | (Hs-Mf) | [mC][fA][fG][mU][fU][mG][fA][mC][mC][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2301-2378 | [mAs][mG][fG][mU][fG][mA][mC][fC][mU][fC] | 848 |
| 2301 | 36 mer | (Hs-Mf) | [mC][fA][fA][mG][fU][mG][fU][mG][mG] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2347-2424- | [mAs][mG][fG][mA][fG][mG][mU][fG][mG][fC] | 849 |
| 2347 | 36 mer | 1172 (Hs- | [mC][fA][fG][mG][fU][mG][fG][mA][mA] | |
| | sense | Mf-Mm) | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2357-2434 | [mCs][mA][fG][mG][fU][mG][mG][fA][mA][fG] | 850 |
| 2357 | 36 mer | (Hs-Mf) | [mU][fA][fA][mA][fA][mU][fC][mU][mG] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2358-2435 | [mAs][mG][fG][mU][fG][mG][mA][fA][mG][fU] | 851 |
| 2358 | 36 mer | (Hs-Mf) | [mA][fA][fA][mA][fU][mC][fU][mG][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2364-2441 | [mAs][mA][fG][mU][fA][mA][mA][fA][mU][fC] | 852 |
| 2364 | 36 mer | (Hs-Mf) | [mU][fG][fA][mG][fA][mA][fG][mC][mU] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2378 (Hs) | [mAs][mA][fG][mC][fU][mU][mG][fA][mC][fU] | 853 |
| 2378 | 36 mer | | [mU][fC][fA][mA][fG][mG][fA][mC][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2459-2536- | [mAs][mA][fG][mA][fU][mU][mG][fA][mA][fA] | 854 |
| 2459 | 36 mer | 1284 (Hs- | [mC][fC][fC][mA][fC][mA][fA][mG][mC][mA] | |
| | sense | Mf-Mm) | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2461-2538- | [mGs][mA][fU][mU][fG][mA][mA][fA][mC][fC] | 855 |
| 2461 | 36 mer | 1286 (Hs- | [mC][fA][fC][mA][fA][mG][fC][mU][mG][mA] | |
| | sense | Mf-Mm) | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2460-2537- | [mAs][mG][mA][mU][mU][mG][mA][A][fA][fC] | 856 |
| 2460 | 36 mer | 1285 (Hs- | [fC][mC][mA][mC][mA][mA][mG][mC][mU] | |
| | sense | Mf-Mm) | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 1479-1556 | [mUs][mG][fG][mU][fC][mA][mG][fU][mA][fA] | 857 |
| 1479 | 36 mer | (Hs-Mf) | [mA][fA][fG][mC][fA][mA][fA][mG][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 1505-1582 | [mGs][mG][fA][mA][fG][mC][mG][fA][mU][fG] | 858 |
| 1505 | 36 mer | (Hs-Mf) | [mA][fC][fA][mA][fA][mA][fA][mA][mG] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2096-2173 | [mCs][mC][fC][mA][fU][mG][mC][fC][mA][fG] | 859 |
| 2096 | 36 mer | (Hs-Mf) | [mA][fC][fC][mU][fG][mA][fA][mG][mA][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2270-2347 | [mCs][mA][fA][mA][fU][mA][mG][fU][mC][fU] | 860 |
| 2270 | 36 mer | (Hs-Mf) | [mA][fC][fA][mA][fA][mC][fC][mA][mG][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2279-2356 | [mUs][mA][fC][mA][fA][mA][mC][fC][mA][fG] | 861 |
| 2279 | 36 mer | (Hs-Mf) | [mU][fU][fG][mA][fC][mC][fU][mG][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2281-2358 | [mCs][mA][fA][mA][fC][mC][mA][fG][mU][fU] | 862 |
| 2281 | 36 mer | (Hs-Mf) | [mG][fA][fC][mC][fU][mG][fA][mG][mC][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2284-2361 | [mAs][mC][fC][mA][fG][mU][mU][fG][mA][fC] | 863 |
| 2284 | 36 mer | (Hs-Mf) | [mC][fU][fG][mA][fG][mC][fA][mA][mG][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2299-2376 | [mCs][mA][fA][mG][fG][mU][mG][fA][mC][fC] | 864 |
| 2299 | 36 mer | (Hs-Mf) | [mU][fC][fC][mA][fA][mG][fU][mG][mU][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2376 (Hs) | [mAs][mG][fA][mA][fG][mC][mU][fU][mG][fA] | 865 |
| 2376 | 36 mer | | [mC][fU][fU][mC][fA][mA][fG][mG][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2379 (Hs) | [mAs][mG][fC][mU][fU][mG][mA][fC][mU][fU] | 866 |
| 2379 | 36 mer | | [mC][fA][fA][mG][fG][mA][fC][mA][mG] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2382 (Hs) | [mUs][mU][fG][mA][fC][mU][mU][fC][mA][fA] | 867 |
| 2382 | 36 mer | | [mG][fG][fA][mC][fA][mG][fA][mG][mU] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2449-2526 | [mAs][mG][fG][mA][fA][mA][mU][fA][mA][fA] | 868 |
| 2449 | 36 mer | (Hs-Mf) | [mA][fA][fG][mA][fU][mU][fG][mA][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2450-2527 | [mGs][mG][fA][mA][fA][mU][mA][fA][mA][fA] | 869 |
| 2450 | 36 mer | (Hs-Mf) | [mA][fG][fA][mU][fU][mG][fA][mA][mA] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2451-2528 | [mGs][mA][fA][mA][fU][mA][mA][fA][mA][fA] | 870 |
| 2451 | 36 mer | (Hs-Mf) | [mG][fA][fU][mU][fG][mA][fA][mA][mC] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2452-2529 | [mAs][mA][fA][mU][fA][mA][mA][fA][mA][fG] | 871 |
| 2452 | 36 mer | (Hs-Mf) | [mA][fU][fU][mG][fA][mA][fA][mC][mC] | |
| | sense | | [mA][mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2453-2530 | [mAs][mA][fU][mA][fA][mA][mA][fA][mG][fA] | 872 |
| 2453 | 36 mer | (Hs-Mf) | [mU][fU][fG][mA][fA][mA][fC][mC][mC][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2454-2531 | [mAs][mU][fA][mA][fA][mA][mA][fG][mA][fU] | 873 |
| 2454 | 36 mer | (Hs-Mf) | [mU][fG][fA][mA][fA][mC][fC][mC][mA][mA] | |
| | sense | | [mG][mC][mA][mG][mC][mC][mG][ademA- | |
| | strand | | GalNAc][ademA-GalNAc][ademA- | |
| | | | GalNAc][mG][mG][mC][mU][mG][mC] | |
| MAPT- | Modified | 2456-2533 | [MePhosphonate-4O- | 874 |
| 2456 | 22 mer | (Hs-Mf) | mUs][fUs][fG][fU][fG][mG][fG][mU][mU][fU] | |
| | antisense | | [mC][mA][mA][fU][mC][fU][mU][mU][fU] | |
| | strand | | [mUs][mGs][mG] | |
| MAPT- | Modified | 2567 (Hs) | [MePhosphonate-4O- | 875 |
| 2567 | 22 mer | | mUs][fAs][fG][fA][fC][mA][fU][mU][mG][fC] | |
| | antisense | | [mU][mG][mA][fG][mA][fU][mG][mC][fC][mGs] | |
| | strand | | [mGs][mG] | |
| MAPT- | Modified | 2723-2800 | [MePhosphonate-4O- | 876 |
| 2723 | 22 mer | (Hs-Mf) | mUs][fCs][fU][fU][fU][mU][fU][mU][mU][fU] | |
| | antisense | | [mU][mC][mC][fA][mC][fA][mC][mU][fC][mUs] | |
| | strand | | [mGs][mG] | |
| MAPT- | Modified | 690-455 | [MePhosphonate-4O- | 877 |
| 0690 | 22 mer | (Hs-Mf) | mUs][fUs][fG][fA][fC][mC][fA][mG][mC][fA] | |
| | antisense | | [mG][mC][mU][fU][mC][fG][mU][mC][fU][mUs] | |
| | strand | | [mGs][mG] | |
| MAPT- | Modified | 1494-1571 | [MePhosphonate-4O- | 878 |
| 1494 | 22 mer | (Hs-Mf) | mUs][fUs][fC][fG][fC][mU][fU][mC][mC][fA] | |
| | antisense | | [mG][mU][mC][fC][mC][fG][mU][mC][fU][mUs] | |
| | strand | | [mGs][mG] | |
| MAPT- | Modified | 1733 (Hs) | [MePhosphonate-4O- | 879 |
| 1733 | 22 mer | | mUs][fCs][fG][fA][fU][mC][fU][mU][mC][fG] | |
| | antisense | | [mU][mU][mU][fU][mA][fC][mC][mA][fU][mCs] | |
| | strand | | [mGs][mG] | |
| MAPT- | Modified | 2273-2350 | [MePhosphonate-4O- | 880 |
| 2273 | 22 mer | (Hs-Mf) | mUs][fCs][fA][fA][fC][mU][fG][mG][mU][fU] | |
| | antisense | | [mU][mG][mU][fA][mG][fA][mC][mU][fA][mUs] | |
| | strand | | [mGs][mG] | |
| MAPT- | Modified | 2274-2351 | [MePhosphonate-4O- | 881 |
| 2274 | 22 mer | (Hs-Mf) | mUs][fUs][fC][fA][fA][mC][fU][mG][mG][fU] | |
| | antisense | | [mU][mU][mG][fU][mA][fG][mA][mC][fU][mAs] | |
| | strand | | [mGs][mG] | |
| MAPT- | Modified | 2276-2353 | [MePhosphonate-4O- | 882 |
| 2276 | 22 mer | (Hs-Mf) | mUs][fGs][fG][fU][fC][mA][fA][mC][mU][fG] | |
| | antisense | | [mG][mU][mU][fU][mG][fU][mA][mG][fA][mCs] | |
| | strand | | [mGs][mG] | |
| MAPT- | Modified | 2301-2378 | [MePhosphonate-4O- | 883 |
| 2301 | 22 mer | (Hs-Mf) | mUs][fCs][fC][fA][fC][mA][fC][mU][mU][fG] | |
| | antisense | | [mG][mA][mG][fG][mU][fC][mA][mC][fC][mUs] | |
| | strand | | [mGs][mG] | |
| MAPT- | Modified | 2347-2424- | [MePhosphonate-4O- | 884 |
| 2347 | 22 mer | 1172 (Hs- | mUs][fUs][fU][fC][fC][mA][fC][mC][mU][fG] | |
| | antisense | Mf-Mm) | [mG][mC][mC][fA][mC][fC][mU][mC][fC][mUs] | |
| | strand | | [mGs][mG] | |
| MAPT- | Modified | 2357-2434 | [MePhosphonate-4O- | 885 |
| 2357 | 22 mer | (Hs-Mf) | mUs][fCs][fA][fG][fA][mU][fU][mU][mU][fA] | |
| | antisense | | [mC][mU][mU][fC][mC][fA][mC][mC][fU][mGs] | |
| | strand | | [mGs][mG] | |
| MAPT- | Modified | 2358-2435 | [MePhosphonate-4O- | 886 |
| 2358 | 22 mer | (Hs-Mf) | mUs][fUs][fC][fA][fG][mA][fU][mU][mU][fU] | |
| | antisense | | [mA][mC][mU][fU][mC][fC][mA][mC][fC][mUs] | |
| | strand | | [mGs][mG] | |
| MAPT- | Modified | 2364-2441 | [MePhosphonate-4O- | 887 |
| 2364 | 22 mer | (Hs-Mf) | mUs][fAs][fG][fC][fU][mU][fC][mU][mC][fA] | |
| | antisense | | [mG][mA][mU][fU][mU][fU][mA][mC][fU][mUs] | |
| | strand | | [mGs][mG] | |
| MAPT- | Modified | 2378 (Hs) | [MePhosphonate-4O- | 888 |
| 2378 | 22 mer | | mUs][fUs][fG][fU][fC][mC][fU][mU][mG][fA] | |
| | antisense | | [mA][mG][mU][fC][mA][fA][mG][mC][fU][mUs] | |
| | strand | | [mGs][mG] | |
| MAPT- | Modified | 2459-2536- | [MePhosphonate-4O- | 889 |
| 2459 | 22 mer | 1284 (Hs- | mUs][fGs][fC][fU][fU][mG][fU][mG][mG][fG] | |
| | antisense | Mf-Mm) | [mU][mU][mU][fC][mA][fA][mU][mC][fU][mUs] | |
| | strand | | [mGs][mG] | |
| MAPT- | Modified | 2461-2538- | [MePhosphonate-4O- | 890 |
| 2461 | 22 mer | 1286 (Hs- | mUs][fCs][fA][fG][fC][mU][fU][mG][mU][fG] | |
| | antisense | Mf-Mm) | [mG][mG][mU][fU][mU][fC][mA][mA][fU][mCs] | |
| | strand | | [mGs][mG] | |
| MAPT- | Modified | 2460-2537- | [MePhosphonate-4O- | 891 |
| 2460 | 22 mer | 1285 (Hs- | mUs][fAs][fGs][fC][fU][mU][fG][mU][mG][fG] | |
| | antisense | Mf-Mm) | [mG][mU][mU][fU][mC][mA][mA][mU][mC] | |
| | strand | | [mUs][mGs][mG] | |
| MAPT- | Modified | 1479-1556 | [MePhosphonate-4O- | 892 |
| 1479 | 22 mer | (Hs-Mf) | mUs][fUs][fC][fU][fU][mU][fG][mC][mU][fU] | |
| | antisense | | [mU][mU][mA][fC][mU][fG][mA][mC][fC][mAs] | |
| | strand | | [mGs][mG] | |
| MAPT- | Modified | 1505-1582 | [MePhosphonate-4O- | 893 |
| 1505 | 22 mer | (Hs-Mf) | mUs][fCs][fU][fU][fU][mU][fU][mU][mG][fU] | |
| | antisense | | [mC][mA][mU][fC][mG][fC][mU][mU][fC][mCs] | |
| | strand | | [mGs][mG] | |
| MAPT- | Modified | 2096-2173 | [MePhosphonate-4O- | 894 |
| 2096 | 22 mer | (Hs-Mf) | mUs][fUs][fC][fU][fU][mC][fA][mG][mG][fU] | |
| | antisense | | [mC][mU][mG][fG][mC][fA][mU][mG][fG][mGs] | |
| | strand | | [mGs][mG] | |
| MAPT- | Modified | 2270-2347 | [MePhosphonate-4O- | 895 |
| 2270 | 22 mer | (Hs-Mf) | mUs][fCs][fU][fG][fG][mU][fU][mU][mG][fU] | |
| | antisense | | [mA][mG][mA][fC][mU][fA][mU][mU][fU][mGs] | |
| | strand | | [mGs][mG] | |
| MAPT- | Modified | 2279-2356 | [MePhosphonate-4O- | 896 |
| 2279 | 22 mer | (Hs-Mf) | mUs][fUs][fC][fA][fG][mG][fU][mC][mA][fA] | |
| | antisense | | [mC][mU][mG][fG][mU][fU][mU][mG][fU][mAs] | |
| | strand | | [mGs][mG] | |
| MAPT- | Modified | 2281-2358 | [MePhosphonate-4O- | 897 |
| 2281 | 22 mer | (Hs-Mf) | mUs][fGs][fC][fU][fC][mA][fG][mG][mU][fC] | |
| | antisense | | [mA][mA][mC][fU][mG][fG][mU][mU][fU][mGs] | |
| | strand | | [mGs][mG] | |
| MAPT- | Modified | 2284-2361 | [MePhosphonate-4O- | 898 |
| 2284 | 22 mer | (Hs-Mf) | mUs][fCs][fU][fU][fG][mC][fU][mC][mA][G] | |
| | antisense | | [mG][mU][mC][fA][mA][fC][mU][mG][fG][mUs] | |
| | strand | | [mGs][mG] | |
| MAPT- | Modified | 2299-2376 | [MePhosphonate-4O- | 899 |
| 2299 | 22 mer | (Hs-Mf) | mUs][fAs][fC][fA][fC][mU][fU][mG][mG][fA] | |
| | antisense | | [mG][mG][mU][fC][mA][fC][mC][mU][fU][mGs] | |
| | strand | | [mGs][mG] | |
| MAPT- | Modified | 2376 (Hs) | [MePhosphonate-4O- | 900 |
| 2376 | 22 mer | | mUs][fUs][fC][fC][fU][mU][fG][mA][mA][fG] | |
| | antisense | | [mU][mC][mA][fA][mG][fC][mU][mU][fC][mUs] | |
| | strand | | [mGs][mG] | |
| MAPT- | Modified | 2379 (Hs) | [MePhosphonate-4O- | 901 |
| 2379 | 22 mer | | mUs][fCs][fU][fG][fU][mC][fC][mU][mU][fG] | |
| | antisense | | [mA][mA][mG][fU][mC][fA][mA][mG][fC][mUs] | |
| | strand | | [mGs][mG] | |
| MAPT- | Modified | 2382 (Hs) | [MePhosphonate-4O- | 902 |
| 2382 | 22 mer | | mUs][fAs][fC][fU][fC][mU][fG][mU][mC][fC] | |
| | antisense | | [mU][mU][mG][fA][mA][fG][mU][mC][fA][mAs] | |
| | strand | | [mGs][mG] | |
| MAPT- | Modified | 2449-2526 | [MePhosphonate-4O- | 903 |
| 2449 | 22 mer | (Hs-Mf) | mUs][fUs][fU][fC][fA][mA][fU][mC][mU][fU] | |
| | antisense | | [mU][mU][mU][fA][mU][fU][mU][mC][fC][mUs] | |
| | strand | | [mGs][mG] | |
| MAPT- | Modified | 2450-2527 | [MePhosphonate-4O- | 904 |
| 2450 | 22 mer | (Hs-Mf) | mUs][fUs][fU][fU][fC][mA][fA][mU][mC][fU] | |
| | antisense | | [mU][mU][mU][fU][mA][fU][mU][mU][fC][mCs] | |
| | strand | | [mGs][mG] | |
| MAPT- | Modified | 2451-2528 | [MePhosphonate-4O- | 905 |
| 2451 | 22 mer | (Hs-Mf) | mUs][fGs][fU][fU][fU][mC][fA][mA][mU][fC] | |
| | antisense | | [mU][mU][mU][fU][mU][fA][mU][mU][fU][mCs] | |
| | strand | | [mGs][mG] | |
| MAPT- | Modified | 2452-2529 | [MePhosphonate-4O- | 906 |
| 2452 | 22 mer | (Hs-Mf) | mUs][fGs][fG][fU][fU][mU][fC][mA][mA][fU] | |
| | antisense | | [mC][mU][mU][fU][mU][fU][mA][mU][fU][mUs] | |
| | strand | | [mGs][mG] | |
| MAPT- | Modified | 2453-2530 | [MePhosphonate-4O- | 907 |
| 2453 | 22 mer | (Hs-Mf) | mUs][fGs][fG][fG][fU][mU][fU][mC][mA][fA] | |
| | antisense | | [mU][mC][mU][fU][mU][fU][mU][mA][fU][mUs] | |
| | strand | | [mGs][mG] | |
| MAPT- | Modified | 2454-2531 | [MePhosphonate-4O- | 908 |
| 2454 | 22 mer | (Hs-Mf) | mUs][fUs][fG][fG][fG][mU][fU][mU][mC][fA] | |
| | antisense | | [mA][mU][mC][fU][mU][fU][mU][mU][fA][mUs] | |
| | strand | | [mGs][mG] | |
| Human | NM_001123066.3 | N/A | ATGGCTGAGCCCCGCCAGGAGTTCGAAG | 909 |
| MAPT | | | TGATGGAAGATCACGCTGGGACGTACGG | |
| RefSeq | | | GTTGGGGGACAGGAAAGATCAGGGGGGC | |
| | | | TACACCATGCACCAAGACCAAGAGGGTG | |
| | | | ACACGGACGCTGGCCTGAAAGAATCTCC | |
| | | | CCTGCAGACCCCCACTGAGGACGGATCT | |
| | | | GAGGAACCGGGCTCTGAAACCTCTGATG | |
| | | | CTAAGAGCACTCCAACAGCGGAAGATGT | |
| | | | GACAGCACCCTTAGTGGATGAGGGAGCT | |
| | | | CCCGGCAAGCAGGCTGCCGCGCAGCCCC | |
| | | | ACACGGAGATCCCAGAAGGAACCACAGC | |
| | | | TGAAGAAGCAGGCATTGGAGACACCCCC | |
| | | | AGCCTGGAAGACGAAGCTGCTGGTCACG | |
| | | | TGACCCAAGAGCCTGAAAGTGGTAAGGT | |
| | | | GGTCCAGGAAGGCTTCCTCCGAGAGCCA | |
| | | | GGCCCCCCAGGTCTGAGCCACCAGCTCA | |
| | | | TGTCCGGCATGCCTGGGGCTCCCCTCCTG | |
| | | | CCTGAGGGCCCCAGAGAGGCCACACGCC | |
| | | | AACCTTCGGGGACAGGACCTGAGGACAC | |
| | | | AGAGGGCGGCCGCCACGCCCCTGAGCTG | |
| | | | CTCAAGCACCAGCTTCTAGGAGACCTGC | |
| | | | ACCAGGAGGGGCCGCCGCTGAAGGGGGC | |
| | | | AGGGGGCAAAGAGAGGCCGGGGAGCAA | |
| | | | GGAGGAGGTGGATGAAGACCGCGACGTC | |
| | | | GATGAGTCCTCCCCCCAAGACTCCCCTCC | |
| | | | CTCCAAGGCCTCCCCAGCCCAAGATGGG | |
| | | | CGGCCTCCCCAGACAGCCGCCAGAGAAG | |
| | | | CCACCAGCATCCCAGGCTTCCCAGCGGA | |
| | | | GGGTGCCATCCCCCTCCCTGTGGATTTCC | |
| | | | TCTCCAAAGTTTCCACAGAGATCCCAGCC | |
| | | | TCAGAGCCCGACGGGCCCAGTGTAGGGC | |
| | | | GGGCCAAAGGGCAGGATGCCCCCCTGGA | |
| | | | GTTCACGTTTCACGTGGAAATCACACCCA | |
| | | | ACGTGCAGAAGGAGCAGGCGCACTCGGA | |
| | | | GGAGCATTTGGGAAGGGCTGCATTTCCA | |
| | | | GGGGCCCCTGGAGAGGGGCCAGAGGCCC | |
| | | | GGGGCCCCTCTTTGGGAGAGGACACAAA | |
| | | | AGAGGCTGACCTTCCAGAGCCCTCTGAA | |
| | | | AAGCAGCCTGCTGCTGCTCCGCGGGGGA | |
| | | | AGCCCGTCAGCCGGGTCCCTCAACTCAA | |
| | | | AGCTCGCATGGTCAGTAAAAGCAAAGAC | |
| | | | GGGACTGGAAGCGATGACAAAAAAGCCA | |
| | | | AGACATCCACACGTTCCTCTGCTAAAACC | |
| | | | TTGAAAAATAGGCCTTGCCTTAGCCCCAA | |
| | | | ACACCCCACTCCTGGTAGCTCAGACCCTC | |
| | | | TGATCCAACCCTCCAGCCCTGCTGTGTGC | |
| | | | CCAGAGCCACCTTCCTCTCCTAAATACGT | |
| | | | CTCTTCTGTCACTTCCCGAACTGGCAGTT | |
| | | | CTGGAGCAAAGGAGATGAAACTCAAGGG | |
| | | | GGCTGATGGTAAAACGAAGATCGCCACA | |
| | | | CCGCGGGGAGCAGCCCCTCCAGGCCAGA | |
| | | | AGGGCCAGGCCAACGCCACCAGGATTCC | |
| | | | AGCAAAAACCCCGCCCGCTCCAAAGACA | |
| | | | CCACCCAGCTCTGCGACTAAGCAAGTCC | |
| | | | AGAGAAGACCACCCCCTGCAGGGCCCAG | |
| | | | ATCTGAGAGAGGTGAACCTCCAAAATCA | |
| | | | GGGGATCGCAGCGGCTACAGCAGCCCCG | |
| | | | GCTCCCCAGGCACTCCCGGCAGCCGCTC | |
| | | | CCGCACCCCGTCCCTTCCAACCCCACCCA | |
| | | | CCCGGGAGCCCAAGAAGGTGGCAGTGGT | |
| | | | CCGTACTCCACCCAAGTCGCCGTCTTCCG | |
| | | | CCAAGAGCCGCCTGCAGACAGCCCCCGT | |
| | | | GCCCATGCCAGACCTGAAGAATGTCAAG | |
| | | | TCCAAGATCGGCTCCACTGAGAACCTGA | |
| | | | AGCACCAGCCGGGAGGCGGGAAGGTGCA | |
| | | | GATAATTAATAAGAAGCTGGATCTTAGC | |
| | | | AACGTCCAGTCCAAGTGTGGCTCAAAGG | |
| | | | ATAATATCAAACACGTCCCGGGAGGCGG | |
| | | | CAGTGTGCAAATAGTCTACAAACCAGTT | |
| | | | GACCTGAGCAAGGTGACCTCCAAGTGTG | |
| | | | GCTCATTAGGCAACATCCATCATAAACC | |
| | | | AGGAGGTGGCCAGGTGGAAGTAAAATCT | |
| | | | GAGAAGCTTGACTTCAAGGACAGAGTCC | |
| | | | AGTCGAAGATTGGGTCCCTGGACAATAT | |
| | | | CACCCACGTCCCTGGCGGAGGAAATAAA | |
| | | | AAGATTGAAACCCACAAGCTGACCTTCC | |
| | | | GCGAGAACGCCAAAGCCAAGACAGACCA | |
| | | | CGGGGCGGAGATCGTGTACAAGTCGCCA | |
| | | | GTGGTGTCTGGGGACACGTCTCCACGGC | |
| | | | ATCTCAGCAATGTCTCCTCCACCGGCAGC | |
| | | | ATCGACATGGTAGACTCGCCCCAGCTCG | |
| | | | CCACGCTAGCTGACGAGGTGTCTGCCTCC | |
| | | | CTGGCCAAGCAGGGTTTGTGA | |
| Mouse | NM_001038609 | N/A | ATGGCTGACCCTCGCCAGGAGTTTGACA | 910 |
| MAPT | | | CAATGGAAGACCATGCTGGAGATTACAC | |
| RefSeq | | | TCTGCTCCAAGACCAAGAAGGAGACATG | |
| | | | GACCATGGCTTAAAAGAGTCTCCCCCAC | |
| | | | AGCCCCCCGCCGATGATGGAGCGGAGGA | |
| | | | ACCAGGGTCGGAGACCTCCGATGCTAAG | |
| | | | AGCACTCCAACTGCTGAAGACGTGACTG | |
| | | | CGCCCCTAGTGGATGAGAGAGCTCCCGA | |
| | | | CAAGCAGGCCGCTGCCCAGCCCCACACG | |
| | | | GAGATCCCAGAAGGAATTACAGCCGAAG | |
| | | | AAGCAGGCATCGGAGACACCCCGAACCA | |
| | | | GGAGGACCAAGCCGCTGGGCATGTGACT | |
| | | | CAAGCTCGTGTGGCCAGCAAAGACAGGA | |
| | | | CAGGAAATGACGAGAAGAAAGCCAAGG | |
| | | | GCGCTGATGGCAAAACCGGGGCGAAGAT | |
| | | | CGCCACACCTCGGGGAGCAGCCTCTCCG | |
| | | | GCCCAGAAGGGCACGTCCAACGCCACCA | |
| | | | GGATCCCGGCCAAGACCACGCCCAGCCC | |
| | | | TAAGACTCCTCCAGGGTCAGGTGAACCA | |
| | | | CCAAAATCCGGAGAACGAAGCGGCTACA | |
| | | | GCAGCCCCGGCTCTCCCGGAACGCCTGG | |
| | | | CAGTCGCTCGCGCACCCCATCCCTACCAA | |
| | | | CACCGCCCACCCGGGAGCCCAAGAAGGT | |
| | | | GGCAGTGGTCCGCACTCCCCCTAAGTCAC | |
| | | | CATCAGCTAGTAAGAGCCGCCTGCAGAC | |
| | | | TGCCCCTGTGCCCATGCCAGACCTAAAG | |
| | | | AATGTCAGGTCGAAGATTGGCTCTACTG | |
| | | | AGAACCTGAAGCACCAGCCAGGAGGTGG | |
| | | | CAAGGTGCAGATAATTAATAAGAAGCTG | |
| | | | GATCTTAGCAACGTCCAGTCCAAGTGTG | |
| | | | GCTCGAAGGATAATATCAAACACGTCCC | |
| | | | GGGTGGAGGCAGTGTGCAAATAGTCTAC | |
| | | | AAGCCGGTGGACCTGAGCAAAGTGACCT | |
| | | | CCAAGTGTGGCTCGTTAGGGAACATCCA | |
| | | | TCACAAGCCAGGAGGTGGCCAGGTGGAA | |
| | | | GTAAAATCAGAGAAGCTGGACTTCAAGG | |
| | | | ACAGAGTCCAGTCGAAGATTGGCTCCTT | |
| | | | GGATAATATCACCCACGTCCCTGGAGGA | |
| | | | GGGAATAAGAAGATTGAAACCCACAAGC | |
| | | | TGACCTTCAGGGAGAATGCCAAAGCCAA | |
| | | | GACAGACCATGGAGCAGAAATTGTGTAT | |
| | | | AAGTCACCCGTGGTGTCTGGGGACACAT | |
| | | | CTCCACGGCACCTCAGCAATGTGTCTTCC | |
| | | | ACGGGCAGCATCGACATGGTGGACTCAC | |
| | | | CACAGCTTGCCACACTAGCCGATGAAGT | |
| | | | GTCTGCTTCCTTGGCCAAGCAGGGTTTGT | |
| | | | GA | |
| Monkey | XM_005584531 | N/A | ATGGCTGAGCCCCGCCAGGAGTTCGATG | 911 |
| MAPT | | | TGATGGAAGATCACGCTGGGACGTACGG | |
| RefSEQ | | | GTTGGGGGACAGGAAAGATCAAGAGGGC | |
| | | | TACACCATGCTCCAAGACCAAGAGGGTG | |
| | | | ACACGGACGCTGGCCTGAAAGAATCTCC | |
| | | | CCTGCAGACCCCCGCTGAGGATGGATCT | |
| | | | GAGGAACTGGGCTCTGAAACCTCTGATG | |
| | | | CTAAGAGCACTCCAACGGCGGAAGCTGA | |
| | | | GGAAGCAGGCATCGGAGACACCCCCAGC | |
| | | | CTGGAAGACGAAGCTGCTGGTCACGTGA | |
| | | | CCCAAGAGGAGTTGAGAGTTCCGGGCCA | |
| | | | GCAGAGGAAGGCACCTGAAAGGCCCCTG | |
| | | | GCCAATGAGATTAGTGCTCACGTCCAGC | |
| | | | CTGGACCCTGCAAAGAGGCCTCTGGGGT | |
| | | | CTCTGGGCTGTGCATGGGGGAGAAAGAG | |
| | | | CCAGAAGCTCCCATCCCACTGACCGCGA | |
| | | | GCCTTCCTCAGCACCGTCCCATTTGCTCA | |
| | | | GCGCCTCCTCCAACAGGAGGCCCTCGAG | |
| | | | AGCCCTCCCAGGAGTGGGGACGAAAAGG | |
| | | | TGGGGACTGGGCCGAGAAGGGTCCGACC | |
| | | | TTTCCGAAGTCCGCCACCCCTGCGTATCT | |
| | | | CCACACAGAGCCTGAAAGTGGTAAGGTG | |
| | | | GTCCAGGAAGTCTTCCTCGGAGAGCCAG | |
| | | | GCCCCCCAGGTCTGAGCCACCAGCTCGT | |
| | | | GTCCAGCATGCCTGGGGCTCCCCTCCTGC | |
| | | | CTGAGGGCCCCAGAGAGGCCACACGCCA | |
| | | | GCCTTCAGGGACAGGACCTGAGGACACA | |
| | | | GAGGGTGGCCAACACGCCCCTGAGCTGC | |
| | | | TCAAGCACCAGCTTCTGGGAGACCTGCA | |
| | | | CCAGGAGGGGCCGCCACTGAAGGGAGCC | |
| | | | GGGGGCAAAGAGAGGCTGGGGAGCAAG | |
| | | | GAGGAGGTGGATGAAGACCGCGACGTCG | |
| | | | ATGAGTCCTCCCCGCAAGACTCCCCTCCA | |
| | | | TCCAGGGTCTCCCCAGTCCAAGATGGGC | |
| | | | AGCCTCCCCAGACAGCCGCCAGAGAAGC | |
| | | | CACCAGCGTCCCAGGCTTCCCAGCGGAG | |
| | | | GGTGCCATTGCCCTCCCTGTGGATTTCCT | |
| | | | CTCCAGAGTTTCCACAGAGATCCCAGCCT | |
| | | | CTGAGCCCGAGGGGCCCAGTGCAGGGTG | |
| | | | GGCTGAAGGGCAGGACATGCCCCCTGAG | |
| | | | TTCACGTTCCACGTGGAAATCACACCCAA | |
| | | | CGTGCAGAAGGAGCAGGCGCACCCGGAG | |
| | | | GAGGATTCGGGAAGGGCTGCATTTCCAG | |
| | | | GGGCTCCTGGAGAGGAGCCAGAGGCCCG | |
| | | | GGGCCCCTCTTTGGGAGAGGACACAAAA | |
| | | | GAGGCTGAGCTTCCAGAGCCCACTGAAA | |
| | | | AGCAGCCTGCTGCTGCTCCGCGGGGAAA | |
| | | | ACCCGTCAGCCGGGTCCCTCAACTCAAA | |
| | | | GCTCGCATGGTCAGTAAAAGCAAAGACG | |
| | | | GGACTGGAAGCGATGACAAAAAAGCCAA | |
| | | | GACATCCACACGTTCCTCTGCTAAAACCT | |
| | | | TGAAAAATAGGCCTTGCCTTAGCCCCAA | |
| | | | ACACCCCACTCCTGGTAGCTCAGACCCTC | |
| | | | TGATCCAACCCTCCAGCCCTGCCGTGTGC | |
| | | | CCAGAGCCACCTTCCTCTCCTAAATACGT | |
| | | | CTCTTCTGTCACTCCCCGAACTGGCAGTT | |
| | | | CTGGAGCAAAGGAGATGAAACTCAAGGG | |
| | | | GGCTGATGGGAAAACGAAGATCGCCACA | |
| | | | CCCCGGGGAGCGGCCCCTCCAGGCCAGA | |
| | | | AGGGCCAAGCCAACGCCACCAGGATTCC | |
| | | | AGCAAAAACCCCGCCCGCCCCAAAGACA | |
| | | | CCACCCAGCTCTGCGACCAAGCAAGTGC | |
| | | | AGAGAAAACCACCCCCTGCAGAGCCCAC | |
| | | | ATCTGAGAGAGGTGAACCTCCAAAATCA | |
| | | | GGGGATCGCAGTGGCTACAGCAGCCCCG | |
| | | | GCTCCCCGGGCACTCCCGGCAGCCGCTC | |
| | | | CCGCACCCCGTCCCTTCCAACCCCTCCAG | |
| | | | CCCGGGAGCCCAAGAAGGTGGCGGTGGT | |
| | | | CCGTACTCCACCTAAGTCGCCGTCTTCCG | |
| | | | CCAAGAGCCGCCTGCAGACAGCCCCCGT | |
| | | | GCCCATGCCAGACCTGAAGAACGTCAAG | |
| | | | TCCAAGATCGGCTCCACCGAGAACCTGA | |
| | | | AGCACCAGCCGGGAGGCGGGAAGGTGCA | |
| | | | GATAATTAATAAGAAGCTGGATCTTAGC | |
| | | | AACGTCCAGTCCAAGTGTGGCTCAAAGG | |
| | | | ATAATATCAAACACGTCCCGGGAGGCGG | |
| | | | CAGTGTGCAAATAGTCTACAAACCAGTT | |
| | | | GACCTGAGCAAGGTGACCTCCAAGTGTG | |
| | | | GCTCATTAGGCAACATCCATCATAAACC | |
| | | | AGGAGGTGGCCAGGTGGAAGTAAAATCT | |
| | | | GAGAAGCTGGACTTCAAGGACAGAGTGC | |
| | | | AGTCGAAGATCGGGTCCCTGGACAATAT | |
| | | | CACCCATGTCCCTGGCGGAGGAAATAAA | |
| | | | AAGATTGAAACCCACAAGCTGACCTTCC | |
| | | | GCGAGAACGCCAAAGCCAAGACAGACCA | |
| | | | CGGGGCGGAAATCGTGTACAAGTCGCCG | |
| | | | GTGGTGTCTGGGGACACGTCTCCACGGC | |
| | | | ACCTCAGCAATGTCTCCTCCACCGGCAGC | |
| | | | ATCGACATGGTAGACTCGCCCCAGCTCG | |
| | | | CCACGCTAGCCGACGAGGTGTCTGCCTCC | |
| | | | CTGGCCAAGCAGGGTTTGTGA | |
| MAPT- | 19 mer | 2141-2218- | GAGAACCUGAAGCACCAGC | 912 |
| 2141 | sense | 966 (Hs- | | |
| | strand | Mf-Mm) | | |
| MAPT- | 19 mer | 2142-2219- | AGAACCUGAAGCACCAGCC | 913 |
| 2142 | sense | 967 (Hs- | | |
| | strand | Mf-Mm) | | |
| MAPT- | 19 mer | 2303-2380- | GUGACCUCCAAGUGUGGCU | 914 |
| 2303 | sense | 1128 (Hs- | | |
| | strand | Mf-Mm) | | |
| MAPT- | 19 mer | 2347-2424- | AGGAGGUGGCCAGGUGGAA | 915 |
| 2347 | sense | 1172 (Hs- | | |
| | strand | Mf-Mm) | | |
| MAPT- | 19 mer | 2349-2426- | GAGGUGGCCAGGUGGAAGU | 916 |
| 2349 | sense | 1174 (Hs- | | |
| | strand | Mf-Mm) | | |
| MAPT- | 19 mer | 2350-2427- | AGGUGGCCAGGUGGAAGUA | 917 |
| 2350 | sense | 1175 (Hs- | | |
| | strand | Mf-Mm) | | |
| MAPT- | 19 mer | 2351-2428- | GGUGGCCAGGUGGAAGUAA | 918 |
| 2351 | sense | 1176 (Hs- | | |
| | strand | Mf-Mm) | | |
| MAPT- | 19 mer | 2352-2429- | GUGGCCAGGUGGAAGUAAA | 919 |
| 2352 | sense | 1177 (Hs- | | |
| | strand | Mf-Mm) | | |
| MAPT- | 19 mer | 2353-2430- | UGGCCAGGUGGAAGUAAAA | 920 |
| 2353 | sense | 1178 (Hs- | | |
| | strand | Mf-Mm) | | |
| MAPT- | 19 mer | 2354-2431- | GGCCAGGUGGAAGUAAAAU | 921 |
| 2354 | sense | 1179 (Hs- | | |
| | strand | Mf-Mm) | | |
| MAPT- | 19 mer | 2355-2432- | GCCAGGUGGAAGUAAAAUC | 922 |
| 2355 | sense | 1180 (Hs- | | |
| | strand | Mf-Mm) | | |
| MAPT- | 19 mer | 2459-2536- | AAGAUUGAAACCCACAAGC | 923 |
| 2459 | sense | 1284 (Hs- | | |
| | strand | Mf-Mm) | | |
| MAPT- | 19 mer | 2460-2537- | AGAUUGAAACCCACAAGCU | 924 |
| 2460 | sense | 1285 (Hs- | | |
| | strand | Mf-Mm) | | |
| MAPT- | 19 mer | 2461-2538- | GAUUGAAACCCACAAGCUG | 925 |
| 2461 | sense | 1286 (Hs- | | |
| | strand | Mf-Mm) | | |
| MAPT- | 19 mer | 2462-2539- | AUUGAAACCCACAAGCUGA | 926 |
| 2462 | sense | 1287 (Hs- | | |
| | strand | Mf-Mm) | | |
| MAPT- | 19 mer | 2463-2540- | UUGAAACCCACAAGCUGAC | 927 |
| 2463 | sense | 1288 (Hs- | | |
| | strand | Mf-Mm) | | |
| MAPT- | 19 mer | 2464-2541- | UGAAACCCACAAGCUGACC | 928 |
| 2464 | sense | 1289 (Hs- | | |
| | strand | Mf-Mm) | | |
| MAPT- | 19 mer | 2465-2542- | GAAACCCACAAGCUGACCU | 929 |
| 2465 | sense | 1290 (Hs- | | |
| | strand | Mf-Mm) | | |
| MAPT- | 19 mer | 2466-2543- | AAACCCACAAGCUGACCUU | 930 |
| 2466 | sense | 1291 (Hs- | | |
| | strand | Mf-Mm) | | |
| MAPT- | 19 mer | 2467-2544- | AACCCACAAGCUGACCUUC | 931 |
| 2467 | sense | 1292 (Hs- | | |
| | strand | Mf-Mm) | | |
| MAPT- | 19 mer | 2495-2572- | GCCAAAGCCAAGACAGACC | 932 |
| 2495 | sense | 1320 (Hs- | | |
| | strand | Mf-Mm) | | |
| MAPT- | 19 mer | 2496-2573- | CCAAAGCCAAGACAGACCA | 933 |
| 2496 | sense | 1321 (Hs- | | |
| | strand | Mf-Mm) | | |
| MAPT- | 19 mer | 3686-3758- | UCUUUGUAAGGACUUGUGC | 934 |
| 3686 | sense | 2505 (Hs- | | |
| | strand | Mf-Mm) | | |
| MAPT- | 19 mer | 3687-3759- | CUUUGUAAGGACUUGUGCC | 935 |
| 3687 | sense | 2506 (Hs- | | |
| | strand | Mf-Mm) | | |
| MAPT- | 19 mer | 3688-3760- | UUUGUAAGGACUUGUGCCU | 936 |
| 3688 | sense | 2507 (Hs- | | |
| | strand | Mf-Mm) | | |
| MAPT- | 19 mer | 3691-3763- | GUAAGGACUUGUGCCUCUU | 937 |
| 3691 | sense | 2510 (Hs- | | |
| | strand | Mf-Mm) | | |
| MAPT- | 19 mer | 3692-3764- | UAAGGACUUGUGCCUCUUG | 938 |
| 3692 | sense | 2511 (Hs- | | |
| | strand | Mf-Mm) | | |
| MAPT- | 19 mer | 3693-3765- | AAGGACUUGUGCCUCUUGG | 939 |
| 3693 | sense | 2512 (Hs- | | |
| | strand | Mf-Mm) | | |
| MAPT- | 19 mer | 4534-4605- | GUUGUAGUUGGAUUUGUCU | 940 |
| 4534 | sense | 3332 (Hs- | | |
| | strand | Mf-Mm) | | |
| MAPT- | 19 mer | 4535-4606- | UUGUAGUUGGAUUUGUCUG | 941 |
| 4535 | sense | 3333 (Hs- | | |
| | strand | Mf-Mm) | | |
| MAPT- | 19 mer | 4536-4607- | UGUAGUUGGAUUUGUCUGU | 942 |
| 4536 | sense | 3334 (Hs- | | |
| | strand | Mf-Mm) | | |
| MAPT- | 19 mer | 4537-4608- | GUAGUUGGAUUUGUCUGUU | 943 |
| 4537 | sense | 3335 (Hs- | | |
| | strand | Mf-Mm) | | |
| MAPT- | 19 mer | 4538-4609- | UAGUUGGAUUUGUCUGUUU | 944 |
| 4538 | sense | 3336 (Hs- | | |
| | strand | Mf-Mm) | | |
| MAPT- | 19 mer | 4566-4637- | UUCACCAGAGUGACUAUGA | 945 |
| 4566 | sense | 3362 (Hs- | | |
| | strand | Mf-Mm) | | |
| MAPT- | 19 mer | 4567-4638- | UCACCAGAGUGACUAUGAU | 946 |
| 4567 | sense | 3363 (Hs- | | |
| | strand | Mf-Mm) | | |
| MAPT- | 19 mer | 4568-4639- | CACCAGAGUGACUAUGAUA | 947 |
| 4568 | sense | 3364 (Hs- | | |
| | strand | Mf-Mm) | | |
| MAPT- | 19 mer | 4569-4640- | ACCAGAGUGACUAUGAUAG | 948 |
| 4569 | sense | 3365 (Hs- | | |
| | strand | Mf-Mm) | | |
| MAPT- | 19 mer | 4570-4641- | CCAGAGUGACUAUGAUAGU | 949 |
| 4570 | sense | 3366 (Hs- | | |
| | strand | Mf-Mm) | | |
| MAPT- | 19 mer | 4571-4642- | CAGAGUGACUAUGAUAGUG | 950 |
| 4571 | sense | 3367 (Hs- | | |
| | strand | Mf-Mm) | | |
| MAPT- | 19 mer | 4572-4643- | AGAGUGACUAUGAUAGUGA | 951 |
| 4572 | sense | 3368 (Hs- | | |
| | strand | Mf-Mm) | | |
| MAPT- | 19 mer | 4573-4644- | GAGUGACUAUGAUAGUGAA | 952 |
| 4573 | sense | 3369 (Hs- | | |
| | strand | Mf-Mm) | | |
| MAPT- | 19 mer | 4574-4645- | AGUGACUAUGAUAGUGAAA | 953 |
| 4574 | sense | 3370 (Hs- | | |
| | strand | Mf-Mm) | | |
| MAPT- | 19 mer | 4575-4646- | GUGACUAUGAUAGUGAAAA | 954 |
| 4575 | sense | 3371 (Hs- | | |
| | strand | Mf-Mm) | | |
| MAPT- | 19 mer | 4576-4647- | UGACUAUGAUAGUGAAAAG | 955 |
| 4576 | sense | 3372 (Hs- | | |
| | strand | Mf-Mm) | | |
| MAPT- | 19 mer | 4577-4648- | GACUAUGAUAGUGAAAAGA | 956 |
| 4577 | sense | 3373 (Hs- | | |
| | strand | Mf-Mm) | | |
| MAPT- | 19 mer | 4578-4649- | ACUAUGAUAGUGAAAAGAA | 957 |
| 4578 | sense | 3374 (Hs- | | |
| | strand | Mf-Mm) | | |
| MAPT- | 19 mer | 4579-4650- | CUAUGAUAGUGAAAAGAAA | 958 |
| 4579 | sense | 3375 (Hs- | | |
| | strand | Mf-Mm) | | |
| MAPT- | 19 mer | 4580-4651- | UAUGAUAGUGAAAAGAAAA | 959 |
| 4580 | sense | 3376 (Hs- | | |
| | strand | Mf-Mm) | | |
| MAPT- | 19 mer | 4605-4677- | AAAAAAAAGGACGCAUGUA | 960 |
| 4605 | sense | 3439 (Hs- | | |
| | strand | Mf-Mm) | | |
| MAPT- | 19 mer | 4606-4678- | AAAAAAAGGACGCAUGUAU | 961 |
| 4606 | sense | 3440 (Hs- | | |
| | strand | Mf-Mm) | | |
| MAPT- | 19 mer | 4607-4679- | AAAAAAGGACGCAUGUAUC | 962 |
| 4607 | sense | 3441 (Hs- | | |
| | strand | Mf-Mm) | | |
| MAPT- | 19 mer | 4608-4680- | AAAAAGGACGCAUGUAUCU | 963 |
| 4608 | sense | 3442 (Hs- | | |
| | strand | Mf-Mm) | | |
| MAPT- | 19 mer | 4609-4681- | AAAAGGACGCAUGUAUCUU | 964 |
| 4609 | sense | 3443 (Hs- | | |
| | strand | Mf-Mm) | | |
| MAPT- | 19 mer | 4610-4682- | AAAGGACGCAUGUAUCUUG | 965 |
| 4610 | sense | 3444 (Hs- | | |
| | strand | Mf-Mm) | | |
| MAPT- | 19 mer | 4611-4683- | AAGGACGCAUGUAUCUUGA | 966 |
| 4611 | sense | 3445 (Hs- | | |
| | strand | Mf-Mm) | | |
| MAPT- | 19 mer | 4612-4684- | AGGACGCAUGUAUCUUGAA | 967 |
| 4612 | sense | 3446 (Hs- | | |
| | strand | Mf-Mm) | | |
| MAPT- | 19 mer | 4613-4685- | GGACGCAUGUAUCUUGAAA | 968 |
| 4613 | sense | 3447 (Hs- | | |
| | strand | Mf-Mm) | | |
| MAPT- | 19 mer | 4614-4686- | GACGCAUGUAUCUUGAAAU | 969 |
| 4614 | sense | 3448 (Hs- | | |
| | strand | Mf-Mm) | | |
| MAPT- | 19 mer | 5969-6024- | UCACUUUAUCAAUAGUUCC | 970 |
| 5969 | sense | 4540 (Hs- | | |
| | strand | Mf-Mm) | | |
| MAPT- | 19 mer | 5970-6025- | CACUUUAUCAAUAGUUCCA | 971 |
| 5970 | sense | 4541 (Hs- | | |
| | strand | Mf-Mm) | | |
| MAPT- | 19 mer | 5971-6026- | ACUUUAUCAAUAGUUCCAU | 972 |
| 5971 | sense | 4542 (Hs- | | |
| | strand | Mf-Mm) | | |
| MAPT- | 19 mer | 5972-6027- | CUUUAUCAAUAGUUCCAUU | 973 |
| 5972 | sense | 4543 (Hs- | | |
| | strand | Mf-Mm) | | |
| MAPT- | 19 mer | 5973-6028- | UUUAUCAAUAGUUCCAUUU | 974 |
| 5973 | sense | 4544 (Hs- | | |
| | strand | Mf-Mm) | | |
| MAPT- | 19 mer | 5974-6029- | UUAUCAAUAGUUCCAUUUA | 975 |
| 5974 | sense | 4545 (Hs- | | |
| | strand | Mf-Mm) | | |
| MAPT- | 19 mer | 5975-6030- | UAUCAAUAGUUCCAUUUAA | 976 |
| 5975 | sense | 4546 (Hs- | | |
| | strand | Mf-Mm) | | |
| MAPT- | 19 mer | 5976-6031- | AUCAAUAGUUCCAUUUAAA | 977 |
| 5976 | sense | 4547 (Hs- | | |
| | strand | Mf-Mm) | | |
| MAPT- | 19 mer | 5977-6032- | UCAAUAGUUCCAUUUAAAU | 978 |
| 5977 | sense | 4548 (Hs- | | |
| | strand | Mf-Mm) | | |
| MAPT- | 19 mer | 5978-6033- | CAAUAGUUCCAUUUAAAUU | 979 |
| 5978 | sense | 4549 (Hs- | | |
| | strand | Mf-Mm) | | |
| MAPT- | 19 mer | 5979-6034- | AAUAGUUCCAUUUAAAUUG | 980 |
| 5979 | sense | 4550 (Hs- | | |
| | strand | Mf-Mm) | | |
| MAPT- | 19 mer | 5980-6035- | AUAGUUCCAUUUAAAUUGA | 981 |
| 5980 | sense | 4551 (Hs- | | |
| | strand | Mf-Mm) | | |
| MAPT- | 19 mer | 5981-6036- | UAGUUCCAUUUAAAUUGAC | 982 |
| 5981 | sense | 4552 (Hs- | | |
| | strand | Mf-Mm) | | |
| MAPT- | 19 mer | 5982-6037- | AGUUCCAUUUAAAUUGACU | 983 |
| 5982 | sense | 4553 (Hs- | | |
| | strand | Mf-Mm) | | |
| MAPT- | 19 mer | 5983-6038- | GUUCCAUUUAAAUUGACUU | 984 |
| 5983 | sense | 4554 (Hs- | | |
| | strand | Mf-Mm) | | |
| MAPT- | 19 mer | 5984-6039- | UUCCAUUUAAAUUGACUUC | 985 |
| 5984 | sense | 4555 (Hs- | | |
| | strand | Mf-Mm) | | |
| MAPT- | 19 mer | 5985-6040- | UCCAUUUAAAUUGACUUCA | 986 |
| 5985 | sense | 4556 (Hs- | | |
| | strand | Mf-Mm) | | |
| MAPT- | 19 mer | 6662-6723- | CUUGCAAGUCCCAUGAUUU | 987 |
| 6662 | sense | 5230 (Hs- | | |
| | strand | Mf-Mm) | | |
| MAPT- | 19 mer | 6663-6724- | UUGCAAGUCCCAUGAUUUC | 988 |
| 6663 | sense | 5231 (Hs- | | |
| | strand | Mf-Mm) | | |
| MAPT- | 19 mer | 6664-6725- | UGCAAGUCCCAUGAUUUCU | 989 |
| 6664 | sense | 5232 (Hs- | | |
| | strand | Mf-Mm) | | |
| MAPT- | 19 mer | 6665-6726- | GCAAGUCCCAUGAUUUCUU | 990 |
| 6665 | sense | 5233 (Hs- | | |
| | strand | Mf-Mm) | | |
| MAPT- | 19 mer | 6800-6861- | GUAAAAGUGAAUUUGGAAA | 991 |
| 6800 | sense | 5365 (Hs- | | |
| | strand | Mf-Mm) | | |
| MAPT- | 19 mer | 6801-6862- | UAAAAGUGAAUUUGGAAAU | 992 |
| 6801 | sense | 5366 (Hs- | | |
| | strand | Mf-Mm) | | |
| MAPT- | 19 mer | 6802-6863- | AAAAGUGAAUUUGGAAAUA | 993 |
| 6802 | sense | 5367 (Hs- | | |
| | strand | Mf-Mm) | | |
| MAPT- | 19 mer | 6803-6864- | AAAGUGAAUUUGGAAAUAA | 994 |
| 6803 | sense | 5368 (Hs- | | |
| | strand | Mf-Mm) | | |
| MAPT- | 19 mer | 6804-6865- | AAGUGAAUUUGGAAAUAAA | 995 |
| 6804 | sense | 5369 (Hs- | | |
| | strand | Mf-Mm) | | |
| MAPT- | 19 mer | 6805-6866- | AGUGAAUUUGGAAAUAAAG | 996 |
| 6805 | sense | 5370 (Hs- | | |
| | strand | Mf-Mm) | | |
| MAPT- | 19 mer | 6806-6867- | GUGAAUUUGGAAAUAAAGU | 997 |
| 6806 | sense | 5371 (Hs- | | |
| | strand | Mf-Mm) | | |
| MAPT- | 19 mer | 6807-6868- | UGAAUUUGGAAAUAAAGUU | 998 |
| 6807 | sense | 5372 (Hs- | | |
| | strand | Mf-Mm) | | |
| MAPT- | 19 mer | 6808-6869- | GAAUUUGGAAAUAAAGUUA | 999 |
| 6808 | sense | 5373 (Hs- | | |
| | strand | Mf-Mm) | | |
| MAPT- | 19 mer | 6809-6870- | AAUUUGGAAAUAAAGUUAU | 1000 |
| 6809 | sense | 5374 (Hs- | | |
| | strand | Mf-Mm) | | |
| MAPT- | 19 mer | 6810-6871- | AUUUGGAAAUAAAGUUAUU | 1001 |
| 6810 | sense | 5375 (Hs- | | |
| | strand | Mf-Mm) | | |
| MAPT- | 19 mer | 6811-6872- | UUUGGAAAUAAAGUUAUUA | 1002 |
| 6811 | sense | 5376 (Hs- | | |
| | strand | Mf-Mm) | | |
| MAPT- | 19 mer | 6812-6873- | UUGGAAAUAAAGUUAUUAC | 1003 |
| 6812 | sense | 5377 (Hs- | | |
| | strand | Mf-Mm) | | |
| MAPT- | 19 mer | 6813-6874- | UGGAAAUAAAGUUAUUACU | 1004 |
| 6813 | sense | 5378 (Hs- | | |
| | strand | Mf-Mm) | | |
| MAPT- | 19 mer | 6814-6875- | GGAAAUAAAGUUAUUACUC | 1005 |
| 6814 | sense | 5379 (Hs- | | |
| | strand | Mf-Mm) | | |
| MAPT- | 19 mer | 6815-6876- | GAAAUAAAGUUAUUACUCU | 1006 |
| 6815 | sense | 5380 (Hs- | | |
| | strand | Mf-Mm) | | |
| MAPT- | 19 mer | 6816-6877- | AAAUAAAGUUAUUACUCUG | 1007 |
| 6816 | sense | 5381 (Hs- | | |
| | strand | Mf-Mm) | | |
| MAPT- | 19 mer | 363 (Hs) | AGGAGUUCGAAGUGAUGGA | 1008 |
| 363 | sense | | | |
| | strand | | | |
| MAPT- | 19 mer | 364 (Hs) | GGAGUUCGAAGUGAUGGAA | 1009 |
| 364 | sense | | | |
| | strand | | | |
| MAPT- | 19 mer | 365 (Hs) | GAGUUCGAAGUGAUGGAAG | 1010 |
| 365 | sense | | | |
| | strand | | | |
| MAPT- | 19 mer | 367 (Hs) | GUUCGAAGUGAUGGAAGAU | 1011 |
| 367 | sense | | | |
| | strand | | | |
| MAPT- | 19 mer | 369 (Hs) | UCGAAGUGAUGGAAGAUCA | 1012 |
| 369 | sense | | | |
| | strand | | | |
| MAPT- | 19 mer | 374-226 | GUGAUGGAAGAUCACGCUG | 1013 |
| 374 | sense | (Hs-Mf) | | |
| | strand | | | |
| MAPT- | 19 mer | 395-247 | ACGUACGGGUUGGGGGACA | 1014 |
| 395 | sense | (Hs-Mf) | | |
| | strand | | | |
| MAPT- | 19 mer | 400-252 | CGGGUUGGGGGACAGGAAA | 1015 |
| 400 | sense | (Hs-Mf) | | |
| | strand | | | |
| MAPT- | 19 mer | 443-295 | CAAGACCAAGAGGGUGACA | 1016 |
| 443 | sense | (Hs-Mf) | | |
| | strand | | | |
| MAPT- | 19 mer | 688-453 | GGAAGACGAAGCUGCUGGU | 1017 |
| 688 | sense | (Hs-Mf) | | |
| | strand | | | |
| MAPT- | 19 mer | 689-454 | GAAGACGAAGCUGCUGGUC | 1018 |
| 689 | sense | (Hs-Mf) | | |
| | strand | | | |
| MAPT- | 19 mer | 690-455 | AAGACGAAGCUGCUGGUCA | 1019 |
| 690 | sense | (Hs-Mf) | | |
| | strand | | | |
| MAPT- | 19 mer | 693-458 | ACGAAGCUGCUGGUCACGU | 1020 |
| 693 | sense | (Hs-Mf) | | |
| | strand | | | |
| MAPT- | 19 mer | 695-460 | GAAGCUGCUGGUCACGUGA | 1021 |
| 695 | sense | (Hs-Mf) | | |
| | strand | | | |
| MAPT- | 19 mer | 696-461 | AAGCUGCUGGUCACGUGAC | 1022 |
| 696 | sense | (Hs-Mf) | | |
| | strand | | | |
| MAPT- | 19 mer | 1475-1552 | CGCAUGGUCAGUAAAAGCA | 1023 |
| 1475 | sense | (Hs-Mf) | | |
| | strand | | | |
| MAPT- | 19 mer | 1476-1553 | GCAUGGUCAGUAAAAGCAA | 1024 |
| 1476 | sense | (Hs-Mf) | | |
| | strand | | | |
| MAPT- | 19 mer | 1479-1556 | UGGUCAGUAAAAGCAAAGA | 1025 |
| 1479 | sense | (Hs-Mf) | | |
| | strand | | | |
| MAPT- | 19 mer | 1480-1557 | GGUCAGUAAAAGCAAAGAC | 1026 |
| 1480 | sense | (Hs-Mf) | | |
| | strand | | | |
| MAPT- | 19 mer | 1481-1558 | GUCAGUAAAAGCAAAGACG | 1027 |
| 1481 | sense | (Hs-Mf) | | |
| | strand | | | |
| MAPT- | 19 mer | 1484-1561 | AGUAAAAGCAAAGACGGGA | 1028 |
| 1484 | sense | (Hs-Mf) | | |
| | strand | | | |
| MAPT- | 19 mer | 1485-1562 | GUAAAAGCAAAGACGGGAC | 1029 |
| 1485 | sense | (Hs-Mf) | | |
| | strand | | | |
| MAPT- | 19 mer | 1492-1569 | CAAAGACGGGACUGGAAGC | 1030 |
| 1492 | sense | (Hs-Mf) | | |
| | strand | | | |
| MAPT- | 19 mer | 1494-1571 | AAGACGGGACUGGAAGCGA | 1031 |
| 1494 | sense | (Hs-Mf) | | |
| | strand | | | |
| MAPT- | 19 mer | 1495-1572 | AGACGGGACUGGAAGCGAU | 1032 |
| 1495 | sense | (Hs-Mf) | | |
| | strand | | | |
| MAPT- | 19 mer | 1498-1575 | CGGGACUGGAAGCGAUGAC | 1033 |
| 1498 | sense | (Hs-Mf) | | |
| | strand | | | |
| MAPT- | 19 mer | 1499-1576 | GGGACUGGAAGCGAUGACA | 1034 |
| 1499 | sense | (Hs-Mf) | | |
| | strand | | | |
| MAPT- | 19 mer | 1500-1577 | GGACUGGAAGCGAUGACAA | 1035 |
| 1500 | sense | (Hs-Mf) | | |
| | strand | | | |
| MAPT- | 19 mer | 1502-1579 | ACUGGAAGCGAUGACAAAA | 1036 |
| 1502 | sense | (Hs-Mf) | | |
| | strand | | | |
| MAPT- | 19 mer | 1503-1580 | CUGGAAGCGAUGACAAAAA | 1037 |
| 1503 | sense | (Hs-Mf) | | |
| | strand | | | |
| MAPT- | 19 mer | 1504-1581 | UGGAAGCGAUGACAAAAAA | 1038 |
| 1504 | sense | (Hs-Mf) | | |
| | strand | | | |
| MAPT- | 19 mer | 1505-1582 | GGAAGCGAUGACAAAAAAG | 1039 |
| 1505 | sense | (Hs-Mf) | | |
| | strand | | | |
| MAPT- | 19 mer | 1506-1583 | GAAGCGAUGACAAAAAAGC | 1040 |
| 1506 | sense | (Hs-Mf) | | |
| | strand | | | |
| MAPT- | 19 mer | 1507-1584 | AAGCGAUGACAAAAAAGCC | 1041 |
| 1507 | sense | (Hs-Mf) | | |
| | strand | | | |
| MAPT- | 19 mer | 1508-1585 | AGCGAUGACAAAAAAGCCA | 1042 |
| 1508 | sense | (Hs-Mf) | | |
| | strand | | | |
| MAPT- | 19 mer | 1509-1586 | GCGAUGACAAAAAAGCCAA | 1043 |
| 1509 | sense | (Hs-Mf) | | |
| | strand | | | |
| MAPT- | 19 mer | 1733 (Hs) | GAUGGUAAAACGAAGAUCG | 1044 |
| 1733 | sense | | | |
| | strand | | | |
| MAPT- | 19 mer | 1796-1873 | AACGCCACCAGGAUUCCAG | 1045 |
| 1796 | sense | (Hs-Mf) | | |
| | strand | | | |
| MAPT- | 19 mer | 1835-1912 | AAGACACCACCCAGCUCUG | 1046 |
| 1835 | sense | (Hs-Mf) | | |
| | strand | | | |
| MAPT- | 19 mer | 1912-1989 | ACCUCCAAAAUCAGGGGAU | 1047 |
| 1912 | sense | (Hs-Mf) | | |
| | strand | | | |
| MAPT- | 19 mer | 2094-2171 | UGCCCAUGCCAGACCUGAA | 1048 |
| 2094 | sense | (Hs-Mf) | | |
| | strand | | | |
| MAPT- | 19 mer | 2096-2173 | CCCAUGCCAGACCUGAAGA | 1049 |
| 2096 | sense | (Hs-Mf) | | |
| | strand | | | |
| MAPT- | 19 mer | 2097-2174 | CCAUGCCAGACCUGAAGAA | 1050 |
| 2097 | sense | (Hs-Mf) | | |
| | strand | | | |
| MAPT- | 19 mer | 2098 (Hs) | CAUGCCAGACCUGAAGAAU | 1051 |
| 2098 | sense | | | |
| | strand | | | |
| MAPT- | 19 mer | 2105 (Hs) | GACCUGAAGAAUGUCAAGU | 1052 |
| 2105 | sense | | | |
| | strand | | | |
| MAPT- | 19 mer | 2106 (Hs) | ACCUGAAGAAUGUCAAGUC | 1053 |
| 2106 | sense | | | |
| | strand | | | |
| MAPT- | 19 mer | 2107 (Hs) | CCUGAAGAAUGUCAAGUCC | 1054 |
| 2107 | sense | | | |
| | strand | | | |
| MAPT- | 19 mer | 2108 (Hs) | CUGAAGAAUGUCAAGUCCA | 1055 |
| 2108 | sense | | | |
| | strand | | | |
| MAPT- | 19 mer | 2109 (Hs) | UGAAGAAUGUCAAGUCCAA | 1056 |
| 2109 | sense | | | |
| | strand | | | |
| MAPT- | 19 mer | 2117-2194 | GUCAAGUCCAAGAUCGGCU | 1057 |
| 2117 | sense | (Hs-Mf) | | |
| | strand | | | |
| MAPT- | 19 mer | 2136 (Hs) | CCACUGAGAACCUGAAGCA | 1058 |
| 2136 | sense | | | |
| | strand | | | |
| MAPT- | 19 mer | 2137 (Hs) | CACUGAGAACCUGAAGCAC | 1059 |
| 2137 | sense | | | |
| | strand | | | |
| MAPT- | 19 mer | 2269-2346 | GCAAAUAGUCUACAAACCA | 1060 |
| 2269 | sense | (Hs-Mf) | | |
| | strand | | | |
| MAPT- | 19 mer | 2270-2347 | CAAAUAGUCUACAAACCAG | 1061 |
| 2270 | sense | (Hs-Mf) | | |
| | strand | | | |
| MAPT- | 19 mer | 2271-2348 | AAAUAGUCUACAAACCAGU | 1062 |
| 2271 | sense | (Hs-Mf) | | |
| | strand | | | |
| MAPT- | 19 mer | 2272-2349 | AAUAGUCUACAAACCAGUU | 1063 |
| 2272 | sense | (Hs-Mf) | | |
| | strand | | | |
| MAPT- | 19 mer | 2273-2350 | AUAGUCUACAAACCAGUUG | 1064 |
| 2273 | sense | (Hs-Mf) | | |
| | strand | | | |
| MAPT- | 19 mer | 2274-2351 | UAGUCUACAAACCAGUUGA | 1065 |
| 2274 | sense | (Hs-Mf) | | |
| | strand | | | |
| MAPT- | 19 mer | 2275-2352 | AGUCUACAAACCAGUUGAC | 1066 |
| 2275 | sense | (Hs-Mf) | | |
| | strand | | | |
| MAPT- | 19 mer | 2276-2353 | GUCUACAAACCAGUUGACC | 1067 |
| 2276 | sense | (Hs-Mf) | | |
| | strand | | | |
| MAPT- | 19 mer | 2277-2354 | UCUACAAACCAGUUGACCU | 1068 |
| 2277 | sense | (Hs-Mf) | | |
| | strand | | | |
| MAPT- | 19 mer | 2278-2355 | CUACAAACCAGUUGACCUG | 1069 |
| 2278 | sense | (Hs-Mf) | | |
| | strand | | | |
| MAPT- | 19 mer | 2279-2356 | UACAAACCAGUUGACCUGA | 1070 |
| 2279 | sense | (Hs-Mf) | | |
| | strand | | | |
| MAPT- | 19 mer | 2280-2357 | ACAAACCAGUUGACCUGAG | 1071 |
| 2280 | sense | (Hs-Mf) | | |
| | strand | | | |
| MAPT- | 19 mer | 2281-2358 | CAAACCAGUUGACCUGAGC | 1072 |
| 2281 | sense | (Hs-Mf) | | |
| | strand | | | |
| MAPT- | 19 mer | 2282-2359 | AAACCAGUUGACCUGAGCA | 1073 |
| 2282 | sense | (Hs-Mf) | | |
| | strand | | | |
| MAPT- | 19 mer | 2283-2360 | AACCAGUUGACCUGAGCAA | 1074 |
| 2283 | sense | (Hs-Mf) | | |
| | strand | | | |
| MAPT- | 19 mer | 2284-2361 | ACCAGUUGACCUGAGCAAG | 1075 |
| 2284 | sense | (Hs-Mf) | | |
| | strand | | | |
| MAPT- | 19 mer | 2286-2363 | CAGUUGACCUGAGCAAGGU | 1076 |
| 2286 | sense | (Hs-Mf) | | |
| | strand | | | |
| MAPT- | 19 mer | 2288-2365 | GUUGACCUGAGCAAGGUGA | 1077 |
| 2288 | sense | (Hs-Mf) | | |
| | strand | | | |
| MAPT- | 19 mer | 2289-2366 | UUGACCUGAGCAAGGUGAC | 1078 |
| 2289 | sense | (Hs-Mf) | | |
| | strand | | | |
| MAPT- | 19 mer | 2291-2368 | GACCUGAGCAAGGUGACCU | 1079 |
| 2291 | sense | (Hs-Mf) | | |
| | strand | | | |
| MAPT- | 19 mer | 2294-2371 | CUGAGCAAGGUGACCUCCA | 1080 |
| 2294 | sense | (Hs-Mf) | | |
| | strand | | | |
| MAPT- | 19 mer | 2299-2376 | CAAGGUGACCUCCAAGUGU | 1081 |
| 2299 | sense | (Hs-Mf) | | |
| | strand | 2300-2377 | | |
| MAPT- | 19 mer | | AAGGUGACCUCCAAGUGUG | 1082 |
| 2300 | sense | (Hs-Mf) | | |
| | strand | 2301-2378 | | |
| MAPT- | 19 mer | | AGGUGACCUCCAAGUGUGG | 1083 |
| 2301 | sense | (Hs-Mf) | | |
| | strand | | | |
| MAPT- | 19 mer | 2308-2385 | CUCCAAGUGUGGCUCAUUA | 1084 |
| 2308 | sense | (Hs-Mf) | | |
| | strand | | | |
| MAPT- | 19 mer | 2316-2393 | GUGGCUCAUUAGGCAACAU | 1085 |
| 2316 | sense | (Hs-Mf) | | |
| | strand | | | |
| MAPT- | 19 mer | 2317-2394 | UGGCUCAUUAGGCAACAUC | 1086 |
| 2317 | sense | (Hs-Mf) | | |
| | strand | | | |
| MAPT- | 19 mer | 2319-2396 | GCUCAUUAGGCAACAUCCA | 1087 |
| 2319 | sense | (Hs-Mf) | | |
| | strand | | | |
| MAPT- | 19 mer | 2320-2397 | CUCAUUAGGCAACAUCCAU | 1088 |
| 2320 | sense | (Hs-Mf) | | |
| | strand | | | |
| MAPT- | 19 mer | 2322-2399 | CAUUAGGCAACAUCCAUCA | 1089 |
| 2322 | sense | (Hs-Mf) | | |
| | strand | | | |
| MAPT- | 19 mer | 2323-2400 | AUUAGGCAACAUCCAUCAU | 1090 |
| 2323 | sense | (Hs-Mf) | | |
| | strand | | | |
| MAPT- | 19 mer | 2324-2401 | UUAGGCAACAUCCAUCAUA | 1091 |
| 2324 | sense | (Hs-Mf) | | |
| | strand | | | |
| MAPT- | 19 mer | 2326-2403 | AGGCAACAUCCAUCAUAAA | 1092 |
| 2326 | sense | (Hs-Mf) | | |
| | strand | | | |
| MAPT- | 19 mer | 2330-2407 | AACAUCCAUCAUAAACCAG | 1093 |
| 2330 | sense | (Hs-Mf) | | |
| | strand | | | |
| MAPT- | 19 mer | 2356-2433 | CCAGGUGGAAGUAAAAUCU | 1094 |
| 2356 | sense | (Hs-Mf) | | |
| | strand | | | |
| MAPT- | 19 mer | 2357-2434 | CAGGUGGAAGUAAAAUCUG | 1095 |
| 2357 | sense | (Hs-Mf) | | |
| | strand | | | |
| MAPT- | 19 mer | 2358-2435 | AGGUGGAAGUAAAAUCUGA | 1096 |
| 2358 | sense | (Hs-Mf) | | |
| | strand | | | |
| MAPT- | 19 mer | 2359-2436 | GGUGGAAGUAAAAUCUGAG | 1097 |
| 2359 | sense | (Hs-Mf) | | |
| | strand | | | |
| MAPT- | 19 mer | 2360-2437 | GUGGAAGUAAAAUCUGAGA | 1098 |
| 2360 | sense | (Hs-Mf) | | |
| | strand | | | |
| MAPT- | 19 mer | 2361-2438 | UGGAAGUAAAAUCUGAGAA | 1099 |
| 2361 | sense | (Hs-Mf) | | |
| | strand | | | |
| MAPT- | 19 mer | 2362-2439 | GGAAGUAAAAUCUGAGAAG | 1100 |
| 2362 | sense | (Hs-Mf) | | |
| | strand | | | |
| MAPT- | 19 mer | 2363-2440 | GAAGUAAAAUCUGAGAAGC | 1101 |
| 2363 | sense | (Hs-Mf) | | |
| | strand | | | |
| MAPT- | 19 mer | 2364-2441 | AAGUAAAAUCUGAGAAGCU | 1102 |
| 2364 | sense | (Hs-Mf) | | |
| | strand | | | |
| MAPT- | 19 mer | 2365 (Hs) | AGUAAAAUCUGAGAAGCUU | 1103 |
| 2365 | sense | | | |
| | strand | | | |
| MAPT- | 19 mer | 2372 (Hs) | UCUGAGAAGCUUGACUUCA | 1104 |
| 2372 | sense | | | |
| | strand | | | |
| MAPT- | 19 mer | 2373 (Hs) | CUGAGAAGCUUGACUUCAA | 1105 |
| 2373 | sense | | | |
| | strand | | | |
| MAPT- | 19 mer | 2374 (Hs) | UGAGAAGCUUGACUUCAAG | 1106 |
| 2374 | sense | | | |
| | strand | | | |
| MAPT- | 19 mer | 2375 (Hs) | GAGAAGCUUGACUUCAAGG | 1107 |
| 2375 | sense | | | |
| | strand | | | |
| MAPT- | 19 mer | 2376 (Hs) | AGAAGCUUGACUUCAAGGA | 1108 |
| 2376 | sense | | | |
| | strand | | | |
| MAPT- | 19 mer | 2377 (Hs) | GAAGCUUGACUUCAAGGAC | 1109 |
| 2377 | sense | | | |
| | strand | | | |
| MAPT- | 19 mer | 2378 (Hs) | AAGCUUGACUUCAAGGACA | 1110 |
| 2378 | sense | | | |
| | strand | | | |
| MAPT- | 19 mer | 2379 (Hs) | AGCUUGACUUCAAGGACAG | 1111 |
| 2379 | sense | | | |
| | strand | | | |
| MAPT- | 19 mer | 2380 (Hs) | GCUUGACUUCAAGGACAGA | 1112 |
| 2380 | sense | | | |
| | strand | | | |
| MAPT- | 19 mer | 2381 (Hs) | CUUGACUUCAAGGACAGAG | 1113 |
| 2381 | sense | | | |
| | strand | | | |
| MAPT- | 19 mer | 2382 (Hs) | UUGACUUCAAGGACAGAGU | 1114 |
| 2382 | sense | | | |
| | strand | | | |
| MAPT- | 19 mer | 2390 (Hs) | AAGGACAGAGUCCAGUCGA | 1115 |
| 2390 | sense | | | |
| | strand | | | |
| MAPT- | 19 mer | 2391 (Hs) | AGGACAGAGUCCAGUCGAA | 1116 |
| 2391 | sense | | | |
| | strand | | | |
| MAPT- | 19 mer | 2414-2491 | GGGUCCCUGGACAAUAUCA | 1117 |
| 2414 | sense | (Hs-Mf) | | |
| | strand | | | |
| MAPT- | 19 mer | 2448-2525 | GAGGAAAUAAAAAGAUUGA | 1118 |
| 2448 | sense | (Hs-Mf) | | |
| | strand | | | |
| MAPT- | 19 mer | 2449-2526 | AGGAAAUAAAAAGAUUGAA | 1119 |
| 2449 | sense | (Hs-Mf) | | |
| | strand | | | |
| MAPT- | 19 mer | 2450-2527 | GGAAAUAAAAAGAUUGAAA | 1120 |
| 2450 | sense | (Hs-Mf) | | |
| | strand | | | |
| MAPT- | 19 mer | 2451-2528 | GAAAUAAAAAGAUUGAAAC | 1121 |
| 2451 | sense | (Hs-Mf) | | |
| | strand | | | |
| MAPT- | 19 mer | 2452-2529 | AAAUAAAAAGAUUGAAACC | 1122 |
| 2452 | sense | (Hs-Mf) | | |
| | strand | | | |
| MAPT- | 19 mer | 2453-2530 | AAUAAAAAGAUUGAAACCC | 1123 |
| 2453 | sense | (Hs-Mf) | | |
| | strand | | | |
| MAPT- | 19 mer | 2454-2531 | AUAAAAAGAUUGAAACCCA | 1124 |
| 2454 | sense | (Hs-Mf) | | |
| | strand | | | |
| MAPT- | 19 mer | 2456-2533 | AAAAAGAUUGAAACCCACA | 1125 |
| 2456 | sense | (Hs-Mf) | | |
| | strand | | | |
| MAPT- | 19 mer | 2457-2534 | AAAAGAUUGAAACCCACAA | 1126 |
| 2457 | sense | (Hs-Mf) | | |
| | strand | | | |
| MAPT- | 19 mer | 2567 (Hs) | CGGCAUCUCAGCAAUGUCU | 1127 |
| 2567 | sense | | | |
| | strand | | | |
| MAPT- | 19 mer | 2598-2675 | GCAUCGACAUGGUAGACUC | 1128 |
| 2598 | sense | (Hs-Mf) | | |
| | strand | | | |
| MAPT- | 19 mer | 2657-2734 | CUGGCCAAGCAGGGUUUGU | 1129 |
| 2657 | sense | (Hs-Mf) | | |
| | strand | | | |
| MAPT- | 19 mer | 2723-2800 | AGAGUGUGGAAAAAAAAAG | 1130 |
| 2723 | sense | (Hs-Mf) | | |
| | strand | | | |
| MAPT- | 19 mer | 2724-2801 | GAGUGUGGAAAAAAAAAGA | 1131 |
| 2724 | sense | (Hs-Mf) | | |
| | strand | | | |
| MAPT- | 19 mer | 2726-2803 | GUGUGGAAAAAAAAAGAAU | 1132 |
| 2726 | sense | (Hs-Mf) | | |
| | strand | | | |
| MAPT- | 19 mer | 2784-2860- | GCAGUUCGGUUAAUUGGUU | 1133 |
| 2784 | sense | 1 mismatch | | |
| | strand | (Hs-Mf) | | |
| MAPT- | 19 mer | 2963-3039 | GGCAAUUCCUUUUGAUUCU | 1134 |
| 2963 | sense | (Hs-Mf) | | |
| | strand | | | |
| MAPT- | 19 mer | 3110-3186 | AGCAACAAAGGAUUUGAAA | 1135 |
| 3110 | sense | (Hs-Mf) | | |
| | strand | | | |
| MAPT- | 19 mer | 3114-3190 | ACAAAGGAUUUGAAACUUG | 1136 |
| 3114 | sense | (Hs-Mf) | | |
| | strand | | | |
| MAPT- | 19 mer | 3116-3192 | AAAGGAUUUGAAACUUGGU | 1137 |
| 3116 | sense | (Hs-Mf) | | |
| | strand | | | |
| MAPT- | 19 mer | 3118-3194 | AGGAUUUGAAACUUGGUGU | 1138 |
| 3118 | sense | (Hs-Mf) | | |
| | strand | | | |
| MAPT- | 19 mer | 3158-3234 | CGAUGUCAACCUUGUGUGA | 1139 |
| 3158 | sense | (Hs-Mf) | | |
| | strand | | | |
| MAPT- | 19 mer | 3503-3576 | AAAGACUGACCUUGAUGUC | 1140 |
| 3503 | sense | (Hs-Mf) | | |
| | strand | | | |
| MAPT- | 19 mer | 3589-3661 | CUCCACAGAAACCCUGUUU | 1141 |
| 3589 | sense | (Hs-Mf) | | |
| | strand | | | |
| MAPT- | 19 mer | 3591-3663 | CCACAGAAACCCUGUUUUA | 1142 |
| 3591 | sense | (Hs-Mf) | | |
| | strand | | | |
| MAPT- | 19 mer | 3592-3664 | CACAGAAACCCUGUUUUAU | 1143 |
| 3592 | sense | (Hs-Mf) | | |
| | strand | | | |
| MAPT- | 19 mer | 3593-3665 | ACAGAAACCCUGUUUUAUU | 1144 |
| 3593 | sense | (Hs-Mf) | | |
| | strand | | | |
| MAPT- | 19 mer | 3594-3666 | CAGAAACCCUGUUUUAUUG | 1145 |
| 3594 | sense | (Hs-Mf) | | |
| | strand | | | |
| MAPT- | 19 mer | 3595-3667 | AGAAACCCUGUUUUAUUGA | 1146 |
| 3595 | sense | (Hs-Mf) | | |
| | strand | | | |
| MAPT- | 19 mer | 3596-3668 | GAAACCCUGUUUUAUUGAG | 1147 |
| 3596 | sense | (Hs-Mf) | | |
| | strand | | | |
| MAPT- | 19 mer | 3597-3669 | AAACCCUGUUUUAUUGAGU | 1148 |
| 3597 | sense | (Hs-Mf) | | |
| | strand | | | |
| MAPT- | 19 mer | 3598-3670 | AACCCUGUUUUAUUGAGUU | 1149 |
| 3598 | sense | (Hs-Mf) | | |
| | strand | | | |
| MAPT- | 19 mer | 3599-3671 | ACCCUGUUUUAUUGAGUUC | 1150 |
| 3599 | sense | (Hs-Mf) | | |
| | strand | | | |
| MAPT- | 19 mer | 3600-3672 | CCCUGUUUUAUUGAGUUCU | 1151 |
| 3600 | sense | (Hs-Mf) | | |
| | strand | | | |
| MAPT- | 19 mer | 3601-3673 | CCUGUUUUAUUGAGUUCUG | 1152 |
| 3601 | sense | (Hs-Mf) | | |
| | strand | | | |
| MAPT- | 19 mer | 3602-3674 | CUGUUUUAUUGAGUUCUGA | 1153 |
| 3602 | sense | (Hs-Mf) | | |
| | strand | 3603-3675 | | |
| MAPT- | 19 mer | | UGUUUUAUUGAGUUCUGAA | 1154 |
| 3603 | sense | (Hs-Mf) | | |
| | strand | | | |
| MAPT- | 19 mer | 3605-3677 | UUUUAUUGAGUUCUGAAGG | 1155 |
| 3605 | sense | (Hs-Mf) | | |
| | strand | | | |
| MAPT- | 19 mer | 3607-3679 | UUAUUGAGUUCUGAAGGUU | 1156 |
| 3607 | sense | (Hs-Mf) | | |
| | strand | | | |
| MAPT- | 19 mer | 3609-3681 | AUUGAGUUCUGAAGGUUGG | 1157 |
| 3609 | sense | (Hs-Mf) | | |
| | strand | | | |
| MAPT- | 19 mer | 3610-3682 | UUGAGUUCUGAAGGUUGGA | 1158 |
| 3610 | sense | (Hs-Mf) | | |
| | strand | | | |
| MAPT- | 19 mer | 3677-3749 | AACCAGUUCUCUUUGUAAG | 1159 |
| 3677 | sense | (Hs-Mf) | | |
| | strand | | | |
| MAPT- | 19 mer | 3678-3750 | ACCAGUUCUCUUUGUAAGG | 1160 |
| 3678 | sense | (Hs-Mf) | | |
| | strand | | | |
| MAPT- | 19 mer | 3679-3751 | CCAGUUCUCUUUGUAAGGA | 1161 |
| 3679 | sense | (Hs-Mf) | | |
| | strand | | | |
| MAPT- | 19 mer | 3680-3752 | CAGUUCUCUUUGUAAGGAC | 1162 |
| 3680 | sense | (Hs-Mf) | | |
| | strand | | | |
| MAPT- | 19 mer | 3958-4030 | CUACUCCAUACUGAGGGUG | 1163 |
| 3958 | sense | (Hs-Mf) | | |
| | strand | | | |
| MAPT- | 19 mer | 3959-4031 | UACUCCAUACUGAGGGUGA | 1164 |
| 3959 | sense | (Hs-Mf) | | |
| | strand | | | |
| MAPT- | 19 mer | 3960-4032 | ACUCCAUACUGAGGGUGAA | 1165 |
| 3960 | sense | (Hs-Mf) | | |
| | strand | | | |
| MAPT- | 19 mer | 3961-4033 | CUCCAUACUGAGGGUGAAA | 1166 |
| 3961 | sense | (Hs-Mf) | | |
| | strand | | | |
| MAPT- | 19 mer | 3965-4037 | AUACUGAGGGUGAAAUUAA | 1167 |
| 3965 | sense | (Hs-Mf) | | |
| | strand | | | |
| MAPT- | 19 mer | 3970-4042 | GAGGGUGAAAUUAAGGGAA | 1168 |
| 3970 | sense | (Hs-Mf) | | |
| | strand | | | |
| MAPT- | 19 mer | 4146-4218 | GGUGUUUCUGCCUUGUUGA | 1169 |
| 4146 | sense | (Hs-Mf) | | |
| | strand | | | |
| MAPT- | 19 mer | 4474-4545 | CUGGAGCAGCUGAACAUAU | 1170 |
| 4474 | sense | (Hs-Mf) | | |
| | strand | | | |
| MAPT- | 19 mer | 4475-4546 | UGGAGCAGCUGAACAUAUA | 1171 |
| 4475 | sense | (Hs-Mf) | | |
| | strand | | | |
| MAPT- | 19 mer | 4477-4548 | GAGCAGCUGAACAUAUACA | 1172 |
| 4477 | sense | (Hs-Mf) | | |
| | strand | | | |
| MAPT- | 19 mer | 4478-4549 | AGCAGCUGAACAUAUACAU | 1173 |
| 4478 | sense | (Hs-Mf) | | |
| | strand | | | |
| MAPT- | 19 mer | 4479-4550 | GCAGCUGAACAUAUACAUA | 1174 |
| 4479 | sense | (Hs-Mf) | | |
| | strand | | | |
| MAPT- | 19 mer | 4480-4551 | CAGCUGAACAUAUACAUAG | 1175 |
| 4480 | sense | (Hs-Mf) | | |
| | strand | | | |
| MAPT- | 19 mer | 4481-4552 | AGCUGAACAUAUACAUAGA | 1176 |
| 4481 | sense | (Hs-Mf) | | |
| | strand | | | |
| MAPT- | 19 mer | 4482-4553 | GCUGAACAUAUACAUAGAU | 1177 |
| 4482 | sense | (Hs-Mf) | | |
| | strand | | | |
| MAPT- | 19 mer | 4485-4556 | GAACAUAUACAUAGAUGUU | 1178 |
| 4485 | sense | (Hs-Mf) | | |
| | strand | | | |
| MAPT- | 19 mer | 4486-4557 | AACAUAUACAUAGAUGUUG | 1179 |
| 4486 | sense | (Hs-Mf) | | |
| | strand | | | |
| MAPT- | 19 mer | 4532 (Hs) | GAGUUGUAGUUGGAUUUGU | 1180 |
| 4532 | sense | | | |
| | strand | | | |
| MAPT- | 19 mer | 4533 (Hs) | AGUUGUAGUUGGAUUUGUC | 1181 |
| 4533 | sense | | | |
| | strand | | | |
| MAPT- | 19 mer | 4539-4610 | AGUUGGAUUUGUCUGUUUA | 1182 |
| 4539 | sense | (Hs-Mf) | | |
| | strand | | | |
| MAPT- | 19 mer | 4540-4611 | GUUGGAUUUGUCUGUUUAU | 1183 |
| 4540 | sense | (Hs-Mf) | | |
| | strand | | | |
| MAPT- | 19 mer | 4541-4612 | UUGGAUUUGUCUGUUUAUG | 1184 |
| 4541 | sense | (Hs-Mf) | | |
| | strand | | | |
| MAPT- | 19 mer | 4543-4614 | GGAUUUGUCUGUUUAUGCU | 1185 |
| 4543 | sense | (Hs-Mf) | | |
| | strand | | | |
| MAPT- | 19 mer | 4544-4615 | GAUUUGUCUGUUUAUGCUU | 1186 |
| 4544 | sense | (Hs-Mf) | | |
| | strand | | | |
| MAPT- | 19 mer | 4545-4616 | AUUUGUCUGUUUAUGCUUG | 1187 |
| 4545 | sense | (Hs-Mf) | | |
| | strand | | | |
| MAPT- | 19 mer | 4546-4617 | UUUGUCUGUUUAUGCUUGG | 1188 |
| 4546 | sense | (Hs-Mf) | | |
| | strand | | | |
| MAPT- | 19 mer | 4547-4618 | UUGUCUGUUUAUGCUUGGA | 1189 |
| 4547 | sense | (Hs-Mf) | | |
| | strand | | | |
| MAPT- | 19 mer | 4548-4619 | UGUCUGUUUAUGCUUGGAU | 1190 |
| 4548 | sense | (Hs-Mf) | | |
| | strand | | | |
| MAPT- | 19 mer | 4549-4620 | GUCUGUUUAUGCUUGGAUU | 1191 |
| 4549 | sense | (Hs-Mf) | | |
| | strand | | | |
| MAPT- | 19 mer | 4550-4621 | UCUGUUUAUGCUUGGAUUC | 1192 |
| 4550 | sense | (Hs-Mf) | | |
| | strand | | | |
| MAPT- | 19 mer | 4551-4622 | CUGUUUAUGCUUGGAUUCA | 1193 |
| 4551 | sense | (Hs-Mf) | | |
| | strand | | | |
| MAPT- | 19 mer | 4552-4623 | UGUUUAUGCUUGGAUUCAC | 1194 |
| 4552 | sense | (Hs-Mf) | | |
| | strand | | | |
| MAPT- | 19 mer | 4554-4625 | UUUAUGCUUGGAUUCACCA | 1195 |
| 4554 | sense | (Hs-Mf) | | |
| | strand | | | |
| MAPT- | 19 mer | 4556-4627 | UAUGCUUGGAUUCACCAGA | 1196 |
| 4556 | sense | (Hs-Mf) | | |
| | strand | | | |
| MAPT- | 19 mer | 4557-4628 | AUGCUUGGAUUCACCAGAG | 1197 |
| 4557 | sense | (Hs-Mf) | | |
| | strand | | | |
| MAPT- | 19 mer | 4558-4629 | UGCUUGGAUUCACCAGAGU | 1198 |
| 4558 | sense | (Hs-Mf) | | |
| | strand | | | |
| MAPT- | 19 mer | 4559-4630 | GCUUGGAUUCACCAGAGUG | 1199 |
| 4559 | sense | (Hs-Mf) | | |
| | strand | | | |
| MAPT- | 19 mer | 4560-4631 | CUUGGAUUCACCAGAGUGA | 1200 |
| 4560 | sense | (Hs-Mf) | | |
| | strand | | | |
| MAPT- | 19 mer | 4561-4632 | UUGGAUUCACCAGAGUGAC | 1201 |
| 4561 | sense | (Hs-Mf) | | |
| | strand | | | |
| MAPT- | 19 mer | 4562-4633 | UGGAUUCACCAGAGUGACU | 1202 |
| 4562 | sense | (Hs-Mf) | | |
| | strand | | | |
| MAPT- | 19 mer | 4563-4634 | GGAUUCACCAGAGUGACUA | 1203 |
| 4563 | sense | (Hs-Mf) | | |
| | strand | | | |
| MAPT- | 19 mer | 4564-4635 | GAUUCACCAGAGUGACUAU | 1204 |
| 4564 | sense | (Hs-Mf) | | |
| | strand | | | |
| MAPT- | 19 mer | 4615-4687 | ACGCAUGUAUCUUGAAAUG | 1205 |
| 4615 | sense | (Hs-Mf) | | |
| | strand | | | |
| MAPT- | 19 mer | 4616-4688 | CGCAUGUAUCUUGAAAUGC | 1206 |
| 4616 | sense | (Hs-Mf) | | |
| | strand | | | |
| MAPT- | 19 mer | 4617-4689 | GCAUGUAUCUUGAAAUGCU | 1207 |
| 4617 | sense | (Hs-Mf) | | |
| | strand | | | |
| MAPT- | 19 mer | 4618-4690 | CAUGUAUCUUGAAAUGCUU | 1208 |
| 4618 | sense | (Hs-Mf) | | |
| | strand | | | |
| MAPT- | 19 mer | 4619-4691 | AUGUAUCUUGAAAUGCUUG | 1209 |
| 4619 | sense | (Hs-Mf) | | |
| | strand | | | |
| MAPT- | 19 mer | 4620-4692 | UGUAUCUUGAAAUGCUUGU | 1210 |
| 4620 | sense | (Hs-Mf) | | |
| | strand | | | |
| MAPT- | 19 mer | 4621-4693 | GUAUCUUGAAAUGCUUGUA | 1211 |
| 4621 | sense | (Hs-Mf) | | |
| | strand | | | |
| MAPT- | 19 mer | 4622-4694 | UAUCUUGAAAUGCUUGUAA | 1212 |
| 4622 | sense | (Hs-Mf) | | |
| | strand | | | |
| MAPT- | 19 mer | 4623-4695 | AUCUUGAAAUGCUUGUAAA | 1213 |
| 4623 | sense | (Hs-Mf) | | |
| | strand | | | |
| MAPT- | 19 mer | 4625-4697 | CUUGAAAUGCUUGUAAAGA | 1214 |
| 4625 | sense | (Hs-Mf) | | |
| | strand | | | |
| MAPT- | 19 mer | 4627-4699 | UGAAAUGCUUGUAAAGAGG | 1215 |
| 4627 | sense | (Hs-Mf) | | |
| | strand | | | |
| MAPT- | 19 mer | 4628-4700 | GAAAUGCUUGUAAAGAGGU | 1216 |
| 4628 | sense | (Hs-Mf) | | |
| | strand | | | |
| MAPT- | 19 mer | 4629-4701 | AAAUGCUUGUAAAGAGGUU | 1217 |
| 4629 | sense | (Hs-Mf) | | |
| | strand | | | |
| MAPT- | 19 mer | 4630-4702 | AAUGCUUGUAAAGAGGUUU | 1218 |
| 4630 | sense | (Hs-Mf) | | |
| | strand | | | |
| MAPT- | 19 mer | 4632-4704 | UGCUUGUAAAGAGGUUUCU | 1219 |
| 4632 | sense | (Hs-Mf) | | |
| | strand | | | |
| MAPT- | 19 mer | 4633-4705 | GCUUGUAAAGAGGUUUCUA | 1220 |
| 4633 | sense | (Hs-Mf) | | |
| | strand | | | |
| MAPT- | 19 mer | 4825-4897 | ACAGGAUUAGGACUGAAGC | 1221 |
| 4825 | sense | (Hs-Mf) | | |
| | strand | | | |
| MAPT- | 19 mer | 4828-4900 | GGAUUAGGACUGAAGCGAU | 1222 |
| 4828 | sense | (Hs-Mf) | | |
| | strand | | | |
| MAPT- | 19 mer | 5682-5743 | GAAGUUCUUGUGCCCUGCU | 1223 |
| 5682 | sense | (Hs-Mf) | | |
| | strand | | | |
| MAPT- | 19 mer | 5958 (Hs) | AAGCUGCUGACUCACUUUA | 1224 |
| 5958 | sense | | | |
| | strand | | | |
| MAPT- | 19 mer | 5959 (Hs) | AGCUGCUGACUCACUUUAU | 1225 |
| 5959 | sense | | | |
| | strand | | | |
| MAPT- | 19 mer | 5961 (Hs) | CUGCUGACUCACUUUAUCA | 1226 |
| 5961 | sense | | | |
| | strand | | | |
| MAPT- | 19 mer | 5963 (Hs) | GCUGACUCACUUUAUCAAU | 1227 |
| 5963 | sense | | | |
| | strand | | | |
| MAPT- | 19 mer | 5964 (Hs) | CUGACUCACUUUAUCAAUA | 1228 |
| 5964 | sense | | | |
| | strand | | | |
| MAPT- | 19 mer | 5965 (Hs) | UGACUCACUUUAUCAAUAG | 1229 |
| 5965 | sense | | | |
| | strand | | | |
| MAPT- | 19 mer | 5966-6021 | GACUCACUUUAUCAAUAGU | 1230 |
| 5966 | sense | (Hs-Mf) | | |
| | strand | | | |
| MAPT- | 19 mer | 5967-6022 | ACUCACUUUAUCAAUAGUU | 1231 |
| 5967 | sense | (Hs-Mf) | | |
| | strand | | | |
| MAPT- | 19 mer | 5968-6023 | CUCACUUUAUCAAUAGUUC | 1232 |
| 5968 | sense | (Hs-Mf) | | |
| | strand | | | |
| MAPT- | 19 mer | 6006-6061 | GGUGAGACUGUAUCCUGUU | 1233 |
| 6006 | sense | (Hs-Mf) | | |
| | strand | | | |
| MAPT- | 19 mer | 6007-6062 | GUGAGACUGUAUCCUGUUU | 1234 |
| 6007 | sense | (Hs-Mf) | | |
| | strand | | | |
| MAPT- | 19 mer | 6008-6063 | UGAGACUGUAUCCUGUUUG | 1235 |
| 6008 | sense | (Hs-Mf) | | |
| | strand | | | |
| MAPT- | 19 mer | 6009-6064 | GAGACUGUAUCCUGUUUGC | 1236 |
| 6009 | sense | (Hs-Mf) | | |
| | strand | | | |
| MAPT- | 19 mer | 6010-6065 | AGACUGUAUCCUGUUUGCU | 1237 |
| 6010 | sense | (Hs-Mf) | | |
| | strand | | | |
| MAPT- | 19 mer | 6011-6066 | GACUGUAUCCUGUUUGCUA | 1238 |
| 6011 | sense | (Hs-Mf) | | |
| | strand | | | |
| MAPT- | 19 mer | 6012-6067 | ACUGUAUCCUGUUUGCUAU | 1239 |
| 6012 | sense | (Hs-Mf) | | |
| | strand | | | |
| MAPT- | 19 mer | 6013-6068 | CUGUAUCCUGUUUGCUAUU | 1240 |
| 6013 | sense | (Hs-Mf) | | |
| | strand | | | |
| MAPT- | 19 mer | 6014-6069 | UGUAUCCUGUUUGCUAUUG | 1241 |
| 6014 | sense | (Hs-Mf) | | |
| | strand | | | |
| MAPT- | 19 mer | 6015-6070 | GUAUCCUGUUUGCUAUUGC | 1242 |
| 6015 | sense | (Hs-Mf) | | |
| | strand | | | |
| MAPT- | 19 mer | 6017-6072 | AUCCUGUUUGCUAUUGCUU | 1243 |
| 6017 | sense | (Hs-Mf) | | |
| | strand | | | |
| MAPT- | 19 mer | 6119-6174 | GCCUCGUAACCCUUUUCAU | 1244 |
| 6119 | sense | (Hs-Mf) | | |
| | strand | | | |
| MAPT- | 19 mer | 6628-6689 | GAGUUUGCCAUGUUGAGCA | 1245 |
| 6628 | sense | (Hs-Mf) | | |
| | strand | | | |
| MAPT- | 19 mer | 6629-6690 | AGUUUGCCAUGUUGAGCAG | 1246 |
| 6629 | sense | (Hs-Mf) | | |
| | strand | | | |
| MAPT- | 19 mer | 6631-6692 | UUUGCCAUGUUGAGCAGGA | 1247 |
| 6631 | sense | (Hs-Mf) | | |
| | strand | | | |
| MAPT- | 19 mer | 6672-6733 | CCAUGAUUUCUUCGGUAAU | 1248 |
| 6672 | sense | (Hs-Mf) | | |
| | strand | | | |
| MAPT- | 19 mer | 6731 (Hs) | GCUUUCUGUCUGUGAAUGU | 1249 |
| 6731 | sense | | | |
| | strand | | | |
| MAPT- | 19 mer | 6732 (Hs) | CUUUCUGUCUGUGAAUGUC | 1250 |
| 6732 | sense | | | |
| | strand | | | |
| MAPT- | 19 mer | 6738-6799 | GUCUGUGAAUGUCUAUAUA | 1251 |
| 6738 | sense | (Hs-Mf) | | |
| | strand | | | |
| MAPT- | 19 mer | 6739-6800 | UCUGUGAAUGUCUAUAUAG | 1252 |
| 6739 | sense | (Hs-Mf) | | |
| | strand | | | |
| MAPT- | 19 mer | 6740-6801 | CUGUGAAUGUCUAUAUAGU | 1253 |
| 6740 | sense | (Hs-Mf) | | |
| | strand | | | |
| MAPT- | 19 mer | 6741-6802 | UGUGAAUGUCUAUAUAGUG | 1254 |
| 6741 | sense | (Hs-Mf) | | |
| | strand | | | |
| MAPT- | 19 mer | 6742-6803 | GUGAAUGUCUAUAUAGUGU | 1255 |
| 6742 | sense | (Hs-Mf) | | |
| | strand | | | |
| MAPT- | 19 mer | 6743-6804 | UGAAUGUCUAUAUAGUGUA | 1256 |
| 6743 | sense | (Hs-Mf) | | |
| | strand | | | |
| MAPT- | 19 mer | 6745-6806 | AAUGUCUAUAUAGUGUAUU | 1257 |
| 6745 | sense | (Hs-Mf) | | |
| | strand | | | |
| MAPT- | 19 mer | 6748-6809 | GUCUAUAUAGUGUAUUGUG | 1258 |
| 6748 | sense | (Hs-Mf) | | |
| | strand | | | |
| MAPT- | 19 mer | 6749-6810 | UCUAUAUAGUGUAUUGUGU | 1259 |
| 6749 | sense | (Hs-Mf) | | |
| | strand | | | |
| MAPT- | 19 mer | 6750-6811 | CUAUAUAGUGUAUUGUGUG | 1260 |
| 6750 | sense | (Hs-Mf) | | |
| | strand | | | |
| MAPT- | 19 mer | 6751-6812 | UAUAUAGUGUAUUGUGUGU | 1261 |
| 6751 | sense | (Hs-Mf) | | |
| | strand | | | |
| MAPT- | 19 mer | 6752-6813 | AUAUAGUGUAUUGUGUGUU | 1262 |
| 6752 | sense | (Hs-Mf) | | |
| | strand | | | |
| MAPT- | 19 mer | 6753-6814 | UAUAGUGUAUUGUGUGUUU | 1263 |
| 6753 | sense | (Hs-Mf) | | |
| | strand | | | |
| MAPT- | 19 mer | 6754-6815 | AUAGUGUAUUGUGUGUUUU | 1264 |
| 6754 | sense | (Hs-Mf) | | |
| | strand | | | |
| MAPT- | 19 mer | 6755-6816 | UAGUGUAUUGUGUGUUUUA | 1265 |
| 6755 | sense | (Hs-Mf) | | |
| | strand | | | |
| MAPT- | 19 mer | 6756-6817 | AGUGUAUUGUGUGUUUUAA | 1266 |
| 6756 | sense | (Hs-Mf) | | |
| | strand | | | |
| MAPT- | 19 mer | 6757-6818 | GUGUAUUGUGUGUUUUAAC | 1267 |
| 6757 | sense | (Hs-Mf) | | |
| | strand | | | |
| MAPT- | 19 mer | 6758-6819 | UGUAUUGUGUGUUUUAACA | 1268 |
| 6758 | sense | (Hs-Mf) | | |
| | strand | | | |
| MAPT- | 19 mer | 6759-6820 | GUAUUGUGUGUUUUAACAA | 1269 |
| 6759 | sense | (Hs-Mf) | | |
| | strand | | | |
| MAPT- | 19 mer | 6760-6821 | UAUUGUGUGUUUUAACAAA | 1270 |
| 6760 | sense | (Hs-Mf) | | |
| | strand | | | |
| MAPT- | 19 mer | 6761-6822 | AUUGUGUGUUUUAACAAAU | 1271 |
| 6761 | sense | (Hs-Mf) | | |
| | strand | | | |
| MAPT- | 19 mer | 6762-6823 | UUGUGUGUUUUAACAAAUG | 1272 |
| 6762 | sense | (Hs-Mf) | | |
| | strand | | | |
| MAPT- | 19 mer | 6763-6824 | UGUGUGUUUUAACAAAUGA | 1273 |
| 6763 | sense | (Hs-Mf) | | |
| | strand | | | |
| MAPT- | 19 mer | 6764-6825 | GUGUGUUUUAACAAAUGAU | 1274 |
| 6764 | sense | (Hs-Mf) | | |
| | strand | | | |
| MAPT- | 19 mer | 6765-6826 | UGUGUUUUAACAAAUGAUU | 1275 |
| 6765 | sense | (Hs-Mf) | | |
| | strand | | | |
| MAPT- | 19 mer | 6766-6827 | GUGUUUUAACAAAUGAUUU | 1276 |
| 6766 | sense | (Hs-Mf) | | |
| | strand | | | |
| MAPT- | 19 mer | 6767-6828 | UGUUUUAACAAAUGAUUUA | 1277 |
| 6767 | sense | (Hs-Mf) | | |
| | strand | | | |
| MAPT- | 19 mer | 6768-6829 | GUUUUAACAAAUGAUUUAC | 1278 |
| 6768 | sense | (Hs-Mf) | | |
| | strand | | | |
| MAPT- | 19 mer | 6769-6830 | UUUUAACAAAUGAUUUACA | 1279 |
| 6769 | sense | (Hs-Mf) | | |
| | strand | | | |
| MAPT- | 19 mer | 6772-6833 | UAACAAAUGAUUUACACUG | 1280 |
| 6772 | sense | (Hs-Mf) | | |
| | strand | | | |
| MAPT- | 19 mer | 6773-6834 | AACAAAUGAUUUACACUGA | 1281 |
| 6773 | sense | (Hs-Mf) | | |
| | strand | | | |
| MAPT- | 19 mer | 6774-6835 | ACAAAUGAUUUACACUGAC | 1282 |
| 6774 | sense | (Hs-Mf) | | |
| | strand | | | |
| MAPT- | 19 mer | 6775-6836 | CAAAUGAUUUACACUGACU | 1283 |
| 6775 | sense | (Hs-Mf) | | |
| | strand | | | |
| MAPT- | 19 mer | 6777-6838 | AAUGAUUUACACUGACUGU | 1284 |
| 6777 | sense | (Hs-Mf) | | |
| | strand | | | |
| MAPT- | 19 mer | 6778-6839 | AUGAUUUACACUGACUGUU | 1285 |
| 6778 | sense | (Hs-Mf) | | |
| | strand | | | |
| MAPT- | 19 mer | 6779-6840 | UGAUUUACACUGACUGUUG | 1286 |
| 6779 | sense | (Hs-Mf) | | |
| | strand | | | |
| MAPT- | 19 mer | 6780-6841 | GAUUUACACUGACUGUUGC | 1287 |
| 6780 | sense | (Hs-Mf) | | |
| | strand | | | |
| MAPT- | 19 mer | 6781 (Hs) | AUUUACACUGACUGUUGCU | 1288 |
| 6781 | sense | | | |
| | strand | | | |
| MAPT- | 19 mer | 6789 (Hs) | UGACUGUUGCUGUAAAAGU | 1289 |
| 6789 | sense | | | |
| | strand | | | |
| MAPT- | 19 mer | 6792 (Hs) | CUGUUGCUGUAAAAGUGAA | 1290 |
| 6792 | sense | | | |
| | strand | | | |
| MAPT- | 19 mer | 6793 (Hs) | UGUUGCUGUAAAAGUGAAU | 1291 |
| 6793 | sense | | | |
| | strand | | | |
| MAPT- | 19 mer | 6795 (Hs) | UUGCUGUAAAAGUGAAUUU | 1292 |
| 6795 | sense | | | |
| | strand | | | |
| MAPT- | 19 mer | 6796 (Hs) | UGCUGUAAAAGUGAAUUUG | 1293 |
| 6796 | sense | | | |
| | strand | | | |
| MAPT- | 19 mer | 6797 (Hs) | GCUGUAAAAGUGAAUUUGG | 1294 |
| 6797 | sense | | | |
| | strand | | | |
| MAPT- | 19 mer | 6798 (Hs) | CUGUAAAAGUGAAUUUGGA | 1295 |
| 6798 | sense | | | |
| | strand | | | |
| MAPT- | 19 mer | 2141-2218- | GCUGGUGCUUCAGGUUCUC | 1296 |
| 2141 | anti- | 966 (Hs- | | |
| | sense | Mf-Mm) | | |
| | strand | | | |
| MAPT- | 19 mer | 2142-2219- | GGCUGGUGCUUCAGGUUCU | 1297 |
| 2142 | anti- | 967 (Hs- | | |
| | sense | Mf-Mm) | | |
| | strand | | | |
| MAPT- | 19 mer | 2303-2380- | AGCCACACUUGGAGGUCAC | 1298 |
| 2303 | anti- | 1128 (Hs- | | |
| | sense | Mf-Mm) | | |
| | strand | | | |
| MAPT- | 19 mer | 2347-2424- | UUCCACCUGGCCACCUCCU | 1299 |
| 2347 | anti- | 1172 (Hs- | | |
| | sense | Mf-Mm) | | |
| | strand | | | |
| MAPT- | 19 mer | 2349-2426- | ACUUCCACCUGGCCACCUC | 1300 |
| 2349 | anti- | 1174 (Hs- | | |
| | sense | Mf-Mm) | | |
| | strand | | | |
| MAPT- | 19 mer | 2350-2427- | UACUUCCACCUGGCCACCU | 1301 |
| 2350 | anti- | 1175 (Hs- | | |
| | sense | Mf-Mm) | | |
| | strand | | | |
| MAPT- | 19 mer | 2351-2428- | UUACUUCCACCUGGCCACC | 1302 |
| 2351 | anti- | 1176 (Hs- | | |
| | sense | Mf-Mm) | | |
| | strand | | | |
| MAPT- | 19 mer | 2352-2429- | UUUACUUCCACCUGGCCAC | 1303 |
| 2352 | anti- | 1177 (Hs- | | |
| | sense | Mf-Mm) | | |
| | strand | | | |
| MAPT- | 19 mer | 2353-2430- | UUUUACUUCCACCUGGCCA | 1304 |
| 2353 | anti- | 1178 (Hs- | | |
| | sense | Mf-Mm) | | |
| | strand | | | |
| MAPT- | 19 mer | 2354-2431- | AUUUUACUUCCACCUGGCC | 1305 |
| 2354 | anti- | 1179 (Hs- | | |
| | sense | Mf-Mm) | | |
| | strand | | | |
| MAPT- | 19 mer | 2355-2432- | GAUUUUACUUCCACCUGGC | 1306 |
| 2355 | anti- | 1180 (Hs- | | |
| | sense | Mf-Mm) | | |
| | strand | | | |
| MAPT- | 19 mer | 2459-2536- | GCUUGUGGGUUUCAAUCUU | 1307 |
| 2459 | anti- | 1284 (Hs- | | |
| | sense | Mf-Mm) | | |
| | strand | | | |
| MAPT- | 19 mer | 2460-2537- | AGCUUGUGGGUUUCAAUCU | 1308 |
| 2460 | anti- | 1285 (Hs- | | |
| | sense | Mf-Mm) | | |
| | strand | | | |
| MAPT- | 19 mer | 2461-2538- | CAGCUUGUGGGUUUCAAUC | 1309 |
| 2461 | anti- | 1286 (Hs- | | |
| | sense | Mf-Mm) | | |
| | strand | | | |
| MAPT- | 19 mer | 2462-2539- | UCAGCUUGUGGGUUUCAAU | 1310 |
| 2462 | anti- | 1287 (Hs- | | |
| | sense | Mf-Mm) | | |
| | strand | | | |
| MAPT- | 19 mer | 2463-2540- | GUCAGCUUGUGGGUUUCAA | 1311 |
| 2463 | anti- | 1288 (Hs- | | |
| | sense | Mf-Mm) | | |
| | strand | | | |
| MAPT- | 19 mer | 2464-2541- | GGUCAGCUUGUGGGUUUCA | 1312 |
| 2464 | anti- | 1289 (Hs- | | |
| | sense | Mf-Mm) | | |
| | strand | | | |
| MAPT- | 19 mer | 2465-2542- | AGGUCAGCUUGUGGGUUUC | 1313 |
| 2465 | anti- | 1290 (Hs- | | |
| | sense | Mf-Mm) | | |
| | strand | | | |
| MAPT- | 19 mer | 2466-2543- | AAGGUCAGCUUGUGGGUUU | 1314 |
| 2466 | anti- | 1291 (Hs- | | |
| | sense | Mf-Mm) | | |
| | strand | | | |
| MAPT- | 19 mer | 2467-2544- | GAAGGUCAGCUUGUGGGUU | 1315 |
| 2467 | anti- | 1292 (Hs- | | |
| | sense | Mf-Mm) | | |
| | strand | | | |
| MAPT- | 19 mer | 2495-2572- | GGUCUGUCUUGGCUUUGGC | 1316 |
| 2495 | anti- | 1320 (Hs- | | |
| | sense | Mf-Mm) | | |
| | strand | | | |
| MAPT- | 19 mer | 2496-2573- | UGGUCUGUCUUGGCUUUGG | 1317 |
| 2496 | anti- | 1321 (Hs- | | |
| | sense | Mf-Mm) | | |
| | strand | | | |
| MAPT- | 19 mer | 3686-3758- | GCACAAGUCCUUACAAAGA | 1318 |
| 3686 | anti- | 2505 (Hs- | | |
| | sense | Mf-Mm) | | |
| | strand | | | |
| MAPT- | 19 mer | 3687-3759- | GGCACAAGUCCUUACAAAG | 1319 |
| 3687 | anti- | 2506 (Hs- | | |
| | sense | Mf-Mm) | | |
| | strand | | | |
| MAPT- | 19 mer | 3688-3760- | AGGCACAAGUCCUUACAAA | 1320 |
| 3688 | anti- | 2507 (Hs- | | |
| | sense | Mf-Mm) | | |
| | strand | | | |
| MAPT- | 19 mer | 3691-3763- | AAGAGGCACAAGUCCUUAC | 1321 |
| 3691 | anti- | 2510 (Hs- | | |
| | sense | Mf-Mm) | | |
| | strand | | | |
| MAPT- | 19 mer | 3692-3764- | CAAGAGGCACAAGUCCUUA | 1322 |
| 3692 | anti- | 2511 (Hs- | | |
| | sense | Mf-Mm) | | |
| | strand | | | |
| MAPT- | 19 mer | 3693-3765- | CCAAGAGGCACAAGUCCUU | 1323 |
| 3693 | anti- | 2512 (Hs- | | |
| | sense | Mf-Mm) | | |
| | strand | | | |
| MAPT- | 19 mer | 4534-4605- | AGACAAAUCCAACUACAAC | 1324 |
| 4534 | anti- | 3332 (Hs- | | |
| | sense | Mf-Mm) | | |
| | strand | | | |
| MAPT- | 19 mer | 4535-4606- | CAGACAAAUCCAACUACAA | 1325 |
| 4535 | anti- | 3333 (Hs- | | |
| | sense | Mf-Mm) | | |
| | strand | | | |
| MAPT- | 19 mer | 4536-4607- | ACAGACAAAUCCAACUACA | 1326 |
| 4536 | anti- | 3334 (Hs- | | |
| | sense | Mf-Mm) | | |
| | strand | | | |
| MAPT- | 19 mer | 4537-4608- | AACAGACAAAUCCAACUAC | 1327 |
| 4537 | anti- | 3335 (Hs- | | |
| | sense | Mf-Mm) | | |
| | strand | | | |
| MAPT- | 19 mer | 4538-4609- | AAACAGACAAAUCCAACUA | 1328 |
| 4538 | anti- | 3336 (Hs- | | |
| | sense | Mf-Mm) | | |
| | strand | | | |
| MAPT- | 19 mer | 4566-4637- | UCAUAGUCACUCUGGUGAA | 1329 |
| 4566 | anti- | 3362 (Hs- | | |
| | sense | Mf-Mm) | | |
| | strand | | | |
| MAPT- | 19 mer | 4567-4638- | AUCAUAGUCACUCUGGUGA | 1330 |
| 4567 | anti- | 3363 (Hs- | | |
| | sense | Mf-Mm) | | |
| | strand | | | |
| MAPT- | 19 mer | 4568-4639- | UAUCAUAGUCACUCUGGUG | 1331 |
| 4568 | anti- | 3364 (Hs- | | |
| | sense | Mf-Mm) | | |
| | strand | | | |
| MAPT- | 19 mer | 4569-4640- | CUAUCAUAGUCACUCUGGU | 1332 |
| 4569 | anti- | 3365 (Hs- | | |
| | sense | Mf-Mm) | | |
| | strand | | | |
| MAPT- | 19 mer | 4570-4641- | ACUAUCAUAGUCACUCUGG | 1333 |
| 4570 | anti- | 3366 (Hs- | | |
| | sense | Mf-Mm) | | |
| | strand | | | |
| MAPT- | 19 mer | 4571-4642- | CACUAUCAUAGUCACUCUG | 1334 |
| 4571 | anti- | 3367 (Hs- | | |
| | sense | Mf-Mm) | | |
| | strand | | | |
| MAPT- | 19 mer | 4572-4643- | UCACUAUCAUAGUCACUCU | 1335 |
| 4572 | anti- | 3368 (Hs- | | |
| | sense | Mf-Mm) | | |
| | strand | | | |
| MAPT- | 19 mer | 4573-4644- | UUCACUAUCAUAGUCACUC | 1336 |
| 4573 | anti- | 3369 (Hs- | | |
| | sense | Mf-Mm) | | |
| | strand | | | |
| MAPT- | 19 mer | 4574-4645- | UUUCACUAUCAUAGUCACU | 1337 |
| 4574 | anti- | 3370 (Hs- | | |
| | sense | Mf-Mm) | | |
| | strand | | | |
| MAPT- | 19 mer | 4575-4646- | UUUUCACUAUCAUAGUCAC | 1338 |
| 4575 | anti- | 3371 (Hs- | | |
| | sense | Mf-Mm) | | |
| | strand | | | |
| MAPT- | 19 mer | 4576-4647- | CUUUUCACUAUCAUAGUCA | 1339 |
| 4576 | anti- | 3372 (Hs- | | |
| | sense | Mf-Mm) | | |
| | strand | | | |
| MAPT- | 19 mer | 4577-4648- | UCUUUUCACUAUCAUAGUC | 1340 |
| 4577 | anti- | 3373 (Hs- | | |
| | sense | Mf-Mm) | | |
| | strand | | | |
| MAPT- | 19 mer | 4578-4649- | UUCUUUUCACUAUCAUAGU | 1341 |
| 4578 | anti- | 3374 (Hs- | | |
| | sense | Mf-Mm) | | |
| | strand | | | |
| MAPT- | 19 mer | 4579-4650- | UUUCUUUUCACUAUCAUAG | 1342 |
| 4579 | anti- | 3375 (Hs- | | |
| | sense | Mf-Mm) | | |
| | strand | | | |
| MAPT- | 19 mer | 4580-4651- | UUUUCUUUUCACUAUCAUA | 1343 |
| 4580 | anti- | 3376 (Hs- | | |
| | sense | Mf-Mm) | | |
| | strand | | | |
| MAPT- | 19 mer | 4605-4677- | UACAUGCGUCCUUUUUUUU | 1344 |
| 4605 | anti- | 3439 (Hs- | | |
| | sense | Mf-Mm) | | |
| | strand | | | |
| MAPT- | 19 mer | 4606-4678- | AUACAUGCGUCCUUUUUUU | 1345 |
| 4606 | anti- | 3440 (Hs- | | |
| | sense | Mf-Mm) | | |
| | strand | | | |
| MAPT- | 19 mer | 4607-4679- | GAUACAUGCGUCCUUUUUU | 1346 |
| 4607 | anti- | 3441 (Hs- | | |
| | sense | Mf-Mm) | | |
| | strand | | | |
| MAPT- | 19 mer | 4608-4680- | AGAUACAUGCGUCCUUUUU | 1347 |
| 4608 | anti- | 3442 (Hs- | | |
| | sense | Mf-Mm) | | |
| | strand | | | |
| MAPT- | 19 mer | 4609-4681- | AAGAUACAUGCGUCCUUUU | 1348 |
| 4609 | anti- | 3443 (Hs- | | |
| | sense | Mf-Mm) | | |
| | strand | | | |
| MAPT- | 19 mer | 4610-4682- | CAAGAUACAUGCGUCCUUU | 1349 |
| 4610 | anti- | 3444 (Hs- | | |
| | sense | Mf-Mm) | | |
| | strand | | | |
| MAPT- | 19 mer | 4611-4683- | UCAAGAUACAUGCGUCCUU | 1350 |
| 4611 | anti- | 3445 (Hs- | | |
| | sense | Mf-Mm) | | |
| | strand | | | |
| MAPT- | 19 mer | 4612-4684- | UUCAAGAUACAUGCGUCCU | 1351 |
| 4612 | anti- | 3446 (Hs- | | |
| | sense | Mf-Mm) | | |
| | strand | | | |
| MAPT- | 19 mer | 4613-4685- | UUUCAAGAUACAUGCGUCC | 1352 |
| 4613 | anti- | 3447 (Hs- | | |
| | sense | Mf-Mm) | | |
| | strand | | | |
| MAPT- | 19 mer | 4614-4686- | AUUUCAAGAUACAUGCGUC | 1353 |
| 4614 | anti- | 3448 (Hs- | | |
| | sense | Mf-Mm) | | |
| | strand | | | |
| MAPT- | 19 mer | 5969-6024- | GGAACUAUUGAUAAAGUGA | 1354 |
| 5969 | anti- | 4540 (Hs- | | |
| | sense | Mf-Mm) | | |
| | strand | | | |
| MAPT- | 19 mer | 5970-6025- | UGGAACUAUUGAUAAAGUG | 1355 |
| 5970 | anti- | 4541 (Hs- | | |
| | sense | Mf-Mm) | | |
| | strand | | | |
| MAPT- | 19 mer | 5971-6026- | AUGGAACUAUUGAUAAAGU | 1356 |
| 5971 | anti- | 4542 (Hs- | | |
| | sense | Mf-Mm) | | |
| | strand | | | |
| MAPT- | 19 mer | 5972-6027- | AAUGGAACUAUUGAUAAAG | 1357 |
| 5972 | anti- | 4543 (Hs- | | |
| | sense | Mf-Mm) | | |
| | strand | | | |
| MAPT- | 19 mer | 5973-6028- | AAAUGGAACUAUUGAUAAA | 1358 |
| 5973 | anti- | 4544 (Hs- | | |
| | sense | Mf-Mm) | | |
| | strand | | | |
| MAPT- | 19 mer | 5974-6029- | UAAAUGGAACUAUUGAUAA | 1359 |
| 5974 | anti- | 4545 (Hs- | | |
| | sense | Mf-Mm) | | |
| | strand | | | |
| MAPT- | 19 mer | 5975-6030- | UUAAAUGGAACUAUUGAUA | 1360 |
| 5975 | anti- | 4546 (Hs- | | |
| | sense | Mf-Mm) | | |
| | strand | | | |
| MAPT- | 19 mer | 5976-6031- | UUUAAAUGGAACUAUUGAU | 1361 |
| 5976 | anti- | 4547 (Hs- | | |
| | sense | Mf-Mm) | | |
| | strand | | | |
| MAPT- | 19 mer | 5977-6032- | AUUUAAAUGGAACUAUUGA | 1362 |
| 5977 | anti- | 4548 (Hs- | | |
| | sense | Mf-Mm) | | |
| | strand | | | |
| MAPT- | 19 mer | 5978-6033- | AAUUUAAAUGGAACUAUUG | 1363 |
| 5978 | anti- | 4549 (Hs- | | |
| | sense | Mf-Mm) | | |
| | strand | | | |
| MAPT- | 19 mer | 5979-6034- | CAAUUUAAAUGGAACUAUU | 1364 |
| 5979 | anti- | 4550 (Hs- | | |
| | sense | Mf-Mm) | | |
| | strand | | | |
| MAPT- | 19 mer | 5980-6035- | UCAAUUUAAAUGGAACUAU | 1365 |
| 5980 | anti- | 4551 (Hs- | | |
| | sense | Mf-Mm) | | |
| | strand | | | |
| MAPT- | 19 mer | 5981-6036- | GUCAAUUUAAAUGGAACUA | 1366 |
| 5981 | anti- | 4552 (Hs- | | |
| | sense | Mf-Mm) | | |
| | strand | | | |
| MAPT- | 19 mer | 5982-6037- | AGUCAAUUUAAAUGGAACU | 1367 |
| 5982 | anti- | 4553 (Hs- | | |
| | sense | Mf-Mm) | | |
| | strand | | | |
| MAPT- | 19 mer | 5983-6038- | AAGUCAAUUUAAAUGGAAC | 1368 |
| 5983 | anti- | 4554 (Hs- | | |
| | sense | Mf-Mm) | | |
| | strand | | | |
| MAPT- | 19 mer | 5984-6039- | GAAGUCAAUUUAAAUGGAA | 1369 |
| 5984 | anti- | 4555 (Hs- | | |
| | sense | Mf-Mm) | | |
| | strand | | | |
| MAPT- | 19 mer | 5985-6040- | UGAAGUCAAUUUAAAUGGA | 1370 |
| 5985 | anti- | 4556 (Hs- | | |
| | sense | Mf-Mm) | | |
| | strand | | | |
| MAPT- | 19 mer | 6662-6723- | AAAUCAUGGGACUUGCAAG | 1371 |
| 6662 | anti- | 5230 (Hs- | | |
| | sense | Mf-Mm) | | |
| | strand | | | |
| MAPT- | 19 mer | 6663-6724- | GAAAUCAUGGGACUUGCAA | 1372 |
| 6663 | anti- | 5231 (Hs- | | |
| | sense | Mf-Mm) | | |
| | strand | | | |
| MAPT- | 19 mer | 6664-6725- | AGAAAUCAUGGGACUUGCA | 1373 |
| 6664 | anti- | 5232 (Hs- | | |
| | sense | Mf-Mm) | | |
| | strand | | | |
| MAPT- | 19 mer | 6665-6726- | AAGAAAUCAUGGGACUUGC | 1374 |
| 6665 | anti- | 5233 (Hs- | | |
| | sense | Mf-Mm) | | |
| | strand | | | |
| MAPT- | 19 mer | 6800-6861- | UUUCCAAAUUCACUUUUAC | 1375 |
| 6800 | anti- | 5365 (Hs- | | |
| | sense | Mf-Mm) | | |
| | strand | | | |
| MAPT- | 19 mer | 6801-6862- | AUUUCCAAAUUCACUUUUA | 1376 |
| 6801 | anti- | 5366 (Hs- | | |
| | sense | Mf-Mm) | | |
| | strand | | | |
| MAPT- | 19 mer | 6802-6863- | UAUUUCCAAAUUCACUUUU | 1377 |
| 6802 | anti- | 5367 (Hs- | | |
| | sense | Mf-Mm) | | |
| | strand | | | |
| MAPT- | 19 mer | 6803-6864- | UUAUUUCCAAAUUCACUUU | 1378 |
| 6803 | anti- | 5368 (Hs- | | |
| | sense | Mf-Mm) | | |
| | strand | | | |
| MAPT- | 19 mer | 6804-6865- | UUUAUUUCCAAAUUCACUU | 1379 |
| 6804 | anti- | 5369 (Hs- | | |
| | sense | Mf-Mm) | | |
| | strand | | | |
| MAPT- | 19 mer | 6805-6866- | CUUUAUUUCCAAAUUCACU | 1380 |
| 6805 | anti- | 5370 (Hs- | | |
| | sense | Mf-Mm) | | |
| | strand | | | |
| MAPT- | 19 mer | 6806-6867- | ACUUUAUUUCCAAAUUCAC | 1381 |
| 6806 | anti- | 5371 (Hs- | | |
| | sense | Mf-Mm) | | |
| | strand | | | |
| MAPT- | 19 mer | 6807-6868- | AACUUUAUUUCCAAAUUCA | 1382 |
| 6807 | anti- | 5372 (Hs- | | |
| | sense | Mf-Mm) | | |
| | strand | | | |
| MAPT- | 19 mer | 6808-6869- | UAACUUUAUUUCCAAAUUC | 1383 |
| 6808 | anti- | 5373 (Hs- | | |
| | sense | Mf-Mm) | | |
| | strand | | | |
| MAPT- | 19 mer | 6809-6870- | AUAACUUUAUUUCCAAAUU | 1384 |
| 6809 | anti- | 5374 (Hs- | | |
| | sense | Mf-Mm) | | |
| | strand | | | |
| MAPT- | 19 mer | 6810-6871- | AAUAACUUUAUUUCCAAAU | 1385 |
| 6810 | anti- | 5375 (Hs- | | |
| | sense | Mf-Mm) | | |
| | strand | | | |
| MAPT- | 19 mer | 6811-6872- | UAAUAACUUUAUUUCCAAA | 1386 |
| 6811 | anti- | 5376 (Hs- | | |
| | sense | Mf-Mm) | | |
| | strand | | | |
| MAPT- | 19 mer | 6812-6873- | GUAAUAACUUUAUUUCCAA | 1387 |
| 6812 | anti- | 5377 (Hs- | | |
| | sense | Mf-Mm) | | |
| | strand | | | |
| MAPT- | 19 mer | 6813-6874- | AGUAAUAACUUUAUUUCCA | 1388 |
| 6813 | anti- | 5378 (Hs- | | |
| | sense | Mf-Mm) | | |
| | strand | | | |
| MAPT- | 19 mer | 6814-6875- | GAGUAAUAACUUUAUUUCC | 1389 |
| 6814 | anti- | 5379 (Hs- | | |
| | sense | Mf-Mm) | | |
| | strand | | | |
| MAPT- | 19 mer | 6815-6876- | AGAGUAAUAACUUUAUUUC | 1390 |
| 6815 | anti- | 5380 (Hs- | | |
| | sense | Mf-Mm) | | |
| | strand | | | |
| MAPT- | 19 mer | 6816-6877- | CAGAGUAAUAACUUUAUUU | 1391 |
| 6816 | anti- | 5381 (Hs- | | |
| | sense | Mf-Mm) | | |
| | strand | | | |
| MAPT- | 19 mer | 363 (Hs) | UCCAUCACUUCGAACUCCU | 1392 |
| 363 | anti- | | | |
| | sense | | | |
| | strand | | | |
| MAPT- | 19 mer | 364 (Hs) | UUCCAUCACUUCGAACUCC | 1393 |
| 364 | anti- | | | |
| | sense | | | |
| | strand | | | |
| MAPT- | 19 mer | 365 (Hs) | CUUCCAUCACUUCGAACUC | 1394 |
| 365 | anti- | | | |
| | sense | | | |
| | strand | | | |
| MAPT- | 19 mer | 367 (Hs) | AUCUUCCAUCACUUCGAAC | 1395 |
| 367 | anti- | | | |
| | sense | | | |
| | strand | | | |
| MAPT- | 19 mer | 369 (Hs) | UGAUCUUCCAUCACUUCGA | 1396 |
| 369 | anti- | | | |
| | sense | | | |
| | strand | | | |
| MAPT- | 19 mer | 374-226 | CAGCGUGAUCUUCCAUCAC | 1397 |
| 374 | anti- | (Hs-Mf) | | |
| | sense | | | |
| | strand | | | |
| MAPT- | 19 mer | 395-247 | UGUCCCCCAACCCGUACGU | 1398 |
| 395 | anti- | (Hs-Mf) | | |
| | sense | | | |
| | strand | | | |
| MAPT- | 19 mer | 400-252 | UUUCCUGUCCCCCAACCCG | 1399 |
| 400 | anti- | (Hs-Mf) | | |
| | sense | | | |
| | strand | | | |
| MAPT- | 19 mer | 443-295 | UGUCACCCUCUUGGUCUUG | 1400 |
| 443 | anti- | (Hs-Mf) | | |
| | sense | | | |
| | strand | | | |
| MAPT- | 19 mer | 688-453 | ACCAGCAGCUUCGUCUUCC | 1401 |
| 688 | anti- | (Hs-Mf) | | |
| | sense | | | |
| | strand | | | |
| MAPT- | 19 mer | 689-454 | GACCAGCAGCUUCGUCUUC | 1402 |
| 689 | anti- | (Hs-Mf) | | |
| | sense | | | |
| | strand | | | |
| MAPT- | 19 mer | 690-455 | UGACCAGCAGCUUCGUCUU | 1403 |
| 690 | anti- | (Hs-Mf) | | |
| | sense | | | |
| | strand | | | |
| MAPT- | 19 mer | 693-458 | ACGUGACCAGCAGCUUCGU | 1404 |
| 693 | anti- | (Hs-Mf) | | |
| | sense | | | |
| | strand | | | |
| MAPT- | 19 mer | 695-460 | UCACGUGACCAGCAGCUUC | 1405 |
| 695 | anti- | (Hs-Mf) | | |
| | sense | | | |
| | strand | | | |
| MAPT- | 19 mer | 696-461 | GUCACGUGACCAGCAGCUU | 1406 |
| 696 | anti- | (Hs-Mf) | | |
| | sense | | | |
| | strand | | | |
| MAPT- | 19 mer | 1475-1552 | UGCUUUUACUGACCAUGCG | 1407 |
| 1475 | anti- | (Hs-Mf) | | |
| | sense | | | |
| | strand | | | |
| MAPT- | 19 mer | 1476-1553 | UUGCUUUUACUGACCAUGC | 1408 |
| 1476 | anti- | (Hs-Mf) | | |
| | sense | | | |
| | strand | | | |
| MAPT- | 19 mer | 1479-1556 | UCUUUGCUUUUACUGACCA | 1409 |
| 1479 | anti- | (Hs-Mf) | | |
| | sense | | | |
| | strand | | | |
| MAPT- | 19 mer | 1480-1557 | GUCUUUGCUUUUACUGACC | 1410 |
| 1480 | anti- | (Hs-Mf) | | |
| | sense | | | |
| | strand | | | |
| MAPT- | 19 mer | 1481-1558 | CGUCUUUGCUUUUACUGAC | 1411 |
| 1481 | anti- | (Hs-Mf) | | |
| | sense | | | |
| | strand | | | |
| MAPT- | 19 mer | 1484-1561 | UCCCGUCUUUGCUUUUACU | 1412 |
| 1484 | anti- | (Hs-Mf) | | |
| | sense | | | |
| | strand | | | |
| MAPT- | 19 mer | 1485-1562 | GUCCCGUCUUUGCUUUUAC | 1413 |
| 1485 | anti- | (Hs-Mf) | | |
| | sense | | | |
| | strand | | | |
| MAPT- | 19 mer | 1492-1569 | GCUUCCAGUCCCGUCUUUG | 1414 |
| 1492 | anti- | (Hs-Mf) | | |
| | sense | | | |
| | strand | | | |
| MAPT- | 19 mer | 1494-1571 | UCGCUUCCAGUCCCGUCUU | 1415 |
| 1494 | anti- | (Hs-Mf) | | |
| | sense | | | |
| | strand | | | |
| MAPT- | 19 mer | 1495-1572 | AUCGCUUCCAGUCCCGUCU | 1416 |
| 1495 | anti- | (Hs-Mf) | | |
| | sense | | | |
| | strand | | | |
| MAPT- | 19 mer | 1498-1575 | GUCAUCGCUUCCAGUCCCG | 1417 |
| 1498 | anti- | (Hs-Mf) | | |
| | sense | | | |
| | strand | | | |
| MAPT- | 19 mer | 1499-1576 | UGUCAUCGCUUCCAGUCCC | 1418 |
| 1499 | anti- | (Hs-Mf) | | |
| | sense | | | |
| | strand | | | |
| MAPT- | 19 mer | 1500-1577 | UUGUCAUCGCUUCCAGUCC | 1419 |
| 1500 | anti- | (Hs-Mf) | | |
| | sense | | | |
| | strand | | | |
| MAPT- | 19 mer | 1502-1579 | UUUUGUCAUCGCUUCCAGU | 1420 |
| 1502 | anti- | (Hs-Mf) | | |
| | sense | | | |
| | strand | | | |
| MAPT- | 19 mer | 1503-1580 | UUUUUGUCAUCGCUUCCAG | 1421 |
| 1503 | anti- | (Hs-Mf) | | |
| | sense | | | |
| | strand | | | |
| MAPT- | 19 mer | 1504-1581 | UUUUUUGUCAUCGCUUCCA | 1422 |
| 1504 | anti- | (Hs-Mf) | | |
| | sense | | | |
| | strand | | | |
| MAPT- | 19 mer | 1505-1582 | CUUUUUUGUCAUCGCUUCC | 1423 |
| 1505 | anti- | (Hs-Mf) | | |
| | sense | | | |
| | strand | | | |
| MAPT- | 19 mer | 1506-1583 | GCUUUUUUGUCAUCGCUUC | 1424 |
| 1506 | anti- | (Hs-Mf) | | |
| | sense | | | |
| | strand | | | |
| MAPT- | 19 mer | 1507-1584 | GGCUUUUUUGUCAUCGCUU | 1425 |
| 1507 | anti- | (Hs-Mf) | | |
| | sense | | | |
| | strand | | | |
| MAPT- | 19 mer | 1508-1585 | UGGCUUUUUUGUCAUCGCU | 1426 |
| 1508 | anti- | (Hs-Mf) | | |
| | sense | | | |
| | strand | | | |
| MAPT- | 19 mer | 1509-1586 | UUGGCUUUUUUGUCAUCGC | 1427 |
| 1509 | anti- | (Hs-Mf) | | |
| | sense | | | |
| | strand | | | |
| MAPT- | 19 mer | 1733 (Hs) | CGAUCUUCGUUUUACCAUC | 1428 |
| 1733 | anti- | | | |
| | sense | | | |
| | strand | | | |
| MAPT- | 19 mer | 1796-1873 | CUGGAAUCCUGGUGGCGUU | 1429 |
| 1796 | anti- | (Hs-Mf) | | |
| | sense | | | |
| | strand | | | |
| MAPT- | 19 mer | 1835-1912 | CAGAGCUGGGUGGUGUCUU | 1430 |
| 1835 | anti- | (Hs-Mf) | | |
| | sense | | | |
| | strand | | | |
| MAPT- | 19 mer | 1912-1989 | AUCCCCUGAUUUUGGAGGU | 1431 |
| 1912 | anti- | (Hs-Mf) | | |
| | sense | | | |
| | strand | | | |
| MAPT- | 19 mer | 2094-2171 | UUCAGGUCUGGCAUGGGCA | 1432 |
| 2094 | anti- | (Hs-Mf) | | |
| | sense | | | |
| | strand | | | |
| MAPT- | 19 mer | 2096-2173 | UCUUCAGGUCUGGCAUGGG | 1433 |
| 2096 | anti- | (Hs-Mf) | | |
| | sense | | | |
| | strand | | | |
| MAPT- | 19 mer | 2097-2174 | UUCUUCAGGUCUGGCAUGG | 1434 |
| 2097 | anti- | (Hs-Mf) | | |
| | sense | | | |
| | strand | | | |
| MAPT- | 19 mer | 2098 (Hs) | AUUCUUCAGGUCUGGCAUG | 1435 |
| 2098 | anti- | | | |
| | sense | | | |
| | strand | | | |
| MAPT- | 19 mer | 2105 (Hs) | ACUUGACAUUCUUCAGGUC | 1436 |
| 2105 | anti- | | | |
| | sense | | | |
| | strand | | | |
| MAPT- | 19 mer | 2106 (Hs) | GACUUGACAUUCUUCAGGU | 1437 |
| 2106 | anti- | | | |
| | sense | | | |
| | strand | | | |
| MAPT- | 19 mer | 2107 (Hs) | GGACUUGACAUUCUUCAGG | 1438 |
| 2107 | anti- | | | |
| | sense | | | |
| | strand | | | |
| MAPT- | 19 mer | 2108 (Hs) | UGGACUUGACAUUCUUCAG | 1439 |
| 2108 | anti- | | | |
| | sense | | | |
| | strand | | | |
| MAPT- | 19 mer | 2109 (Hs) | UUGGACUUGACAUUCUUCA | 1440 |
| 2109 | anti- | | | |
| | sense | | | |
| | strand | | | |
| MAPT- | 19 mer | 2117-2194 | AGCCGAUCUUGGACUUGAC | 1441 |
| 2117 | anti- | (Hs-Mf) | | |
| | sense | | | |
| | strand | | | |
| MAPT- | 19 mer | 2136 (Hs) | UGCUUCAGGUUCUCAGUGG | 1442 |
| 2136 | anti- | | | |
| | sense | | | |
| | strand | | | |
| MAPT- | 19 mer | 2137 (Hs) | GUGCUUCAGGUUCUCAGUG | 1443 |
| 2137 | anti- | | | |
| | sense | | | |
| | strand | | | |
| MAPT- | 19 mer | 2269-2346 | UGGUUUGUAGACUAUUUGC | 1444 |
| 2269 | anti- | (Hs-Mf) | | |
| | sense | | | |
| | strand | | | |
| MAPT- | 19 mer | 2270-2347 | CUGGUUUGUAGACUAUUUG | 1445 |
| 2270 | anti- | (Hs-Mf) | | |
| | sense | | | |
| | strand | | | |
| MAPT- | 19 mer | 2271-2348 | ACUGGUUUGUAGACUAUUU | 1446 |
| 2271 | anti- | (Hs-Mf) | | |
| | sense | | | |
| | strand | | | |
| MAPT- | 19 mer | 2272-2349 | AACUGGUUUGUAGACUAUU | 1447 |
| 2272 | anti- | (Hs-Mf) | | |
| | sense | | | |
| | strand | | | |
| MAPT- | 19 mer | 2273-2350 | CAACUGGUUUGUAGACUAU | 1448 |
| 2273 | anti- | (Hs-Mf) | | |
| | sense | | | |
| | strand | | | |
| MAPT- | 19 mer | 2274-2351 | UCAACUGGUUUGUAGACUA | 1449 |
| 2274 | anti- | (Hs-Mf) | | |
| | sense | | | |
| | strand | | | |
| MAPT- | 19 mer | 2275-2352 | GUCAACUGGUUUGUAGACU | 1450 |
| 2275 | anti- | (Hs-Mf) | | |
| | sense | | | |
| | strand | | | |
| MAPT- | 19 mer | 2276-2353 | GGUCAACUGGUUUGUAGAC | 1451 |
| 2276 | anti- | (Hs-Mf) | | |
| | sense | | | |
| | strand | | | |
| MAPT- | 19 mer | 2277-2354 | AGGUCAACUGGUUUGUAGA | 1452 |
| 2277 | anti- | (Hs-Mf) | | |
| | sense | | | |
| | strand | | | |
| MAPT- | 19 mer | 2278-2355 | CAGGUCAACUGGUUUGUAG | 1453 |
| 2278 | anti- | (Hs-Mf) | | |
| | sense | | | |
| | strand | | | |
| MAPT- | 19 mer | 2279-2356 | UCAGGUCAACUGGUUUGUA | 1454 |
| 2279 | anti- | (Hs-Mf) | | |
| | sense | | | |
| | strand | | | |
| MAPT- | 19 mer | 2280-2357 | CUCAGGUCAACUGGUUUGU | 1455 |
| 2280 | anti- | (Hs-Mf) | | |
| | sense | | | |
| | strand | | | |
| MAPT- | 19 mer | 2281-2358 | GCUCAGGUCAACUGGUUUG | 1456 |
| 2281 | anti- | (Hs-Mf) | | |
| | sense | | | |
| | strand | | | |
| MAPT- | 19 mer | 2282-2359 | UGCUCAGGUCAACUGGUUU | 1457 |
| 2282 | anti- | (Hs-Mf) | | |
| | sense | | | |
| | strand | | | |
| MAPT- | 19 mer | 2283-2360 | UUGCUCAGGUCAACUGGUU | 1458 |
| 2283 | anti- | (Hs-Mf) | | |
| | sense | | | |
| | strand | | | |
| MAPT- | 19 mer | 2284-2361 | CUUGCUCAGGUCAACUGGU | 1459 |
| 2284 | anti- | (Hs-Mf) | | |
| | sense | | | |
| | strand | | | |
| MAPT- | 19 mer | 2286-2363 | ACCUUGCUCAGGUCAACUG | 1460 |
| 2286 | anti- | (Hs-Mf) | | |
| | sense | | | |
| | strand | | | |
| MAPT- | 19 mer | 2288-2365 | UCACCUUGCUCAGGUCAAC | 1461 |
| 2288 | anti- | (Hs-Mf) | | |
| | sense | | | |
| | strand | | | |
| MAPT- | 19 mer | 2289-2366 | GUCACCUUGCUCAGGUCAA | 1462 |
| 2289 | anti- | (Hs-Mf) | | |
| | sense | | | |
| | strand | | | |
| MAPT- | 19 mer | 2291-2368 | AGGUCACCUUGCUCAGGUC | 1463 |
| 2291 | anti- | (Hs-Mf) | | |
| | sense | | | |
| | strand | | | |
| MAPT- | 19 mer | 2294-2371 | UGGAGGUCACCUUGCUCAG | 1464 |
| 2294 | anti- | (Hs-Mf) | | |
| | sense | | | |
| | strand | | | |
| MAPT- | 19 mer | 2299-2376 | ACACUUGGAGGUCACCUUG | 1465 |
| 2299 | anti- | (Hs-Mf) | | |
| | sense | | | |
| | strand | | | |
| MAPT- | 19 mer | 2300-2377 | CACACUUGGAGGUCACCUU | 1466 |
| 2300 | anti- | (Hs-Mf) | | |
| | sense | | | |
| | strand | | | |
| MAPT- | 19 mer | 2301-2378 | CCACACUUGGAGGUCACCU | 1467 |
| 2301 | anti- | (Hs-Mf) | | |
| | sense | | | |
| | strand | | | |
| MAPT- | 19 mer | 2308-2385 | UAAUGAGCCACACUUGGAG | 1468 |
| 2308 | anti- | (Hs-Mf) | | |
| | sense | | | |
| | strand | | | |
| MAPT- | 19 mer | 2316-2393 | AUGUUGCCUAAUGAGCCAC | 1469 |
| 2316 | anti- | (Hs-Mf) | | |
| | sense | | | |
| | strand | | | |
| MAPT- | 19 mer | 2317-2394 | GAUGUUGCCUAAUGAGCCA | 1470 |
| 2317 | anti- | (Hs-Mf) | | |
| | sense | | | |
| | strand | | | |
| MAPT- | 19 mer | 2319-2396 | UGGAUGUUGCCUAAUGAGC | 1471 |
| 2319 | anti- | (Hs-Mf) | | |
| | sense | | | |
| | strand | | | |
| MAPT- | 19 mer | 2320-2397 | AUGGAUGUUGCCUAAUGAG | 1472 |
| 2320 | anti- | (Hs-Mf) | | |
| | sense | | | |
| | strand | | | |
| MAPT- | 19 mer | 2322-2399 | UGAUGGAUGUUGCCUAAUG | 1473 |
| 2322 | anti- | (Hs-Mf) | | |
| | sense | | | |
| | strand | | | |
| MAPT- | 19 mer | 2323-2400 | AUGAUGGAUGUUGCCUAAU | 1474 |
| 2323 | anti- | (Hs-Mf) | | |
| | sense | | | |
| | strand | | | |
| MAPT- | 19 mer | 2324-2401 | UAUGAUGGAUGUUGCCUAA | 1475 |
| 2324 | anti- | (Hs-Mf) | | |
| | sense | | | |
| | strand | | | |
| MAPT- | 19 mer | 2326-2403 | UUUAUGAUGGAUGUUGCCU | 1476 |
| 2326 | anti- | (Hs-Mf) | | |
| | sense | | | |
| | strand | | | |
| MAPT- | 19 mer | 2330-2407 | CUGGUUUAUGAUGGAUGUU | 1477 |
| 2330 | anti- | (Hs-Mf) | | |
| | sense | | | |
| | strand | | | |
| MAPT- | 19 mer | 2356-2433 | AGAUUUUACUUCCACCUGG | 1478 |
| 2356 | anti- | (Hs-Mf) | | |
| | sense | | | |
| | strand | | | |
| MAPT- | 19 mer | 2357-2434 | CAGAUUUUACUUCCACCUG | 1479 |
| 2357 | anti- | (Hs-Mf) | | |
| | sense | | | |
| | strand | | | |
| MAPT- | 19 mer | 2358-2435 | UCAGAUUUUACUUCCACCU | 1480 |
| 2358 | anti- | (Hs-Mf) | | |
| | sense | | | |
| | strand | | | |
| MAPT- | 19 mer | 2359-2436 | CUCAGAUUUUACUUCCACC | 1481 |
| 2359 | anti- | (Hs-Mf) | | |
| | sense | | | |
| | strand | | | |
| MAPT- | 19 mer | 2360-2437 | UCUCAGAUUUUACUUCCAC | 1482 |
| 2360 | anti- | (Hs-Mf) | | |
| | sense | | | |
| | strand | | | |
| MAPT- | 19 mer | 2361-2438 | UUCUCAGAUUUUACUUCCA | 1483 |
| 2361 | anti- | (Hs-Mf) | | |
| | sense | | | |
| | strand | | | |
| MAPT- | 19 mer | 2362-2439 | CUUCUCAGAUUUUACUUCC | 1484 |
| 2362 | anti- | (Hs-Mf) | | |
| | sense | | | |
| | strand | | | |
| MAPT- | 19 mer | 2363-2440 | GCUUCUCAGAUUUUACUUC | 1485 |
| 2363 | anti- | (Hs-Mf) | | |
| | sense | | | |
| | strand | | | |
| MAPT- | 19 mer | 2364-2441 | AGCUUCUCAGAUUUUACUU | 1486 |
| 2364 | anti- | (Hs-Mf) | | |
| | sense | | | |
| | strand | | | |
| MAPT- | 19 mer | 2365 (Hs) | AAGCUUCUCAGAUUUUACU | 1487 |
| 2365 | anti- | | | |
| | sense | | | |
| | strand | | | |
| MAPT- | 19 mer | 2372 (Hs) | UGAAGUCAAGCUUCUCAGA | 1488 |
| 2372 | anti- | | | |
| | sense | | | |
| | strand | | | |
| MAPT- | 19 mer | 2373 (Hs) | UUGAAGUCAAGCUUCUCAG | 1489 |
| 2373 | anti- | | | |
| | sense | | | |
| | strand | | | |
| MAPT- | 19 mer | 2374 (Hs) | CUUGAAGUCAAGCUUCUCA | 1490 |
| 2374 | anti- | | | |
| | sense | | | |
| | strand | | | |
| MAPT- | 19 mer | 2375 (Hs) | CCUUGAAGUCAAGCUUCUC | 1491 |
| 2375 | anti- | | | |
| | sense | | | |
| | strand | | | |
| MAPT- | 19 mer | 2376 (Hs) | UCCUUGAAGUCAAGCUUCU | 1492 |
| 2376 | anti- | | | |
| | sense | | | |
| | strand | | | |
| MAPT- | 19 mer | 2377 (Hs) | GUCCUUGAAGUCAAGCUUC | 1493 |
| 2377 | anti- | | | |
| | sense | | | |
| | strand | | | |
| MAPT- | 19 mer | 2378 (Hs) | UGUCCUUGAAGUCAAGCUU | 1494 |
| 2378 | anti- | | | |
| | sense | | | |
| | strand | | | |
| MAPT- | 19 mer | 2379 (Hs) | CUGUCCUUGAAGUCAAGCU | 1495 |
| 2379 | anti- | | | |
| | sense | | | |
| | strand | | | |
| MAPT- | 19 mer | 2380 (Hs) | UCUGUCCUUGAAGUCAAGC | 1496 |
| 2380 | anti- | | | |
| | sense | | | |
| | strand | | | |
| MAPT- | 19 mer | 2381 (Hs) | CUCUGUCCUUGAAGUCAAG | 1497 |
| 2381 | anti- | | | |
| | sense | | | |
| | strand | | | |
| MAPT- | 19 mer | 2382 (Hs) | ACUCUGUCCUUGAAGUCAA | 1498 |
| 2382 | anti- | | | |
| | sense | | | |
| | strand | | | |
| MAPT- | 19 mer | 2390 (Hs) | UCGACUGGACUCUGUCCUU | 1499 |
| 2390 | anti- | | | |
| | sense | | | |
| | strand | | | |
| MAPT- | 19 mer | 2391 (Hs) | UUCGACUGGACUCUGUCCU | 1500 |
| 2391 | anti- | | | |
| | sense | | | |
| | strand | | | |
| MAPT- | 19 mer | 2414-2491 | UGAUAUUGUCCAGGGACCC | 1501 |
| 2414 | anti- | (Hs-Mf) | | |
| | sense | | | |
| | strand | | | |
| MAPT- | 19 mer | 2448-2525 | UCAAUCUUUUUAUUUCCUC | 1502 |
| 2448 | anti- | (Hs-Mf) | | |
| | sense | | | |
| | strand | | | |
| MAPT- | 19 mer | 2449-2526 | UUCAAUCUUUUUAUUUCCU | 1503 |
| 2449 | anti- | (Hs-Mf) | | |
| | sense | | | |
| | strand | | | |
| MAPT- | 19 mer | 2450-2527 | UUUCAAUCUUUUUAUUUCC | 1504 |
| 2450 | anti- | (Hs-Mf) | | |
| | sense | | | |
| | strand | | | |
| MAPT- | 19 mer | 2451-2528 | GUUUCAAUCUUUUUAUUUC | 1505 |
| 2451 | anti- | (Hs-Mf) | | |
| | sense | | | |
| | strand | | | |
| MAPT- | 19 mer | 2452-2529 | GGUUUCAAUCUUUUUAUUU | 1506 |
| 2452 | anti- | (Hs-Mf) | | |
| | sense | | | |
| | strand | | | |
| MAPT- | 19 mer | 2453-2530 | GGGUUUCAAUCUUUUUAUU | 1507 |
| 2453 | anti- | (Hs-Mf) | | |
| | sense | | | |
| | strand | | | |
| MAPT- | 19 mer | 2454-2531 | UGGGUUUCAAUCUUUUUAU | 1508 |
| 2454 | anti- | (Hs-Mf) | | |
| | sense | | | |
| | strand | | | |
| MAPT- | 19 mer | 2456-2533 | UGUGGGUUUCAAUCUUUUU | 1509 |
| 2456 | anti- | (Hs-Mf) | | |
| | sense | | | |
| | strand | | | |
| MAPT- | 19 mer | 2457-2534 | UUGUGGGUUUCAAUCUUUU | 1510 |
| 2457 | anti- | (Hs-Mf) | | |
| | sense | | | |
| | strand | | | |
| MAPT- | 19 mer | | AGACAUUGCUGAGAUGCCG | 1511 |
| 2567 | anti- | 2567 (Hs) | | |
| | sense | | | |
| | strand | | | |
| MAPT- | 19 mer | 2598-2675 | GAGUCUACCAUGUCGAUGC | 1512 |
| 2598 | anti- | (Hs-Mf) | | |
| | sense | | | |
| | strand | | | |
| MAPT- | 19 mer | 2657-2734 | ACAAACCCUGCUUGGCCAG | 1513 |
| 2657 | anti- | (Hs-Mf) | | |
| | sense | | | |
| | strand | | | |
| MAPT- | 19 mer | 2723-2800 | CUUUUUUUUUCCACACUCU | 1514 |
| 2723 | anti- | (Hs-Mf) | | |
| | sense | | | |
| | strand | | | |
| MAPT- | 19 mer | 2724-2801 | UCUUUUUUUUUCCACACUC | 1515 |
| 2724 | anti- | (Hs-Mf) | | |
| | sense | | | |
| | strand | | | |
| MAPT- | 19 mer | 2726-2803 | AUUCUUUUUUUUUCCACAC | 1516 |
| 2726 | anti- | (Hs-Mf) | | |
| | sense | | | |
| | strand | | | |
| MAPT- | 19 mer | 2784-2860- | AACCAAUUAACCGAACUGC | 1517 |
| 2784 | anti- | 1 mismatch | | |
| | sense | (Hs-Mf) | | |
| | strand | | | |
| MAPT- | 19 mer | 2963-3039 | AGAAUCAAAAGGAAUUGCC | 1518 |
| 2963 | anti- | (Hs-Mf) | | |
| | sense | | | |
| | strand | | | |
| MAPT- | 19 mer | 3110-3186 | UUUCAAAUCCUUUGUUGCU | 1519 |
| 3110 | anti- | (Hs-Mf) | | |
| | sense | | | |
| | strand | | | |
| MAPT- | 19 mer | 3114-3190 | CAAGUUUCAAAUCCUUUGU | 1520 |
| 3114 | anti- | (Hs-Mf) | | |
| | sense | | | |
| | strand | | | |
| MAPT- | 19 mer | 3116-3192 | ACCAAGUUUCAAAUCCUUU | 1521 |
| 3116 | anti- | (Hs-Mf) | | |
| | sense | | | |
| | strand | | | |
| MAPT- | 19 mer | 3118-3194 | ACACCAAGUUUCAAAUCCU | 1522 |
| 3118 | anti- | (Hs-Mf) | | |
| | sense | | | |
| | strand | | | |
| MAPT- | 19 mer | 3158-3234 | UCACACAAGGUUGACAUCG | 1523 |
| 3158 | anti- | (Hs-Mf) | | |
| | sense | | | |
| | strand | | | |
| MAPT- | 19 mer | 3503-3576 | GACAUCAAGGUCAGUCUUU | 1524 |
| 3503 | anti- | (Hs-Mf) | | |
| | sense | | | |
| | strand | | | |
| MAPT- | 19 mer | 3589-3661 | AAACAGGGUUUCUGUGGAG | 1525 |
| 3589 | anti- | (Hs-Mf) | | |
| | sense | | | |
| | strand | | | |
| MAPT- | 19 mer | 3591-3663 | UAAAACAGGGUUUCUGUGG | 1526 |
| 3591 | anti- | (Hs-Mf) | | |
| | sense | | | |
| | strand | | | |
| MAPT- | 19 mer | 3592-3664 | AUAAAACAGGGUUUCUGUG | 1527 |
| 3592 | anti- | (Hs-Mf) | | |
| | sense | | | |
| | strand | | | |
| MAPT- | 19 mer | 3593-3665 | AAUAAAACAGGGUUUCUGU | 1528 |
| 3593 | anti- | (Hs-Mf) | | |
| | sense | | | |
| | strand | | | |
| MAPT- | 19 mer | 3594-3666 | CAAUAAAACAGGGUUUCUG | 1529 |
| 3594 | anti- | (Hs-Mf) | | |
| | sense | | | |
| | strand | | | |
| MAPT- | 19 mer | 3595-3667 | UCAAUAAAACAGGGUUUCU | 1530 |
| 3595 | anti- | (Hs-Mf) | | |
| | sense | | | |
| | strand | | | |
| MAPT- | 19 mer | 3596-3668 | CUCAAUAAAACAGGGUUUC | 1531 |
| 3596 | anti- | (Hs-Mf) | | |
| | sense | | | |
| | strand | | | |
| MAPT- | 19 mer | 3597-3669 | ACUCAAUAAAACAGGGUUU | 1532 |
| 3597 | anti- | (Hs-Mf) | | |
| | sense | | | |
| | strand | | | |
| MAPT- | 19 mer | 3598-3670 | AACUCAAUAAAACAGGGUU | 1533 |
| 3598 | anti- | (Hs-Mf) | | |
| | sense | | | |
| | strand | | | |
| MAPT- | 19 mer | 3599-3671 | GAACUCAAUAAAACAGGGU | 1534 |
| 3599 | anti- | (Hs-Mf) | | |
| | sense | | | |
| | strand | | | |
| MAPT- | 19 mer | 3600-3672 | AGAACUCAAUAAAACAGGG | 1535 |
| 3600 | anti- | (Hs-Mf) | | |
| | sense | | | |
| | strand | | | |
| MAPT- | 19 mer | 3601-3673 | CAGAACUCAAUAAAACAGG | 1536 |
| 3601 | anti- | (Hs-Mf) | | |
| | sense | | | |
| | strand | | | |
| MAPT- | 19 mer | 3602-3674 | UCAGAACUCAAUAAAACAG | 1537 |
| 3602 | anti- | (Hs-Mf) | | |
| | sense | | | |
| | strand | | | |
| MAPT- | 19 mer | 3603-3675 | UUCAGAACUCAAUAAAACA | 1538 |
| 3603 | anti- | (Hs-Mf) | | |
| | sense | | | |
| | strand | | | |
| MAPT- | 19 mer | 3605-3677 | CCUUCAGAACUCAAUAAAA | 1539 |
| 3605 | anti- | (Hs-Mf) | | |
| | sense | | | |
| | strand | | | |
| MAPT- | 19 mer | 3607-3679 | AACCUUCAGAACUCAAUAA | 1540 |
| 3607 | anti- | (Hs-Mf) | | |
| | sense | | | |
| | strand | | | |
| MAPT- | 19 mer | 3609-3681 | CCAACCUUCAGAACUCAAU | 1541 |
| 3609 | anti- | (Hs-Mf) | | |
| | sense | | | |
| | strand | | | |
| MAPT- | 19 mer | 3610-3682 | UCCAACCUUCAGAACUCAA | 1542 |
| 3610 | anti- | (Hs-Mf) | | |
| | sense | | | |
| | strand | | | |
| MAPT- | 19 mer | 3677-3749 | CUUACAAAGAGAACUGGUU | 1543 |
| 3677 | anti- | (Hs-Mf) | | |
| | sense | | | |
| | strand | | | |
| MAPT- | 19 mer | 3678-3750 | CCUUACAAAGAGAACUGGU | 1544 |
| 3678 | anti- | (Hs-Mf) | | |
| | sense | | | |
| | strand | | | |
| MAPT- | 19 mer | 3679-3751 | UCCUUACAAAGAGAACUGG | 1545 |
| 3679 | anti- | (Hs-Mf) | | |
| | sense | | | |
| | strand | | | |
| MAPT- | 19 mer | 3680-3752 | GUCCUUACAAAGAGAACUG | 1546 |
| 3680 | anti- | (Hs-Mf) | | |
| | sense | | | |
| | strand | | | |
| MAPT- | 19 mer | 3958-4030 | CACCCUCAGUAUGGAGUAG | 1547 |
| 3958 | anti- | (Hs-Mf) | | |
| | sense | | | |
| | strand | | | |
| MAPT- | 19 mer | 3959-4031 | UCACCCUCAGUAUGGAGUA | 1548 |
| 3959 | anti- | (Hs-Mf) | | |
| | sense | | | |
| | strand | | | |
| MAPT- | 19 mer | 3960-4032 | UUCACCCUCAGUAUGGAGU | 1549 |
| 3960 | anti- | (Hs-Mf) | | |
| | sense | | | |
| | strand | | | |
| MAPT- | 19 mer | 3961-4033 | UUUCACCCUCAGUAUGGAG | 1550 |
| 3961 | anti- | (Hs-Mf) | | |
| | sense | | | |
| | strand | | | |
| MAPT- | 19 mer | 3965-4037 | UUAAUUUCACCCUCAGUAU | 1551 |
| 3965 | anti- | (Hs-Mf) | | |
| | sense | | | |
| | strand | | | |
| MAPT- | 19 mer | 3970-4042 | UUCCCUUAAUUUCACCCUC | 1552 |
| 3970 | anti- | (Hs-Mf) | | |
| | sense | | | |
| | strand | | | |
| MAPT- | 19 mer | 4146-4218 | UCAACAAGGCAGAAACACC | 1553 |
| 4146 | anti- | (Hs-Mf) | | |
| | sense | | | |
| | strand | | | |
| MAPT- | 19 mer | 4474-4545 | AUAUGUUCAGCUGCUCCAG | 1554 |
| 4474 | anti- | (Hs-Mf) | | |
| | sense | | | |
| | strand | | | |
| MAPT- | 19 mer | 4475-4546 | UAUAUGUUCAGCUGCUCCA | 1555 |
| 4475 | anti- | (Hs-Mf) | | |
| | sense | | | |
| | strand | | | |
| MAPT- | 19 mer | 4477-4548 | UGUAUAUGUUCAGCUGCUC | 1556 |
| 4477 | anti- | (Hs-Mf) | | |
| | sense | | | |
| | strand | | | |
| MAPT- | 19 mer | 4478-4549 | AUGUAUAUGUUCAGCUGCU | 1557 |
| 4478 | anti- | (Hs-Mf) | | |
| | sense | | | |
| | strand | | | |
| MAPT- | 19 mer | 4479-4550 | UAUGUAUAUGUUCAGCUGC | 1558 |
| 4479 | anti- | (Hs-Mf) | | |
| | sense | | | |
| | strand | | | |
| MAPT- | 19 mer | 4480-4551 | CUAUGUAUAUGUUCAGCUG | 1559 |
| 4480 | anti- | (Hs-Mf) | | |
| | sense | | | |
| | strand | | | |
| MAPT- | 19 mer | 4481-4552 | UCUAUGUAUAUGUUCAGCU | 1560 |
| 4481 | anti- | (Hs-Mf) | | |
| | sense | | | |
| | strand | | | |
| MAPT- | 19 mer | 4482-4553 | AUCUAUGUAUAUGUUCAGC | 1561 |
| 4482 | anti- | (Hs-Mf) | | |
| | sense | | | |
| | strand | | | |
| MAPT- | 19 mer | 4485-4556 | AACAUCUAUGUAUAUGUUC | 1562 |
| 4485 | anti- | (Hs-Mf) | | |
| | sense | | | |
| | strand | | | |
| MAPT- | 19 mer | 4486-4557 | CAACAUCUAUGUAUAUGUU | 1563 |
| 4486 | anti- | (Hs-Mf) | | |
| | sense | | | |
| | strand | | | |
| MAPT- | 19 mer | 4532 (Hs) | ACAAAUCCAACUACAACUC | 1564 |
| 4532 | anti- | | | |
| | sense | | | |
| | strand | | | |
| MAPT- | 19 mer | 4533 (Hs) | GACAAAUCCAACUACAACU | 1565 |
| 4533 | anti- | | | |
| | sense | | | |
| | strand | | | |
| MAPT- | 19 mer | 4539-4610 | UAAACAGACAAAUCCAACU | 1566 |
| 4539 | anti- | (Hs-Mf) | | |
| | sense | | | |
| | strand | | | |
| MAPT- | 19 mer | 4540-4611 | AUAAACAGACAAAUCCAAC | 1567 |
| 4540 | anti- | (Hs-Mf) | | |
| | sense | | | |
| | strand | | | |
| MAPT- | 19 mer | 4541-4612 | CAUAAACAGACAAAUCCAA | 1568 |
| 4541 | anti- | (Hs-Mf) | | |
| | sense | | | |
| | strand | | | |
| MAPT- | 19 mer | 4543-4614 | AGCAUAAACAGACAAAUCC | 1569 |
| 4543 | anti- | (Hs-Mf) | | |
| | sense | | | |
| | strand | | | |
| MAPT- | 19 mer | 4544-4615 | AAGCAUAAACAGACAAAUC | 1570 |
| 4544 | anti- | (Hs-Mf) | | |
| | sense | | | |
| | strand | | | |
| MAPT- | 19 mer | 4545-4616 | CAAGCAUAAACAGACAAAU | 1571 |
| 4545 | anti- | (Hs-Mf) | | |
| | sense | | | |
| | strand | | | |
| MAPT- | 19 mer | 4546-4617 | CCAAGCAUAAACAGACAAA | 1572 |
| 4546 | anti- | (Hs-Mf) | | |
| | sense | | | |
| | strand | | | |
| MAPT- | 19 mer | 4547-4618 | UCCAAGCAUAAACAGACAA | 1573 |
| 4547 | anti- | (Hs-Mf) | | |
| | sense | | | |
| | strand | | | |
| MAPT- | 19 mer | 4548-4619 | AUCCAAGCAUAAACAGACA | 1574 |
| 4548 | anti- | (Hs-Mf) | | |
| | sense | | | |
| | strand | | | |
| MAPT- | 19 mer | 4549-4620 | AAUCCAAGCAUAAACAGAC | 1575 |
| 4549 | anti- | (Hs-Mf) | | |
| | sense | | | |
| | strand | | | |
| MAPT- | 19 mer | 4550-4621 | GAAUCCAAGCAUAAACAGA | 1576 |
| 4550 | anti- | (Hs-Mf) | | |
| | sense | | | |
| | strand | | | |
| MAPT- | 19 mer | 4551-4622 | UGAAUCCAAGCAUAAACAG | 1577 |
| 4551 | anti- | (Hs-Mf) | | |
| | sense | | | |
| | strand | | | |
| MAPT- | 19 mer | 4552-4623 | GUGAAUCCAAGCAUAAACA | 1578 |
| 4552 | anti- | (Hs-Mf) | | |
| | sense | | | |
| | strand | | | |
| MAPT- | 19 mer | 4554-4625 | UGGUGAAUCCAAGCAUAAA | 1579 |
| 4554 | anti- | (Hs-Mf) | | |
| | sense | | | |
| | strand | | | |
| MAPT- | 19 mer | 4556-4627 | UCUGGUGAAUCCAAGCAUA | 1580 |
| 4556 | anti- | (Hs-Mf) | | |
| | sense | | | |
| | strand | | | |
| MAPT- | 19 mer | 4557-4628 | CUCUGGUGAAUCCAAGCAU | 1581 |
| 4557 | anti- | (Hs-Mf) | | |
| | sense | | | |
| | strand | | | |
| MAPT- | 19 mer | 4558-4629 | ACUCUGGUGAAUCCAAGCA | 1582 |
| 4558 | anti- | (Hs-Mf) | | |
| | sense | | | |
| | strand | | | |
| MAPT- | 19 mer | 4559-4630 | CACUCUGGUGAAUCCAAGC | 1583 |
| 4559 | anti- | (Hs-Mf) | | |
| | sense | | | |
| | strand | | | |
| MAPT- | 19 mer | 4560-4631 | UCACUCUGGUGAAUCCAAG | 1584 |
| 4560 | anti- | (Hs-Mf) | | |
| | sense | | | |
| | strand | | | |
| MAPT- | 19 mer | 4561-4632 | GUCACUCUGGUGAAUCCAA | 1585 |
| 4561 | anti- | (Hs-Mf) | | |
| | sense | | | |
| | strand | | | |
| MAPT- | 19 mer | 4562-4633 | AGUCACUCUGGUGAAUCCA | 1586 |
| 4562 | anti- | (Hs-Mf) | | |
| | sense | | | |
| | strand | | | |
| MAPT- | 19 mer | 4563-4634 | UAGUCACUCUGGUGAAUCC | 1587 |
| 4563 | anti- | (Hs-Mf) | | |
| | sense | | | |
| | strand | | | |
| MAPT- | 19 mer | 4564-4635 | AUAGUCACUCUGGUGAAUC | 1588 |
| 4564 | anti- | (Hs-Mf) | | |
| | sense | | | |
| | strand | | | |
| MAPT- | 19 mer | 4615-4687 | CAUUUCAAGAUACAUGCGU | 1589 |
| 4615 | anti- | (Hs-Mf) | | |
| | sense | | | |
| | strand | | | |
| MAPT- | 19 mer | 4616-4688 | GCAUUUCAAGAUACAUGCG | 1590 |
| 4616 | anti- | (Hs-Mf) | | |
| | sense | | | |
| | strand | | | |
| MAPT- | 19 mer | 4617-4689 | AGCAUUUCAAGAUACAUGC | 1591 |
| 4617 | anti- | (Hs-Mf) | | |
| | sense | | | |
| | strand | | | |
| MAPT- | 19 mer | 4618-4690 | AAGCAUUUCAAGAUACAUG | 1592 |
| 4618 | anti- | (Hs-Mf) | | |
| | sense | | | |
| | strand | | | |
| MAPT- | 19 mer | 4619-4691 | CAAGCAUUUCAAGAUACAU | 1593 |
| 4619 | anti- | (Hs-Mf) | | |
| | sense | | | |
| | strand | | | |
| MAPT- | 19 mer | 4620-4692 | ACAAGCAUUUCAAGAUACA | 1594 |
| 4620 | anti- | (Hs-Mf) | | |
| | sense | | | |
| | strand | | | |
| MAPT- | 19 mer | 4621-4693 | UACAAGCAUUUCAAGAUAC | 1595 |
| 4621 | anti- | (Hs-Mf) | | |
| | sense | | | |
| | strand | | | |
| MAPT- | 19 mer | 4622-4694 | UUACAAGCAUUUCAAGAUA | 1596 |
| 4622 | anti- | (Hs-Mf) | | |
| | sense | | | |
| | strand | | | |
| MAPT- | 19 mer | 4623-4695 | UUUACAAGCAUUUCAAGAU | 1597 |
| 4623 | anti- | (Hs-Mf) | | |
| | sense | | | |
| | strand | | | |
| MAPT- | 19 mer | 4625-4697 | UCUUUACAAGCAUUUCAAG | 1598 |
| 4625 | anti- | (Hs-Mf) | | |
| | sense | | | |
| | strand | | | |
| MAPT- | 19 mer | 4627-4699 | CCUCUUUACAAGCAUUUCA | 1599 |
| 4627 | anti- | (Hs-Mf) | | |
| | sense | | | |
| | strand | | | |
| MAPT- | 19 mer | 4628-4700 | ACCUCUUUACAAGCAUUUC | 1600 |
| 4628 | anti- | (Hs-Mf) | | |
| | sense | | | |
| | strand | | | |
| MAPT- | 19 mer | 4629-4701 | AACCUCUUUACAAGCAUUU | 1601 |
| 4629 | anti- | (Hs-Mf) | | |
| | sense | | | |
| | strand | | | |
| MAPT- | 19 mer | 4630-4702 | AAACCUCUUUACAAGCAUU | 1602 |
| 4630 | anti- | (Hs-Mf) | | |
| | sense | | | |
| | strand | | | |
| MAPT- | 19 mer | 4632-4704 | AGAAACCUCUUUACAAGCA | 1603 |
| 4632 | anti- | (Hs-Mf) | | |
| | sense | | | |
| | strand | | | |
| MAPT- | 19 mer | 4633-4705 | UAGAAACCUCUUUACAAGC | 1604 |
| 4633 | anti- | (Hs-Mf) | | |
| | sense | | | |
| | strand | | | |
| MAPT- | 19 mer | 4825-4897 | GCUUCAGUCCUAAUCCUGU | 1605 |
| 4825 | anti- | (Hs-Mf) | | |
| | sense | | | |
| | strand | | | |
| MAPT- | 19 mer | 4828-4900 | AUCGCUUCAGUCCUAAUCC | 1606 |
| 4828 | anti- | (Hs-Mf) | | |
| | sense | | | |
| | strand | | | |
| MAPT- | 19 mer | 5682-5743 | AGCAGGGCACAAGAACUUC | 1607 |
| 5682 | anti- | (Hs-Mf) | | |
| | sense | | | |
| | strand | | | |
| MAPT- | 19 mer | 5958 (Hs) | UAAAGUGAGUCAGCAGCUU | 1608 |
| 5958 | anti- | | | |
| | sense | | | |
| | strand | | | |
| MAPT- | 19 mer | 5959 (Hs) | AUAAAGUGAGUCAGCAGCU | 1609 |
| 5959 | anti- | | | |
| | sense | | | |
| | strand | | | |
| MAPT- | 19 mer | 5961 (Hs) | UGAUAAAGUGAGUCAGCAG | 1610 |
| 5961 | anti- | | | |
| | sense | | | |
| | strand | | | |
| MAPT- | 19 mer | 5963 (Hs) | AUUGAUAAAGUGAGUCAGC | 1611 |
| 5963 | anti- | | | |
| | sense | | | |
| | strand | | | |
| MAPT- | 19 mer | 5964 (Hs) | UAUUGAUAAAGUGAGUCAG | 1612 |
| 5964 | anti- | | | |
| | sense | | | |
| | strand | | | |
| MAPT- | 19 mer | 5965 (Hs) | CUAUUGAUAAAGUGAGUCA | 1613 |
| 5965 | anti- | | | |
| | sense | | | |
| | strand | | | |
| MAPT- | 19 mer | 5966-6021 | ACUAUUGAUAAAGUGAGUC | 1614 |
| 5966 | anti- | (Hs-Mf) | | |
| | sense | | | |
| | strand | | | |
| MAPT- | 19 mer | 5967-6022 | AACUAUUGAUAAAGUGAGU | 1615 |
| 5967 | anti- | (Hs-Mf) | | |
| | sense | | | |
| | strand | | | |
| MAPT- | 19 mer | 5968-6023 | GAACUAUUGAUAAAGUGAG | 1616 |
| 5968 | anti- | (Hs-Mf) | | |
| | sense | | | |
| | strand | | | |
| MAPT- | 19 mer | 6006-6061 | AACAGGAUACAGUCUCACC | 1617 |
| 6006 | anti- | (Hs-Mf) | | |
| | sense | | | |
| | strand | | | |
| MAPT- | 19 mer | 6007-6062 | AAACAGGAUACAGUCUCAC | 1618 |
| 6007 | anti- | (Hs-Mf) | | |
| | sense | | | |
| | strand | | | |
| MAPT- | 19 mer | 6008-6063 | CAAACAGGAUACAGUCUCA | 1619 |
| 6008 | anti- | (Hs-Mf) | | |
| | sense | | | |
| | strand | | | |
| MAPT- | 19 mer | 6009-6064 | GCAAACAGGAUACAGUCUC | 1620 |
| 6009 | anti- | (Hs-Mf) | | |
| | sense | | | |
| | strand | | | |
| MAPT- | 19 mer | 6010-6065 | AGCAAACAGGAUACAGUCU | 1621 |
| 6010 | anti- | (Hs-Mf) | | |
| | sense | | | |
| | strand | | | |
| MAPT- | 19 mer | 6011-6066 | UAGCAAACAGGAUACAGUC | 1622 |
| 6011 | anti- | (Hs-Mf) | | |
| | sense | | | |
| | strand | | | |
| MAPT- | 19 mer | 6012-6067 | AUAGCAAACAGGAUACAGU | 1623 |
| 6012 | anti- | (Hs-Mf) | | |
| | sense | | | |
| | strand | | | |
| MAPT- | 19 mer | 6013-6068 | AAUAGCAAACAGGAUACAG | 1624 |
| 6013 | anti- | (Hs-Mf) | | |
| | sense | | | |
| | strand | | | |
| MAPT- | 19 mer | 6014-6069 | CAAUAGCAAACAGGAUACA | 1625 |
| 6014 | anti- | (Hs-Mf) | | |
| | sense | | | |
| | strand | | | |
| MAPT- | 19 mer | 6015-6070 | GCAAUAGCAAACAGGAUAC | 1626 |
| 6015 | anti- | (Hs-Mf) | | |
| | sense | | | |
| | strand | | | |
| MAPT- | 19 mer | 6017-6072 | AAGCAAUAGCAAACAGGAU | 1627 |
| 6017 | anti- | (Hs-Mf) | | |
| | sense | | | |
| | strand | | | |
| MAPT- | 19 mer | 6119-6174 | AUGAAAAGGGUUACGAGGC | 1628 |
| 6119 | anti- | (Hs-Mf) | | |
| | sense | | | |
| | strand | | | |
| MAPT- | 19 mer | 6628-6689 | UGCUCAACAUGGCAAACUC | 1629 |
| 6628 | anti- | (Hs-Mf) | | |
| | sense | | | |
| | strand | | | |
| MAPT- | 19 mer | 6629-6690 | CUGCUCAACAUGGCAAACU | 1630 |
| 6629 | anti- | (Hs-Mf) | | |
| | sense | | | |
| | strand | | | |
| MAPT- | 19 mer | 6631-6692 | UCCUGCUCAACAUGGCAAA | 1631 |
| 6631 | anti- | (Hs-Mf) | | |
| | sense | | | |
| | strand | | | |
| MAPT- | 19 mer | 6672-6733 | AUUACCGAAGAAAUCAUGG | 1632 |
| 6672 | anti- | (Hs-Mf) | | |
| | sense | | | |
| | strand | | | |
| MAPT- | 19 mer | 6731 (Hs) | ACAUUCACAGACAGAAAGC | 1633 |
| 6731 | anti- | | | |
| | sense | | | |
| | strand | | | |
| MAPT- | 19 mer | 6732 (Hs) | GACAUUCACAGACAGAAAG | 1634 |
| 6732 | anti- | | | |
| | sense | | | |
| | strand | | | |
| MAPT- | 19 mer | 6738-6799 | UAUAUAGACAUUCACAGAC | 1635 |
| 6738 | anti- | (Hs-Mf) | | |
| | sense | | | |
| | strand | | | |
| MAPT- | 19 mer | 6739-6800 | CUAUAUAGACAUUCACAGA | 1636 |
| 6739 | anti- | (Hs-Mf) | | |
| | sense | | | |
| | strand | | | |
| MAPT- | 19 mer | 6740-6801 | ACUAUAUAGACAUUCACAG | 1637 |
| 6740 | anti- | (Hs-Mf) | | |
| | sense | | | |
| | strand | | | |
| MAPT- | 19 mer | 6741-6802 | CACUAUAUAGACAUUCACA | 1638 |
| 6741 | anti- | (Hs-Mf) | | |
| | sense | | | |
| | strand | | | |
| MAPT- | 19 mer | 6742-6803 | ACACUAUAUAGACAUUCAC | 1639 |
| 6742 | anti- | (Hs-Mf) | | |
| | sense | | | |
| | strand | | | |
| MAPT- | 19 mer | 6743-6804 | UACACUAUAUAGACAUUCA | 1640 |
| 6743 | anti- | (Hs-Mf) | | |
| | sense | | | |
| | strand | | | |
| MAPT- | 19 mer | 6745-6806 | AAUACACUAUAUAGACAUU | 1641 |
| 6745 | anti- | (Hs-Mf) | | |
| | sense | | | |
| | strand | | | |
| MAPT- | 19 mer | 6748-6809 | CACAAUACACUAUAUAGAC | 1642 |
| 6748 | anti- | (Hs-Mf) | | |
| | sense | | | |
| | strand | | | |
| MAPT- | 19 mer | 6749-6810 | ACACAAUACACUAUAUAGA | 1643 |
| 6749 | anti- | (Hs-Mf) | | |
| | sense | | | |
| | strand | | | |
| MAPT- | 19 mer | 6750-6811 | CACACAAUACACUAUAUAG | 1644 |
| 6750 | anti- | (Hs-Mf) | | |
| | sense | | | |
| | strand | | | |
| MAPT- | 19 mer | 6751-6812 | ACACACAAUACACUAUAUA | 1645 |
| 6751 | anti- | (Hs-Mf) | | |
| | sense | | | |
| | strand | | | |
| MAPT- | 19 mer | 6752-6813 | AACACACAAUACACUAUAU | 1646 |
| 6752 | anti- | (Hs-Mf) | | |
| | sense | | | |
| | strand | | | |
| MAPT- | 19 mer | 6753-6814 | AAACACACAAUACACUAUA | 1647 |
| 6753 | anti- | (Hs-Mf) | | |
| | sense | | | |
| | strand | | | |
| MAPT- | 19 mer | 6754-6815 | AAAACACACAAUACACUAU | 1648 |
| 6754 | anti- | (Hs-Mf) | | |
| | sense | | | |
| | strand | | | |
| MAPT- | 19 mer | 6755-6816 | UAAAACACACAAUACACUA | 1649 |
| 6755 | anti- | (Hs-Mf) | | |
| | sense | | | |
| | strand | | | |
| MAPT- | 19 mer | 6756-6817 | UUAAAACACACAAUACACU | 1650 |
| 6756 | anti- | (Hs-Mf) | | |
| | sense | | | |
| | strand | | | |
| MAPT- | 19 mer | 6757-6818 | GUUAAAACACACAAUACAC | 1651 |
| 6757 | anti- | (Hs-Mf) | | |
| | sense | | | |
| | strand | | | |
| MAPT- | 19 mer | 6758-6819 | UGUUAAAACACACAAUACA | 1652 |
| 6758 | anti- | (Hs-Mf) | | |
| | sense | | | |
| | strand | | | |
| MAPT- | 19 mer | 6759-6820 | UUGUUAAAACACACAAUAC | 1653 |
| 6759 | anti- | (Hs-Mf) | | |
| | sense | | | |
| | strand | | | |
| MAPT- | 19 mer | 6760-6821 | UUUGUUAAAACACACAAUA | 1654 |
| 6760 | anti- | (Hs-Mf) | | |
| | sense | | | |
| | strand | | | |
| MAPT- | 19 mer | 6761-6822 | AUUUGUUAAAACACACAAU | 1655 |
| 6761 | anti- | (Hs-Mf) | | |
| | sense | | | |
| | strand | | | |
| MAPT- | 19 mer | 6762-6823 | CAUUUGUUAAAACACACAA | 1656 |
| 6762 | anti- | (Hs-Mf) | | |
| | sense | | | |
| | strand | | | |
| MAPT- | 19 mer | 6763-6824 | UCAUUUGUUAAAACACACA | 1657 |
| 6763 | anti- | (Hs-Mf) | | |
| | sense | | | |
| | strand | | | |
| MAPT- | 19 mer | 6764-6825 | AUCAUUUGUUAAAACACAC | 1658 |
| 6764 | anti- | (Hs-Mf) | | |
| | sense | | | |
| | strand | | | |
| MAPT- | 19 mer | 6765-6826 | AAUCAUUUGUUAAAACACA | 1659 |
| 6765 | anti- | (Hs-Mf) | | |
| | sense | | | |
| | strand | | | |
| MAPT- | 19 mer | 6766-6827 | AAAUCAUUUGUUAAAACAC | 1660 |
| 6766 | anti- | (Hs-Mf) | | |
| | sense | | | |
| | strand | | | |
| MAPT- | 19 mer | 6767-6828 | UAAAUCAUUUGUUAAAACA | 1661 |
| 6767 | anti- | (Hs-Mf) | | |
| | sense | | | |
| | strand | | | |
| MAPT- | 19 mer | 6768-6829 | GUAAAUCAUUUGUUAAAAC | 1662 |
| 6768 | anti- | (Hs-Mf) | | |
| | sense | | | |
| | strand | | | |
| MAPT- | 19 mer | 6769-6830 | UGUAAAUCAUUUGUUAAAA | 1663 |
| 6769 | anti- | (Hs-Mf) | | |
| | sense | | | |
| | strand | | | |
| MAPT- | 19 mer | 6772-6833 | CAGUGUAAAUCAUUUGUUA | 1664 |
| 6772 | anti- | (Hs-Mf) | | |
| | sense | | | |
| | strand | | | |
| MAPT- | 19 mer | 6773-6834 | UCAGUGUAAAUCAUUUGUU | 1665 |
| 6773 | anti- | (Hs-Mf) | | |
| | sense | | | |
| | strand | | | |
| MAPT- | 19 mer | 6774-6835 | GUCAGUGUAAAUCAUUUGU | 1666 |
| 6774 | anti- | (Hs-Mf) | | |
| | sense | | | |
| | strand | | | |
| MAPT- | 19 mer | 6775-6836 | AGUCAGUGUAAAUCAUUUG | 1667 |
| 6775 | anti- | (Hs-Mf) | | |
| | sense | | | |
| | strand | | | |
| MAPT- | 19 mer | 6777-6838 | ACAGUCAGUGUAAAUCAUU | 1668 |
| 6777 | anti- | (Hs-Mf) | | |
| | sense | | | |
| | strand | | | |
| MAPT- | 19 mer | 6778-6839 | AACAGUCAGUGUAAAUCAU | 1669 |
| 6778 | anti- | (Hs-Mf) | | |
| | sense | | | |
| | strand | | | |
| MAPT- | 19 mer | 6779-6840 | CAACAGUCAGUGUAAAUCA | 1670 |
| 6779 | anti- | (Hs-Mf) | | |
| | sense | | | |
| | strand | | | |
| MAPT- | 19 mer | 6780-6841 | GCAACAGUCAGUGUAAAUC | 1671 |
| 6780 | anti- | (Hs-Mf) | | |
| | sense | | | |
| | strand | | | |
| MAPT- | 19 mer | 6781 (Hs) | AGCAACAGUCAGUGUAAAU | 1672 |
| 6781 | anti- | | | |
| | sense | | | |
| | strand | | | |
| MAPT- | 19 mer | 6789 (Hs) | ACUUUUACAGCAACAGUCA | 1673 |
| 6789 | anti- | | | |
| | sense | | | |
| | strand | | | |
| MAPT- | 19 mer | 6792 (Hs) | UUCACUUUUACAGCAACAG | 1674 |
| 6792 | anti- | | | |
| | sense | | | |
| | strand | | | |
| MAPT- | 19 mer | 6793 (Hs) | AUUCACUUUUACAGCAACA | 1675 |
| 6793 | anti- | | | |
| | sense | | | |
| | strand | | | |
| MAPT- | 19 mer | 6795 (Hs) | AAAUUCACUUUUACAGCAA | 1676 |
| 6795 | anti- | | | |
| | sense | | | |
| | strand | | | |
| MAPT- | 19 mer | 6796 (Hs) | CAAAUUCACUUUUACAGCA | 1677 |
| 6796 | anti- | | | |
| | sense | | | |
| | strand | | | |
| MAPT- | 19 mer | 6797 (Hs) | CCAAAUUCACUUUUACAGC | 1678 |
| 6797 | anti- | | | |
| | sense | | | |
| | strand | | | |
| MAPT- | 19 mer | 6798 (Hs) | UCCAAAUUCACUUUUACAG | 1679 |
| 6798 | anti- | | | |
| | sense | | | |
| | strand | | | |
| Stem | N/A | N/A | GCAGCCGAAAGGCUGC | 1680 |
| loop | | | | |
| MAPT- | 20 mer | 2357-2434 | CAGGUGGAAGUAAAAUCUGA | 1681 |
| 2357 | sense | (Hs-Mf) | | |
| | strand | | | |
| MAPT- | 20 mer | 2357-2434 | [ademCs-C16][mA][fG][mG][fU][mG][mG] | 1682 |
| 2357 | sense | (Hs-Mf) | [fA][mA][fG][mU][fA][fA][mA][fA][mU][fC] | |
| | strand | | [mUs][mGs][mA] | |
| Forward | 3′ assay | | GAAGATTGGGTCCCTGGA | 1683 |
| Primer | | | | |
| Reverse | 3′ assay | | TGTCTTGGCTTTGGCGTT | 1684 |
| Primer | | | | |
| Probe | 3′ assay | | 6FAM- CGG AAG GTC/ZEN/AGC TTG TGG | 1685 |
| | | | GTT TCA | |
| Forward | 5′ assay | | CACCACAGCCACCTTCTC | 1686 |
| Primer | | | | |
| Reverse | 5′ assay | | CTTCCATCACTTCGAACTCCT | 1687 |
| Primer | | | | |
| Probe | 5′ assay | | CGT CCT CGC/ZEN/CTC TGT CGA CTA | 1688 |

1-117. (canceled)

118. An RNAi oligonucleotide for reducing microtubule-associated protein tau (MAPT) gene expression, the oligonucleotide comprising a sense strand and an antisense strand, wherein the sense strand and the antisense strand form a duplex region, wherein the sense strand is:
5′-[mCs][mA][fG][mG][fU][mG][mG][fA][mA][fG][mU][fA][fA][mA][fA][mU][fC][mU][mG][mA][mG][mC][mA][mG][mC][mC][mG][ademA-GalNAc][ademA-GalNAc][ademA-GalNAc][mG][mG][mC][mU][mG][mC]-3′ (SEQ ID NO: 850), and the antisense strand is 5′-[MePhosphonate-40-mUs][fCs][fA][fG][fA][mU][fU][mU][mU][fA][mC][mU][mU][fC][mC][fA][mC][mC][fU][mGs][mGs][mG]-3′ (SEQ ID NO: 885), wherein mC, mA, mG, and mU=2′-OMe ribonucleosides; fA, fC, fG, and fU=2′-F ribonucleosides; s=phosphorothioate; MePhosphonate-40-mUs= 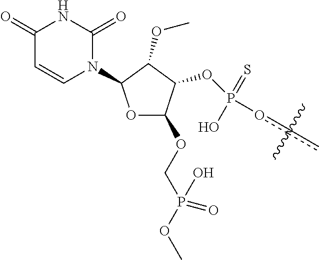
and ademA-GalNAc=

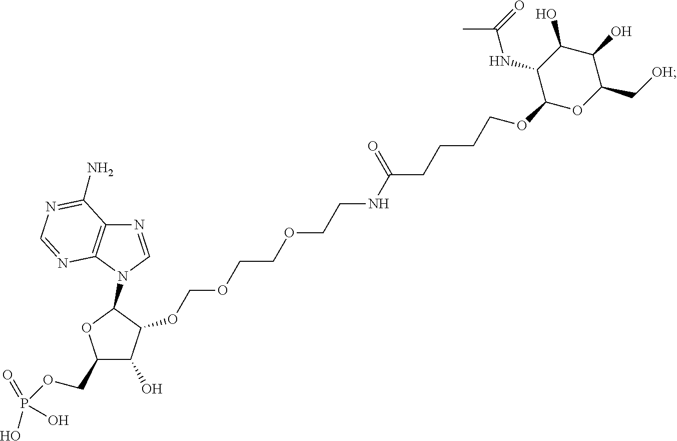 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

119. A pharmaceutical composition comprising:
(i) the RNAi oligonucleotide of claim 118; and
(ii) a pharmaceutically acceptable carrier, delivery agent or excipient.

120. The pharmaceutical composition of claim 119, wherein the carrier comprises phosphate buffered saline.

121. The pharmaceutical composition of claim 119, wherein the carrier comprises water.

122. A method of treating a subject having a disease, disorder, or condition associated with MAPT gene expression, comprising administering to the subject a therapeutically effective amount of the RNAi oligonucleotide of claim 118, or a pharmaceutical composition thereof.

123. The method of claim 122, wherein the disease, disorder, or condition associated with MAPT gene expression is Alzheimer's disease (AD), frontotemporal dementia, progressive supranuclear palsy (PSP), Tau protein associated diseases, primary age-related tauopathy, chronic traumatic encephalopathy, corticobasal degeneration, lytico-bodig disease, ganglioglioma, meningioangiomatosis, Parkinson's Disease (PD), postencephalitic parkinsonism, or subacute sclerosing panencephalitis.

124. The method of claim 122, wherein the RNAi oligonucleotide, or a pharmaceutical composition thereof, is administered subcutaneously.

125. The method of claim 122, wherein the RNAi oligonucleotide, or a pharmaceutical composition thereof, is administered in combination with a second composition or therapeutic agent.

126. A method for reducing MAPT gene expression in a subject, the method comprising administering to the subject the RNAi oligonucleotide of claim 118, or a pharmaceutical composition thereof.

127. The method of claim 126, wherein reducing MAPT gene expression comprises reducing an amount or level of MAPT mRNA, an amount or level of Tau protein, or both.

128. The method of claim 126, wherein the subject has a disease, disorder, or condition associated with MAPT gene expression.

129. The method of claim 128, wherein the disease, disorder or condition associated with MAPT gene expression is Alzheimer's disease (AD), frontotemporal dementia, progressive supranuclear palsy (PSP), Tau protein associated diseases, primary age-related tauopathy, chronic traumatic encephalopathy, corticobasal degeneration, lytico-bodig disease, ganglioglioma, meningioangiomatosis, Parkinson's Disease (PD), postencephalitic parkinsonism, or subacute sclerosing panencephalitis.

130. The method of claim 126, wherein MAPT is reduced in tissue of one or more regions of the central nervous system (CNS), wherein the tissue is associated with AD.

131. The method of claim 130, wherein the tissue associated with AD is selected from prefrontal cortex, motor cortex, temporal cortex, parietal cortex, and hippocampus.

132. The method of claim 126, wherein MAPT is reduced in tissue of one or more regions of the central nervous system (CNS), wherein the tissue is associated with PSP.

133. The method of claim 132, wherein the tissue associated with PSP is selected from caudate nucleus, globus pallidus, thalamus, midbrain tegmentum, substantia nigra, pons, cerebellar white matter, cerebellar dentate nucleus, medulla, cervical spinal cord, thoracic spinal cord, and lumbar spinal cord.

134. The method of claim 126, wherein MAPT is reduced in one or more regions of the CNS selected from cervical spinal cord, thoracic spinal cord, lumbar spinal cord, frontal cortex, temporal cortex, cerebellum, midbrain, occipital cortex, parietal cortex, hippocampus, caudate nucleus, thalamus, brainstem, motor cortex, globus pallidus, midbrain tegmentum, substantia nigra, pons, cerebellar white matter, and cerebellar dentate nucleus.

135. The method of claim 126, wherein the RNAi oligonucleotide, or a pharmaceutical composition thereof, is administered in combination with a second composition or therapeutic agent.

136. A method for reducing MAPT gene expression in a cell or a population of cells, the method comprising contacting the cell or the population of cells with the RNAi oligonucleotide of claim 118, or a pharmaceutical composition thereof.
